(19) United States
(12) Patent Application Publication
(10) Pub. No.: US 2023/0348528 A1
(43) Pub. Date: Nov. 2, 2023

(54) METHODS FOR PRODUCING AND/OR ENRICHING RECOMBINANT ANTIGEN-BINDING MOLECULES

(71) Applicant: Chugai Seiyaku Kabushiki Kaisha, Tokyo (JP)

(72) Inventor: Tatsuya KAWA, Shizuoka (JP)

(21) Appl. No.: 17/797,540

(22) PCT Filed: Feb. 5, 2021
(86) PCT No.: PCT/JP2021/004206

§ 371 Date: Aug. 4, 2022

(30) Foreign Application Priority Data
Feb. 5, 2020 (JP) 2020-017755

(51) Int. Cl. C07K 1/107

(52) U.S. Cl. CPC C07K 1/107; C07K 2317/624; C07K 2317/522

(57) ABSTRACT

An objective of the present invention is to provide novel antigen-binding molecules that have activity of regulating, e.g., interaction between antigen molecules. The present invention relates to antigen-binding molecules containing a first antigen-binding domain and a second antigen-binding domain which are capable of being linked with each other via at least one disulfide bond formed between the two antigen-binding domains, and methods for producing such antigen-binding molecules. More particularly, the invention relates to methods for increasing or enriching a preferred form of antibody proteins, and methods for eliminating disulfide heterogeneity of recombinant antibody proteins.

### TECHNICAL FIELD

The present invention relates to antigen-binding molecules containing a first antigen-binding domain and a second antigen-binding domain which are capable of being linked with each other via at least one disulfide bond formed between the two antigen-binding domains, and methods for producing such antigen-binding molecules. More particularly, the invention relates to methods for increasing or enriching a preferred form of antibody proteins, and methods for eliminating disulfide heterogeneity of recombinant antibody proteins.

### BACKGROUND ART

Antibodies are proteins which specifically bind to an antigen with high affinity. It is known that various molecules ranging from low-molecular compounds to proteins can be antigens. Since the technique for producing monoclonal antibodies was developed, antibody modification techniques have advanced, making it easy to obtain antibodies that recognize a particular molecule. Now the antibody modification techniques are not only for modifying proteins themselves, but have also expanded into a field that aims at addition of new functions where conjugation with low molecular compounds is contemplated. For example, cysteine-engineered antibodies, which contain a free cysteine amino acid in the heavy chain or light chain, are used as antibody-drug conjugates (ADCs) for medical purposes (PTL 1).

Antibodies are drawing attention as pharmaceuticals because they are highly stable in blood plasma and have less side effects. Not only do antibodies bind to an antigen and exhibit agonistic or antagonistic effects, but they also induce cytotoxic activity mediated by effector cells (also referred to as effector functions) including ADCC (Antibody Dependent Cell Cytotoxicity), ADCP (Antibody Dependent Cell Phagocytosis), and CDC (Complement Dependent Cytotoxicity). Taking advantage of these antibody functions, pharmaceuticals for cancer, immune diseases, chronic disease, infections, etc. have been developed (NPL 1).

For example, pharmaceuticals utilizing an agonist antibody against a costimulatory molecule promoting activation of cytotoxic T cells have been developed as anti-cancer agents (NPL 2). Recently, immune checkpoint-inhibiting antibodies with antagonist activity on co-inhibitory molecules were found to be useful as anticancer agents. This finding led to the launch of a series of antibody pharmaceuticals inhibiting the interaction of CTLA4/CD80 or PD-1/PD-L1: Ipilimumab, Nivolumab, Pembrolizumab, and Atezolizumab (NPL 1).

However, such antibodies sometimes do not sufficiently exert expected effects in their original native IgG form. Therefore, second generation antibody pharmaceuticals, in which the functions of the native IgG antibody have been artificially enhanced or added, or diminished or deleted, depending on the purpose of use, have been developed. The second generation antibody pharmaceuticals include, for example, antibodies with enhanced or deleted effector functions (NPL 3), antibodies binding to an antigen in an pH-dependent manner (NPL 4), and antibodies binding to two or more different antigens per molecule (antibodies binding to two different antigens are generally referred to as “bispecific antibodies”) (NPL 5).

Bispecific antibodies are expected to be more effective pharmaceuticals. For example, antibodies with enhanced antitumor activity which crosslink a cytotoxic T cell with a cancer cell by binding to a protein expressed on the cell membrane of the T cell as one antigen and to a cancer antigen as the other antigen have been developed (NPL 7, NPL 8, and PTL 2). The previously reported bispecific antibodies include molecules with two antibody Fab domains each having a different sequence (common light chain bispecific antibodies and hybrid hybridomas), molecules with an additional antigen-binding site attached to the N or C terminus of antibody (DVD-Ig and scFv-IgG), molecules with one Fab domain binding to two antigens (Two-in-one IgG), molecules in which the loop regions of the CH3 domain have been engineered to form new antigen-binding sites (Fcab) (NPL 9), and molecules with tandem Fab-Fab (NPL 10).

Meanwhile, antibodies with effector functions readily cause side effects by acting even on normal cells that express a target antigen at low levels. Thus, efforts have been made to allow antibody pharmaceuticals to exert their effector functions specifically on target tissue. Previously reported examples are antibodies whose binding activity changes upon binding to a cell metabolite (PTL 3), antibodies which become capable of binding to an antigen upon protease cleavage (PTL 4), and a technology that regulates antibody-mediated crosslinking between chimeric antigen receptor T cells and cancer cells by addition of a compound (ABT-737) (NPL 11).

Agonist antibodies may be difficult to obtain depending on the target. In particular, for membrane proteins such as G-protein-coupled receptors, many different techniques have been developed (NPL 12). Thus, there is a demand for simple methods for enhancing the agonistic effect of antibodies on such targets. Known existing methods include, for example, a method of crosslinking an anti-DR4 (Death Receptor 4) or anti-DR5 (Death Receptor 5) antibody (NPL13), a method of multimerizing nanobodies of anti-DR5 (Death Receptor 5) antibody (NPL 14), a method of converting an anti-thrombopoietin receptor antibody into a covalent diabody, sc(Fv)₂ (NPL 15), a method of changing the IgG subclass of anti-CD40 antibody (NPL 16), a method of hexamerizing an anti-CD20 antibody (NPL 17), and a method of producing a circular, antibody-like molecule (PTL 5). In addition, reported methods using bispecific antibodies include, for example, a method of using a combination of two appropriate anti-erythropoietin antibodies against different epitopes as a bispecific antibody (NPL 18), a method of using a combination of an antibody for guide functions and an antibody for effector functions as a bispecific antibody (NPL 19), and a method of introducing Cys residues into multiple antibody fragments specific for different epitopes and conjugating them (NPL 20, NPL 21, and PTL 6).

### CITATION LIST

### Patent Literature

[PTL 1] WO 2016/040856
[PTL 2] WO 2008/157379
[PTL 3] WO 2013/180200
[PTL 4] WO 2009/025846
[PTL 5] WO 2017/191101
[PTL 6] WO 2018/027204

### Non Patent Literature

[NPL 1] Nature Reviews Drug Discovery (2018) 17, 197-223
[NPL 2] Clinical and Experimental Immunology (2009) 157, 9-19
[NPL 3] Current Pharmaceutical Biotechnology (2016) 17, 1298-1314
[NPL 4] Nature Biotechnology (2010) 28, 1203-1208
[NPL 5] MAbs (2012) 4, 182-197
[NPL 6] Nature Reviews Immunology (2010) 10, 301-316
[NPL 7] Sci Transl Med (2017) 9(410), eaa14291
[NPL 8] Blood (2011) 117(17): 4403-4404
[NPL 9] Protein Eng Des Sel (2010) 23(4), 289-297
[NPL 10] J Immunol (2016) 196(7): 3199-3211
[NPL 11] Nature Chemical Biology (2018) 14, 112-117
[NPL 12] Exp Mol Med (2016) 48(2): e207
[NPL 13] Nature Reviews Drug Discovery (2008) 7, 1001-1012
[NPL 14] MAbs (2014) 6(6): 1560-1570
[NPL 15] Blood (2005) 105(2): 562-566
[NPL 16] J Biol Chem (2008) 283(23): 16206-16215
[NPL 17] PLoS Biol (2016) 14(1): e1002344
[NPL 18] Proc Natl Acad Sci USA (2012) 109(39): 15728-15733
[NPL 19] Scientific Reports (2018) 8, Article number: 766
[NPL 20] PLoS One (2012) 7(12): e51817
[NPL 21] Nucleic Acids Res (2010) 38(22): 8188-8195

### SUMMARY OF INVENTION

### Technical Problem

An objective of the present invention is to provide novel antigen-binding molecules (for example, an IgG antibody) that have activity of regulating interaction between two or more antigen molecules, and/or methods for producing or using such antigen-binding molecules. More particularly, the present invention solves the issues that conventional antibody (e.g. wild type IgG) has uncontrolled flexibility of the two antigen-binding domains (e.g. two Fab arms) by means of introducing one or more engineered disulfide bond(s) between the two antigen-binding domains (two Fabs) of the antibody through introducing mutation in the heavy and/or light chain. Specifically, by introducing one or more thiol-containing amino acid (e.g. cysteine and methionine) at each of the two antigen-binding domains (two Fabs) of the antibody, such antibody is capable of forming one or more disulfide bond between the two antigen-binding domains (two Fabs).

### Solution to Problem

An antigen-binding molecule of the present invention contains a first antigen-binding domain and a second antigen-binding domain which are “capable of being linked” with each other via at least one disulfide bond between the two antigen-binding domains. The at least one disulfide bond is “capable of being formed” between the two antigen-binding domains, e.g., between amino acid residues which are not in a hinge region. The terms “capable of being linked” and “capable of being formed” include cases where the disulfide bond has already been formed, and cases where the disulfide bond has not been formed but will be formed later under suitable conditions.

In one non-limiting aspect, the one or more engineered disulfide bond(s) between the two Fabs of the IgG antibody enables controls of the flexibility, the distance, and/or the cell binding orientation (i.e. cis or trans) of the two Fab arms, thereby improving activity, and/or safety of the IgG antibody compared to corresponding wild type IgG antibody without the one or more engineered disulfide bond(s). In one non-limiting aspect, the one or more engineered disulfide bond(s) between the two Fabs of the IgG improves the agonistic activity of the IgG antibody compared to corresponding wild type IgG antibody without the one or more engineered disulfide bond(s). In addition, in another non-limiting aspect, the one or more engineered disulfide bond(s) between the two Fabs of the IgG improves the resistance of the IgG antibody to protease digestion, compared to corresponding wild type IgG antibody without the one or more engineered disulfide bond(s).

While preparing the antibody capable of forming one or more engineered disulfide bond(s) between the two Fabs of the antibody, the inventors further found the several conformational isoforms of the same antibody (same sequence) but with different disulfide structures, in particular the isoform having the “paired cysteines” and the isoform having the “free or unpaired cysteines” (i.e., two structural isoforms), can be generated during recombinant antibody production in mammalian cell. Therefore, another aspect of the present invention is directed to providing efficient and facile production, purification and analysis of the antibody having one or more engineered disulfide bond(s) between the two Fabs of the antibody. More particularly, the invention describes methods for increasing structural homogeneity and relative abundance of the antibody in the “paired cysteines” form, i.e. having one or more engineered disulfide bond(s) formed between the two Fabs of the antibody. In other words, the invention describes methods for decreasing relative abundance of the antibody in the “free or unpaired cysteines” form, i.e. having no engineered disulfide bond formed between the two Fabs of the antibody.

As described in further detail hereinbelow, in some embodiments of the invention, the addition of reducing agent can facilitate the formation of one or more engineered disulfide bond(s) in the antibody and thus produce structurally homogeneous of the molecule.

More specifically, the present invention provides the following:
[1] A method for (i) producing an antibody preparation, (ii) purifying an antibody having a desired conformation, or (iii) improving homogeneity of an antibody preparation;
   said method comprising contacting an antibody preparation with a reducing reagent, wherein the antibody comprises a first antigen-binding domain and a second antigen-binding domain which are capable of being linked with each other via at least one disulfide bond, wherein said at least one disulfide bond is capable of being formed between amino acid residues which are not in a hinge region.
[2] A method for (i) producing an antibody preparation, (ii) purifying an antibody preparation, or (iii) improving homogeneity of an antibody preparation; comprising isolating a fraction of the antibody having a desired conformation via one or more chromatography steps selected from the group consisting of: reversed-phase chromatography, size-exclusion chromatography, ion-exchange chromatography, hydrophobic interaction chromatography, affinity chromatography, and electrophoresis; wherein said antibody having a desired conformation is characterized by having at least one disulfide bond formed between amino acid residues which are not in a hinge region.
[2A] The method of [2], wherein one or more chromatography steps is ion exchange chromatography (IEC) and/or hydrophobic interaction chromatography (HIC), or mixed-mode chromatography of IEC and HIC.
[3] The method of any one of [1]-[2A], wherein said antibody preparation comprises two or more structural isoforms which differ by at least one disulfide bond formed between amino acid residues which are not in a hinge region.
[3A] The method of [3], wherein said antibody preparation comprises two structural isoforms which differ by at least one disulfide bond formed between amino acid residues which are not in a hinge region.
[3B] The method of any one of [1] to [3A], wherein said method preferentially enriches or increases the population of an antibody structural isoform having at least one disulfide bond formed between amino acid residues which is not in a hinge region.
[3C] The method of any one of [1] to [3B], wherein said method produces a homogenous antibody preparation having at least 50%, 60%, 70%, 80%, 90%, preferably at least 95% molar ratio of said antibody having at least one disulfide bond formed between amino acid residues which are not in a hinge region.
[3D] The method of any one of [1] to [3C], wherein each of said first antigen-binding domain and second antigen-binding domain comprises a hinge region, or does not comprise a hinge region.
[3E] The method of any of [1] to [3D], wherein said amino acid residues which are not in a hinge region are introduced or engineered cysteines.
[3F] The method of any of [1] to [3E], wherein said at least one disulfide bond is an interchain disulfide bond.
[3I] The method of any of [1] to [3F], wherein said at least one disulfide bond is an engineered disulfide bond which is not present in a wild type IgG.
[4] The method of any of [1] to [3J],
   wherein said at least one disulfide bond is formed between a CH1 region, a CL region, a VL region, a VH region and/or a VHH region of the first antigen-binding domain and the second antigen-binding domain.
[5] The method of any of [1] to [4], wherein said at least one disulfide bond is formed between a CH1 region of the first antigen-binding domain and a CH1 region of the second antigen-binding domain.
[5.1] The method of [5], wherein said at least one disulfide bond is formed between the antigen-binding domains at any one of positions 119 to 123, 131 to 140, 148 to 150, 155 to 167, 174 to 178, 188 to 197, and 201 to 214, according to EU numbering, in the CH1 region.
[5.2] The method of [5], wherein said at least one disulfide bond is formed between the antigen-binding domains at any one of positions 119, 122, 123, 131, 132, 133, 134, 135, 136, 137, 138, 139, 140, 148, 150, 155, 156, 157, 159, 160, 161, 162, 163, 164, 165, 167, 174, 176, 177, 178, 188, 189, 190, 191, 192, 193, 194, 195, 196, 197, 201, 203, 205, 206, 207, 208, 211, 212, 213, 214, according to EU numbering, in the CH1 region.
[5.3] The method of [5], wherein said at least one disulfide bond is formed between the antigen-binding domains at any one of positions 134, 135, 136, 137, 191, 192, 193, 194, 195, or 196, according to EU numbering, in the CH1 region.
[5.4] The method of [5], wherein said at least one disulfide bond is formed between the antigen-binding domains at any one of positions 135, 136, or 191, according to EU numbering, in the CH1 region.
[5.5] The method of [5], wherein said at least one disulfide bond is formed between the amino acid residues in the first antigen-binding domain and the second antigen-binding domain, selected from the group consisting of positions 119, 120, 121, 122, and 123 according to EU numbering.
[5.6] The method of [5], wherein said at least one disulfide bond is formed between the amino acid residues in the first antigen-binding domain and the second antigen-binding domain, selected from the group consisting of positions 131, 132, 133, 134, 135, 136, 137, 138, 139, and 140 according to EU numbering.
[5.7] The method of [5], wherein said at least one disulfide bond is formed between the amino acid residues in the first antigen-binding domain and the second antigen-binding domain, selected from the group consisting of positions 148, 149, and 150 according to EU numbering.
[5.8] The method of [5], wherein said at least one disulfide bond is formed between the amino acid residues in the first antigen-binding domain and the second antigen-binding domain, selected from the group consisting of positions 155, 156, 157, 158, 159, 160, 161, 162, 163, 164, 165, 166, and 167 according to EU numbering.
[5.9] The method of [5], wherein said at least one disulfide bond is formed between the amino acid residues in the first antigen-binding domain and the second antigen-binding domain, selected from the group consisting of positions 174, 175, 176, 177, and 178 according to EU numbering.
[5.10] The method of [5], wherein said at least one disulfide bond is formed between the amino acid residues in the first antigen-binding domain and the second antigen-binding domain, selected from the group consisting of positions 188, 189, 190, 191, 192, 193, 194, 195, 196, and 197 according to EU numbering.
[5.11] The method of [5], wherein said at least one disulfide bond is formed between the amino acid residues in the first antigen-binding domain and the second antigen-binding domain, selected from the group consisting of positions 201, 202, 203, 204, 205, 206, 207, 208, 209, 210, 211, 212, 213, and 214 according to EU numbering.
[5.12] The method of [5], wherein the difference between the positions of the amino acid residues in the first antigen-binding domain and the second antigen-binding domain is three amino acids or less.
[5.13] The method of [5], wherein said at least one of the disulfide bonds linking the two antigen-binding domains is formed by linking an amino acid residue at position 135 according to EU numbering in the CH1 region of the first antigen-binding domain with an amino acid residue at any one of positions 132 to 138 according to EU numbering in the CH1 region of the second antigen-binding domain.
[5.14] The method of [5], wherein said at least one of the disulfide bonds linking the two antigen-binding domains is formed by linking an amino acid residue at position 136 according to EU numbering in the CH1 region of the first antigen-binding domain with an amino acid residue at any one of positions 133 to 139 according to EU numbering in the CH1 region of the second antigen-binding domain.
[5.15] The method of [5], wherein said at least one of the disulfide bonds linking the two antigen-binding domains is formed by linking an amino acid residue at position 191 according to EU numbering in the CH1 region of the first antigen-binding domain with an amino acid residue at any one of positions 188 to 194 according to EU numbering in the CH1 region of the second antigen-binding domain.
[5.16] The method of [5], wherein one disulfide bond is formed between the two antigen-binding domains at position 135, according to EU numbering, in the CH1 region.
[5.17] The method of [5], wherein one disulfide bond is formed between the two antigen-binding domains at position 136, according to EU numbering, in the CH1 region.
[5.18] The method of [5], wherein one disulfide bond is formed between the two antigen-binding domains at position 191, according to EU numbering, in the CH1 region.
[5A] The method of [5], wherein the subclass of the CH1 region is gamma 1, gamma 2, gamma 3, gamma 4, alpha 1, alpha 2, mu, delta, or epsilon.
[6] The method of [5]-[5A], wherein one disulfide bond is formed between the amino acid residues at position 191 according to EU numbering in the respective CH1 regions of the first antigen-binding domain and the second antigen-binding domain.
[6A] The method of [6], wherein additional one, two or more disulfide bond(s) is/are formed between the first antigen-binding domain and the second antigen-binding domain via the amino acid residues at the following positions according to EU numbering in each of the respective CH1 regions of the first antigen-binding domain and the second antigen-binding domain:
   (a) between amino acid residues at any position of 131 to 138, 194 and 195 in each of the two antigen-binding domains;
   (b) between the amino acid residues at position 131 in each of the two antigen-binding domains, and between the amino acid residues at position 194 in each of the two antigen-binding domains;
   (c) between the amino acid residues at position 132 in each of the two antigen-binding domains, and between the amino acid residues at position 194 in each of the two antigen-binding domains;
   (d) between the amino acid residues at position 133 in each of the two antigen-binding domains, and between the amino acid residues at position 194 in each of the two antigen-binding domains;
   (e) between the amino acid residues at position 134 in each of the two antigen-binding domains, and between the amino acid residues at position 194 in each of the two antigen-binding domains;
   (f) between the amino acid residues at position 135 in each of the two antigen-binding domains, and between the amino acid residues at position 194 in each of the two antigen-binding domains;
   (g) between the amino acid residues at position 136 in each of the two antigen-binding domains, and between the amino acid residues at position 194 in each of the two antigen-binding domains;
   (h) between the amino acid residues at position 137 in each of the two antigen-binding domains, and between the amino acid residues at position 194 in each of the two antigen-binding domains;
   (i) between the amino acid residues at position 138 in each of the two antigen-binding domains, and between the amino acid residues at position 194 in each of the two antigen-binding domains;
   (j) between the amino acid residues at position 131 in each of the two antigen-binding domains, and between the amino acid residues at position 195 in each of the two antigen-binding domains;
   (k) between the amino acid residues at position 132 in each of the two antigen-binding domains, and between the amino acid residues at position 195 in each of the two antigen-binding domains;
   (l) between the amino acid residues at position 133 in each of the two antigen-binding domains, and between the amino acid residues at position 195 in each of the two antigen-binding domains;
   (m) between the amino acid residues at position 134 in each of the two antigen-binding domains, and between the amino acid residues at position 195 in each of the two antigen-binding domains;
   (n) between the amino acid residues at position 135 in each of the two antigen-binding domains, and between the amino acid residues at position 195 in each of the two antigen-binding domains;
   (o) between the amino acid residues at position 136 in each of the two antigen-binding domains, and between the amino acid residues at position 195 in each of the two antigen-binding domains;
   (p) between the amino acid residues at position 137 in each of the two antigen-binding domains, and between the amino acid residues at position 195 in each of the two antigen-binding domains; and
   (q) between the amino acid residues at position 138 in each of the two antigen-binding domains, and between the amino acid residues at position 195 in each of the two antigen-binding domains.
[6B] The method of [6] or [6A], wherein any one of the first and second antigen-binding domains comprises one, two or more charged amino acid residues at position 136-138 (according to EU numbering) in the respective CH1 region; and the other antigen-binding domain of the first and second antigen-binding domains comprises one, two or more oppositely charged amino acid residues at position 193-195 (according to EU numbering) in the respective CH1 region.
[6C] The method of [6] or [6A], wherein any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more negatively charged amino acid residues at position 193-195 (according to EU numbering) in the respective CH1 region.
[6D] The method of [6] or [6A], wherein any one of the first and second antigen-binding domains comprises one, two or more negatively charged amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region.
[6E] The method of [6] or [6A], wherein any one of the first and second antigen-binding domains comprises one, two or more of the following amino acid residues in the respective CH1 region (according to EU numbering):
   (a) the amino acid residue at position 136 is glutamic acid (E) or aspartic acid (D);
   (b) the amino acid residue at position 137 is glutamic acid (E) or aspartic acid (D);
   (c) the amino acid residue at position 138 is glutamic acid (E) or aspartic acid (D); and the other antigen-binding domain of the first and second antigen-binding domains comprises one, two or more of the following amino acid residues in the respective CH1 region (according to EU numbering):
   (d) the amino acid residue at position 193 is lysine (K), arginine (R), or histidine (H);
   (e) the amino acid residue at position 194 is lysine (K), arginine (R), or histidine (H); and
   (f) the amino acid residue at position 195 is lysine (K), arginine (R), or histidine (H).
[6F-1] The method of [6] or [6A], wherein any one of the first and second antigen-binding domains comprises one or more of the following amino acid residues in the respective CH1 region (according to EU numbering):
   (a) the amino acid residue at position 136 is lysine (K), arginine (R), or histidine (H);
   (b) the amino acid residue at position 137 is lysine (K), arginine (R), or histidine (H);
   (c) the amino acid residue at position 138 is lysine (K), arginine (R), or histidine (H); and the other antigen-binding domain out of the first and second antigen-binding domains comprises one or more of the following amino acid residues in the respective CH1 region (according to EU numbering):
   (d) the amino acid residue at position 193 is glutamic acid (E) or aspartic acid (D);
   (e) the amino acid residue at position 194 is glutamic acid (E) or aspartic acid (D); and
   (f) the amino acid residue at position 195 is glutamic acid (E) or aspartic acid (D).
[6F-2] The method of [6] or [6A], wherein each of the first and second antigen-binding domains comprises any of the specific charged mutation combination in the respective CH1 region (according to EU numbering) as listed in Tables 7, Table 82 or Table 85.
[6G] The method of [6] or [6A], wherein any one of the first and second antigen-binding domains comprises one, two or more hydrophobic amino acid residues at position 136-138 (according to EU numbering) in the respective CH1 region; and the other antigen-binding domain out of the first and second antigen-binding domains comprises one, two or more hydrophobic amino acid residues at position 193-195 (according to EU numbering) in the respective CH1 region.
[6H] The method of [6G], wherein said hydrophobic amino acid residue(s) is/are alanine (Ala), valine (Val), leucine (Leu), isoleucine (Ile), phenylalanine (Phe), and/or tryptophan (Trp).
[6I] The method of [6] or [6A], wherein any one of the first and second antigen-binding domains comprises one “knob” amino acid residues at position 136-138 (according to EU numbering) in the respective CH1 region; and the other antigen-binding domain out of the first and second antigen-binding domains comprises one, two or more “hole” amino acid residues at position 193-195 (according to EU numbering) in the respective CH1 region.
[6J] The method of [6] or [6A], wherein any one of the first and second antigen-binding domains comprises one, two or more “hole” amino acid residues at position 136-138 (according to EU numbering) in the respective CH1 region; and the other antigen-binding domain out of the first and second antigen-binding domains comprises one “knob” amino acid residues at position 193-195 (according to EU numbering) in the respective CH1 region.
[6K] The method of [6I] or [6J], wherein said “knob” amino acid residue(s) is/are selected from the group consisting of tryptophan (Trp) and phenylalanine (Phe); and said “hole” amino acid residue(s) is/are selected from the group consisting of alanine (Ala), valine (Val), threonine (T) or serine (S).
[6L] The method of [6] or [6A], wherein any one of the first and second antigen-binding domains comprises one, two or more aromatic amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region.
[6M] The method of [6] or [6A], wherein any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more aromatic amino acid residues at position 193-195 (according to EU numbering) in the respective CH1 region.
[6N-1] The method of [6L] or [6M], wherein said aromatic amino acid residue(s) is/are selected from the group consisting of tryptophan (Trp), tyrosine (Tyr), histidine (His), and phenylalanine (Phe); and said positively charged amino acid residue(s) is/are selected from the group consisting of lysine (K), arginine (R), or histidine (H).
[6N-2] The method of [6] or [6A], wherein each of the first and second antigen-binding domains comprises any of the specific hydrophobic amino acid mutation combination in the respective CH1 region (according to EU numbering) as listed in Table 10.
[7] The method of any one of [1] to [4], wherein said at least one disulfide bond is formed between a CL region of the first antigen-binding domain and a CL region of the second antigen-binding domain.
[7.1] The method of [7], wherein the amino acid residue from which the at least one disulfide bond between the two antigen-binding domains is formed is present at any one of positions 108 to 112, 121 to 128, 151 to 156, 184 to 190, 195 to 196, 200 to 203, and 208 to 213, according to Kabat numbering, in the CL region.
[7.2] The method of [7], wherein the amino acid residue from which the at least one disulfide bond between the two antigen-binding domains is formed is present at a position selected from the group consisting of positions 108, 109, 112, 121, 123, 126, 128, 151, 152, 153, 156, 184, 186, 188, 189, 190, 195, 196, 200, 201, 202, 203, 208, 210, 211, 212, and 213 according to Kabat numbering in the CL region.
[7.3] The method of [7], wherein the amino acid residue from which the at least one disulfide bonds between the two antigen-binding domains is formed is present at position 126 according to Kabat numbering in the CL region.
[7.4] The method of [7], wherein at least one of the disulfide bonds linking the two antigen-binding domains is formed by linking an amino acid residue in the CL region of the first antigen-binding domain with an amino acid residue in the CL region of the second antigen-binding domain.
[7.5] The method of [7], wherein the amino acid residue from which the at least one disulfide bond between the two antigen-binding domains is formed is present at a position independently selected from the group consisting of positions 108, 109, 110, 111, and 112 according to Kabat numbering.
[7.6] The method of [7], wherein the amino acid residue from which the at least one disulfide bond between the two antigen-binding domains is formed is present at a position independently selected from the group consisting of positions 151, 152, 153, 154, 155, and 156 according to Kabat numbering.
[7.7] The method of [7], wherein the amino acid residue from which the at least one disulfide bond between the two antigen-binding domains is formed is present at a position independently selected from the group consisting of positions 184, 185, 186, 187, 188, 189, and 190 according to Kabat numbering.
[7.8] The method of [7], wherein the amino acid residue from which the at least one disulfide bond between the two antigen-binding domains is formed is present at a position independently selected from the group consisting of positions 200, 201, 202, and 203 according to Kabat numbering.
[7.9] The method of [7], wherein the amino acid residue from which the at least one disulfide bond between the two antigen-binding domains is formed is present at a position independently selected from the group consisting of positions 208, 209, 210, 211, 212, and 213 according to Kabat numbering.
[7.10] The method of [7] to [7.9], wherein the difference between the positions of the amino acid residues from which the at least one disulfide bond between the two antigen-binding domains is formed is three amino acids or less.
[7.11] The method of [7], wherein said at least one of the bonds linking the two antigen-binding domains is formed by linking amino acid residues at position 126 according to Kabat numbering in the CL region of the two antigen-binding domains with each other.
[8] The method of any one of [1] to [4], wherein said at least one of the disulfide bond is formed by linking an amino acid residue in a CH1 region of the first antigen-binding domain with an amino acid residue in a CL region of the second antigen-binding domain.
[8.1] The method of [8], wherein the amino acid residue in the CH1 region is selected from the group consisting of positions 188, 189, 190, 191, 192, 193, 194, 195, 196, and 197 according to EU numbering, and the amino acid residue in the CL region is selected from the group consisting of positions 121, 122, 123, 124, 125, 126, 127, and 128 according to Kabat numbering.
[8.2] The method of [8], wherein at least one of the disulfide bond linking the two antigen-binding domains is formed by linking an amino acid residue at position 191 according to EU numbering in the CH1 region of the first antigen-binding domain with an amino acid residue at position 126 according to Kabat numbering in the CL region of the second antigen-binding domain.
[8A] The method of [7] to [8], wherein the subclass of the CL region is kappa or lambda.
[9] The method of any one of [1] to [4], wherein said at least one disulfide bond is formed between a variable region of the first antigen-binding domain and the second antigen-binding domain.
[9.1] The method of [9], wherein the amino acid residue from which the at least one disulfide bond between the antigen-binding domains is formed is present within a VH region.
[9.2] The method of [9], wherein the amino acid residue from which the at least one disulfide bond between the antigen-binding domains is formed is present at a position selected from the group consisting of positions 6, 8, 16, 20, 25, 26, 28, 74, and 82b according to Kabat numbering in the VH region.
[9.3] The method of [9], wherein the amino acid residue from which the at least one disulfide bond between the antigen-binding domains is formed is present within a VL region.
[9.4] The method of [9], wherein the amino acid residue from which the at least one disulfide bond between the antigen-binding domains is formed is present at a position selected from the group consisting of positions 21, 27, 58, 77, 100, 105, and 107 according to Kabat numbering in the VL region (subclass kappa).
[9.5] The method of [9], wherein the amino acid residue from which the at least one disulfide bond between the antigen-binding domains is formed is present at a position selected from the group consisting of positions 6, 19, 33, and 34 according to Kabat numbering in the VL region (subclass lambda).
[9A] The method of [4], wherein the amino acid residue from which the at least one disulfide bond between the two antigen-binding domains is formed is present within a VHH region.
[9B] The method of [9A], wherein the amino acid residue from which the at least one disulfide bond between the antigen-binding domains is formed is present at a position selected from the group consisting of positions 4, 6, 7, 8, 9, 10, 11, 12, 14, 15, 17, 20, 24, 27, 29, 38, 39, 40, 41, 43, 44, 45, 46, 47, 48, 49, 67, 69, 71, 78, 80, 82, 82c, 85, 88, 91, 93, 94, and 107 according to Kabat numbering in the VHH region.
[10] The method of any one of [1] to [9B], characterized by one or more of the following:
   (a) wherein said at least one disulfide bond restricts the antigen binding orientation of the two antigen-binding domains to cis antigen-binding (i.e. binding to two antigens on the same cell), or restrict binding of the two antigen binding domains to two antigens which are spatially close to each other;
   (b) wherein said at least one disulfide bond holds the first antigen-binding domain and the second antigen-binding domain spatially closer to each other, as compared to a same corresponding antibody which does not have said at least one disulfide bond;
   (c) wherein said at least one disulfide bond reduce the flexibility and/or mobility of first antigen-binding domain and the second antigen-binding domain, as compared to a corresponding same antibody which does not have said at least one disulfide bond;
   (d) wherein said at least one disulfide bond increases resistance of the antibody to protease cleavage, as compared to a corresponding same antibody which does not have said at least one disulfide bond;
   (e) wherein said at least one disulfide bond enhances or reduces interaction between two antigen molecules bound by the antigen-binding molecule, as compared to a corresponding same antibody which does not have said at least one disulfide bond;
   (f) wherein said method produces an antibody preparation which is more homogeneous than the same antibody preparation that has not been treated by said method;
   (g) wherein said method produces an antibody preparation having increase in its biological activity compared to the same antibody that has not been treated by said method;
   (h) wherein said method produces an antibody having enhanced activity of holding two antigen molecules at spatially close positions compared to the same antibody that has not been treated by said method;
   (i) wherein said method produces an antibody having enhanced stability compared to the same antibody that has not been treated by said method; and
   (j) wherein said method preferentially enriches an antibody having at least one disulfide bond formed outside of hinge regions and said preferentially enriched form has a pharmaceutically desirable property selected from any of (a) to (i) above, as compared to a preparation that has not been treated by said method.
[11] The method of any one of [1] to [10], wherein each of the first and second antigen-binding domains has a Fab, Fab′, scFab, Fv, scFv, or VHH structure.
[11A] The method of [11], wherein the first and second antigen-binding domains each comprises a Fab and a hinge region, forming a F(ab′)2 structure.
[12] The method of any one of [1] to [11A], wherein the antigen-binding molecule further comprises an Fc region.
[12A] The method of [12], wherein the Fc region is a Fc region having reduced binding activity against Fc gamma R as compared with that of the Fc region of a wild-type human IgG1 antibody.
[13] The method of any one of [1] to [12A], wherein said antibody is an IgG antibody, preferably an IgG1, IgG2, IgG3 or IgG4 antibody.
[14] The method of any one of [1] to [13], wherein both the first and second antigen-binding domains bind to the same antigen.
[14A] The method of any one of [1] to [13], wherein both the first and second antigen-binding domains bind to the same epitope on said antigen.
[14B] The method of any one of [1] to [13], wherein each of the first and second antigen-binding domains binds to a different epitope on said antigen.
[14C] The method of any one of [1] to [13], wherein each of the first and second antigen-binding domains binds to a different antigen.
[14D] The method of any one of [1] to [13], wherein both the first and second antigen-binding domains have the same amino acid sequence.
[14E] The method of any one of [1] to [13], wherein each of the first and second antigen-binding domains has a different amino acid sequence.
[14F] The method of any one of [1] to [14E], wherein at least one of two antigens to which the first and second antigen-binding domains bind is a soluble protein.
[14G] The method of any one of [1] to [14E], wherein at least one of two antigens to which the first and second antigen-binding domains bind is a membrane protein.
[14H] The method of any one of [1] to [14G], which has activity of regulating interaction between two antigen molecules.
[14I] The method of [14H], which is capable of enhancing or diminishing interaction between two antigen molecules as compared to a same corresponding antibody which does not have said at least one disulfide bond.
[14J] The method of any one of [14H] to [14I], wherein the two antigen molecules are a ligand and a receptor thereof, respectively, and wherein the antibody has activity of promoting activation of the receptor by the ligand.
[14K] The method of any one of [14H] to [14I], wherein the two antigen molecules are an enzyme and a substrate thereof, respectively, and wherein the antigen-binding molecule has activity of promoting catalytic reaction of the enzyme with the substrate.
[14L] The method of any one of [14H] to [14I], wherein both of the two antigen molecules are proteins present on cellular surfaces, and wherein the antibody has activity of promoting interaction between a cell expressing the first antigen and a cell expressing the second antigen.
[14M] The method of any of [14L], wherein the cell expressing the first antigen is a cell with cytotoxic activity, and the cell expressing the second antigen is a target cell thereof, and wherein the antibody promotes damage of said target cell by said cell with cytotoxic activity.
[14N] The method of [14M], wherein the cell with cytotoxic activity is a T cell, NK cell, monocyte, or macrophage.
[14O] The method of [14N], wherein the antibody having said at least one disulfide bond enhances or diminishes activation of two antigen molecules as compared to a same corresponding antibody which does not have said at least one disulfide bond.
[14P] The method of any one of [14] to [14O], wherein the antigen molecules are selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.
[15] The method of any one of [14] to [14P], wherein the first antigen-binding domain and the second antigen-binding domain are each capable of binding to CD3.
[16] The method of any one of [1] to [15], wherein the pH of said reducing reagent contacting with the antibody is from about 3 to about 10.
[16A] The method of [16], wherein the pH of said reducing reagent contacting with the antibody is about 6, 7 or 8.
[16B] The method of [16], wherein the pH of said reducing reagent contacting with the antibody is about 7.
[16C] The method of [16], wherein the pH of said reducing reagent contacting with the antibody is about 3.
[17] The method of any one of [1] to [16B], wherein the reducing agent is selected from the group consisting of TCEP, 2-MEA, DTT, Cysteine, GSH and Na2SO3.
[17A] The method of [17], wherein the reducing agent is TCEP.
[18] The method of any one of [17] to [17A], wherein the concentration of the reducing agent is from about 0.01 mM to about 100 mM.
[19] The method of [18], wherein the concentration of the reducing agent is about 0.01, 0.05, 0.1, 0.25, 0.5, 1, 2.5, 5, 10, 25, 50, 100 mM, preferably about 0.01 mM to 25 mM.
[20] The method of any one of [1] to [19], wherein the contacting step is performed for at least 30 minutes.
[20A] The method of any one of [1] to [19], wherein the contacting step is performed for about 2 to about 48 hours.
[20B] The method of any one of [1] to [19], wherein the contacting step is performed for about 2 hours or about 16 hours.
[21] The method of any one of [1] to [20B], wherein the contacting step is performed at a temperature of about 20 degrees C. to 37 degrees C., preferably at 23 degrees C., 25 degrees C. or 37 degrees C., more preferably at 23 degrees C.
[22] The method of any one of [1] to [21], wherein said antibody is at least partially purified prior to said contacting step with reducing agent.
[22A] The method of [22], wherein said antibody is partially purified by affinity chromatography (preferably Protein A chromatography) prior to said contacting.
[23] The method of any one of [1] to [22], wherein the concentration of the antibody is from about 1 mg/ml and about 50 mg/ml.
[23A] The method of [23], wherein the concentration of the antibody is about 1 mg/ml or about 20 mg/ml.
[24] The method of any one of [1] to [23], further comprising isolating a fraction of the contacted antibody having a desired conformation.
[24A] The method of [24], wherein the procedure for said isolating is selected from the group consisting of: reversed-phase chromatography HPLC, size-exclusion chromatography, ion-exchange chromatography, hydrophobic interaction chromatography, affinity chromatography, dialysis and electrophoresis.
[24B] The method of [24], wherein the procedure for said isolating is ion exchange chromatography (IEC) and/or hydrophobic interaction chromatography (HIC).
[24C] The method of any one of [1] to [24B], further comprising a step of removing the reducing agent, preferably by dialysis, more preferably by a chromatography method.
[25] A preparation of an IgG antibody prepared according to the method of any one of [1] to [24B], said preparation having a homogeneous population of said IgG antibody having at least one disulfide bond outside of the hinge regions.
[26] A preparation of an IgG antibody prepared according to the method of any one of [1] to [25], said preparation having at least 50%, 60%, 70%, 80%, 90%, preferably at least 95% molar ratio of said IgG antibody having at least one disulfide bond outside of the hinge regions.
[27] The preparation of [25] or [26], further comprising a pharmaceutically acceptable carrier, excipient or diluent.
[28] A pharmaceutical composition comprising a homogeneous population of antibody as defined in [25] and a pharmaceutically acceptable carrier, excipient or diluent.

In another aspect, the present invention also provides the following:
[1] An antigen-binding molecule comprising a first antigen-binding domain and a second antigen-binding domain, wherein the two antigen-binding domains are linked with each other via one or more bonds.
[2] The antigen-binding molecule of [1], wherein at least one of the bonds linking the two antigen-binding domains is a covalent bond.
[3] The antigen-binding molecule of [2], wherein the covalent bond is formed by direct crosslinking of an amino acid residue in the first antigen-binding domain with an amino acid residue in the second antigen-binding domain.
[4] The antigen-binding molecule of [3], wherein the crosslinked amino acid residues are cysteine.
[5] The antigen-binding molecule of [4], wherein the formed covalent bond is a disulfide bond.
[6] The antigen-binding molecule of [2], wherein the covalent bond is formed by crosslinking of an amino acid residue in the first antigen-binding domain with an amino acid residue in the second antigen-binding domain via a crosslinking agent.
[7] The antigen-binding molecule of [6], wherein the crosslinking agent is an amine-reactive crosslinking agent.
[8] The antigen-binding molecule of [7], wherein the crosslinked amino acid residues are lysine.
[9] The antigen-binding molecule of [1], wherein at least one of the bonds linking the two antigen-binding domains is a noncovalent bond.
[10] The antigen-binding molecule of [9], wherein the noncovalent bond is an ionic bond, hydrogen bond, or hydrophobic bond.
[11] The antigen-binding molecule of [10], wherein the ionic bond is formed between an acidic amino acid and a basic amino acid.
[12] The antigen-binding molecule of [11], wherein the acidic amino acid is aspartic acid (Asp) or glutamic acid (Glu), and the basic amino acid is histidine (His), lysine (Lys), or arginine (Arg).
[13] The antigen-binding molecule of any one of [1] to [12], wherein at least one of amino acid residues from which the bonds between the antigen-binding domains originate is an artificially-introduced mutated amino acid residue.
[14] The antigen-binding molecule of [13], wherein the mutated amino acid residue is a cysteine residue.
[15] The antigen-binding molecule of any one of [1] to [14], wherein at least one of the first and second antigen-binding domains has, by itself, activity of binding to an antigen.
[16] The antigen-binding molecule of any one of [1] to [15], wherein the first and second antigen-binding domains are both antigen-binding domains of the same type.
[17] The antigen-binding molecule of any one of [1] to [16], wherein at least one of the bonds linking the two antigen-binding domains is formed by linking amino acid residues present at the same position on the first antigen-binding domain and the second antigen-binding domain with each other.
[18] The antigen-binding molecule of any one of [1] to [16], wherein at least one of the bonds linking the two antigen-binding domains is formed by linking amino acid residues present at different positions on the first antigen-binding domain and the second antigen-binding domain with each other.
[19] The antigen-binding molecule of any one of [1] to [18], wherein at least one of the first and second antigen-binding domains comprises an antibody fragment which binds to a particular antigen.
[20] The antigen-binding molecule of [19], wherein the antibody fragment is a Fab, Fab′, scFab, Fv, scFv, or single domain antibody.
[21] The antigen-binding molecule of [19] or [20], wherein at least one of amino acid residues from which the bonds between the antigen-binding domains originate is present within the antibody fragment.
[22] The antigen-binding molecule of [21], wherein the amino acid residue from which the bonds between the antigen-binding domains originate is present within a constant region.
[23] The antigen-binding molecule of [22], wherein the constant region is derived from human.
[24] The antigen-binding molecule of [22] or [23], wherein the amino acid residue from which the bonds between the antigen-binding domains originate is present within a CH1 region.
[25] The antigen-binding molecule of [24], wherein the subclass of the CH1 region is gamma 1, gamma 2, gamma 3, gamma 4, alpha 1, alpha 2, mu, delta, or epsilon.
[26] The antigen-binding molecule of [24] or [25], wherein the amino acid residue from which the bonds between the antigen-binding domains originate is present at any one of positions 119 to 123, 131 to 140, 148 to 150, 155 to 167, 174 to 178, 188 to 197, 201 to 214, and 218 to 219, according to EU numbering, in the CH1 region.
[27] The antigen-binding molecule of [26], wherein the amino acid residue from which the bonds between the antigen-binding domains originate is present at a position selected from the group consisting of positions 119, 122, 123, 131, 132, 133, 134, 135, 136, 137, 138, 139, 140, 148, 150, 155, 156, 157, 159, 160, 161, 162, 163, 164, 165, 167, 174, 176, 177, 178, 188, 189, 190, 191, 192, 193, 194, 195, 196, 197, 201, 203, 205, 206, 207, 208, 211, 212, 213, 214, 218, and 219, according to EU numbering, in the CH1 region.
[28] The antigen binding molecule of [27], wherein the amino acid residue from which the bonds between the antigen-binding domains originate is present at position 134, 135, 136, 137, 191, 192, 193, 194, 195, or 196, according to EU numbering, in the CH1 region.
[29] The antigen binding molecule of [28], wherein the amino acid residue from which the bonds between the antigen-binding domains originate is present at position 135, 136, or 191, according to EU numbering, in the CH1 region.
[30] The antigen binding molecule of any one of [24] to [29], wherein at least one of the bonds linking the two antigen-binding domains is formed by linking an amino acid residue in the CH1 region of the first antigen-binding domain with an amino acid residue in the CH1 region of the second antigen-binding domain.
[31] The antigen binding molecule of [30], wherein the amino acid residues in the first antigen-binding domain and the second antigen-binding domain are each independently selected from the group consisting of positions 119, 120, 121, 122, and 123 according to EU numbering.
[32] The antigen binding molecule of [30], wherein the amino acid residues in the first antigen-binding domain and the second antigen-binding domain are each independently selected from the group consisting of positions 131, 132, 133, 134, 135, 136, 137, 138, 139, and 140 according to EU numbering.
[33] The antigen binding molecule of [30], wherein the amino acid residues in the first antigen-binding domain and the second antigen-binding domain are each independently selected from the group consisting of positions 148, 149, and 150 according to EU numbering.
[34] The antigen binding molecule of [30], wherein the amino acid residues in the first antigen-binding domain and the second antigen-binding domain are each independently selected from the group consisting of positions 155, 156, 157, 158, 159, 160, 161, 162, 163, 164, 165, 166, and 167 according to EU numbering.
[35] The antigen binding molecule of [30], wherein the amino acid residues in the first antigen-binding domain and the second antigen-binding domain are each independently selected from the group consisting of positions 174, 175, 176, 177, and 178 according to EU numbering.
[36] The antigen binding molecule of [30], wherein the amino acid residues in the first antigen-binding domain and the second antigen-binding domain are each independently selected from the group consisting of positions 188, 189, 190, 191, 192, 193, 194, 195, 196, and 197 according to EU numbering.
[37] The antigen binding molecule of [30], wherein the amino acid residues in the first antigen-binding domain and the second antigen-binding domain are each independently selected from the group consisting of positions 201, 202, 203, 204, 205, 206, 207, 208, 209, 210, 211, 212, 213, and 214 according to EU numbering.
[38] The antigen binding molecule of [30], wherein the amino acid residues in the first antigen-binding domain and the second antigen-binding domain are each independently selected from the group consisting of positions 218 and 219 according to EU numbering.
[39] The antigen binding molecule of any one of [30] to [38], wherein the difference between the positions of the amino acid residues in the first antigen-binding domain and the second antigen-binding domain is three amino acids or less.
[40] The antigen-binding molecule of [39], wherein at least one of the bonds linking the two antigen-binding domains is formed by linking an amino acid residue at position 135 according to EU numbering in the CH1 region of the first antigen-binding domain with an amino acid residue at any one of positions 132 to 138 according to EU numbering in the CH1 region of the second antigen-binding domain.
[41] The antigen-binding molecule of [39], wherein at least one of the bonds linking the two antigen-binding domains is formed by linking an amino acid residue at position 136 according to EU numbering in the CH1 region of the first antigen-binding domain with an amino acid residue at any one of positions 133 to 139 according to EU numbering in the CH1 region of the second antigen-binding domain.
[42] The antigen-binding molecule of [39], wherein at least one of the bonds linking the two antigen-binding domains is formed by linking an amino acid residue at position 191 according to EU numbering in the CH1 region of the first antigen-binding domain with an amino acid residue at any one of positions 188 to 194 according to EU numbering in the CH1 region of the second antigen-binding domain.
[43] The antigen-binding molecule of [40], wherein at least one of the bonds linking the two antigen-binding domains is formed by linking amino acid residues at position 135 according to EU numbering in the CH1 region of the two antigen-binding domains with each other.
[44] The antigen-binding molecule of [41], wherein at least one of the bonds linking the two antigen-binding domains is formed by linking amino acid residues at position 136 according to EU numbering in the CH1 region of the two antigen-binding domains with each other.
[45] The antigen-binding molecule of [42], wherein at least one of the bonds linking the two antigen-binding domains is formed by linking amino acid residues at position 191 according to EU numbering in the CH1 region of the two antigen-binding domains with each other.
[45A] The antigen-binding molecule of [42], wherein one disulfide bond is formed between the amino acid residues at position 191 according to EU numbering in the respective CH1 regions of the first antigen-binding domain and the second antigen-binding domain.
[45B] The antigen-binding molecule of [45A], wherein additional one, two or more disulfide bond(s) is/are formed between the first antigen-binding domain and the second antigen-binding domain via the amino acid residues at the following positions according to EU numbering in each of the respective CH1 regions of the first antigen-binding domain and the second antigen-binding domain:
   (a) between amino acid residues at any position of 131 to 138, 194 and 195 in each of the two antigen-binding domains;
   (b) between the amino acid residues at position 131 in each of the two antigen-binding domains, and between the amino acid residues at position 194 in each of the two antigen-binding domains;
   (c) between the amino acid residues at position 132 in each of the two antigen-binding domains, and between the amino acid residues at position 194 in each of the two antigen-binding domains;
   (d) between the amino acid residues at position 133 in each of the two antigen-binding domains, and between the amino acid residues at position 194 in each of the two antigen-binding domains;
   (e) between the amino acid residues at position 134 in each of the two antigen-binding domains, and between the amino acid residues at position 194 in each of the two antigen-binding domains;
   (f) between the amino acid residues at position 135 in each of the two antigen-binding domains, and between the amino acid residues at position 194 in each of the two antigen-binding domains;
   (g) between the amino acid residues at position 136 in each of the two antigen-binding domains, and between the amino acid residues at position 194 in each of the two antigen-binding domains;
   (h) between the amino acid residues at position 137 in each of the two antigen-binding domains, and between the amino acid residues at position 194 in each of the two antigen-binding domains;
   (i) between the amino acid residues at position 138 in each of the two antigen-binding domains, and between the amino acid residues at position 194 in each of the two antigen-binding domains;
   (j) between the amino acid residues at position 131 in each of the two antigen-binding domains, and between the amino acid residues at position 195 in each of the two antigen-binding domains;
   (k) between the amino acid residues at position 132 in each of the two antigen-binding domains, and between the amino acid residues at position 195 in each of the two antigen-binding domains;
   (l) between the amino acid residues at position 133 in each of the two antigen-binding domains, and between the amino acid residues at position 195 in each of the two antigen-binding domains;
   (m) between the amino acid residues at position 134 in each of the two antigen-binding domains, and between the amino acid residues at position 195 in each of the two antigen-binding domains;
   (n) between the amino acid residues at position 135 in each of the two antigen-binding domains, and between the amino acid residues at position 195 in each of the two antigen-binding domains;
   (o) between the amino acid residues at position 136 in each of the two antigen-binding domains, and between the amino acid residues at position 195 in each of the two antigen-binding domains;
   (p) between the amino acid residues at position 137 in each of the two antigen-binding domains, and between the amino acid residues at position 195 in each of the two antigen-binding domains; and
   (q) between the amino acid residues at position 138 in each of the two antigen-binding domains, and between the amino acid residues at position 195 in each of the two antigen-binding domains.
[45C] The antigen-binding molecule of [45A] or [45B], wherein any one of the first and second antigen-binding domains comprises one, two or more charged amino acid residues at position 136-138 (according to EU numbering) in the respective CH1 region; and the other antigen-binding domain out of the first and second antigen-binding domains comprises one, two or more oppositely charged amino acid residues at position 193-195 (according to EU numbering) in the respective CH1 region.
[45D] The antigen-binding molecule of [45A] or [45B], wherein any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more negatively charged amino acid residues at position 193-195 (according to EU numbering) in the respective CH1 region.
[45E] The antigen-binding molecule of [45A] or [45B], wherein any one of the first and second antigen-binding domains comprises one, two or more negatively charged amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region.
[45F] The antigen-binding molecule of [45A] or [45B], wherein any one of the first and second antigen-binding domains comprises one, two or more of the following amino acid residues in the respective CH1 region (according to EU numbering):
   (a) the amino acid residue at position 136 is glutamic acid (E) or aspartic acid (D);
   (b) the amino acid residue at position 137 is glutamic acid (E) or aspartic acid (D);
   (c) the amino acid residue at position 138 is glutamic acid (E) or aspartic acid (D); and the other antigen-binding domain out of the first and second antigen-binding domains comprises one, two or more of the following amino acid residues in the respective CH1 region (according to EU numbering):
   (d) the amino acid residue at position 193 is lysine (K), arginine (R), or histidine (H);
   (e) the amino acid residue at position 194 is lysine (K), arginine (R), or histidine (H); and
   (f) the amino acid residue at position 195 is lysine (K), arginine (R), or histidine (H).
[45G-1] The antigen-binding molecule of [45A] or [45B], wherein any one of the first and second antigen-binding domains comprises one or more of the following amino acid residues in the respective CH1 region (according to EU numbering):
   (a) the amino acid residue at position 136 is lysine (K), arginine (R), or histidine (H);
   (b) the amino acid residue at position 137 is lysine (K), arginine (R), or histidine (H);
   (c) the amino acid residue at position 138 is lysine (K), arginine (R), or histidine (H); and the other antigen-binding domain out of the first and second antigen-binding domains comprises one or more of the following amino acid residues in the respective CH1 region (according to EU numbering):
   (d) the amino acid residue at position 193 is glutamic acid (E) or aspartic acid (D);
   (e) the amino acid residue at position 194 is glutamic acid (E) or aspartic acid (D); and
   (f) the amino acid residue at position 195 is glutamic acid (E) or aspartic acid (D).
[45G-2] The antigen-binding molecule of [45A] or [45B], wherein each of the first and second antigen-binding domains comprises any of the specific charged mutation combination in the respective CH1 region (according to EU numbering) as listed in Tables 7, Table 82 or Table 85.
[45H] The antigen-binding molecule of [45A] or [45B], wherein any one of the first and second antigen-binding domains comprises one, two or more hydrophobic amino acid residues at position 136-138 (according to EU numbering) in the respective CH1 region; and the other antigen-binding domain out of the first and second antigen-binding domains comprises one, two or more hydrophobic amino acid residues at position 193-195 (according to EU numbering) in the respective CH1 region.
[45I-1] The antigen-binding molecule of [45H], wherein said hydrophobic amino acid residue(s) is/are alanine (Ala), valine (Val), leucine (Leu), isoleucine (Ile), phenylalanine (Phe), and/or tryptophan (Trp).
[45I-2] The method of [45A] or [45B], wherein each of the first and second antigen-binding domains comprises any of the specific hydrophobic amino acid mutation combination in the respective CH1 region (according to EU numbering) as listed in Table 10.
[45J] The antigen-binding molecule of [45A] or [45B], wherein any one of the first and second antigen-binding domains comprises one “knob” amino acid residues at position 136-138 (according to EU numbering) in the respective CH1 region; and the other antigen-binding domain out of the first and second antigen-binding domains comprises one, two or more “hole” amino acid residues at position 193-195 (according to EU numbering) in the respective CH1 region.
[45K] The antigen-binding molecule of [45A] or [45B], wherein any one of the first and second antigen-binding domains comprises one, two or more “hole” amino acid residues at position 136-138 (according to EU numbering) in the respective CH1 region; and the other antigen-binding domain out of the first and second antigen-binding domains comprises one “knob” amino acid residues at position 193-195 (according to EU numbering) in the respective CH1 region.
[45L] The antigen-binding molecule of [45J] or [45K], wherein said “knob” amino acid residue(s) is/are selected from the group consisting of tryptophan (Trp) and phenylalanine (Phe); and said “hole” amino acid residue(s) is/are selected from the group consisting of alanine (Ala), valine (Val), threonine (T) or serine (S).
[45M] The antigen-binding molecule of [45A] or [45B], wherein any one of the first and second antigen-binding domains comprises one, two or more aromatic amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region.
[45N] The antigen-binding molecule of [45A] or [45B], wherein any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more aromatic amino acid residues at position 193-195 (according to EU numbering) in the respective CH1 region.
[45O] The antigen-binding molecule of [45M] or [45N], wherein said aromatic amino acid residue(s) is/are selected from the group consisting of tryptophan (Trp), tyrosine (Tyr), histidine (His), and phenylalanine (Phe); and said positively charged amino acid residue(s) is/are selected from a group consisting of lysine (K), arginine (R), or histidine (H).
[46] The antigen-binding molecule of [22] or [23], wherein the amino acid residue from which the bonds between the antigen-binding domains originate is present within a CL region.
[47] The antigen-binding molecule of [46], wherein the subclass of the CL region is kappa or lambda.
[48] The antigen-binding molecule of [46] or [47], wherein the amino acid residue from which the bonds between the antigen-binding domains originate is present at any one of positions 108 to 112, 121 to 128, 151 to 156, 184 to 190, 195 to 196, 200 to 203, and 208 to 213, according to Kabat numbering, in the CL region.
[49] The antigen-binding molecule of [48], wherein the amino acid residue from which the bonds between the antigen-binding domains originate is present at a position selected from the group consisting of positions 108, 109, 112, 121, 123, 126, 128, 151, 152, 153, 156, 184, 186, 188, 189, 190, 195, 196, 200, 201, 202, 203, 208, 210, 211, 212, and 213 according to Kabat numbering in the CL region.
[50] The antigen-binding molecule of [49], wherein the amino acid residue from which the bonds between the antigen-binding domains originate is present at position 126 according to Kabat numbering in the CL region.
[51] The antigen-binding molecule of any one of [46] to [50], wherein at least one of the bonds linking the two antigen-binding domains is formed by linking an amino acid residue in the CL region of the first antigen-binding domain with an amino acid residue in the CL region of the second antigen-binding domain.
[52] The antigen-binding molecule of [51], wherein the amino acid residues in the first antigen-binding domain and the second antigen-binding domain are each independently selected from the group consisting of positions 108, 109, 110, 111, and 112 according to Kabat numbering.
[53] The antigen-binding molecule of [51], wherein the amino acid residues in the first antigen-binding domain and the second antigen-binding domain are each independently selected from the group consisting of positions 121, 122, 123, 124, 125, 126, 127, and 128 according to Kabat numbering.
[54] The antigen-binding molecule of [51], wherein the amino acid residues in the first antigen-binding domain and the second antigen-binding domain are each independently selected from the group consisting of positions 151, 152, 153, 154, 155, and 156 according to Kabat numbering.
[55] The antigen-binding molecule of [51], wherein the amino acid residues in the first antigen-binding domain and the second antigen-binding domain are each independently selected from the group consisting of positions 184, 185, 186, 187, 188, 189, and 190 according to Kabat numbering.
[56] The antigen-binding molecule of [51], wherein the amino acid residues in the first antigen-binding domain and the second antigen-binding domain are each independently selected from the group consisting of positions 195 and 196 according to Kabat numbering.
[57] The antigen-binding molecule of [51], wherein the amino acid residues in the first antigen-binding domain and the second antigen-binding domain are each independently selected from the group consisting of positions 200, 201, 202, and 203 according to Kabat numbering.
[58] The antigen-binding molecule of [51], wherein the amino acid residues in the first antigen-binding domain and the second antigen-binding domain are each independently selected from the group consisting of positions 208, 209, 210, 211, 212, and 213 according to Kabat numbering.
[59] The antigen-binding molecule of any one of [51] to [58], wherein the difference between the positions of the amino acid residues in the first antigen-binding domain and the second antigen-binding domain is three amino acids or less
[60] The antigen-binding molecule of [59], wherein at least one of the bonds linking the two antigen-binding domains is formed by linking amino acid residues at position 126 according to Kabat numbering in the CL region of the two antigen-binding domains with each other.
[61] The antigen-binding molecule of any one of [24] to [29] and [46] to [50], wherein at least one of the bonds linking the two antigen-binding domains is formed by linking an amino acid residue in the CH1 region of the first antigen-binding domain with an amino acid residue in the CL region of the second antigen-binding domain.
[62] The antigen-binding molecule of [61], wherein the amino acid residue in the CH1 region is selected from the group consisting of positions 188, 189, 190, 191, 192, 193, 194, 195, 196, and 197 according to EU numbering, and the amino acid residue in the CL region is selected from the group consisting of positions 121, 122, 123, 124, 125, 126, 127, and 128 according to Kabat numbering.
[63] The antigen-binding molecule of [62], wherein at least one of the bonds linking the two antigen-binding domains is formed by linking an amino acid residue at position 191 according to EU numbering in the CH1 region of the first antigen-binding domain with an amino acid residue at position 126 according to Kabat numbering in the CL region of the second antigen-binding domain.
[64] The antigen-binding molecule of [21], wherein the amino acid residue from which the bonds between the antigen-binding domains originate is present within a variable region.
[65] The antigen-binding molecule of [64], wherein the amino acid residue from which the bonds between the antigen-binding domains originate is present within a VH region.
[66] The antigen-binding molecule of [65], wherein the amino acid residue from which the bonds between the antigen-binding domains originate is present at a position selected from the group consisting of positions 6, 8, 16, 20, 25, 26, 28, 74, and 82b according to Kabat numbering in the VH region.
[67] The antigen-binding molecule of [64], wherein the amino acid residue from which the bonds between the antigen-binding domains originate is present within a VL region.
[68] The antigen-binding molecule of [67], wherein the amino acid residue from which the bonds between the antigen-binding domains originate is present at a position selected from the group consisting of positions 21, 27, 58, 77, 100, 105, and 107 according to Kabat numbering in the VL region (subclass kappa).
[69] The antigen-binding molecule of [67], wherein the amino acid residue from which the bonds between the antigen-binding domains originate is present at a position selected from the group consisting of positions 6, 19, 33, and 34 according to Kabat numbering in the VL region (subclass lambda).
[70] The antigen-binding molecule of [64], wherein the amino acid residue from which the bonds between the antigen-binding domains originate is present within a VHH region.
[71] The antigen-binding molecule of [70], wherein the amino acid residue from which the bonds between the antigen-binding domains originate is present at a position selected from the group consisting of positions 4, 6, 7, 8, 9, 10, 11, 12, 14, 15, 17, 20, 24, 27, 29, 38, 39, 40, 41, 43, 44, 45, 46, 47, 48, 49, 67, 69, 71, 78, 80, 82, 82c, 85, 88, 91, 93, 94, and 107 according to Kabat numbering in the VHH region.
[72] The antigen-binding molecule of any one of [1] to [18], wherein at least one of the first and second antigen-binding domains comprises a non-antibody protein binding to a particular antigen, or a fragment thereof.
[73] The antigen-binding molecule of [72], wherein the non-antibody protein is either of a pair of a ligand and a receptor which specifically bind to each other.
[74] The antigen-binding molecule of any one of [1] to [73], wherein the antigen-binding domains comprise a hinge region.
[75] The antigen-binding molecule of [74], wherein at least one of cysteine residues present within the wild-type hinge region is substituted with another amino acid residue.
[76] The antigen-binding molecule of [75], wherein the cysteine residue is present at positions 226 and/or 229 according to EU numbering in the hinge region.
[77] The antigen-binding molecule of [74] or [76], wherein at least one of amino acid residues from which the bonds between the antigen-binding domains originate is present within the hinge region.
[78] The antigen-binding molecule of [77], wherein the amino acid residue from which the bonds between the antigen-binding domains originate is present at a position selected from the group consisting of positions 216, 218, and 219 according to EU numbering in the hinge region.
[79] The antigen-binding molecule of any one of [1] to [78], wherein the first antigen-binding domain and the second antigen-binding domain are linked with each other via two or more bonds.
[80] The antigen-binding molecule of [79], wherein at least one of amino acid residues from which the bonds between the antigen-binding domains originate is an amino acid residue present in a wild-type sequence.
[81] The antigen-binding molecule of [80], wherein the amino acid residue from which the bonds between the antigen-binding domains originate is present within a hinge region.
[82] The antigen-binding molecule of [81], wherein the amino acid residue from which the bonds between the antigen-binding domains originate is a cysteine residue in the hinge region.
[83] The antigen-binding molecule of any one of [80] to [82], wherein at least one of the bonds linking the two antigen-binding domains is a disulfide bond formed by crosslinking of cysteine residues present within the hinge region with each other.
[84] The antigen-binding molecule of [83], wherein the cysteine residues are present at positions 226 and/or 229 according to EU numbering in the hinge region.
[85] The antigen-binding molecule of any one of [79] to [84], wherein at least one of amino acid residues from which the bonds between the antigen-binding domains originate is present within the antibody fragment, and at least one of the amino acid residues is present within the hinge region.
[86] The antigen-binding molecule of [85], wherein the first and second antigen-binding domains each comprise a Fab and a hinge region, and wherein the antigen-binding molecule comprising the two antigen-binding domains is F(ab′)2. [87] The antigen-binding molecule of any one of [1] to [86], wherein the antigen-binding domains comprise an Fc region.
[88] The antigen-binding molecule of [87], wherein one or more amino acid mutations promoting multimerization of Fc regions are introduced into the Fc region.
[89] The antigen-binding molecule of [88], wherein the amino acid mutations promoting the multimerization comprise an amino acid mutation at at least one position selected from the group consisting of positions 247, 248, 253, 254, 310, 311, 338, 345, 356, 359, 382, 385, 386, 430, 433, 434, 436, 437, 438, 439, 440, and 447 according to EU numbering.
[90] The antigen-binding molecule of [88] or [89], wherein the multimerization is hexamerization.
[91] The antigen-binding molecule of any one of [87] to [90], which is a full-length antibody.

In another aspect, the present invention also provides the following:
[92] The antigen-binding molecule of any one of [1] to [91], wherein both the first and second antigen-binding domains bind to the same antigen.
[93] The antigen-binding molecule of [92], wherein both the first and second antigen-binding domains bind to the same epitope on said antigen.
[94] The antigen-binding molecule of [92], wherein each of the first and second antigen-binding domains binds to a different epitope on said antigen.
[95] The antigen-binding molecule of any one of [1] to [91], wherein each of the first and second antigen-binding domains binds to a different antigen.
[96] The antigen-binding molecule of [93], wherein both the first and second antigen-binding domains have the same amino acid sequence.
[97] The antigen-binding molecule of any one of [93] to [95], wherein each of the first and second antigen-binding domains has a different amino acid sequence.
[98] The antigen-binding molecule of any one of [1] to [91], wherein at least one of two antigens to which the first and second antigen-binding domains bind is a soluble protein.
[99] The antigen-binding molecule of any one of [1] to [91], wherein at least one of two antigens to which the first and second antigen-binding domains bind is a membrane protein.

In another aspect, the present invention also provides the following:
[100] The antigen-binding molecule of any one of [1] to [99], which has activity of regulating interaction between two antigen molecules.
[101] The antigen-binding molecule of [100], which is capable of enhancing or diminishing interaction between two antigen molecules as compared to a control antigen-binding molecule, wherein the control antigen-binding molecule differs from the antigen-binding molecule of [100] only in that the control antigen-binding molecule has one less bond between the two antigen-binding domains.
[102] The antigen-binding molecule of [100] or [101], wherein the two antigen molecules are a ligand and a receptor thereof, respectively, and wherein the antigen-binding molecule has activity of promoting activation of the receptor by the ligand.
[103] The antigen-binding molecule of [100] or [101], wherein the two antigen molecules are an enzyme and a substrate thereof, respectively, and wherein the antigen-binding molecule has activity of promoting catalytic reaction of the enzyme with the substrate.
[104] The antigen-binding molecule of [100] or [101], wherein both of the two antigen molecules are proteins present on cellular surfaces, and wherein the antigen-binding molecule has activity of promoting interaction between a cell expressing the first antigen and a cell expressing the second antigen.
[105] The antigen-binding molecule of [104], wherein the cell expressing the first antigen is a cell with cytotoxic activity, and the cell expressing the second antigen is a target cell thereof, and wherein the antigen-binding molecule promotes damage of said target cell by said cell with cytotoxic activity.
[106] The antigen-binding molecule of [105], wherein the cell with cytotoxic activity is a T cell, NK cell, monocyte, or macrophage.
[107] The antigen-binding molecule of any one of [1] to [99], which has activity of regulating activation of two antigen molecules which are activated by association with each other.
[108] The antigen-binding molecule of [107], which enhances or diminishes activation of two antigen molecules as compared to a control antigen-binding molecule, wherein the control antigen-binding molecule differs from the antigen-binding molecule of [107] only in that the control antigen-binding molecule has one less bond between the two antigen-binding domains.
[109] The antigen-binding molecule of [107] or [108], wherein the antigen molecules are selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.
[110] The antigen-binding molecule of any one of [1] to [99], which has activity of holding two antigen molecules at spatially close positions.
[111] The antigen-binding molecule of [110], which is capable of holding two antigen molecules at closer positions than a control antigen-binding molecule, wherein the control antigen-binding molecule differs from the antigen-binding molecule of [110] only in that the control antigen-binding molecule has one less bond between the two antigen-binding domains.
[112] The antigen-binding molecule of any one of [1] to [99], wherein the two antigen-binding domains are at spatially close positions and/or the mobility of the two antigen-binding domains is reduced.
[113] The antigen-binding molecule of [112], wherein the two antigen-binding domains are at closer positions and/or the two antigen-binding domains have less mobility than a control antigen-binding molecule, wherein the control antigen-binding molecule differs from the antigen-binding molecule of [112] only in that the control antigen-binding molecule has one less bond between the two antigen-binding domains.
[114] The antigen-binding molecule of any one of [1] to [99], which has resistance to protease cleavage.
[115] The antigen-binding molecule of [114], which has increased resistance to protease cleavage as compared to a control antigen-binding molecule, wherein the control antigen-binding molecule differs from the antigen-binding molecule of [114] only in that the control antigen-binding molecule has one less bond between the two antigen-binding domains.
[116] The antigen-binding molecule of [115], wherein the proportion of the full-length molecule remaining after protease treatment is increased as compared to the control antigen-binding molecule.
[117] The antigen-binding molecule of [115] or [116], wherein the proportion of a particular fragment produced after protease treatment is reduced as compared to the control antigen-binding molecule.
[118] The antigen-binding molecule of any one of [1] to [99], wherein when the molecule is treated with a protease, a dimer of the antigen-binding domains or fragments thereof is excised.
[119] The antigen-binding molecule of [118], wherein when the control antigen-binding molecule is treated with said protease, monomers of the antigen-binding domains or fragments thereof are excised, and wherein the control antigen-binding molecule differs from the antigen-binding molecule of [118] only in that the control antigen-binding molecule has one less bond between the two antigen-binding domains.
[120] The antigen-binding molecule of [118] or [119], wherein the protease cleaves the hinge region.
[121] The antigen binding molecule of any one of [101] to [106], [108] to [109], [111], [113], [115] to [117], and [119] to [120], wherein the one less bond is a bond formed originating from a mutated amino acid residue.
[122] The antigen-binding molecule of [121], wherein the mutated amino acid residue is a cysteine residue.

In another aspect, the present invention also provides the following:
[123] A pharmaceutical composition comprising the antigen-binding molecule of any one of [1] to [122] and a pharmaceutically acceptable carrier.

In another aspect, the present invention also provides the following:
[124] A method for regulating interaction between two antigen molecules, comprising:
   (a) providing an antigen-binding molecule comprising two antigen-binding domains,
   (b) adding to the antigen-binding molecule at least one bond which links the two antigen-binding domains with each other, and
   (c) contacting the antigen-binding molecule produced in (b) with the two antigen molecules.
[125] A method for regulating activity of two antigen molecules which are activated by association with each other, comprising:
   (a) providing an antigen-binding molecule comprising two antigen-binding domains,
   (b) adding to the antigen-binding molecule at least one bond which links the two antigen-binding domains with each other, and
   (c) contacting the antigen-binding molecule produced in (b) with the two antigen molecules.
[126] A method for holding two antigen molecules at spatially close positions, comprising:
   (a) providing an antigen-binding molecule comprising two antigen-binding domains,
   (b) adding to the antigen-binding molecule at least one bond which links the two antigen-binding domains with each other, and
   (c) contacting the antigen-binding molecule produced in (b) with the two antigen molecules.
[127] A method for placing two antigen-binding domains at spatially close positions and/or reducing the mobility of the two antigen-binding domains, comprising:
   (a) providing an antigen-binding molecule comprising two antigen-binding domains, and
   (b) adding to the antigen-binding molecule at least one bond which links the two antigen-binding domains with each other.
[128] A method for increasing resistance of an antigen-binding molecule to protease cleavage, comprising:
   (a) providing an antigen-binding molecule comprising two antigen-binding domains, and
   (b) adding to the antigen-binding molecule at least one bond which links the two antigen-binding domains with each other.

In another aspect, the present invention also provides the following:
[129] A method for producing an antigen-binding molecule which has activity of regulating interaction between two antigen molecules, comprising:
   (a) providing a nucleic acid encoding a polypeptide comprising a first antigen-binding domain and a nucleic acid encoding a polypeptide comprising a second antigen-binding domain,
   (b) introducing a mutation into the nucleic acids encoding the two antigen-binding domains such that at least one bond linking the two antigen-binding domains is added,
   (c) introducing the nucleic acids produced in (b) into a host cell,
   (d) culturing the host cell such that the two polypeptides are expressed, and
   (e) obtaining an antigen-binding molecule which is a polypeptide comprising the first and second antigen-binding domains, wherein the two antigen-binding domains are linked with each other via one or more bonds.
[130] A method for producing an antigen-binding molecule which has activity of regulating activation of two antigen molecules which are activated by association with each other, comprising:
   (a) providing a nucleic acid encoding a polypeptide comprising a first antigen-binding domain and a nucleic acid encoding a polypeptide comprising a second antigen-binding domain,
   (b) introducing a mutation into the nucleic acids encoding the two antigen-binding domains such that at least one bond linking the two antigen-binding domains is added,
   (c) introducing the nucleic acids produced in (b) into a host cell,
   (d) culturing the host cell such that the two polypeptides are expressed, and
   (e) obtaining an antigen-binding molecule which is a polypeptide comprising the first and second antigen-binding domains, wherein the two antigen-binding domains are linked with each other via one or more bonds.
[131] A method for producing an antigen-binding molecule which has activity of holding two antigen molecules at spatially close positions, comprising:
   (a) providing a nucleic acid encoding a polypeptide comprising a first antigen-binding domain and a nucleic acid encoding a polypeptide comprising a second antigen-binding domain,
   (b) introducing a mutation into the nucleic acids encoding the two antigen-binding domains such that at least one bond linking the two antigen-binding domains is added,
   (c) introducing the nucleic acids produced in (b) into a host cell,
   (d) culturing the host cell such that the two polypeptides are expressed, and
   (e) obtaining an antigen-binding molecule which is a polypeptide comprising the first and second antigen-binding domains, wherein the two antigen-binding domains are linked with each other via one or more bonds.
[132] A method for producing an antigen-binding molecule in which two antigen-binding domains are present at spatially close positions and/or the mobility of the two antigen binding domains is reduced, comprising:
   (a) providing a nucleic acid encoding a polypeptide comprising a first antigen-binding domain and a nucleic acid encoding a polypeptide comprising a second antigen-binding domain,
   (b) introducing a mutation into the nucleic acids encoding the two antigen-binding domains such that at least one bond linking the two antigen-binding domains is added,
   (c) introducing the nucleic acids produced in (b) into a host cell,
   (d) culturing the host cell such that the two polypeptides are expressed, and
   (e) obtaining an antigen-binding molecule which is a polypeptide comprising the first and second antigen-binding domains, wherein the two antigen-binding domains are linked with each other via one or more bonds.
[133] A method for producing an antigen-binding molecule which has increased resistance to protease cleavage, comprising:
   (a) providing a nucleic acid encoding a polypeptide comprising a first antigen-binding domain and a nucleic acid encoding a polypeptide comprising a second antigen-binding domain,
   (b) introducing a mutation into the nucleic acids encoding the two antigen-binding domains such that at least one bond linking the two antigen-binding domains is added,
   (c) introducing the nucleic acids produced in (b) into a host cell,
   (d) culturing the host cell such that the two polypeptides are expressed, and
   (e) obtaining an antigen-binding molecule which is a polypeptide comprising the first and second antigen-binding domains, wherein the two antigen-binding domains are linked with each other via one or more bonds.

In another aspect, the present invention also provides the following:
[134] A method for identifying a novel pair of protein molecules which are activated by association with each other, comprising:
   (a) providing two arbitrary protein molecules,
   (b) producing, by the method of any one of [129] to [133], an antigen-binding molecule comprising two antigen-binding domains which respectively bind to the two protein molecules,
   (c) contacting the antigen-binding molecule produced in (b) with the two protein molecules, and
   (d) assessing whether or not the two protein molecules are activated.
[135] The method of [134], wherein at least one of the protein molecules is selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

### BRIEF DESCRIPTION OF DRAWINGS

FIG. 1 shows a non-reducing SDS-PAGE gel image for analyzing OKT3 and its variants with the cysteine substitution (see Example 1). Two broken lines indicate upper and lower bands. The lower band can be considered to correspond to the antibody having one or more engineered disulfide bond(s) formed between the CH1 regions.

FIG. 2 shows a non-reducing SDS-PAGE gel image for analyzing OKT3 variants with the cysteine substitution and OKT3-KiH (see Example 1). Two broken lines indicate upper and lower bands.

FIG. 3 shows a non-reducing SDS-PAGE gel image for analyzing OKT3-KiH variants with the cysteine substitution (see Example 1). Two broken lines indicate upper and lower bands.

FIG. 4 shows a non-reducing SDS-PAGE gel image for analyzing OKT3-KiH variants with the cysteine substitution (see Example 1). Two broken lines indicate upper and lower bands.

FIG. 5 shows an image of non-reducing SDS-PAGE gel in which 2-MEA concentrations of each sample are described (left panel); and a graph showing the lower band to upper band ratio (crosslinking ratio or crosslinking %) of each sample (right panel) (see Example 4). 20 mg/mL of the antibody was reacted by mixing with 2-MEA of different concentrations. The leftmost bar and dotted line represent the lower band to upper band ratio (crosslinking ratio or crosslinking %) of the control (0 mM 2-MEA). Numbers in the bars are the values of the lower band to upper band ratios (crosslinking ratio or crosslinking %).

FIG. 6 shows an image of non-reducing SDS-PAGE gel in which 2-MEA concentrations of each sample are described (upper panel); and a graph showing the lower band to upper band ratio (crosslinking ratio or crosslinking %) of each sample (lower panel) (see Example 4). 20 mg/mL of the antibody was reacted by mixing with 2-MEA of different concentrations. The leftmost bar and dotted line represent the lower band to upper band ratio (crosslinking ratio or crosslinking %) of the control (0 mM 2-MEA). Numbers in the bars are the values of the lower band to upper band ratio (crosslinking ratio or crosslinking %).

FIG. 7 shows an image of non-reducing SDS-PAGE gel in which 2-MEA concentrations of each sample are described (left panel); and a graph showing the lower band to upper band ratio (crosslinking ratio or crosslinking %) of each sample (right panel) (see Example 4). 1 mg/mL of the antibody was reacted by mixing with 2-MEA of different concentrations. The leftmost bar and dotted line represent the lower band to upper band ratio (crosslinking ratio or crosslinking %) of the control (0 mM 2-MEA). Numbers in the bars are the value of the lower band to upper band ratio (crosslinking ratio or crosslinking %).

FIG. 8 shows an image of non-reducing SDS-PAGE gel in which 2-MEA concentrations of each sample are described (upper panel); and a graph showing the lower band to upper band ratio (crosslinking ratio or crosslinking %) of each sample (lower panel) (see Example 4). 1 mg/mL of the antibody was reacted by mixing with 2-MEA of different concentrations. The leftmost bar and dotted line represent the lower band to upper band ratio (crosslinking ratio or crosslinking %) of the control (0 mM 2-MEA). Numbers in the bars are the values of the lower band to upper band ratio (crosslinking ratio or crosslinking %).

FIG. 9 shows an image of non-reducing SDS-PAGE gel in which TCEP concentrations of each sample are described (left panel); and a graph showing the lower band to upper band ratio (crosslinking ratio or crosslinking %) of each sample (right panel) (see Example 5). 20 mg/mL of the antibody was reacted by mixing with TCEP of different concentrations. The leftmost bar and dotted line represent the lower band to upper band ratio (crosslinking ratio or crosslinking %) of the control (0 mM TCEP). Numbers in the bars are the values of the lower band to upper band ratio (crosslinking ratio or crosslinking %).

FIG. 10 shows an image of non-reducing SDS-PAGE gel in which TCEP concentrations of each sample are described (upper panel); and a graph showing the lower band to upper band ratio (crosslinking ratio or crosslinking %) of each sample (lower panel) (see Example 5). 20 mg/mL of the antibody was reacted by mixing with TCEP of each concentration. N.D. means that no band was detected. The leftmost bar and dotted line represent the lower band to upper band ratio (crosslinking ratio or crosslinking %) of the control (0 mM TCEP). Numbers in the bars are the values of the lower band to upper band ratio (crosslinking ratio or crosslinking %).

FIG. 11 shows an image of non-reducing SDS-PAGE gel in which TCEP concentrations of each sample are described (upper panel); and a graph showing the lower band to upper band ratio (crosslinking ratio or crosslinking %) of each sample (lower panel) (see Example 5). 1 mg/mL of the antibody was reacted by mixing with TCEP of each concentration. N.D. means that no band was detected. The leftmost bar and dotted line represent the lower band to upper band ratio (crosslinking ratio or crosslinking %) of the control (0 mM TCEP). Numbers in the bars are the values of the lower band to upper band ratio (crosslinking ratio or crosslinking %).

FIG. 12 shows an image of non-reducing SDS-PAGE gel in which reagent concentrations of each sample are described (upper panel); and a graph showing the lower band to upper band ratio (crosslinking ratio or crosslinking %) of samples reacted with DTT (left) or Cysteine (right) (see Example 6). 20 mg/mL of the antibody was reacted by mixing with DTT or Cysteine of each concentration. The leftmost bar and dotted line represent the lower band to upper band ratio (crosslinking ratio or crosslinking %) of the control (without reducing agent). Numbers in the bars are the values of the lower band to upper band ratio (crosslinking ratio or crosslinking %).

FIG. 13 shows an image of non-reducing SDS-PAGE gel in which reagent concentrations of each sample are described (upper panel); and a graph showing the lower band to upper band ratio (crosslinking ratio or crosslinking %) of samples reacted with GSH (left) or Na₂SO₃ (right) (lower panel) (see Example 6). 20 mg/mL of the antibody was reacted by mixing with GSH or Na₂SO₃ of each concentration. The leftmost bar and dotted line represent the lower band to upper band ratio (crosslinking ratio or crosslinking %) of the control (without reducing agent). Numbers in the bars are the values of the lower band to upper band ratio (crosslinking ratio or crosslinking %).

FIG. 14 shows an image of non-reducing SDS-PAGE gel (see Example 7). 20 mg/mL of the antibody was reacted by mixing with 2-MEA or TCEP in pH 3, 4, and 5 conditions. Buffer pH of each sample is described in the figure. Lanes 3, 6 and 9: without reducing agent. Lanes 4, 7 and 10: mixed with 1 mM 2-MEA. Lanes 5, 8, and 11: mixed with 0.25 mM TCEP.

FIG. 15 shows an image of non-reducing SDS-PAGE gel (see Example 7). 20 mg/mL of the antibody was reacted by mixing with 2-MEA or TCEP in pH 6, 7 and 8 conditions. Buffer pH of each sample is described in the figure. Lanes 3, 6 and 9: without reducing agent. Lanes 4, 7 and 10: mixed with 1 mM 2-MEA. Lanes 5, 8, and 11: mixed with 0.25 mM TCEP.

FIG. 16 shows a graph showing the lower band to upper band ratio (crosslinking ratio) of the antibody samples in FIGS. 14 and 15 (see Example 7). For each pH, the leftmost (white) bar represents the lower band to upper band ratio (crosslinking ratio) of the control (without reducing agent treatment). The middle (shaded) bars represent the lower band to upper band ratio (crosslinking ratio) of samples mixed with 1 mM 2-MEA. The rightmost (black) bars represent the lower band to upper band ratio (crosslinking ratio) of samples mixed with 0.25 mM TCEP. Numbers in the bars are the values of the lower band to upper band ratio (crosslinking ratio).

FIG. 17 shows a chromatogram of cation exchange chromatography performed on the OKT3.S191C antibody sample as described in Example 8-1.

FIG. 18 shows a gel image of the non-reducing SDS-PAGE analysis of the OKT3.S191C antibody sample separated by cation exchange chromatography as described in Example 8-1. Lanes 5 and 10: OKT3.S191C (non-fractionated). Lane 6: mixture of RA3 and RA4. Lane 7: mixture of RA5 and RA6. Lane 8: mixture of RA7 and RA8. Lane 9: mixture of RA9 and RA10.

FIG. 19 shows a chromatogram of cation exchange chromatography performed on the OKT3.S191C0110 antibody sample as described in Example 8-2.

FIG. 20 shows a gel image of the non-reducing SDS-PAGE analysis of the OKT3.S191C0110 antibody sample separated by cation exchange chromatography as described in Example 8-2. Lane 3: OKT3.S191C0110 (non-fractionated). Lane 4: mixture of RA4 and RA5. Lane 5: mixture of RA6 and RA7. Lane 6: mixture of RA8 and RA9. Lane 7: mixture of RA10 and RA11. Lane 8: mixture of RB11 and RB10. Lane 9: mixture of RB8 and RB7. Lane 10: mixture of RB6 and RB5. Lane 11: mixture of RB4 and RB3.

FIG. 21 depicts examples of modified antibodies in which the Fabs are crosslinked with each other as described in Reference Example 1. The figure schematically shows structural differences between a wild-type antibody (WT) and a modified antibody in which the CH1 regions of antibody H chain are crosslinked with each other (HH type), a modified antibody in which the CL regions of antibody L chain are crosslinked with each other (LL type), and a modified antibody in which the CH1 region of antibody H chain is crosslinked with the CL region of antibody L chain (HL or LH type).

FIG. 22 shows the results of assaying the CD3-mediated agonist activity of a wild-type anti-CD3 epsilon antibody molecule (CD3-G4s) and modified antibody molecules produced by linking the Fab-Fab of the wild-type molecule via an additional disulfide bond (CD3-G4sLL, CD3-G4sHH), as described in Reference Example 4-3.

FIG. 23 shows the results of assaying the CD3-mediated agonist activity of a wild-type anti-CD3 epsilon antibody molecule (OKT3-G1s) and modified antibody molecules produced by linking the Fab-Fab of the wild-type molecule via an additional disulfide bond (OKT3-G1sLL, OKT3-G1sHH), as described in Reference Example 4-3.

FIG. 24 shows the results of assaying the CD3- and/or CD28-mediated agonist activity of a wild-type anti-CD3 epsilon antibody molecule (CD3-G1s), an anti-CD28 antibody molecule (CD28-G1s), and an anti-CD3 epsilon×anti-CD28 bispecific antibody (CD3//CD28-G1s), and modified antibody molecules produced by linking the Fab-Fab of the bispecific antibody via an additional disulfide bond (CD3//CD28-G1sLL, CD3//CD28-G1sHH, CD3//CD28-G1sLH, CD3//CD28-G1sHL), as described in Reference Example 4-3.

FIG. 25 shows the results of assaying the CD3- and/or CD28-mediated agonist activity of a wild-type anti-CD3 epsilon antibody molecule (OKT3-G1s), an anti-CD28 antibody molecule (CD28-G1s), and an anti-CD3 epsilon×anti-CD28 bispecific antibody (OKT3//CD28-G1s), and modified antibody molecules produced by linking the Fab-Fab of the bispecific antibody via an additional disulfide bond (OKT3//CD28-G1sHH, OKT3//CD28-G1sHL), as described in Reference Example 4-3.

FIG. 26 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (1/8). Each protease-treated antibody was applied to non-reducing capillary electrophoresis, followed by band detection with an anti-kappa chain antibody.

FIG. 27 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (2/8). Each protease-treated antibody was applied to non-reducing capillary electrophoresis, followed by band detection with an anti-kappa chain antibody.

FIG. 28 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (3/8). Each protease-treated antibody was applied to non-reducing capillary electrophoresis, followed by band detection with an anti-kappa chain antibody.

FIG. 29 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (4/8). Each protease-treated antibody was applied to non-reducing capillary electrophoresis, followed by band detection with an anti-kappa chain antibody.

FIG. 30 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (5/8). Each protease-treated antibody was applied to non-reducing capillary electrophoresis, followed by band detection with an anti-kappa chain antibody.

FIG. 31 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (6/8). Each protease-treated antibody was applied to non-reducing capillary electrophoresis, followed by band detection with an anti-kappa chain antibody.

FIG. 32 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (7/8). Each protease-treated antibody was applied to non-reducing capillary electrophoresis, followed by band detection with an anti-kappa chain antibody.

FIG. 33 shows the results of protease treatment of an anti-IL6R antibody (MRA), modified antibodies produced by introducing a cysteine substitution into the heavy chain variable region of the anti-IL6R antibody (MRAH.xxx-G1T4), and modified antibodies produced by introducing a cysteine substitution into the heavy chain constant region of the anti-IL6R antibody (MRAH-G1T4.xxx), as described in Reference Example 5-2 (8/8). Each protease-treated antibody was applied to non-reducing capillary electrophoresis, followed by band detection with an anti-kappa chain antibody.

FIG. 34 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (1/10). Each protease-treated antibody was applied to non-reducing capillary electrophoresis, followed by band detection with an anti-kappa chain antibody.

FIG. 35 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (2/10). Each protease-treated antibody was applied to non-reducing capillary electrophoresis, followed by band detection with an anti-kappa chain antibody.

FIG. 36 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (3/10). Each protease-treated antibody was applied to non-reducing capillary electrophoresis, followed by band detection with an anti-kappa chain antibody.

FIG. 37 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (4/10). Each protease-treated antibody was applied to non-reducing capillary electrophoresis, followed by band detection with an anti-kappa chain antibody.

FIG. 38 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (5/10). Each protease-treated antibody was applied to non-reducing capillary electrophoresis, followed by band detection with an anti-kappa chain antibody.

FIG. 39 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (6/10). Each protease-treated antibody was applied to non-reducing capillary electrophoresis, followed by band detection with an anti-kappa chain antibody.

FIG. 40 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (7/10). Each protease-treated antibody was applied to non-reducing capillary electrophoresis, followed by band detection with an anti-kappa chain antibody.

FIG. 41 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (8/10). Each protease-treated antibody was applied to non-reducing capillary electrophoresis, followed by band detection with an anti-kappa chain antibody.

FIG. 42 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (9/10). Each protease-treated antibody was applied to non-reducing capillary electrophoresis, followed by band detection with an anti-kappa chain antibody.

FIG. 43 shows the results of protease treatment of an anti-IL6R antibody (MRA), modified antibodies produced by introducing a cysteine substitution into the light chain variable region of the anti-IL6R antibody (MRAL.xxx-k0), and modified antibodies produced by introducing a cysteine substitution into the light chain constant region of the anti-IL6R antibody (MRAL-k0.xxx), as described in Reference Example 6-2 (10/10). Each protease-treated antibody was applied to non-reducing capillary electrophoresis, followed by band detection with an anti-kappa chain antibody.

FIG. 44 shows the results of protease treatment of an anti-IL6R antibody (MRA) and a modified antibody produced by introducing a cysteine substitution into the light chain constant region of the anti-IL6R antibody (MRAL-k0.K126C), as described in Reference Example 7-2. Each protease-treated antibody was applied to non-reducing capillary electrophoresis, followed by band detection with an anti-kappa chain antibody or an anti-human Fc antibody.

FIG. 45 shows the correspondence between the molecular weight of each band obtained by protease treatment of the antibody sample and its putative structure, as described in Reference Example 7-2. It is also noted below the structure of each molecule whether the molecule may react with an anti-kappa chain antibody or an anti-Fc antibody (whether a band is detected in the electrophoresis of FIG. 44).

FIG. 46 shows the results of assaying the CD3-mediated agonist activity of an anti-CD3 antibody molecule (OKT3), modified antibody molecules produced by linking the Fab-Fab of that antibody molecule via an additional disulfide bond (H_T135C, H_S136C, H_S191C, and L_K126C), and an anti-KLH antibody molecule (IC17) (negative control), as described in Reference Example 13-4.

FIG. 47 shows the results of assaying the CD3-mediated agonist activity of an anti-CD3 antibody molecule (OKT3), a modified antibody molecule produced by introducing Knobs-into-Holes (KiH) modifications, which facilitate heterodimerization, into the heavy chain constant region of OKT3 (OKT3_KiH), modified antibody molecules produced by linking the Fab-Fab of that antibody molecule via an additional disulfide bond (H_S191C_KiH, H_S191C/V188C_KiH, H_S191C/P189C_KiH, H_S191C/S190C_KiH, H_S191C/S192C_KiH, H_S191C/L193C_KiH, H_S191C/G194C_KiH), and an anti-KLH antibody (IC17) (negative control), as described in Reference Example 14-4.

FIG. 48 shows the results of assaying the CD3-mediated agonist activity of an anti-CD3 antibody molecule (OKT3), a modified antibody molecule produced by linking the Fab-Fab of that antibody molecule via an additional disulfide bond (H_S191C), a modified antibody molecule produced by introducing Knobs-into-Holes (KiH) modifications, which facilitate heterodimerization, into the heavy chain constant region of OKT3 (OKT3_KiH), a modified antibody molecule produced by linking the Fab-Fab of that antibody molecule via an additional disulfide bond (H_S191C_KiH), modified antibody molecules produced by introducing a positively-charged amino acid substitution into one of the heavy chain constant regions of OKT3_KiH and introducing a negatively-charged amino acid substitution into the other heavy chain constant region (0004//0004, 0004//0006), modified antibody molecules produced by introducing a positively- or negatively-charged amino acid substitution into one of the heavy chain constant regions of OKT3_KiH (0004//OKT3, OKT3//0004, OKT3//0006), and an anti-KLH antibody molecule (IC17) (negative control), as described in Reference Example 15-4.

FIG. 49 shows the results of assaying the CD3-mediated agonist activity of an anti-CD3 antibody molecule (OKT3), modified antibody molecules produced by removing a disulfide bond in the hinge region of that antibody molecule (dh1, dh2, dh3), modified antibody molecules produced by linking the Fab-Fab of those molecules via an additional disulfide bond (H_S191C_dh1, H_S191C_dh2, H_S191C_dh3), and an anti-KLH antibody molecule (IC17) (negative control) as described in Reference Example 16-4.

FIG. 50 shows the results of assaying the CD3-mediated agonist activity of an anti-CD3 monospecific antibody molecule (OKT3-G1s), a modified antibody molecule produced by linking the Fab-Fab of that antibody molecule via an additional disulfide bond (OKT3-G1sHH), a modified antibody molecule produced by linking the Fab-Fab of an anti-CD3 monospecific antibody (CD3-G1s) via an additional disulfide bond (CD3-G1sLL), an anti-CD3 biparatopic antibody molecule (CD3//OKT3-G1s), modified antibody molecules produced by linking the Fab-Fab of that antibody molecule via an additional disulfide bond (CD3//OKT3-G1sHH, CD3//OKT3-G1sLH), and a combination of CD3-G1sLL and OKT3-G1s (CD3-G1sLL+OKT3-G1s), as described in Reference Example 20.

FIG. 51A shows the results of assaying the CD3- and/or PD1-mediated agonist activity of anti-CD3×anti-PD1 bispecific antibodies and modified antibody molecules produced by linking the Fab-Fab of those antibodies via an additional disulfide bond, as described in Reference Example 22-1. FIG. 51A shows the agonist activity of an anti-CD3×anti-PD1 bispecific antibody molecule (OKT3//117-G1silent) which is composed of an anti-CD3 antibody (OKT3) and an anti-PD1 antibody (117), and modified antibody molecules produced by linking the Fab-Fab of that antibody molecule via an additional disulfide bond (OKT3//117-G1silentHH, OKT3//117-G1silentHL, OKT3//117-G1silentLL).

FIG. 51B shows the results of assaying the CD3- and/or PD1-mediated agonist activity of anti-CD3×anti-PD1 bispecific antibodies and modified antibody molecules produced by linking the Fab-Fab of those antibodies via an additional disulfide bond, as described in Reference Example 22-1. FIG. 51B shows the agonist activity of an anti-CD3×anti-PD1 bispecific antibody molecule (OKT3//10-G1silent) which is composed of an anti-CD3 antibody (OKT3) and an anti-PD1 antibody (10), and modified antibody molecules produced by linking the Fab-Fab of that antibody molecule via an additional disulfide bond (OKT3//10-G1silentHH, OKT3//10-G1silentHL).

FIG. 51C shows the results of assaying the CD3- and/or PD1-mediated agonist activity of anti-CD3×anti-PD1 bispecific antibodies and modified antibody molecules produced by linking the Fab-Fab of those antibodies via an additional disulfide bond, as described in Reference Example 22-1. FIG. 51C shows the agonist activity of an anti-CD3×anti-PD1 bispecific antibody molecule (CD3//949-G1silent) which is composed of an anti-CD3 antibody (CD3) and an anti-PD1 antibody (949), and modified antibody molecules produced by linking the Fab-Fab of that antibody molecule via an additional disulfide bond (CD3//949-G1silentLH, CD3//949-G1silentHH, CD3//949-G1silentLL, CD3//949-G1silentHL).

FIG. 51D shows the results of assaying the CD3- and/or PD1-mediated agonist activity of anti-CD3×anti-PD1 bispecific antibodies and modified antibody molecules produced by linking the Fab-Fab of those antibodies via an additional disulfide bond, as described in Reference Example 22-1. FIG. 51D shows the agonist activity of an anti-CD3×anti-PD1 bispecific antibody molecule (OKT3//949-G1silent) which is composed of an anti-CD3 antibody (OKT3) and an anti-PD1 antibody (949), and modified antibody molecules produced by linking the Fab-Fab of that antibody molecule via an additional disulfide bond (OKT3//949-G1silentHL, OKT3//949-G1silentHH, OKT3//949-G1silentLL).

FIG. 52 shows the results of assaying the CD3- and/or PD1-mediated agonist activity of an anti-CD3×anti-PD1 bispecific antibody molecule (OKT3//949-G1silent) which is composed of an anti-CD3 antibody (OKT3) and an anti-PD1 antibody (949), and modified antibody molecules produced by linking the Fab-Fab of that antibody molecule via an additional disulfide bond (OKT3//949-G1silentHH, OKT3//949-G1silentHL, OKT3//949-G1silentLH, OKT3//949-G1silentLL), as described in Reference Example 22-2.

FIG. 53A shows the results of evaluating the T cell-dependent inhibitory effect on cancer cell growth when using a CD28/CD3 clamping bispecific antibody and a GPC3/binding-attenuated CD3 bispecific antibody in combination, as described in Reference Example 23-1. When the above-mentioned CD28/CD3 clamping bispecific antibody and GPC3/binding-attenuated CD3 bispecific antibody are combined and allowed to act in the presence of target cells (GPC3-expressing cancer cells) and effector cells (T cells), the GPC3/binding-attenuated CD3 bispecific antibody brings the target cell and the effector cell close together, and the CD28/CD3 clamping bispecific antibody activates the effector cell. FIG. 53A shows the inhibitory effect on cancer cell growth when a GPC3/binding-attenuated CD3 bispecific antibody molecule (GPC3/attCE115) was used as an antibody to target T cells to cancer cells, and a GPC3/CD3 clamping bispecific antibody molecule (GPC3/clamp CD3), a KLH/CD3 clamping bispecific antibody molecule (KLH/clamp CD3), a CD28/CD3 clamping bispecific antibody molecule (CD28/clamp CD3), or a modified antibody molecule produced by linking the Fab-Fab of that antibody molecule via an additional disulfide bond (CD28/clamp CD3_HH) was used as an antibody for activating T cells.

FIG. 53B shows, as with FIG. 53A, the results of evaluating the T cell-dependent inhibitory effect on cancer cell growth when using a CD28/CD3 clamping bispecific antibody and a GPC3/binding-attenuated CD3 bispecific antibody in combination, as described in Reference Example 23-1. FIG. 53B shows the inhibitory effect on cancer cell growth when a modified antibody molecule produced by linking the Fab-Fab of the GPC3/binding-attenuated CD3 bispecific antibody via an additional disulfide bond (GPC3/attCE115_LL) was used as an antibody to target T cells to cancer cells, and a GPC3/CD3 clamping bispecific antibody molecule (GPC3/clamp CD3), a KLH/CD3 clamping bispecific antibody molecule (KLH/clamp CD3), a CD28/CD3 clamping bispecific antibody molecule (CD28/clamp CD3), or a modified antibody molecule produced by linking the Fab-Fab of that antibody molecule via an additional disulfide bond (CD28/clamp CD3_HH) was used as an antibody for activating T cells.

FIG. 54A shows the results of evaluating cytokine production from T cells when a CD28/CD3 clamping bispecific antibody and a GPC3/binding-attenuated CD3 bispecific antibody were used in combination as described in Reference Example 23-2. When the above-mentioned CD28/CD3 clamping bispecific antibody and GPC3/binding-attenuated CD3 bispecific antibody are used in combination in the presence of target cells (GPC3-expressing cancer cells) and effector cells (T cells), the GPC3/binding-attenuated CD3 bispecific antibody brings the target cell and the effector cell close together, and the CD28/CD3 clamping bispecific antibody activates the effector cell. FIG. 54A shows the level of IL-6 production when a GPC3/binding-attenuated CD3 bispecific antibody molecule (GPC3/attCE115) and a modified antibody molecule produced by linking the Fab-Fab of the CD28/CD3 clamping bispecific antibody via an additional disulfide bond (CD28/clamp CD3_HH) were used each alone or in combination in the presence of target cells (GPC3-expressing cancer cells) and effector cells (T cells).

FIG. 54B shows, as with FIG. 54A, the results of evaluating cytokine production from T cells when a CD28/CD3 clamping bispecific antibody and a GPC3/binding-attenuated CD3 bispecific antibody were used in combination as described in Reference Example 23-2. FIG. 54B shows the level of IL-6 production when a GPC3/binding-attenuated CD3 bispecific antibody molecule (GPC3/attCE115) and a modified antibody molecule produced by linking the Fab-Fab of the CD28/CD3 clamping bispecific antibody via an additional disulfide bond (CD28/clamp CD3_HH) were used each alone or in combination in the presence of effector cells (T cells) only.

FIG. 54C shows, as with FIG. 54A, the results of evaluating cytokine production from T cells when a CD28/CD3 clamping bispecific antibody and a GPC3/binding-attenuated CD3 bispecific antibody were used in combination as described in Reference Example 23-2. FIG. 54C shows the cancer cell growth inhibitory effect when a GPC3/binding-attenuated CD3 bispecific antibody molecule (GPC3/attCE115) and a modified antibody molecule produced by linking the Fab-Fab of the CD28/CD3 clamping bispecific antibody via an additional disulfide bond (CD28/clamp CD3_HH) were used each alone or in combination in the presence of target cells (GPC3-expressing cancer cells) and effector cells (T cells).

FIG. 55A is a schematic diagram showing the mechanism of action of the T cell-dependent cancer cell growth inhibition when a CD28/CD3 clamping bispecific antibody and a GPC3/binding-attenuated CD3 bispecific antibody are used in combination, as described in Reference Examples 23-1 (“epsilon” in the diagrams indicates CD3 epsilon). FIG. 55A shows the mechanism of action of the cancer cell growth inhibition when a CD28/CD3 clamping bispecific antibody and a GPC3/binding-attenuated CD3 bispecific antibody are used in combination in the presence of target cells (GPC3-expressing cancer cells) and effector cells (T cells).

FIG. 55B is a schematic diagram showing the mechanism of action of the T cell-dependent cancer cell growth inhibition when a CD28/CD3 clamping bispecific antibody and a GPC3/binding-attenuated CD3 bispecific antibody are used in combination, as described in Reference Examples 23-1 (epsilon in the diagrams indicates CD3 epsilon). FIG. 55B shows the mechanism of action of the cancer cell growth inhibition when a modified antibody molecule which has been modified to introduce an additional disulfide bond into the Fab-Fab of a CD28/CD3 clamping bispecific antibody, and a GPC3/binding-attenuated CD3 bispecific antibody, are used in combination in the presence of target cells (GPC3-expressing cancer cells) and effector cells (T cells).

FIG. 56A is a schematic diagram showing the mechanism of action of the cytokine production from T cells when a CD28/CD3 clamping bispecific antibody and a GPC3/binding-attenuated CD3 bispecific antibody are used in combination, as described in Reference Examples 23-2 (epsilon in the diagrams indicates CD3 epsilon). FIG. 56A shows the mechanism of action of the cytokine production when a modified antibody molecule which has been modified to introduce an additional disulfide bond into the Fab-Fab of a CD28/CD3 clamping bispecific antibody, and a GPC3/binding-attenuated CD3 bispecific antibody, are used in combination in the presence of target cells (GPC3-expressing cancer cells) and effector cells (T cells).

FIG. 56B is a schematic diagram showing the mechanism of action of the cytokine production from T cells when a CD28/CD3 clamping bispecific antibody and a GPC3/binding-attenuated CD3 bispecific antibody are used in combination, as described in Reference Examples 23-2 (epsilon in the diagrams indicates CD3 epsilon). FIG. 56B shows the mechanism of action of the cytokine production when a modified antibody molecule which has been modified to introduce an additional disulfide bond into the Fab-Fab of a CD28/CD3 clamping bispecific antibody, and a GPC3/binding-attenuated CD3 bispecific antibody, are used in combination in the presence of effector cells (T cells) only.

FIG. 57A shows the results of assaying the agonist activity of a CD8/CD28 bispecific antibody molecule (CD8/CD28-P587), and modified antibody molecules produced by linking the Fab-Fab of that antibody via an additional disulfide bond (CD8/CD28-P587(HH), CD8/CD28-P587(LL), CD8/CD28-P587(HL), CD8/CD28-P587(LH)) as described in Reference Example 24. An anti-KLH antibody molecule (KLH-P587) was used as a negative control. The results obtained by using peripheral blood mononuclear cells (PBMC) from two different donors are shown (upper panel: donor A, lower panel: donor B). FIG. 57A shows the proportion of divided regulatory T cells (Treg) in PBMCs.

FIG. 57B shows the results of assaying the agonist activity of a CD8/CD28 bispecific antibody molecule (CD8/CD28-P587), and modified antibody molecules produced by linking the Fab-Fab of that antibody via an additional disulfide bond (CD8/CD28-P587(HH), CD8/CD28-P587(LL), CD8/CD28-P587(HL), CD8/CD28-P587(LH)) as described in Reference Example 24. FIG. 57B shows the proportion of divided CD8 alpha-positive T cells in PBMCs.

FIG. 58 shows chromatograms of cation exchange chromatography (CIEX) performed on the antibody sample of OKT3 variants with charged amino acid substitution as described in Example 9-3.

FIG. 59 shows chromatograms of cation exchange chromatography (CIEX) performed on the antibody sample of OKT3 variants with charged amino acid substitution as described in Examples 2-2 and Examples 9-3.

FIG. 60 shows a scatter diagram of lower band-to-upper band ratio (non-reducing SDS-PAGE gel image) of OKT3 and MRA antibody variants produced in Example 10-1. Y-axis represents the ratio of the lower band to upper band of MRA variants sample as shown in Table 87, whereas X-axis represents the ratio of the lower band to upper band of OKT3 variants sample as shown in Table 87.

FIG. 61A shows chromatograms of cation exchange chromatography (CIEX) performed on the antibody sample of OKT3 variants with charged amino acid substitution as described in Example 10-3.

FIG. 61B shows chromatograms of cation exchange chromatography (CIEX) performed on the antibody sample of MRA variants with charged amino acid substitution as described in Example 10-3.

FIG. 62A is a schematic diagram showing the effect of additional amino acid mutation for enhancement of Fab crosslinking of the engineered disulfide bond. (Left) G1T4.S191C variant with cysteine substitution e.g. at the S191C of CH1 (EU numbering) contain mixtures of cross-linked and non-cross-linked antibodies. (Middle) G1T4.S191C variants which comprise additional amino acid mutation X (X can be either charged amino acid, hydrophobic amino acid or Knob-hole amino acids) shows higher proportion of cross-linked antibodies. (Right) Amino acid position at CH1-CH1 interface (EU numbering) in which additional amino acid mutation X (X can be either charged amino acid, hydrophobic amino acid or Knob-hole amino acids) can facilitate the crosslinking of the engineered disulfide bond.

FIG. 62B is a schematic diagram showing the effect of additional mutation for separation between crosslinked and non-crosslinked Fabs by chromatography methods such as CIEX.

### DESCRIPTION OF EMBODIMENTS

### I. Definitions

Herein, the term “antigen-binding molecule” refers, in its broadest sense, to a molecule that specifically binds to an antigenic determinant (epitope). In one embodiment, the antigen-binding molecule is an antibody, antibody fragment, or antibody derivative. In one embodiment, the antigen-binding molecule is a non-antibody protein, or a fragment thereof, or a derivative thereof.

Herein, “antigen-binding domain” refers to a region that specifically binds and is complementary to the whole or a portion of an antigen. Herein, an antigen-binding molecule comprises an antigen-binding domain. When the molecular weight of an antigen is large, an antigen-binding domain can only bind to a particular portion of the antigen. The particular portion is called “epitope”. In one embodiment, an antigen-binding domain comprises an antibody fragment which binds to a particular antigen. An antigen-binding domain can be provided from one or more antibody variable domains. In a non-limiting embodiment, the antigen-binding domains comprise both the antibody light chain variable region (VL) and antibody heavy chain variable region (VH). Examples of such antigen-binding domains include “single-chain Fv (scFv)”, “single-chain antibody”, “Fv”, “single-chain Fv2 (scFv2)”, “Fab”, and “Fab′”. In other embodiments, an antigen-binding domain comprises a non-antibody protein which binds to a particular antigen, or a fragment thereof. In a specific embodiment, an antigen-binding domain comprises a hinge region.

In the present invention, “specifically binds” means binding in a state where one of the molecules involved in specific binding does not show any significant binding to molecules other than a single or a number of binding partner molecules. Furthermore, it is also used when an antigen-binding domain is specific to a particular epitope among multiple epitopes contained in an antigen. When an epitope bound by an antigen-binding domain is contained in multiple different antigens, antigen-binding molecules comprising the antigen-binding domain can bind to various antigens that have the epitope.

In the present disclosure, the recitation “binds to the same epitope” means that the epitopes to which two antigen-binding domains bind at least partially overlap each other. The degree of the overlap is, but not limited to, at least 10% or more, preferably 20% or more, 30% or more, 40% or more, 50% or more, 60% or more, 70% or more, and 80% or more, particularly preferably 90% or more, and most preferably 100%.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

In one embodiment of the present invention, constant regions are preferably antibody constant regions, more preferably IgG1, IgG2, IgG3, and IgG4-type antibody constant regions, and even more preferably human IgG1, IgG2, IgG3, and IgG4-type antibody constant regions. Furthermore, in another embodiment of the present invention, constant regions are preferably heavy chain constant regions, more preferably IgG1, IgG2, IgG3, and IgG4-type heavy chain constant regions, and even more preferably human IgG1, IgG2, IgG3, and IgG4-type heavy chain constant regions. The amino acid sequences of the human IgG1 constant region, the human IgG2 constant region, the human IgG3 constant region, and the human IgG4 constant region are known. For the constant regions of human IgG1, human IgG2, human IgG3, and human IgG4, a plurality of allotype sequences with genetic polymorphism are described in Sequences of proteins of immunological interest, NIH Publication No. 91-3242, and any of them can be used in the present invention. Amino acid-modified constant regions of the present invention may contain other amino acid mutations or modifications, as long as they include an amino acid mutation of the present invention.

The term “hinge region” denotes an antibody heavy chain polypeptide portion in a wild-type antibody heavy chain that joins the CH1 domain and the CH2 domain, e.g., from about position 216 to about position 230 according to the EU numbering system, or from about position 226 to about position 243 according to the Kabat numbering system. It is known that in a native IgG antibody, cysteine residue at position 220 according to EU numbering in the hinge region forms a disulfide bond with cysteine residue at position 214 in the antibody light chain. It is also known that between the two antibody heavy chains, disulfide bonds are formed between cysteine residues at position 226 and between cysteine residues at position 229 according to EU numbering in the hinge region. In general, a “hinge region” is defined as extending from human IgG1 from 216 to 238 (EU numbering) or from 226 to 251 (Kabat numbering). This hinge can be further divided into three different regions, an upper hinge, a central hinge and a lower hinge. In human IgG1 antibodies, these regions are generally defined as follows:
Upper hinge: 216-225 (EU numbering) or 226-238 (Kabat numbering),
Central hinge: 226-230 (EU numbering) or 239-243 (Kabat numbering),
Lower hinge: 231-238 (EU numbering) or 244-251 (Kabat numbering).

The hinge region of other IgG isotypes can be aligned with the IgG1 sequence by placing the first and last cysteine residues that form an interheavy chain SS bond in the same position (e.g., Brekke et al., 1995, Immunol (See Table 1 of Today 16: 85-90). A hinge region herein includes wild-type hinge regions as well as variants in which amino acid residue(s) in a wild-type hinge region is altered by substitution, addition, or deletion.

The term “disulfide bond formed between amino acids which are not in a hinge region” (or “disulfide bond formed between amino acids outside of a hinge region”) means disulfide bond formed, connected or linked through amino acids located in any antibody region which is outside of the “hinge region” defined above. For example, such disulfide bond is formed, connected or linked through amino acids in any position in an antibody other than in a hinge region (e.g., from about position 216 to about position 230 according to the EU numbering system, or from about position 226 to about position 243 according to the Kabat numbering system). In some embodiments, such disulfide bond is formed, connected or linked through amino acids located in a CH1 region, a CL region, a VL region, a VH region and/or a VHH region. In some embodiments, such disulfide bond is formed, connected or linked through amino acids located in positions 119 to 123, 131 to 140, 148 to 150, 155 to 167, 174 to 178, 188 to 197, 201 to 214, according to EU numbering, in the CH1 region. In some embodiments, such disulfide bond is formed, connected or linked through amino acids located in positions 119, 122, 123, 131, 132, 133, 134, 135, 136, 137, 138, 139, 140, 148, 150, 155, 156, 157, 159, 160, 161, 162, 163, 164, 165, 167, 174, 176, 177, 178, 188, 189, 190, 191, 192, 193, 194, 195, 196, 197, 201, 203, 205, 206, 207, 208, 211, 212, 213, 214 according to EU numbering, in the CH1 region. In some embodiments, such disulfide bond is formed, connected or linked through amino acids located in positions 188, 189, 190, 191, 192, 193, 194, 195, 196, and 197, according to EU numbering, in the CH1 region. In one preferred embodiment, such disulfide bond is formed, connected or linked through amino acids located in position 191, according to EU numbering, in the CH1 region.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); single chain Fabs (scFabs); single domain antibodies; and multispecific antibodies formed from antibody fragments.

By “contacting” is meant subjecting to, exposing to, in solution. The antibody, protein or polypeptide can be contacted with the reducing reagents while also bound to a solid support (e.g., an affinity column or a chromatography matrix). Preferably, the solution is buffered. In order to maximize the yield of antibody/protein with a desired conformation, the pH of the solution is chosen to protect the stability of the antibody/protein and to be optimal for disulfide exchange. In the practice of the invention, the pH of the solution is preferably not strongly acidic. Thus, some pH ranges are greater than pH 5, preferably about pH 6 to about pH 11, more preferably from about pH 7 to about pH 10, and still more preferably from about pH 6 to about pH 8. In one non-limiting embodiment of the invention, the optimal pH was found to be about pH 7. However, the optimal pH for a particular embodiment of the invention can be easily determined experimentally by those skilled in the art.

The term “reduction reagent” and “reducing agent” is used interchangeably. In some embodiments, said reducing agents are free thiols. The reducing reagent is preferably comprised of a compound from the group consisting of glutathione (GSH), dithiothreitol (DTT), 2-mercaptoethanol, 2-aminoethanethiol (2-MEA), TCEP (tris(2-carboxyethyl)phosphine), dithionitrobenzoate, cysteine and Na₂SO₃. In some embodiments, TCEP, 2-MEA, DTT, cysteine, GSH or Na₂SO₃ can be used. In some preferred embodiments, 2-MEA can be used. In some preferred embodiments, TCEP can be used.

The reducing agent may be added to the fermentation media in which the cells producing the recombinant protein are grown. In additional embodiments, the reducing agent also may be added to the LC mobile phase during the LC separation step for separating the recombinant protein. In certain embodiments, the protein is immobilized to a stationary phase of the LC column and the reducing agents are part of the mobile phase. In specific embodiments, the untreated IgG antibody may elute as a heterogeneous mixture as indicated by the number of peaks. The use of the reduction/oxidation coupling reagent produces a simpler and more uniform peak pattern. It is contemplated that this more uniform peak of interest may be isolated as a more homogeneous preparation of the IgG.

The reducing agent is present at a concentration that is sufficient to increase the relative proportion of the desired conformation (e.g., the “paired cysteines” form of an antibody which has one or more engineered disulfide bond(s) formed between the two Fabs of the antibody, e.g., between amino acid residues which are not in the hinge region). The optimal absolute concentration and molar ratio of the reducing agent depends upon the concentration of total IgG and in some circumstances the specific IgG subclass. When used for preparing IgG1 molecules it also will depend on the number and accessibility of the unpaired cysteines in the protein. Generally, the concentration of free thiols from the reducing agent can be from about 0.05 mM to about 100 mM, more preferably about 0.1 mM to about 50 mM, and still more preferably about 0.2 mM to about 20 mM. In some preferred embodiments, the concentration of the reducing agent is 0.01, 0.05, 0.1, 0.25, 0.5, 1, 2.5, 5, 10, 25, 50, 100 mM. In some preferred embodiments, 0.05 mM to 1 mM of 2-MEA can be used. In some preferred embodiments, 0.01 mM to 25 mM TCEP can be used.

Contacting the preparation of recombinant protein with a reducing agent is performed for a time sufficient to increase the relative proportion of the desired conformation. Any relative increase in proportion is desirable, including for, example, at least 10%, 20%, 30%, 40%, 50%, 60%, 70% and even 80% or 90% of the protein with an undesired conformation is converted to protein with the desired conformation. The contacting may be performed by providing the reducing agent to the fermentation medium in which the protein is being generated. Alternatively, the contacting takes place upon partial purification of the protein from the cell culture in which it is generated. In still other embodiments, the contacting is performed after the protein has been eluted from the chromatography column but before any further processing. Essentially, the contacting may be performed at any stage during preparation, purification, storage or formulation of the antibody. In some embodiments, partial purification by affinity chromatography (e.g., Protein A chromatography) may be conducted prior to the contacting.

The contacting may be also performed with antibodies attached to a stationary phase of a chromatographic columns, while the reducing agent are a part of the mobile phase; In this case the contacting may be performed as a part of chromatographic purification procedure. Examples of representative chromatographic refolding processes may include size exclusion (SEC); solvent exchange during reversible adsorption on protein A column; hydrophobic interaction chromatography (HIC); immobilized metal affinity chromatography (IMAC); reversed-phase chromatography (RPC); use of immobilized folding catalyst, such as GroE1, GroES or other proteins with folding properties. The on-column refolding is attractive because it is easily automated using commercially available preparative chromatographic systems. The refolding on column of recombinant proteins produced in microbial cell was recently reviewed in (Li et al., 2004).

If the contacting step is performed on a partially or highly purified preparation of recombinant protein, the contacting step can be performed for as short as about 1 hour to about 4 hours, and as long as about 6 hours to about 4 days. It has been found that a contacting step of about 2 to about 48 hours, or about 16 hours works well. The contacting step can also take place during another step, such as on a solid phase or during filtering or any other step in purification.

The methods of the invention can be performed over a wide temperature range. For example, the methods of the invention have been successfully carried out at temperatures from about 4 degrees Celsius (“degrees C.”) to about 37 degrees C., however the best results were achieved at lower temperatures. A typical temperature for contacting a partially or fully purified preparation of the recombinant protein is about 4 degrees C. to about 25 degrees C. (ambient), or preferably at 23 degrees C., but can also be performed at lower temperatures and at higher temperature.

In addition, it is contemplated that the method may be performed at high pressure. Previously, high hydrostatic pressures (1000-2000 bar), combined with low, nondenaturing concentrations of guanidine hydrochloride below 1M has been used to disaggregate (solubilize) and refold several denatured proteins produced by E-coli as inclusion bodies that included human growth hormone and lysozyme, and b-lactamase (St John et al., Proc Natl Acad Sci USA, 96:13029-13033 (1999)). B-lactamase was refolded at high yields of active protein, even without added GdmHCl. In another study (Seefeldt et al., Protein Sci, 13:2639-2650 (2004)), the refolding yield of mammalian cell produced protein bikunin obtained with high pressure modulated refolding at 2000 bas was 70% by RP HPLC, significantly higher than the value of 55% (by RP-HPLC) obtained with traditional guanidine hydrochloride “dilution-refolding”. These findings indicate that high hydrostatic pressure facilitates disruption of inter- and intra-molecular interactions, leading to protein unfolding and disaggregation. The interaction of the high pressure on protein is similar to the interaction of proteins with chaotropic agents. Thus, it is contemplated that in the methods of the invention, instead of using chaotropic agents, high pressure is used for protein unfolding. Of course, a combination of high pressure and chaotropic agents also may be used in some instances.

The preparation of recombinant antibody/protein can be contacted with the reducing agent in various volumes as appropriate. For example, the methods of the invention have been carried out successfully at the analytical laboratory-scale (1-50 mL), preparative-scale (50 mL-10 L) and manufacturing-scale (10 L or more). The methods of the invention can be carried out on both small and large scale with reproducibility. As such, the concentration of antibody may be an industrial quantity (in terms of weight in grams) (e.g., an industrial amount of a specific IgG) or alternatively may be in milligram quantities. In specific embodiments, the concentration of the recombinant antibody in the reaction mixture is from about 1 mg/ml and about 50 mg/ml, more specifically, 10 mg/ml, 15 mg/ml or 20 mg/ml. The recombinant IgG1 molecules in these concentrations are particularly contemplated.

In certain embodiments, the proteins produced using media contain reducing agent are further processed in a separate processing step which employs chaotropic denaturants such as, for example, sodium dodecyl sulfate (SDS), urea or guanidium hydrochloride (GuHCl). Significant amounts of chaotropic agents are needed to observe perceptible unfolding. In some embodiments the processing step uses between 0.1M and 2 M chaotrope that produces an effect equivalent to the use of 0.1 M to 2M guanidine hydrochloride. In a specific embodiment, the oxidative refolding is achieved in the presence of approximately 1.0 M guanidine hydrochloride or an amount of other chaotropic agent that produces the same or similar amount of refolding as 1M guanidine hydrochloride. In some embodiments, the methods use between about 1.5 M and 0.5 M chaotrope. The amount of chaotropic agent used is based on the structural stability of the protein in the presence of the said chaotrope. One needs to have enough chaotrope present to perturb the local tertiary structure and/or quaternary structure of domain interactions of the protein, but less than that required to fully unfold secondary structure of the molecule and/or individual domains. To determine the point at which a protein will start to unfold by equilibrium denaturation, one practiced in the art would titrate a chaotrope into a solution containing the protein and monitor structure by a technique such as circular dichroism or fluorescence. There are other parameters that could be used to unfold or slightly perturb the structure of a protein that may be used instead of a chaotrope. Temperature and pressure are two fundamental parameters that have been previously used to alter the structure of a protein and may be used in place of a chaotropic agent while contacting with a redox agent. The inventors contemplate that any parameter that has been shown to denature or perturb a protein structure may be used by a person practiced in the art in place of a chaotropic agent.

Disulfide exchange can be quenched in any way known to those of skill in the art. For example, the reducing agent can be removed or its concentration can be reduced through a purification step, and/or it can be chemically inactivated by, e.g., acidifying the solution. Typically, when the reaction is quenched by acidification, the pH of the solution containing the reducing agent will be brought down below pH 7. In some embodiment, the pH is brought to below pH 6. Generally, the pH is reduced to between about pH 2 and about pH 6.

In some embodiments, removing the reducing agent may be conducted by dialysis, buffer exchange or any chromatography method described herein.

The term by “preferentially enriched (or increased)” means an increase in relative abundance of a desired form, or increase in relative proportion of a desired form, or increase the population of a desired form (structural isoform). In some embodiments, the methods described herein increase relative abundance of an antibody structural isoform such as an antibody having at least one disulfide bond formed between amino acid residues outside of the hinge region. In one embodiment, said at least one disulfide bond is formed between the amino acid residues at position 191 according to EU numbering in the respective CH1 regions of the first antigen-binding domain and the second antigen-binding domain. In certain embodiment, said methods produce a homogenous antibody preparation having at least 50%, 60%, 70%, 80%, 90%, preferably at least 95% molar ratio of said antibody having at least one disulfide bond formed outside of the hinge region.

A “homogeneous” population of an antibody means an antibody population that comprises largely a single form of the antibody, for example, at least 50%, 60%, 70%, 80% or more, preferably at least 90%, 95%, 96%, 97%, 99% or 100% of the antibody in the solution or composition is in the properly folded form. Similarly, a “homogeneous” population of an antibody having at least one disulfide bond formed outside of the hinge region means a population of said antibody which comprises largely a single, properly folded form, for example, at least 50%, 60%, 70%, 80% or more, preferably at least 90%, 95%, 96%, 97%, 99% or 100% molar ratio of said antibody having at least one disulfide bond formed outside of the hinge region. In one preferred embodiment, said “homogeneous” population of an antibody comprises at least one disulfide bond which is formed between the amino acid residues at position 191 according to EU numbering in the respective CH1 regions of the first antigen-binding domain and the second antigen-binding domain (i.e. “paired cysteines” at the position 191 according to EU number in the CH1 region).

In preferred embodiments, the methods of the present invention produce a homogeneous antibody population or a homogeneous antibody preparation by the steps described herein.

Determining whether an antibody population is homogenous, and the relative abundance or proportions of a conformation of a protein/antibody in a mixture, can be done using any of a variety of analytical and/or qualitative techniques. If the two conformations resolve differently during separation techniques such as chromatography, electrophoresis, filtering or other purification technique, then the relative proportion of a conformation in the mixture can be determined using such purification techniques. For example, at least two different conformations of the recombinant IgG could be resolved by way of hydrophobic interaction chromatography. Further, since far UV Circular Dichroism has been used to estimate secondary structure composition of proteins (Perczel et al., 1991, Protein Engrg. 4:669-679), such a technique can determine whether alternative conformations of a protein are present. Still another technique used to determine conformation is fluorescence spectroscopy which can be employed to ascertain complementary differences in tertiary structure assignable to tryptophan and tyrosine fluorescence. Other techniques that can be used to determine differences in conformation and, hence, the relative proportions of a conformation, are on-line SEC to measure aggregation status, differential scanning calorimetry to measure melting transitions (Tm's) and component enthalpies, and chaotrope unfolding. Yet another technique that can be used to determine differences in conformation and, hence, the relative proportions of a conformation is LC/MS detection to determine the heterogeneity of the protein.

Alternatively, if there is a difference in activity between the conformations of the antibody/protein, determining the relative proportion of a conformation in the mixture can be done by way of an activity assay (e.g., binding to a ligand, enzymatic activity, biological activity, etc.). Biological activity of the protein also could be used. Alternatively, the binding assays can be used in which the activity is expressed as activity units/mg of protein.

In some embodiments described in detail herein below, the invention uses IEC chromatography, to determine the heterogeneity of the antibody/protein. In such a case, the antibody is purified or considered to be “homogenous”, which means that no polypeptide peaks or fractions corresponding to other polypeptides are detectable upon analysis by IEC chromatography. In certain embodiments, the antibody is purified or considered to be “homogenous” such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, and/or (if the polypeptide is radiolabeled) by auto radiography.

Herein, examples of conditions of SDS-PAGE analysis are as follows. Sample Buffer Solution without 2-mercaptoethanol (×4) may be used for preparation of electrophoresis samples. The samples may be treated for 10 minutes under the condition of specimen concentration 50 or 100 microgram/mL and 70 degrees C., and then subjected to non-reducing SDS-PAGE. In non-reducing SDS-PAGE, electrophoresis may be carried out for 90 minutes at 125 V, using a 4% SDS-PAGE gel. Then, the gel may be stained with CBB, and the gel image may be captured, and the bands may be quantified using an imaging device. In the gel image, several, for example, two bands, i.e., “upper band” and “lower band”, may be observed for an antibody variant sample. In this case, the molecular weight of the upper band may correspond to that of the parent antibody (before modification). Structural changes such as crosslinking via disulfide bonds of Fabs may be caused by cysteine substitution, which may result in the change in electrophoretic mobility. In this case, the lower band may be considered to correspond to the antibody having one or more engineered disulfide bond(s) formed between the CH1 regions. Antibody variant samples with additional cysteine substitutions may show a higher lower band to upper band ratio, compared to control samples. Additional cysteine substitutions may enhance/promote disulfide bond crosslinking of Fabs; and may increase the percentage or structural homogeneity of an antibody preparation having an engineered disulfide bond formed at a mutated position; and may decrease the percentage of an antibody preparation having no engineered disulfide bond formed at the mutated position. Herein, the term “lower band to upper band ratio” refers to a ratio between the quantities/intensities of the lower and upper bands that may be quantified during the above-mentioned SDS-PAGE experiments.

### Variable Fragment (Fv)

Herein, the term “variable fragment (Fv)” refers to the minimum unit of an antibody-derived antigen-binding domain that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)2

Herein, the terms “scFv”, “single-chain antibody”, and “sc(Fv)2” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an antigen-binding domain in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)2 is a single-chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)2 preferably includes, for example, a bispecific sc(Fv)2 that recognizes two epitopes present in a single antigen as disclosed in the Journal of Immunology (1994) 152(11), 5368-5374. sc(Fv)2 can be produced by methods known to those skilled in the art. For example, sc(Fv)2 can be produced by linking scFv by a linker such as a peptide linker.

Herein, the forms of an antigen-binding domain forming an sc(Fv)2 include an antibody in which the two VH units and two VL units are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Example order of the form is listed below.
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

### Fab, F(ab′)2, and Fab′

“Fab” consists of a single light chain, and a CH1 region and variable region from a single heavy chain. The heavy chain of a wild-type Fab molecule cannot form disulfide bonds with another heavy chain molecule. Herein, in addition to wild-type Fab molecules, Fab variants in which amino acid residue(s) in a wild-type Fab molecule is altered by substitution, addition, or deletion are also included. In a specific embodiment, mutated amino acid residue(s) comprised in Fab variants (e.g., cysteine residue(s) or lysine residue(s) after substitution, addition, or insertion) can form disulfide bond(s) with another heavy chain molecule or a portion thereof (e.g., Fab molecule).

scFab is an antigen-binding domain in which a single light chain, and a CH1 region and variable region from a single heavy chain which form Fab are linked together by a peptide linker. The light chain, and the CH1 region and variable region from the heavy chain can be retained in close proximity by the peptide linker.

“F(ab′)2” or “Fab” is produced by treating an immunoglobulin (monoclonal antibody) with a protease such as pepsin and papain, and refers to an antibody fragment generated by digesting an immunoglobulin (monoclonal antibody) at near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)2” consists of two light chains and two heavy chains comprising the constant region of a CH1 domain and a portion of CH2 domains so that disulfide bonds are formed between the two heavy chains. The F(ab′)2 disclosed herein can be preferably produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)2 under an appropriate setup enzyme reaction condition such as pH. Such proteases include, for example, pepsin and ficin.

### Single Domain Antibodies

Herein, those referred to by the term “single domain antibodies” are not particularly limited in their structure, as long as the domain can exert antigen-binding activity by itself. Ordinary antibodies exemplified by IgG antibodies exert antigen-binding activity in a state where a variable region is formed by the pairing of VH and VL. In contrast, a single domain antibody is known to be able to exert antigen-binding activity by its own domain structure alone without pairing with another domain. Single domain antibodies usually have a relatively low molecular weight and exist in the form of a monomer.

Examples of a single domain antibody include, but are not limited to, antigen binding molecules which naturally lack light chains, such as VHH of Camelidae animals and V_{NAR} of sharks, and antibody fragments comprising the whole or a portion of an antibody VH domain or the whole or a portion of an antibody VL domain. Examples of a single domain antibody which is an antibody fragment comprising the whole or a portion of an antibody VH/VL domain include, but are not limited to, artificially prepared single domain antibodies originating from a human antibody VH or a human antibody VL as described, e.g., in U.S. Pat. No. 6,248,516 B1. In some embodiments of the present invention, one single domain antibody has three CDRs (CDR1, CDR2, and CDR3).

Single domain antibodies can be obtained from animals capable of producing single domain antibodies or by immunizing animals capable of producing single domain antibodies. Examples of animals capable of producing single domain antibodies include, but are not limited to, camelids and transgenic animals into which gene(s) for the capability of producing a single domain antibody has been introduced. Camelids include camel, llama, alpaca, dromedary, guanaco, and such. Examples of a transgenic animal into which gene(s) for the capability of producing a single domain antibody has been introduced include, but are not limited to, the transgenic animals described in International Publication No. WO2015/143414 or US Patent Publication No. US2011/0123527 A1. Humanized single chain antibodies can also be obtained, by replacing framework sequences of a single domain antibody obtained from an animal with human germline sequences or sequences similar thereto. A humanized single domain antibody (e.g., humanized VHH) is one embodiment of the single domain antibody of the present invention.

Alternatively, single domain antibodies can be obtained from polypeptide libraries containing single domain antibodies by ELISA, panning, and such. Examples of polypeptide libraries containing single domain antibodies include, but are not limited to, naive antibody libraries obtained from various animals or humans (e.g., Methods in Molecular Biology 2012 911 (65-78) and Biochimica et Biophysica Acta—Proteins and Proteomics 2006 1764:8 (1307-1319)), antibody libraries obtained by immunizing various animals (e.g., Journal of Applied Microbiology 2014 117:2 (528-536)), and synthetic antibody libraries prepared from antibody genes of various animals or humans (e.g., Journal of Biomolecular Screening 2016 21:1 (35-43), Journal of Biological Chemistry 2016 291:24 (12641-12657), and AIDS 2016 30:11 (1691-1701)).

“Binding activity” refers to the strength of the sum total of noncovalent interactions between one or more binding sites of a molecule (e.g., an antibody) and its binding partner (e.g., an antigen). Herein, binding activity is not strictly limited to a 1:1 interaction between members of a binding pair (e.g., antibody and antigen). For example, when the members of a binding pair reflect a monovalent 1:1 interaction, the binding activity refers to the intrinsic binding affinity (affinity). When a member of a binding pair is capable of both monovalent binding and multivalent binding, the binding activity is the sum of each binding strength. The binding activity of a molecule X for its partner Y can generally be represented by the dissociation constant (KD) or “amount of bound analyte per unit amount of ligand”. Binding activity can be measured by common methods known in the art, including those described herein.

An “agonist” antigen-binding molecule or “agonist” antibody, as used herein, is an antigen-binding molecule or antibody which significantly potentiates a biological activity of the antigen it binds.

A “blocking” antigen-binding molecule or “blocking” antibody, or an “antagonist” antigen-binding molecule or “antagonist” antibody, as used herein, is an antigen-binding molecule or antibody which significantly inhibits (either partially or completely) a biological activity of the antigen it binds.

The phrase “substantially reduced” or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., KD values).

The term “substantially similar” or “substantially the same,” as used herein, refers to a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., KD values).

The terms “pharmaceutical formulation” and “pharmaceutical composition” refer to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### I. Antigen-Binding Molecule

I In an aspect, the present disclosure is partly based on the discovery that various activities of an antigen-binding molecule that contains a first antigen-binding domain and a second antigen-binding domain in which the antigen-binding domains are linked with each other via one or more bonds, are enhanced or diminished compared to a control antigen-binding molecule containing antigen-binding domains without the linkage or linked via less bonds. In certain embodiments, an antigen-binding molecule that has activity of holding two or more antigen molecules at spatially close positions is provided. The antigen-binding molecule of the present disclosure is useful, for example, in that it can regulate the activation of two antigen molecules which are activated by association with each other. In certain other embodiments, an antigen-binding molecule that has acquired resistance to protease digestion by the linkage between the antigen-binding domains is provided.

### A. Exemplary Antigen-Binding Molecules

### <Structures of Antigen-Binding Molecules>

In an aspect, the present disclosure provides an antigen-binding molecule comprising a first antigen-binding domain and a second antigen-binding domain, and the antigen-binding domains are linked with each other via one or more bonds.

In an embodiment of the above aspects, at least one of the one or more bonds linking the two antigen-binding domains is a covalent bond. In certain embodiments, the covalent bond is formed by direct crosslinking of an amino acid residue in the first antigen-binding domain and an amino acid residue in the second antigen-binding domain. The crosslinked amino acid residues are, for example, cysteine, and the formed covalent bond is, for example, a disulfide bond.

In certain other embodiments, the covalent bond is formed by crosslinking of an amino acid residue in the first antigen-binding domain and an amino acid residue in the second antigen-binding domain via a crosslinking agent. The crosslinking agent is, for example, an amine-reactive crosslinking agent, and the crosslinked amino acid residues are, for example, lysine.

In an embodiment of the above aspects, at least one of the one or more bonds linking the antigen-binding domains is a noncovalent bond. In certain embodiments, the noncovalent bond is an ionic bond, hydrogen bond, or hydrophobic bond. The ionic bond is formed, for example, between an acidic amino acid and a basic amino acid. The acidic amino acid is, for example, aspartic acid (Asp) or glutamic acid (Glu). The basic amino acid is, for example, histidine (His), lysine (Lys), or arginine (Arg).

Amino acid residues from which the bonds between the antigen-binding domains (the bonds which link two antigen-binding domains) originate are respectively present in the first and second antigen-binding domains, and the bonds between the antigen-binding domains are formed by linking these amino acid residues. In an embodiment of the above aspects, at least one of the amino acid residues from which the bond between the antigen-binding domains originates is an artificially introduced mutated amino acid residue and, for example, it is an artificially introduced cysteine residue. Such a mutated amino acid residue can be introduced into a wild-type antigen-binding domain by, for example, a method of amino acid substitution. The present specification discloses the sites of amino acid residues from which the bond between the antigen-binding domains can originate for each of the CH1, CL, and hinge regions as constant regions and the VH, VL, and VHH regions as variable regions when the antigen-binding domains comprise, for example, an antibody fragment, and for example, cysteine residues can be introduced into such sites.

In an embodiment of the above aspects, at least one of the first and second antigen-binding domains has, by itself, activity of binding to an antigen (i.e., a single antigen-binding domain independently has antigen-binding activity). In certain embodiments, each of the first and second antigen-binding domains has, by itself, activity of binding to an antigen.

In an embodiment of the above aspects, the first and second antigen-binding domains are both antigen-binding domains of the same type. As stated below, examples of proteins that constitute the antigen-binding domains include polypeptides derived from an antibody or a non-antibody protein, and fragments thereof (for example, a Fab, Fab′, scFab, Fv, scFv, and single domain antibody). From the viewpoint of such molecular forms, when the structures of the proteins constituting the first and second antigen-binding domains are identical, the antigen-binding domains are determined to be of the same type.

In an embodiment of the above aspects, the at least one bond which links the first antigen-binding domain and the second antigen-binding domain may be formed by linking amino acid residues present at the same position in the first antigen-binding domain and in the second antigen-binding domain with each other, or it may be formed by linking amino acid residues present at a respectively different position with each other.

Positions of amino acid residues in the antigen-binding domain can be shown according to the Kabat numbering or EU numbering system (also called the EU index) described in Kabat et al., Sequences of Proteins of Immunological Interest, 5th Ed. Public Health Service, National Institutes of Health, Bethesda, M D, 1991. For example, if the amino acid residues from which the bonds between the first and second antigen-binding domains originate are present at an identical position corresponding in the antigen-binding domains, the position of these amino acid residues can be indicated as the same number according to the Kabat numbering or EU numbering system. Alternatively, if the amino acid residues from which the bonds between the first and second antigen-binding domains originate are present at different positions which are not corresponding in the antigen-binding domains, the positions of these amino acid residues can be indicated as different numbers according to the Kabat numbering or EU numbering system.

In an embodiment of the above aspects, at least one of the first and second antigen-binding domains comprises an antibody fragment which binds to a specific antigen. In certain embodiments, the antibody fragment is a Fab, Fab′, scFab, Fv, scFv, or single domain antibody. In certain embodiments, at least one of the amino acid residues from which the bonds between the antigen-binding domains originate is present in an antibody fragment.

In an embodiment of the above aspects, at least one of amino acid residues from which the bonds between the antigen-binding domains originate is present within a constant region. In certain embodiments, the amino acid residue is present within a CH1 region, and for example, it is present at any of positions 119 to 123, 131 to 140, 148 to 150, 155 to 167, 174 to 178, 188 to 197, 201 to 214, and 218 to 219 according to EU numbering in the CH1 region. In certain embodiments, the amino acid residue is present at a position selected from the group consisting of positions 119, 122, 123, 131, 132, 133, 134, 135, 136, 137, 138, 139, 140, 148, 150, 155, 156, 157, 159, 160, 161, 162, 163, 164, 165, 167, 174, 176, 177, 178, 188, 189, 190, 191, 192, 193, 194, 195, 196, 197, 201, 203, 205, 206, 207, 208, 211, 212, 213, 214, 218, and 219 according to EU numbering in the CH1 region. In certain embodiments, the amino acid residue is present at position 134, 135, 136, 137, 191, 192, 193, 194, 195, or 196 according to EU numbering in the CH1 region. In certain embodiments, the amino acid residue is present at position 135, 136, or 191 according to EU numbering in the CH1 region.

In an embodiment of the above aspects, the constant region is derived from human. In certain embodiments, the subclass of the heavy chain constant region is any of IgG1, IgG2, IgG3, IgG4, IgA1, IgA2, IgM, IgD, and IgE. In certain embodiments, the subclass of the CH1 region is any of gamma 1, gamma 2, gamma 3, gamma 4, alpha 1, alpha 2, mu, delta, and epsilon.

In an embodiment of the above aspects, the at least one bond which links the first antigen-binding domain and the second antigen-binding domain is formed by linking an amino acid residue in the CH1 region of the first antigen-binding domain and an amino acid residue in the CH1 region of the second antigen-binding domain. In certain embodiments, the amino acid residues in the first antigen-binding domain and the second antigen-binding domain are each independently selected from the group consisting of positions 119, 120, 121, 122, and 123 according to EU numbering. In certain embodiments, the amino acid residues in the first antigen-binding domain and the second antigen-binding domain are each independently selected from the group consisting of positions 131, 132, 133, 134, 135, 136, 137, 138, 139, and 140 according to EU numbering. In certain embodiments, the amino acid residues in the first antigen-binding domain and the second antigen-binding domain are each independently selected from the group consisting of positions 148, 149, and 150 according to EU numbering. In certain embodiments, the amino acid residues in the first antigen-binding domain and the second antigen-binding domain are each independently selected from the group consisting of positions 155, 156, 157, 158, 159, 160, 161, 162, 163, 164, 165, 166, and 167 according to EU numbering. In certain embodiments, the amino acid residues in the first antigen-binding domain and the second antigen-binding domain are each independently selected from the group consisting of positions 174, 175, 176, 177, and 178 according to EU numbering. In certain embodiments, the amino acid residues in the first antigen-binding domain and the second antigen-binding domain are each independently selected from the group consisting of positions 188, 189, 190, 191, 192, 193, 194, 195, 196, and 197 according to EU numbering. In certain embodiments, the amino acid residues in the first antigen-binding domain and the second antigen-binding domain are each independently selected from the group consisting of positions 201, 202, 203, 204, 205, 206, 207, 208, 209, 210, 211, 212, 213, and 214 according to EU numbering. In certain embodiments, the amino acid residues in the first antigen-binding domain and the second antigen-binding domain are each independently selected from the group consisting of positions 218 and 219 according to EU numbering.

In an embodiment of the above aspects, the difference in the positions of the amino acid residues from which the bonds originate in each of the first antigen-binding domain and the second antigen-binding domain is three amino acids or less. This means that when the position of the amino acid residue from which a bond originates in the CH1 region of the first antigen-binding domain and the position of the amino acid residue from which the bond originates in the CH1 region of the second antigen-binding domain are respectively compared according to EU numbering, the difference (i.e., distance) is three amino acids or less. In certain embodiments, the at least one bond which links the first antigen-binding domain and the second antigen-binding domain is formed by linking the amino acid residue at position 135 according to EU numbering in the CH1 region of the first antigen-binding domain and an amino acid residue at any of positions 132 to 138 according to EU numbering in the CH1 region of the second antigen-binding domain. In certain embodiments, the at least one bond which links the first antigen-binding domain and the second antigen-binding domain is formed by linking the amino acid residue at position 136 according to EU numbering in the CH1 region of the first antigen-binding domain and an amino acid residue at any of positions 133 to 139 according to EU numbering in the CH1 region of the second antigen-binding domain.

In certain embodiments, the at least one bond which links the first antigen-binding domain and the second antigen-binding domain is formed by linking the amino acid residue at position 191 according to EU numbering in the CH1 region of the first antigen-binding domain and an amino acid residue at any of positions 188 to 194 according to EU numbering in the CH1 region of the second antigen-binding domain. In an exemplary embodiment, the at least one bond which links the first antigen-binding domain and the second antigen-binding domain is formed by linking the amino acid residues at position 135 according to EU numbering in the CH1 regions of the two antigen-binding domains with each other. In an exemplary embodiment, the at least one bond which links the first antigen-binding domain and the second antigen-binding domain is formed by linking the amino acid residues at position 136 according to EU numbering in the CH1 regions of the two antigen-binding domains with each other. In an exemplary embodiment, the at least one bond which links the first antigen-binding domain and the second antigen-binding domain is formed by linking the amino acid residues at position 191 according to EU numbering in the CH1 regions of the two antigen-binding domains with each other.

In an embodiment of the above aspects, at least one of amino acid residues from which the bonds between the antigen-binding domains originate is present within a CL region, and for example, it is present at any of positions 108 to 112, 121 to 128, 151 to 156, 184 to 190, 195 to 196, 200 to 203, and 208 to 213 according to Kabat numbering in the CL region. In certain embodiments, the amino acid residue is present at a position selected from the group consisting of positions 108, 109, 112, 121, 123, 126, 128, 151, 152, 153, 156, 184, 186, 188, 189, 190, 195, 196, 200, 201, 202, 203, 208, 210, 211, 212, and 213 according to Kabat numbering in the CL region. In certain embodiments, the amino acid residue is present at position 126 according to Kabat numbering in the CL region.

In an embodiment of the above aspects, the constant region is derived from human. In certain embodiments, the subclass of the CL region is kappa or lambda.

In an embodiment of the above aspects, the at least one bond which links the first antigen-binding domain and the second antigen-binding domain is formed by linking an amino acid residue in the CL region of the first antigen-binding domain and an amino acid residue in the CL region of the second antigen-binding domain. In certain embodiments, the amino acid residues in the first antigen-binding domain and the second antigen-binding domain are each independently selected from the group consisting of positions 108, 109, 110, 111, and 112 according to Kabat numbering. In certain embodiments, the amino acid residues in the first antigen-binding domain and the second antigen-binding domain are each independently selected from the group consisting of positions 121, 122, 123, 124, 125, 126, 127, and 128 according to Kabat numbering. In certain embodiments, the amino acid residues in the first antigen-binding domain and the second antigen-binding domain are each independently selected from the group consisting of positions 151, 152, 153, 154, 155, and 156 according to Kabat numbering. In certain embodiments, the amino acid residues in the first antigen-binding domain and the second antigen-binding domain are each independently selected from the group consisting of positions 184, 185, 186, 187, 188, 189, and 190 according to Kabat numbering. In certain embodiments, the amino acid residues in the first antigen-binding domain and the second antigen-binding domain are each independently selected from the group consisting of positions 195 and 196 according to Kabat numbering. In certain embodiments, the amino acid residues in the first antigen-binding domain and the second antigen-binding domain are each independently selected from the group consisting of positions 200, 201, 202, and 203 according to Kabat numbering. In certain embodiments, the amino acid residues in the first antigen-binding domain and the second antigen-binding domain are each independently selected from the group consisting of positions 208, 209, 210, 211, 212, and 213 according to Kabat numbering.

In an embodiment of the above aspects, the difference in (i.e., distance between) the positions of the amino acid residues from which the bonds originate in each of the first antigen-binding domain and the second antigen-binding domain is three amino acids or less. This means that when the position of the amino acid residue from which a bond originates in the CL region of the first antigen-binding domain and the position of the amino acid residue from which the bond originates in the CL region of the second antigen-binding domain are respectively compared according to EU numbering, the difference (i.e., distance) is three amino acids or less. In an exemplary embodiment, the at least one bond which links the first antigen-binding domain and the second antigen-binding domain is formed by linking the amino acid residues at position 126 according to Kabat numbering in the CL regions of the two antigen-binding domains with each other.

In an embodiment of the above aspects, the at least one bond which links the first antigen-binding domain and the second antigen-binding domain is formed by linking an amino acid residue in the CH1 region of the first antigen-binding domain and an amino acid residue in the CL region of the second antigen-binding domain. In certain embodiments, the amino acid residues in the CH1 region of the first antigen-binding domain are selected from the group consisting of positions 188, 189, 190, 191, 192, 193, 194, 195, 196, and 197 according to EU numbering, and the amino acid residues in the CL region of the second antigen-binding domain are selected from the group consisting of positions 121, 122, 123, 124, 125, 126, 127, and 128 according to Kabat numbering. In an exemplary embodiment, the at least one bond which links the first antigen-binding domain and the second antigen-binding domain is formed by linking the amino acid residue at position 191 according to EU numbering in the CH1 region of the first antigen-binding domain and the amino acid residue at position 126 according to Kabat numbering in the CL region of the second antigen-binding domain.

In an embodiment of the above aspects, at least one of amino acid residues from which the bonds between the antigen-binding domains originate is present within a variable region. In certain embodiments, the amino acid residue is present within a VH region, and for example, it is present at a position selected from the group consisting of positions 6, 8, 16, 20, 25, 26, 28, 74, and 82b according to Kabat numbering in the VH region. In certain embodiments, the amino acid residue is present within a VL region, and for example, it is present at a position selected from the group consisting of positions 21, 27, 58, 77, 100, 105, and 107 according to Kabat numbering in the VL region (subclass kappa) and positions 6, 19, 33, and 34 according to Kabat numbering in the VL region (subclass lambda). In certain embodiments, the amino acid residue is present within a VHH region, and for example, it is present at a position selected from the group consisting of positions 4, 6, 7, 8, 9, 10, 11, 12, 14, 15, 17, 20, 24, 27, 29, 38, 39, 40, 41, 43, 44, 45, 46, 47, 48, 49, 67, 69, 71, 78, 80, 82, 82c, 85, 88, 91, 93, 94, and 107 according to Kabat numbering in the VHH region.

In an embodiment of the above aspects, at least one of the first and second antigen-binding domains comprises a non-antibody protein binding to a particular antigen, or a fragment thereof. In certain embodiments, the non-antibody protein is either of a pair of a ligand and a receptor which specifically bind to each other. Such receptors include, for example, receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

In an embodiment of the above aspects, the first and/or second antigen-binding domains comprise a hinge region. In certain embodiments, at least one of the cysteine residues present within a wild-type hinge region is substituted to another amino acid residue. Such cysteine residues are present, for example, at positions 226 and/or 229 according to EU numbering in the wild-type hinge region. In certain embodiments, at least one of amino acid residues from which the bonds between the antigen-binding domains originate is present within a hinge region and, for example, it is present at a position selected from the group consisting of positions 216, 218, and 219 according to EU numbering in the hinge region.

In an embodiment of the above aspects, the first antigen-binding domain and the second antigen-binding domain are linked with each other via two or more bonds.

In certain embodiments, at least one of amino acid residues from which the bonds between the antigen-binding domains originate is an amino acid residue present in a wild-type sequence and, for example, it is a cysteine residue in a wild-type hinge region. In certain embodiments, the at least one bond which links the first antigen-binding domain and the second antigen-binding domain is a disulfide bond formed by crosslinking of cysteine residues present within wild-type hinge regions with each other. Such cysteine residues are present, for example, at positions 226 and/or 229 according to EU numbering of a wild-type hinge region.

In certain embodiments, at least one of amino acid residues from which the bonds between the antigen-binding domains originate is present within an antibody fragment, and at least one is present within a hinge region. In an exemplary embodiment, the antigen-binding molecule of the present disclosure is F(ab′)2 in which both the first and second antigen-binding domains comprise a Fab and a hinge region.

In an embodiment of the above aspects, the antigen-binding molecule of the present disclosure further comprises an Fc region, and for example, it is a full-length antibody. In certain embodiments, one or more amino acid mutations promoting multimerization of Fc regions are introduced into the Fc region of the antigen-binding molecule of the present disclosure. Such amino acid mutations include, for example, the amino acid mutations at at least one position selected from the group consisting of positions 247, 248, 253, 254, 310, 311, 338, 345, 356, 359, 382, 385, 386, 430, 433, 434, 436, 437, 438, 439, 440, and 447 according to EU numbering (see, e.g., WO 2016/164480). In certain embodiments, the multimerization is hexamerization.

### <Antigens Bound by Antigen-Binding Molecules>

In an embodiment of the above aspects, both the first and second antigen-binding domains bind to the same antigen. In certain embodiments, both the first and second antigen-binding domains bind to the same epitope on the same antigen. In certain other embodiments, each of the first and second antigen-binding domains binds to a different epitope on the same antigen. In certain embodiments, the antigen-binding molecule of the present disclosure is a biparatopic antigen-binding molecule (for example, a biparatopic antibody) that targets one specific antigen. In another embodiment of the above aspects, each of the first and second antigen-binding domains binds to a different antigen.

In another embodiment of the above aspects, the antigen-binding molecule of the present disclosure is a clamping antigen-binding molecule (for example, a clamping antibody). A clamping antigen-binding molecule in the present specification means an antigen-binding molecule which specifically binds to an antigen/antigen-binding molecule complex formed between a given antigen A and an antigen-binding molecule which binds to antigen A, and which thereby increases the binding activity toward antigen A of the antigen-binding molecule that binds to antigen A (or alternatively, stabilizes the antigen/antigen-binding molecule complex formed by antigen A and the antigen-binding molecule that binds to antigen A). For example, a CD3 clamping antibody specifically binds to the antigen-antibody complex formed between CD3 and an antibody with reduced binding ability toward CD3 (binding-attenuated CD3 antibody) and can thereby increase the binding activity of the binding-attenuated CD3 antibody toward CD3 (or alternatively, stabilize the antigen-antibody complex formed by CD3 and the binding-attenuated CD3 antibody). In certain embodiments, the first and/or second antigen-binding domains in the antigen-binding molecule of the present disclosure can be antigen-binding domains (clamping antigen-binding domains) from clamping antigen-binding molecules.

In an embodiment of the above aspects, both the first and second antigen-binding domains have the same amino acid sequence. In another embodiment, each of the first and second antigen-binding domains has a different amino acid sequence.

In an embodiment of the above aspects, at least one of two antigens to which the first and second antigen-binding domains bind is a soluble protein or a membrane protein.

### <Functions of Antigen-Binding Molecules>

In an embodiment of the above aspects, the antigen-binding molecule of the present disclosure has activity of holding two antigen molecules at spatially close positions. In certain embodiments, the antigen-binding molecule of the present disclosure is capable of holding two antigen molecules at closer positions than a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region).

In another embodiment of the above aspects, the antigen-binding molecule of the present disclosure has activity of regulating interaction between two antigen molecules. Without being bound by a particular theory, the activity of regulating interaction is thought to be resulted from holding two antigen molecules at spatially closer positions by the antigen-binding molecule of the present disclosure. In certain embodiments, the antigen-binding molecule of the present disclosure is capable of enhancing or diminishing interaction between two antigen molecules as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region).

In certain embodiments, the two antigen molecules bound by the antigen-binding molecule of the present disclosure are a ligand and a receptor thereof, respectively, and the antigen-binding molecule of the present disclosure has activity of promoting activation of the receptor by the ligand. In certain other embodiment, the two antigen molecules bound by the antigen-binding molecule of the present disclosure are an enzyme and a substrate thereof, respectively, and the antigen-binding molecule of the present disclosure has activity of promoting catalytic reaction of the enzyme with the substrate.

Further, in certain other embodiments, both of the two antigen molecules bound by the antigen-binding molecule of the present disclosure are antigens (for example, proteins) present on cellular surfaces, and the antigen-binding molecule of the present disclosure has activity of promoting interaction between a cell expressing the first antigen and a cell expressing the second antigen. For example, the cell expressing the first antigen and the cell expressing the second antigen are, respectively, a cell with cytotoxic activity and a target cell thereof, and the antigen-binding molecule of the present disclosure promotes damage of the target cell by the cell with cytotoxic activity. The cell with cytotoxic activity is, for example, a T cell, NK cell, monocyte, or macrophage.

In an embodiment of the above aspects, the antigen-binding molecule of the present disclosure has activity of regulating activation of two antigen molecules which are activated by association with each other. Without being bound by a particular theory, the activity of regulating activation is thought to be resulted from holding two antigen molecules at spatially closer positions by the antigen-binding molecule of the present disclosure. In certain embodiments, the antigen-binding molecule of the present disclosure can enhance or diminish activation of two antigen molecules as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). For example, such antigen molecules are selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

In an embodiment of the above aspects, in the antigen-binding molecule of the present disclosure, two antigen-binding domains are present at spatially close positions and/or the mobility of the two antigen-binding domains is reduced. In certain embodiments, as compared with a control antigen-binding molecule, the antigen-binding molecule of the present disclosure has two antigen-binding domains that are present at closer positions and/or the mobility of the two antigen-binding domains is more reduced, and the control antigen-binding molecule differs from the antigen-binding molecule of the present disclosure only in that it has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region).

In an embodiment of the above aspects, the antigen-binding molecule of the present disclosure has resistance to protease cleavage. In certain embodiments, the antigen-binding molecule of the present disclosure has increased resistance to protease cleavage as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). In certain embodiments, in the antigen-binding molecule of the present disclosure, the proportion of the full-length molecule (for example, full-length IgG molecule) remaining after protease treatment is increased as compared to the control antigen-binding molecule. In certain embodiments, in the antigen-binding molecule of the present disclosure, the proportion of a particular fragment (for example, Fab monomer) produced after protease treatment is reduced as compared to the control antigen-binding molecule.

In an embodiment of the above aspects, when the antigen-binding molecule of the present disclosure is treated with a protease, a dimer of the antigen-binding domains or fragments thereof (for example, crosslinked Fab dimer) is excised. In certain embodiments, when the control antigen-binding molecule, which differs from the antigen-binding molecule of the present disclosure only in that it has one less bond between the two antigen-binding domains, is treated with the protease, monomers of the antigen-binding domains or fragments thereof are excised. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). In these embodiments, the protease can cleave the hinge region of the antigen-binding molecule.

In a further embodiment, the control antigen-binding molecule differs from the antigen-binding molecule of the present disclosure only in that it has one less bond between the two antigen-binding domains, and the one less bond is a bond which is formed originating from mutated amino acid residues. The mutated amino acid residues are, for example, artificially introduced cysteine residues.

### <Pharmaceutical Compositions >

In an aspect, the present disclosure provides a pharmaceutical composition comprising the antigen-binding molecule of the present disclosure and a pharmaceutically acceptable carrier.

### <Use of Antigen-Binding Molecules>

In an aspect, the present disclosure provides a method for holding two antigen molecules at spatially close positions, comprising:
(a) providing an antigen-binding molecule comprising two antigen-binding domains,
(b) adding to the antigen-binding molecule at least one bond which links the two antigen-binding domains with each other, and
(c) contacting the antigen-binding molecule produced in (b) with the two antigen molecules. In certain embodiments, the two antigen-binding domains in the antigen-binding molecule recited in (a) above may be linked with each other via one or more bonds, and in this case, some or all of the one or more bonds are bonds in which the amino acid residues from which the bonds between the antigen-binding domains originate are derived from amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for holding two antigen molecules at spatially close positions which comprises contacting two antigen molecules with the antigen-binding molecule or pharmaceutical composition of the present disclosure. The present disclosure further provides an antigen-binding molecule or pharmaceutical composition of the present disclosure for use in holding two antigen molecules at spatially close positions.

In another aspect, the present disclosure provides a method for regulating interaction between two antigen molecules, comprising:
(a) providing an antigen-binding molecule comprising two antigen-binding domains,
(b) adding to the antigen-binding molecule at least one bond which links the two antigen-binding domains with each other, and
(c) contacting the antigen-binding molecule produced in (b) with the two antigen molecules. In certain embodiments, the two antigen-binding domains in the antigen-binding molecule recited in (a) above may be linked with each other via one or more bonds, and in this case, some or all of the one or more bonds are bonds in which the amino acid residues from which the bonds between the antigen-binding domains originate are derived from amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for regulating interaction between two antigen molecules which comprises contacting two antigen molecules with the antigen-binding molecule or pharmaceutical composition of the present disclosure. The present disclosure further provides an antigen-binding molecule or pharmaceutical composition of the present disclosure for use in regulating interaction between two antigen molecules.

Further, in another aspect, the present disclosure provides a method for regulating activity of two antigen molecules which are activated by association with each other, comprising:
(a) providing an antigen-binding molecule comprising two antigen-binding domains,
(b) adding to the antigen-binding molecule at least one bond which links the two antigen-binding domains with each other, and
(c) contacting the antigen-binding molecule produced in (b) with the two antigen molecules. In certain embodiments, the two antigen-binding domains in the antigen-binding molecule recited in (a) above may be linked with each other via one or more bonds, and in this case, some or all of the one or more bonds are bonds in which the amino acid residues from which the bonds between the antigen-binding domains originate are derived from amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for regulating activity of two antigen molecules which are activated by association with each other, which comprises contacting two antigen molecules with the antigen-binding molecule or pharmaceutical composition of the present disclosure. The present disclosure further provides an antigen-binding molecule or pharmaceutical composition of the present disclosure for use in regulating activity of two antigen molecules which are activated by association with each other.

Further, in another aspect, the present disclosure provides a method for placing two antigen-binding domains at spatially close positions and/or reducing the mobility of two antigen-binding domains, comprising:
(a) providing an antigen-binding molecule comprising two antigen-binding domains, and
(b) adding to the antigen-binding molecule at least one bond which links the two antigen-binding domains with each other. In certain embodiments, the two antigen-binding domains in the antigen-binding molecule recited in (a) above may be linked with each other via one or more bonds, and in this case, some or all of the one or more bonds are bonds in which the amino acid residues from which the bonds between the antigen-binding domains originate are derived from amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Furthermore, in another aspect, the present disclosure provides a method for increasing resistance of an antigen-binding molecule to protease cleavage, comprising:
(a) providing an antigen-binding molecule comprising two antigen-binding domains, and
(b) adding to the antigen-binding molecule at least one bond which links the two antigen-binding domains with each other. In certain embodiments, the two antigen-binding domains in the antigen-binding molecule recited in (a) above may be linked with each other via one or more bonds, and in this case, some or all of the one or more bonds are bonds in which the amino acid residues from which the bonds between the antigen-binding domains originate are derived from amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

The antigen-binding molecule used in these various methods may have the characteristics of the antigen-binding molecules described herein.

### <Methods for Producing Antigen-Binding Molecules>

In an aspect, the present disclosure provides a method for producing an antigen-binding molecule which has activity of holding two antigen molecules at spatially close positions, comprising:
(a) providing a nucleic acid encoding a polypeptide comprising a first antigen-binding domain and a nucleic acid encoding a polypeptide comprising a second antigen-binding domain,
(b) introducing a mutation into the nucleic acids encoding the two antigen-binding domains such that at least one bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via one or more bonds; and preferably further comprising a step of contacting the antibody preparation with a reducing reagent.

In certain embodiments, said contacting with a reducing agent (“said contacting step”) preferentially enriches or increases the population of an antibody structural isoform having at least one disulfide bond formed between amino acid residues which are not in a hinge region. In certain embodiments, said method produces a homogenous antibody preparation having at least 50%, 60%, 70%, 80%, 90%, preferably at least 95% molar ratio of said antibody having at least one disulfide bond formed between amino acid residues which are not in a hinge region.

In certain embodiments, the pH of said reducing reagent contacting with the antibody is from about 3 to about 10. In certain embodiments, the pH of said reducing reagent contacting with the antibody is about 6, 7 or 8. In some embodiments, the pH of said reducing reagent contacting with the antibody is about 7 or about 3.

In certain embodiments, the reducing agent is selected from the group consisting of TCEP, 2-MEA, DTT, Cysteine, GSH and Na₂SO₃. In some preferred embodiments, the reducing agent is TCEP. In certain embodiments, the concentration of the reducing agent is from about 0.01 mM to about 100 mM.

In some preferred embodiments, the concentration of the reducing agent is about 0.01, 0.05, 0.1, 0.25, 0.5, 1, 2.5, 5, 10, 25, 50, 100 mM, preferably about 0.01 mM to 25 mM. In one preferred embodiment, the reducing agent is 0.01 mM to 25 mM of TCEP.

In certain embodiments, the contacting step with a reducing agent is performed for at least 30 minutes. In certain embodiments, the contacting step is performed for about 2 to about 48 hours. In some preferred embodiments, the contacting step is performed for about 2 hours or about 16 hours.

In certain embodiments, the contacting step is performed at a temperature of about 20 degrees C. to 37 degrees C., preferably at 23 degrees C., 25 degrees C. or 37 degrees C., more preferably at 23 degrees C. In certain embodiments, said antibody is partially purified by affinity chromatography (preferably Protein A chromatography) prior to said contacting. In certain embodiments, the concentration of the antibody is from about 1 mg/ml and about 50 mg/ml. In some preferred embodiments, the concentration of the antibody is about 1 mg/ml or about 20 mg/ml.

In certain embodiments, said contacting step preferentially enriches or increases the population of an antibody structural isoform having at least one disulfide bond formed between amino acid residues which are not in a hinge region. In certain embodiments, said contacting step produces a homogenous antibody preparation having at least 50%, 60%, 70%, 80%, 90%, preferably at least 95% molar ratio of said antibody having at least one disulfide bond formed between amino acid residues which are not in a hinge region.

In certain embodiments, said contacting step produces an antibody preparation which is more homogeneous than the same antibody preparation that has not been treated by said method.

In certain embodiments, said contacting step produces an antibody preparation having increase in its biological activity compared to the same antibody that has not been treated by said method.

In certain embodiments, said contacting step produces an antibody having enhanced activity of holding two antigen molecules at spatially close positions compared to the same antibody that has not been treated by said method.

In certain embodiments, said contacting step produces an antibody having enhanced stability compared to the same antibody that has not been treated by said method.

In certain embodiments, said contacting step preferentially enriches antibody having at least one disulfide bond formed outside of hinge regions and said preferentially enriched form has a pharmaceutically desirable property selected from any of (a) to (e) below, as compared to a preparation that has not been treated by said contacting step:
(a) wherein said at least one disulfide bond restricts the antigen binding orientation of the two antigen-binding domains to cis antigen-binding (i.e. binding to two antigens on the same cell), or restrict binding of the two antigen binding domains to two antigens which are spatially close to each other;
(b) wherein said at least one disulfide bond holds the first antigen-binding domain and the second antigen-binding domain spatially closer to each other, as compared to a same corresponding antibody which does not have said at least one disulfide bond;
(c) wherein said at least one disulfide bond reduce the flexibility and/or mobility of first antigen-binding domain and the second antigen-binding domain, as compared to a corresponding same antibody which does not have said at least one disulfide bond;
(d) wherein said at least one disulfide bond increases resistance of the antibody to protease cleavage, as compared to a corresponding same antibody which does not have said at least one disulfide bond; or
(e) wherein said at least one disulfide bond enhances or reduces interaction between two antigen molecules bound by the antigen-binding molecule, as compared to a corresponding same antibody which does not have said at least one disulfide bond.

In certain embodiments, each of the two antigen-binding domains recited in (a) above may comprise one or more amino acid residues from which the bonds for linking the two antigen-binding domains originate, and in this case, some or all of the one or more amino acid residues from which the bond between the antigen-binding domains originates are amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

In another aspect, the present disclosure provides a method for producing an antigen-binding molecule which has activity of regulating interaction between two antigen molecules, comprising:
(a) providing a nucleic acid encoding a polypeptide comprising a first antigen-binding domain and a nucleic acid encoding a polypeptide comprising a second antigen-binding domain,
(b) introducing a mutation into the nucleic acids encoding the two antigen-binding domains such that at least one bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via one or more bonds.

In certain embodiments, each of the two antigen-binding domains recited in (a) above may comprise one or more amino acid residues from which the bonds for linking the two antigen-binding domains originate, and in this case, some or all of the one or more amino acid residues from which the bond between the antigen-binding domains originates are amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Further, in another aspect, the present disclosure provides a method for producing an antigen-binding molecule which has activity of regulating activation of two antigen molecules which are activated by association with each other, comprising:
(a) providing a nucleic acid encoding a polypeptide comprising a first antigen-binding domain and a nucleic acid encoding a polypeptide comprising a second antigen-binding domain,
(b) introducing a mutation into the nucleic acids encoding the two antigen-binding domains such that at least one bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via one or more bonds.

In certain embodiments, each of the two antigen-binding domains recited in (a) above may comprise one or more amino acid residues from which the bonds for linking the two antigen-binding domains originate, and in this case, some or all of the one or more amino acid residues from which the bond between the antigen-binding domains originates are amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Further, in another aspect, the present disclosure provides a method for producing an antigen-binding molecule in which two antigen-binding domains are present at spatially close positions and/or the mobility of two antigen-binding domains is reduced, comprising:
(a) providing a nucleic acid encoding a polypeptide comprising a first antigen-binding domain and a nucleic acid encoding a polypeptide comprising a second antigen-binding domain,
(b) introducing a mutation into the nucleic acids encoding the two antigen-binding domains such that at least one bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via one or more bonds.

In certain embodiments, each of the two antigen-binding domains recited in (a) above may comprise one or more amino acid residues from which the bonds for linking the two antigen-binding domains originate, and in this case, some or all of the one or more amino acid residues from which the bond between the antigen-binding domains originates are amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Furthermore, in another aspect, the present disclosure provides a method for producing an antigen-binding molecule which has increased resistance to protease cleavage, comprising:
(a) providing a nucleic acid encoding a polypeptide comprising a first antigen-binding domain and a nucleic acid encoding a polypeptide comprising a second antigen-binding domain,
(b) introducing a mutation into the nucleic acids encoding the two antigen-binding domains such that at least one bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via one or more bonds.

In certain embodiments, each of the two antigen-binding domains recited in (a) above may comprise one or more amino acid residues from which the bonds for linking the two antigen-binding domains originate, and in this case, some or all of the one or more amino acid residues from which the bond between the antigen-binding domains originates are amino acid residues which are present in a wild-type Fab or hinge region (for example, cysteine residues in the hinge region). In a further embodiment, said at least one bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

The antigen-binding molecule produced in these various aspects may have the characteristics of the antigen-binding molecules described herein.

### <Methods of Screening for Antigen-Binding Molecules>

In another aspect, the present disclosure provides a method for identifying a novel pair of protein molecules which are activated by association with each other, comprising:
(a) providing two arbitrary protein molecules,
(b) producing, by the production method of the present disclosure, an antigen-binding molecule comprising two antigen-binding domains which respectively bind to the two protein molecules,
(c) contacting the antigen-binding molecule produced in (b) with the two protein molecules, and
(d) assessing whether or not the two protein molecules are activated.

In certain embodiments, at least one of the two protein molecules is selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

### A. Exemplary Antigen-Binding Molecules

### <Structures of Antigen-Binding Molecules>

In an aspect, the present disclosure provides an antigen-binding molecule comprising a first antigen-binding domain and a second antigen-binding domain, and the antigen-binding domains are linked with each other via two or more bonds. In an embodiment, at least one of the first and second antigen-binding domains has, by itself, activity of binding to an antigen (i.e., a single antigen-binding domain independently has antigen-binding activity). In certain embodiments, each of the first and second antigen-binding domains has, by itself, activity of binding to an antigen.

In an embodiment of the above aspects, at least one of the first and second antigen-binding domains comprises an antibody fragment which binds to a particular antigen. In certain embodiments, the first and/or second antigen-binding domains comprise a hinge region. Amino acid residues from which the bonds between the antigen-binding domains originate are respectively present in the first and second antigen-binding domains, and the bonds between the antigen-binding domains are formed by linking these amino acid residues. In certain embodiments, at least one of amino acid residues from which the bonds between the antigen-binding domains originate is present within the antibody fragment. In certain embodiments, at least one of amino acid residues from which the bonds between the antigen-binding domains originate is present within a hinge region. In certain embodiments, at least one of amino acid residues from which the bonds between the antigen-binding domains originate is present within the antibody fragment, and at least one of the amino acid residues is present within a hinge region.

In an embodiment of the above aspects, in at least one of the first and second antigen-binding domains, multiple amino acid residues from which the bonds between the antigen-binding domains originate are present at positions at a distance of seven amino acids or more from each other in the primary structure. This means that, between any two amino acid residues of the above multiple amino acid residues, six or more amino acid residues which are not said amino acid residues are present. In certain embodiments, combinations of multiple amino acid residues from which the bonds between the antigen-binding domains originate include a pair of amino acid residues which are present at positions at a distance of less than seven amino acids in the primary structure. In certain embodiments, if the first and second antigen-binding domains are linked each other via three or more bonds, the bonds between the antigen-binding domains may originate from three or more amino acid residues including a pair of amino acid residues which are present at positions at a distance of seven amino acids or more in the primary structure.

In certain embodiments, amino acid residues present at the same position in the first antigen-binding domain and in the second antigen-binding domain are linked with each other to form a bond. In certain embodiments, amino acid residues present at a different position in the first antigen-binding domain and in the second antigen-binding domain are linked with each other to form a bond.

Positions of amino acid residues in the antigen-binding domain can be shown according to the Kabat numbering or EU numbering system (also called the EU index) described in Kabat et al., Sequences of Proteins of Immunological Interest, 5th Ed. Public Health Service, National Institutes of Health, Bethesda, M D, 1991. For example, if the amino acid residues from which the bonds between the first and second antigen-binding domains originate are present at an identical position corresponding in the antigen-binding domains, the position of these amino acid residues can be indicated as the same number according to the Kabat numbering or EU numbering system. Alternatively, if the amino acid residues from which the bonds between the first and second antigen-binding domains originate are present at different positions which are not corresponding in the antigen-binding domains, the positions of these amino acid residues can be indicated as different numbers according to the Kabat numbering or EU numbering system.

In an embodiment of the above aspects, at least one of the two or more bonds linking the antigen-binding domains is a covalent bond. In certain embodiments, the covalent bond is formed by direct crosslinking of an amino acid residue in the first antigen-binding domain and an amino acid residue in the second antigen-binding domain. The crosslinked amino acid residues are, for example, cysteine, and the formed covalent bond is, for example, a disulfide bond. At least one of the crosslinked cysteine residues may be present within a hinge region.

In certain other embodiments, the covalent bond is formed by crosslinking of an amino acid residue in the first antigen-binding domain and an amino acid residue in the second antigen-binding domain via a crosslinking agent. The crosslinking agent is, for example, an amine-reactive crosslinking agent, and the crosslinked amino acid residues are, for example, lysine.

In an embodiment of the above aspects, at least one of the two or more bonds linking the antigen-binding domains is a noncovalent bond. In certain embodiments, the noncovalent bond is an ionic bond, hydrogen bond, or hydrophobic bond.

In an embodiment of the above aspects, the antibody fragment is a Fab, Fab′, scFab, Fv, scFv, or single domain antibody.

In an embodiment of the above aspects, at least one of amino acid residues from which the bonds between the antigen-binding domains originate is present within a constant region. In certain embodiments, the amino acid residue is present within a CH1 region, and for example, it is present at a position selected from the group consisting of positions 119, 122, 123, 131, 132, 133, 134, 135, 136, 137, 139, 140, 148, 150, 155, 156, 157, 159, 160, 161, 162, 163, 165, 167, 174, 176, 177, 178, 190, 191, 192, 194, 195, 197, 213, and 214 according to EU numbering in the CH1 region. In an exemplary embodiment, the amino acid residue is present at position 191 according to EU numbering in the CH1 region, and the amino acid residues at position 191 according to EU numbering in the CH1 region of the two antigen-binding domains are linked with each other to form a bond.

In some embodiments of the above aspects, one disulfide bond is formed between the amino acid residues at position 191 according to EU numbering in the respective CH1 regions of the first antigen-binding domain and the second antigen-binding domain.

In some embodiments of the above aspects, additional one, two or more disulfide bond(s) is/are formed between the first antigen-binding domain and the second antigen-binding domain via the amino acid residues at the following positions according to EU numbering in each of the respective CH1 regions of the first antigen-binding domain and the second antigen-binding domain:
(a) between amino acid residues at any position of 131 to 138, 194 and 195 in each of the two antigen-binding domains;
(b) between the amino acid residues at position 131 in each of the two antigen-binding domains, and between the amino acid residues at position 194 in each of the two antigen-binding domains;
(c) between the amino acid residues at position 132 in each of the two antigen-binding domains, and between the amino acid residues at position 194 in each of the two antigen-binding domains;
(d) between the amino acid residues at position 133 in each of the two antigen-binding domains, and between the amino acid residues at position 194 in each of the two antigen-binding domains;
(e) between the amino acid residues at position 134 in each of the two antigen-binding domains, and between the amino acid residues at position 194 in each of the two antigen-binding domains;
(f) between the amino acid residues at position 135 in each of the two antigen-binding domains, and between the amino acid residues at position 194 in each of the two antigen-binding domains;
(g) between the amino acid residues at position 136 in each of the two antigen-binding domains, and between the amino acid residues at position 194 in each of the two antigen-binding domains;
(h) between the amino acid residues at position 137 in each of the two antigen-binding domains, and between the amino acid residues at position 194 in each of the two antigen-binding domains;
(i) between the amino acid residues at position 138 in each of the two antigen-binding domains, and between the amino acid residues at position 194 in each of the two antigen-binding domains;
(j) between the amino acid residues at position 131 in each of the two antigen-binding domains, and between the amino acid residues at position 195 in each of the two antigen-binding domains;
(k) between the amino acid residues at position 132 in each of the two antigen-binding domains, and between the amino acid residues at position 195 in each of the two antigen-binding domains;
(l) between the amino acid residues at position 133 in each of the two antigen-binding domains, and between the amino acid residues at position 195 in each of the two antigen-binding domains;
(m) between the amino acid residues at position 134 in each of the two antigen-binding domains, and between the amino acid residues at position 195 in each of the two antigen-binding domains;
(n) between the amino acid residues at position 135 in each of the two antigen-binding domains, and between the amino acid residues at position 195 in each of the two antigen-binding domains;
(o) between the amino acid residues at position 136 in each of the two antigen-binding domains, and between the amino acid residues at position 195 in each of the two antigen-binding domains;
(p) between the amino acid residues at position 137 in each of the two antigen-binding domains, and between the amino acid residues at position 195 in each of the two antigen-binding domains; and
(q) between the amino acid residues at position 138 in each of the two antigen-binding domains, and between the amino acid residues at position 195 in each of the two antigen-binding domains.

In some embodiments of the above aspects, any one of the first and second antigen-binding domains comprises one, two or more charged amino acid residues at position 136-138 (according to EU numbering) in the respective CH1 region; and the other antigen-binding domain out of the first and second antigen-binding domains comprises one, two or more oppositely charged amino acid residues at position 193-195 (according to EU numbering) in the respective CH1 region.

In some embodiments of the above aspects, any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more negatively charged amino acid residues at position 193-195 (according to EU numbering) in the respective CH1 region.

In some embodiments of the above aspects, any one of the first and second antigen-binding domains comprises one, two or more negatively charged amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region.

In some embodiments of the above aspects, any one of the first and second antigen-binding domains comprises one, two or more of the following amino acid residues in the respective CH1 region (according to EU numbering):
(a) the amino acid residue at position 136 which is glutamic acid (E) or aspartic acid (D);
(b) the amino acid residue at position 137 which is glutamic acid (E) or aspartic acid (D);
(c) the amino acid residue at position 138 which is glutamic acid (E) or aspartic acid (D); and
the other antigen-binding domain of the first and second antigen-binding domains comprises one, two or more of the following amino acid residues in the respective CH1 region (according to EU numbering):
(d) the amino acid residue at position 193 which is lysine (K), arginine (R), or histidine (H);
(e) the amino acid residue at position 194 which is lysine (K), arginine (R), or histidine (H); and
(f) the amino acid residue at position 195 which is lysine (K), arginine (R), or histidine (H).

In some embodiments of the above as aspects, any one of the first and second antigen-binding domains comprises one or more of the following amino acid residues in the respective CH1 region (according to EU numbering):
(a) the amino acid residue at position 136 which is lysine (K), arginine (R), or histidine (H);
(b) the amino acid residue at position 137 which is lysine (K), arginine (R), or histidine (H);
(c) the amino acid residue at position 138 which is lysine (K), arginine (R), or histidine (H); and
   the other antigen-binding domain of the first and second antigen-binding domains comprises one or more of the following amino acid residues in the respective CH1 region (according to EU numbering):
(d) the amino acid residue at position 193 which is glutamic acid (E) or aspartic acid (D);
(e) the amino acid residue at position 194 which is glutamic acid (E) or aspartic acid (D); and
(f) the amino acid residue at position 195 which is glutamic acid (E) or aspartic acid (D).

In some embodiments of the above aspects, any one of the first and second antigen-binding domains comprises one, two or more hydrophobic amino acid residues at position 136-138 (according to EU numbering) in the respective CH1 region; and the other antigen-binding domain out of the first and second antigen-binding domains comprises one, two or more hydrophobic amino acid residues at position 193-195 (according to EU numbering) in the respective CH1 region.

In some embodiments of the above aspects, the hydrophobic amino acid residue(s) is/are alanine (Ala), valine (Val), leucine (Leu), isoleucine (Ile), phenylalanine (Phe), and/or tryptophan (Trp).

In some embodiments of the above aspects, any one of the first and second antigen-binding domains comprises one “knob” amino acid residues at position 136-138 (according to EU numbering) in the respective CH1 region; and the other antigen-binding domain out of the first and second antigen-binding domains comprises one, two or more “hole” amino acid residues at position 193-195 (according to EU numbering) in the respective CH1 region. In some embodiments of the above aspects, any one of the first and second antigen-binding domains comprises one, two or more “hole” amino acid residues at position 136-138 (according to EU numbering) in the respective CH1 region; and the other antigen-binding domain out of the first and second antigen-binding domains comprises one “knob” amino acid residues at position 193-195 (according to EU numbering) in the respective CH1 region. In some embodiments, said “knob” amino acid residue(s) is/are selected from the group consisting of tryptophan (Trp) and phenylalanine (Phe); and said “hole” amino acid residue(s) is/are selected from the group consisting of alanine (Ala), valine (Val), threonine (T) or serine (S).

In some embodiments of the above aspects, any one of the first and second antigen-binding domains comprises one, two or more aromatic amino acid residues at position 136-138 (according to EU numbering) in the respective CH1 region; and the other antigen-binding domain out of the first and second antigen-binding domains comprises one, two or more positively charged amino acid residues at position 193-195 (according to EU numbering) in the respective CH1 region. In some embodiments of the above aspects, any one of the first and second antigen-binding domains comprises one, two or more positively charged amino acid residues at position 136-138 (according to EU numbering) in the respective CH1 region; and the other antigen-binding domain out of the first and second antigen-binding domains comprises one, two or more aromatic amino acid residues at position 193-195 (according to EU numbering) in the respective CH1 region. In some embodiments, said aromatic amino acid residue(s) is/are selected from the group consisting of tryptophan (Trp), tyrosine (Tyr), histidine (His), and phenylalanine (Phe); and said positively charged amino acid residue(s) is/are selected from a group consisting of lysine (K), arginine (R), or histidine (H).

In certain embodiments, at least one of amino acid residues from which the bonds between the antigen-binding domains originate is present within a hinge region, and for example, it is present at a position selected from the group consisting of positions 216, 218, and 219 according to EU numbering in the hinge region.

In certain embodiments, at least one of amino acid residues from which the bonds between the antigen-binding domains originate is present within a CL region, and for example, it is present at a position selected from the group consisting of positions 109, 112, 121, 126, 128, 151, 152, 153, 156, 184, 186, 188, 190, 200, 201, 202, 203, 208, 210, 211, 212, and 213 according to EU numbering in the CL region. In an exemplary embodiment, the amino acid residue is present at position 126 according to EU numbering in the CL region, and the amino acid residues at position 126 according to EU numbering in the CL region of the two antigen-binding domains are linked with each other to form a bond.

In certain embodiments, an amino acid residue in the CH1 region of the first antigen-binding domain and an amino acid residue in the CL region of the second antigen-binding domain are linked to form a bond. In an exemplary embodiment, an amino acid residue at position 191 according to EU numbering in the CH1 region of the first antigen-binding domain and an amino acid residue at position 126 according to EU numbering in the CL region of the second antigen-binding domain are linked to form a bond.

In an embodiment of the above aspects, the constant region is derived from human. In certain embodiments, the subclass of the heavy chain constant region is any of IgG1, IgG2, IgG3, IgG4, IgA1, IgA2, IgM, IgD, and IgE. In certain embodiments, the subclass of the CH1 region is any of gamma 1, gamma 2, gamma 3, gamma 4, alpha 1, alpha 2, mu, delta, and epsilon. In certain embodiments, the subclass of the CL region is kappa or lambda.

In an embodiment of the above aspects, at least one of amino acid residues from which the bonds between the antigen-binding domains originate is present within a variable region. In certain embodiments, the amino acid residue is present within a VH region, and for example, it is present at a position selected from the group consisting of positions 8, 16, 28, 74, and 82b according to Kabat numbering in the VH region. In certain embodiments, the amino acid residue is present within a VL region, and for example, it is present at a position selected from the group consisting of positions 100, 105, and 107 according to Kabat numbering in the VL region.

In an embodiment of the above aspects, both the first and second antigen-binding domains comprise a Fab and a hinge region.

In certain embodiments, at least one of amino acid residues from which the bonds between the antigen-binding domains originate is an amino acid residue present in a wild-type Fab or hinge region, and for example, it is a cysteine residue in the hinge region. Examples of such cysteine residues include the cysteine residues at positions 226 and 229 according to EU numbering.

In certain other embodiments, at least one of amino acid residues from which the bonds between the antigen-binding domains originate is a mutated amino acid residue which is not present in a wild-type Fab or hinge region, and for example, it is a cysteine residue which is not present in a wild-type Fab or hinge region. Such a mutated amino acid residue can be introduced into a wild-type Fab or hinge region by, for example, a method of amino acid substitution. The present specification discloses the sites of amino acid residues from which the bonds between the antigen-binding domains can originate for each of the CH1, hinge, CL, VH, and VL regions, and for example, cysteine residues can be introduced into such sites.

Alternatively, in another embodiment, an amino acid residue that is present in a wild-type Fab or hinge region and which is involved in a bond between the antigen-binding domains (for example, a cysteine residue) can be substituted with another amino acid or deleted. Examples of such cysteine residues include the cysteine residues at positions 220, 226, and 229 according to EU numbering in the hinge region, and the cysteine residue at position 214 in the CL region.

In certain embodiments, the antigen-binding molecule of the present disclosure is F(ab′)2 in which both the first and second antigen-binding domains comprise a Fab and a hinge region.

In an embodiment of the above aspects, at least one of the first and second antigen-binding domains comprises a non-antibody protein binding to a particular antigen, or a fragment thereof. In certain embodiments, the non-antibody protein is either of a pair of a ligand and a receptor which specifically bind to each other. Such receptors include, for example, receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

In an embodiment of the above aspects, the antigen-binding molecule of the present disclosure further comprises an Fc region, and for example, it is a full-length antibody. In certain embodiments, one or more amino acid mutations promoting multimerization of Fc regions are introduced into the Fc region of the antigen-binding molecule of the present disclosure. Such amino acid mutations include, for example, the amino acid mutations at at least one position selected from the group consisting of positions 247, 248, 253, 254, 310, 311, 338, 345, 356, 359, 382, 385, 386, 430, 433, 434, 436, 437, 438, 439, 440, and 447 according to EU numbering (see, e.g., WO 2016/164480). In certain embodiments, the multimerization is hexamerization.

### <Antigens Bound by Antigen-Binding Molecules>

In an embodiment of the above aspects, both the first and second antigen-binding domains bind to the same antigen. In certain embodiments, both the first and second antigen-binding domains bind to the same epitope on the same antigen. In certain other embodiments, each of the first and second antigen-binding domains binds to a different epitope on the same antigen. In certain embodiments, the antigen-binding molecule of the present disclosure is a biparatopic antigen-binding molecule (for example, biparatopic antibody) that targets one specific antigen.

In an embodiment of the above aspects, each of the first and second antigen-binding domains binds to a different antigen.

In another embodiment of the above aspects, the antigen-binding molecule of the present disclosure is a clamping antigen-binding molecule (for example, clamping antibody). Herein, a clamping antigen-binding molecule refers to an antigen-binding molecule that specifically binds to an antigen/antigen-binding molecule complex formed by a certain antigen A and an antigen-binding molecule binding to the antigen A, and thereby increases the activity of the antigen-binding molecule binding to the antigen A to bind the antigen A (or stabilize the antigen/antigen-binding molecule complex formed by the antigen A and the antigen-binding molecule binding to the antigen A). For example, a CD3 clamping antibody is able to bind to an antigen-antibody complex formed by CD3 and an antibody with attenuated binding ability to CD3 (binding-attenuated CD3 antibody), and thereby increase the CD3-binding activity of the binding-attenuated CD3 antibody (or stabilize the antigen-antibody complex formed by CD3 and the binding-attenuated CD3 antibody). In certain embodiments, the first and/or second antigen-binding domains in the antigen-binding molecule of the present disclosure may be antigen-binding domains derived from clamping antigen-binding molecules (clamping antigen-binding domains).

In an embodiment of the above aspects, both the first and second antigen-binding domains have the same amino acid sequence. In another embodiment, each of the first and second antigen-binding domains has a different amino acid sequence.

In an embodiment of the above aspects, at least one of two antigens to which the first and second antigen-binding domains bind is a soluble protein or a membrane protein.

### <Functions of Antigen-Binding Molecules>

In an embodiment of the above aspects, the antigen-binding molecule of the present disclosure has activity of holding two antigen molecules at spatially close positions. In certain embodiments, the antigen-binding molecule of the present disclosure is capable of holding two antigen molecules at closer positions than a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region).

In an embodiment of the above aspects, the antigen-binding molecule of the present disclosure has activity of regulating interaction between two antigen molecules. Without being bound by a particular theory, the activity of regulating interaction is thought to be resulted from holding two antigen molecules at spatially closer positions by the antigen-binding molecule of the present disclosure. In certain embodiments, the antigen-binding molecule of the present disclosure is capable of enhancing or diminishing interaction between two antigen molecules as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region).

In certain embodiments, the two antigen molecules bound by the antigen-binding molecule of the present disclosure are a ligand and a receptor thereof, respectively, and the antigen-binding molecule of the present disclosure has activity of promoting activation of the receptor by the ligand. In certain other embodiment, the two antigen molecules bound by the antigen-binding molecule of the present disclosure are an enzyme and a substrate thereof, respectively, and the antigen-binding molecule of the present disclosure has activity of promoting catalytic reaction of the enzyme with the substrate.

Further, in certain other embodiments, both of the two antigen molecules bound by the antigen-binding molecule of the present disclosure are antigens (for example, proteins) present on cellular surfaces, and the antigen-binding molecule of the present disclosure has activity of promoting interaction between a cell expressing the first antigen and a cell expressing the second antigen. For example, the cell expressing the first antigen and the cell expressing the second antigen are, respectively, a cell with cytotoxic activity and a target cell thereof, and the antigen-binding molecule of the present disclosure promotes damage of the target cell by the cell with cytotoxic activity. The cell with cytotoxic activity is, for example, a T cell, NK cell, monocyte, or macrophage.

In an embodiment of the above aspects, the antigen-binding molecule of the present disclosure has activity of regulating activation of two antigen molecules which are activated by association with each other. Without being bound by a particular theory, the activity of regulating activation is thought to be resulted from holding two antigen molecules at spatially closer positions by the antigen-binding molecule of the present disclosure. In certain embodiments, the antigen-binding molecule of the present disclosure can enhance or diminish activation of two antigen molecules as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). For example, such antigen molecules are selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

In an embodiment of the above aspects, the antigen-binding molecule of the present disclosure has resistance to protease cleavage. In certain embodiments, the antigen-binding molecule of the present disclosure has increased resistance to protease cleavage as compared to a control antigen-binding molecule, and the control antigen-binding molecule differs from the antigen-binding molecule of the present disclosure only in that the control antigen-binding molecule has one less bond between the two antigen-binding domains. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). In certain embodiments, in the antigen-binding molecule of the present disclosure, the proportion of the full-length molecule (for example, full-length IgG molecule) remaining after protease treatment is increased as compared to the control antigen-binding molecule. In certain embodiments, in the antigen-binding molecule of the present disclosure, the proportion of a particular fragment (for example, Fab monomer) produced after protease treatment is reduced as compared to the control antigen-binding molecule.

In an embodiment of the above aspects, when the antigen-binding molecule of the present disclosure is treated with a protease, a dimer of the antigen-binding domains or fragments thereof (for example, crosslinked Fab dimer) is excised. In certain embodiments, when the control antigen-binding molecule, which differs from the antigen-binding molecule of the present disclosure only in that it has one less bond between the two antigen-binding domains, is treated with the protease, monomers of the antigen-binding domains or fragments thereof are excised. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). In these embodiments, the protease can cleave the hinge region of the antigen-binding molecule.

### <Pharmaceutical Compositions >

In an aspect, the present disclosure provides a pharmaceutical composition comprising the antigen-binding molecule of the present disclosure and a pharmaceutically acceptable carrier.

### <Use of Antigen-Binding Molecules>

In an aspect, the present disclosure provides a method for holding two antigen molecules at spatially close positions, comprising:
(a) providing an antigen-binding molecule comprising two antigen-binding domains, wherein the two antigen-binding domains are linked with each other via one or more bonds,
(b) adding to the antigen-binding molecule another bond which links the two antigen-binding domains with each other, and
(c) contacting the antigen-binding molecule produced in (b) with the two antigen-binding molecules.

In certain embodiments, some or all of the one or more bonds recited in (a) above are bonds in which the amino acid residues from which the bonds between the antigen-binding domains originate are derived from amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for holding two antigen molecules at spatially close positions which comprises contacting two antigen molecules with the antigen-binding molecule or pharmaceutical composition of the present disclosure. The present disclosure further provides an antigen-binding molecule or pharmaceutical composition of the present disclosure for use in holding two antigen molecules at spatially close positions.

In another aspect, the present disclosure provides a method for regulating interaction between two antigen molecules, comprising:
(a) providing an antigen-binding molecule comprising two antigen-binding domains, wherein the two antigen-binding domains are linked with each other via one or more bonds,
(b) adding to the antigen-binding molecule another bond which links the two antigen-binding domains with each other, and
(c) contacting the antigen-binding molecule produced in (b) with the two antigen-binding molecules.

In certain embodiments, some or all of the one or more bonds recited in (a) above are bonds in which the amino acid residues from which the bonds between the antigen-binding domains originate are derived from amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for regulating interaction between two antigen molecules which comprises contacting two antigen molecules with the antigen-binding molecule or pharmaceutical composition of the present disclosure. The present disclosure further provides an antigen-binding molecule or pharmaceutical composition of the present disclosure for use in regulating interaction between two antigen molecules.

Further, in another aspect, the present disclosure provides a method for regulating activity of two antigen molecules which are activated by association with each other, comprising:
(a) providing an antigen-binding molecule comprising two antigen-binding domains, wherein the two antigen-binding domains are linked with each other via one or more bonds,
(b) adding to the antigen-binding molecule another bond which links the two antigen-binding domains with each other, and
(c) contacting the antigen-binding molecule produced in (b) with the two antigen-binding molecules.

In certain embodiments, some or all of the one or more bonds recited in (a) above are bonds in which the amino acid residues from which the bonds between the antigen-binding domains originate are derived from amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region). The present disclosure also provides a method for regulating activity of two antigen molecules which are activated by association with each other, which comprises contacting two antigen molecules with the antigen-binding molecule or pharmaceutical composition of the present disclosure. The present disclosure further provides an antigen-binding molecule or pharmaceutical composition of the present disclosure for use in regulating activity of two antigen molecules which are activated by association with each other.

Furthermore, in another aspect, the present disclosure provides a method for increasing resistance of an antigen-binding molecule to protease cleavage, comprising:
(a) providing an antigen-binding molecule comprising two antigen-binding domains, wherein the two antigen-binding domains are linked with each other via one or more bonds, and
(b) adding to the antigen-binding molecule another bond which links the two antigen-binding domains with each other.

In certain embodiments, some or all of the one or more bonds recited in (a) above are bonds in which the amino acid residues from which the bonds between the antigen-binding domains originate are derived from amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

The antigen-binding molecule used in these various methods may have the characteristics of the antigen-binding molecules described herein.

### <Methods for Producing Antigen-Binding Molecules>

In an aspect, the present disclosure provides a method for producing an antigen-binding molecule which has activity of holding two antigen molecules at spatially close positions, comprising:
(a) providing a nucleic acid encoding a polypeptide comprising a first antigen-binding domain and a nucleic acid encoding a polypeptide comprising a second antigen-binding domain, wherein each of the two antigen-binding domains comprises one or more amino acid residues from which a bond for linking the two antigen-binding domains originates,
(b) introducing a mutation into the nucleic acids encoding the two antigen-binding domains such that another bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via two or more bonds.

In certain embodiments, some or all of the one or more amino acid residues recited in (a) above from which the bond between the antigen-binding domains originates are amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

In another aspect, the present disclosure provides a method for producing an antigen-binding molecule which has activity of regulating interaction between two antigen molecules, comprising:
(a) providing a nucleic acid encoding a polypeptide comprising a first antigen-binding domain and a nucleic acid encoding a polypeptide comprising a second antigen-binding domain, wherein each of the two antigen-binding domains comprises one or more amino acid residues from which a bond for linking the two antigen-binding domains originates,
(b) introducing a mutation into the nucleic acids encoding the two antigen-binding domains such that another bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via two or more bonds.

In certain embodiments, some or all of the one or more amino acid residues recited in (a) above from which the bond between the antigen-binding domains originates are amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Further, in another aspect, the present disclosure provides a method for producing an antigen-binding molecule which has activity of regulating activation of two antigen molecules which are activated by association with each other, comprising:
(a) providing a nucleic acid encoding a polypeptide comprising a first antigen-binding domain and a nucleic acid encoding a polypeptide comprising a second antigen-binding domain, wherein each of the two antigen-binding domains comprises one or more amino acid residues from which a bond for linking the two antigen-binding domains originates,
(b) introducing a mutation into the nucleic acids encoding the two antigen-binding domains such that another bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via two or more bonds.

In certain embodiments, some or all of the one or more amino acid residues recited in (a) above from which the bond between the antigen-binding domains originates are amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

Furthermore, in another aspect, the present disclosure provides a method for producing an antigen-binding molecule which has increased resistance to protease cleavage, comprising:
(a) providing a nucleic acid encoding a polypeptide comprising a first antigen-binding domain and a nucleic acid encoding a polypeptide comprising a second antigen-binding domain, wherein each of the two antigen-binding domains comprises one or more amino acid residues from which a bond for linking the two antigen-binding domains originates,
(b) introducing a mutation into the nucleic acids encoding the two antigen-binding domains such that another bond linking the two antigen-binding domains is added,
(c) introducing the nucleic acids produced in (b) into a host cell,
(d) culturing the host cell such that the two polypeptides are expressed, and
(e) obtaining an antigen-binding molecule which is a polypeptide comprising the first and second antigen-binding domains, wherein the two antigen-binding domains are linked with each other via two or more bonds.

In certain embodiments, some or all of the one or more amino acid residues recited in (a) above from which the bond between the antigen-binding domains originates are amino acid residues which are present in a wild-type Fab or hinge region (for example, cysteine residues in the hinge region). In a further embodiment, said another bond recited in (b) above is a bond in which the amino acid residues from which the bond between the antigen-binding domains originates are derived from mutated amino acid residues which are not present in a wild-type Fab or hinge region (for example, cysteine residues which are not present in the wild-type Fab or hinge region).

The antigen-binding molecule produced in these various aspects may have the characteristics of the antigen-binding molecules described herein.

### <Methods of Screening for Antigen-Binding Molecules>

In another aspect, the present disclosure provides a method for identifying a novel pair of protein molecules which are activated by association with each other, comprising:
(a) providing two arbitrary protein molecules,
(b) producing, by the production method of the present disclosure, an antigen-binding molecule comprising two antigen-binding domains which respectively bind to the two protein molecules, wherein the antigen-binding molecule has activity of holding the two protein molecules at close positions,
(c) contacting the antigen-binding molecule produced in (b) with the two protein molecules, and
(d) assessing whether or not the two protein molecules are activated.

In certain embodiments, at least one of the protein molecules is selected from the group consisting of receptors belonging to cytokine receptor superfamilies, G protein-coupled receptors, ion channel receptors, tyrosine kinase receptors, immune checkpoint receptors, antigen receptors, CD antigens, costimulatory molecules, and cell adhesion molecules.

### <Linkage of Antigen-Binding Domains>

In a non-limiting embodiment, two or more antigen-binding domains contained in an antigen-binding molecule of the present disclosure are linked with each other via one or more bonds. In a preferred embodiment, an antigen-binding domain contained in an antigen-binding molecule of the present disclosure has, by itself, activity to bind to an antigen. In such an embodiment, the antigen-binding molecule of the present disclosure containing two antigen-binding domains can bind to two or more antigen molecules; the antigen-binding molecule of the present disclosure containing three antigen-binding domains can bind to three or more antigen molecules; the antigen-binding molecule of the present disclosure containing four antigen-binding domains can bind to four or more antigen molecules; and the antigen-binding molecule of the present disclosure containing N antigen-binding domains can bind to N or more antigen molecules.

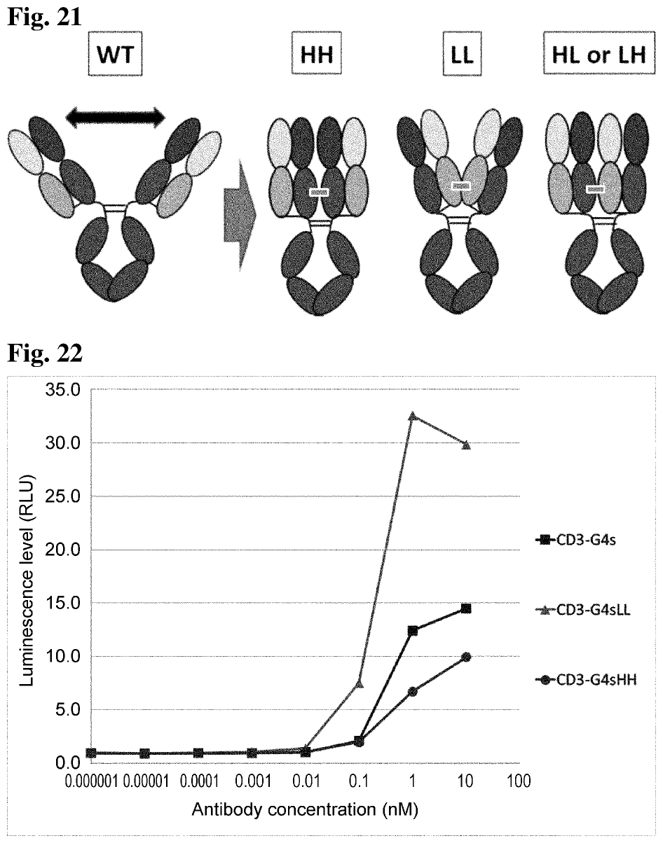

In certain embodiments, at least one of the bonds between the antigen-binding domains contained in an antigen-binding molecule of the present disclosure is different from a bond found in a naturally-occurring antibody (for example, in a wild-type Fab or hinge region). Examples of the bonds found between the antigen-binding domains of a naturally-occurring antibody (for example, naturally-occurring IgG antibody) include disulfide bonds in the hinge region. Bonds between amino acid residues positioned in a region other than the hinge region may be bonds between amino acid residues within an antibody fragment (for example, Fab), and they include bonds between the heavy chains (HH), bonds between the light chains (LL), and bonds between the heavy and light chains (HL or LH) (see FIG. 21). Examples of the amino acid residues in the heavy or light chain from which the bonds between the antigen-binding domains originate include amino acid residues at the above-mentioned positions within the variable region (VH region or VL region) or within the constant region (CH1 region, hinge region, or CL region).

In a non-limiting embodiment, the bonds between the antigen-binding domains may originate from multiple amino acid residues present at positions separate from each other in the primary structure in at least one of two or more antigen-binding domains contained in an antigen-binding molecule of the present disclosure. The distance between the multiple amino acid residues is a distance that allows the achievement of the structures of two or more, sufficiently close antigen-binding domains as a result of linkage between the antigen-binding domains by the bonds which originate from the amino acid residues. The distance between the multiple amino acid residues may be, for example, 4 amino acids or more, 5 amino acids or more, 6 amino acids or more, 7 amino acids or more, 8 amino acids or more, 9 amino acids or more, 10 amino acids or more, 11 amino acids or more, 12 amino acids or more, 13 amino acids or more, 14 amino acids or more, 15 amino acids or more, 20 amino acids or more, 25 amino acids or more, 30 amino acids or more, 35 amino acids or more, 40 amino acids or more, 45 amino acids or more, 50 amino acids or more, 60 amino acids or more, 70 amino acids or more, 80 amino acids or more, 90 amino acids or more, 100 amino acids or more, 110 amino acids or more, 120 amino acids or more, 130 amino acids or more, 140 amino acids or more, 150 amino acids or more, 160 amino acids or more, 170 amino acids or more, 180 amino acids or more, 190 amino acids or more, 200 amino acids or more, 210 amino acids or more, or 220 amino acids or more.

Further, the number of the bonds between the antigen-binding domains and the number of the amino acid residues from which the bonds originate are a number that allows the achievement of the structures of two or more, sufficiently close antigen-binding domains as a result of linkage between the antigen-binding domains by the bonds. The number may be, for example, two or more, three or more, four or more, five or more, six or more, seven or more, eight or more, nine or more, or ten or more.

In certain embodiments, as long as the structures of two or more, sufficiently close antigen-binding domains are achieved as a result of linkage between the antigen-binding domains by three or more bonds which respectively originate from three or more amino acid residues in the antigen-binding domains, the distance in the primary structure between any two amino acid residues selected from the three amino acid residues may be seven amino acids or more in at least one amino acid residue pair, and may be less than seven amino acids in the remainder of amino acid residue pairs.

In connection with antigen-binding domains contained in antigen-binding molecules of the present disclosure, “sufficiently close” means that two or more antigen-binding domains are close to the extent that this is sufficient for achieving the desired functions (activities) of the antigen-binding molecule of the present disclosure. Examples of the desired functions (activities) include activity of holding two antigen molecules at spatially close positions; activity of regulating interaction between two antigen molecules; activity of promoting activation of a receptor by a ligand; activity of promoting catalytic reaction of an enzyme with a substrate; activity of promoting interaction between a cell expressing a first antigen and a cell expressing a second antigen; activity of promoting damage of a target cell by a cell with cytotoxic activity (such as a T cell, NK cell, monocyte, macrophage); activity of regulating activation of two antigen molecules which are activated by association with each other; and resistance to protease cleavage of the antigen-binding molecules.

In a non-limiting embodiment, the bond between the antigen-binding domains contained in an antigen-binding molecule of the present disclosure may be a covalent bond or a non-covalent bond. The covalent bond may be a covalent bond formed by directly crosslinking an amino acid residue in a first antigen-binding domain and an amino acid residue of a second antigen-binding domain, for example, a disulfide bond between cysteine residues. The directly crosslinked amino acid residue may be present in an antibody fragment such as Fab, or within a hinge region.

In another embodiment, a covalent bond is formed by crosslinking an amino acid residue in a first antigen-binding domain and an amino acid residue of a second antigen-binding domain via a crosslinking agent. For example, when an amine-reactive crosslinking agent is used for crosslinking, the crosslinkage can be made via a free amino group of the N-terminal amino acid of the antigen-binding domain, or a primary amine of the side chain of a lysine residue in the antigen-binding domain. Amine-reactive crosslinking agents include a functional group that forms a chemical bond with a primary amine, such as isothiocyanate, isocyanate, acyl azide, NHS ester, sulfonyl chloride, aldehyde, glyoxal, epoxide, oxirane, carbonate, aryl halide, imide ester, carbodiimide, anhydride, and fluoroester. Representative examples include DSG (disuccinimidyl glutarate), DSS (disuccinimidyl suberate), BS3 (bis(sulfosuccinimidyl) suberate), DSP (dithiobis(succinimidyl propionate)), DTSSP (3,3′-dithiobis (sulfosuccinimidyl propionate)), DST (disuccinimidyl tartrate), BSOCOES (bis(2-(succinimidooxycarbonyloxy) ethyl)sulfone), EGS (ethylene glycol bis(succinimidyl succinate)), Sulfo-EGS (ethylene glycol bis(sulfosuccinimidyl succinate)), DMA (dimethyl adipimidate), DMP (dimethyl pimelimidate), DMS (dimethyl suberimidate), and DFDNB (1,5-difluoro-2,4-dinitrobenzene). Examples of other crosslinking agents include carboxyl/amine-reactive, sulfhydryl-reactive, aldehyde-reactive, and light-reactive crosslinking agents.

The non-covalent bond for linking the antigen-binding domains may be an ionic bond, hydrogen bond, or hydrophobic bond.

Whether the number of the bonds between the antigen-binding domains is larger than that of a control antigen-binding molecule (e.g., an antigen-binding molecule having a structure substantially similar to a naturally-occurring antibody structure) can be assessed by, for example, the following method. First, an antigen-binding molecule of interest and a control antigen-binding molecule are treated with a protease that cuts out the antigen-binding domain (for example, a protease that cleaves the N-terminal side of the crosslinkage site of the hinge regions such as papain and Lys-C), and then subjected to non-reducing electrophoresis. Next, an antibody that recognizes a part of the antigen-binding domain (for example, anti-kappa chain HRP-labelled antibody) is used to detect fragments which are present after the protease treatment. When only a monomer of the antigen-binding domain (for example, Fab monomer) is detected for the control antigen-binding molecule, and a multimer of the antigen-binding domain (for example, Fab dimer) is detected for the antigen-binding molecule of interest, then it can be assessed that the number of the bonds between the antigen-binding domains of the antigen-binding molecule of interest is larger than that of the control antigen-binding molecule.

The formation of a disulfide bond between cysteines in a modified antigen-binding molecule produced by introducing cysteines into a control antigen-binding molecule can be assessed by, for example, the following method. First, an antigen-binding molecule of interest is incubated with chymotrypsin in 20 mM phosphate buffer (pH7.0), and then the mass of peptides expected to be generated from the amino acid sequence of each antibody is detected by LC/MS. If a component corresponding to the theoretical mass of a peptide that should be generated when the newly-introduced cysteines form a disulfide bond is detected, the introduced cysteines can be assessed as having formed a disulfide bond. Moreover, if this component becomes undetectable when the sample containing the above-mentioned antigen-binding molecule is analyzed after adding an agent for reducing disulfide bonds (for example, tris(2-carboxyethyl)phosphine) to the sample, the correctness of the above assessment will be further strongly verified.

### <Resistance to Protease Cleavage>

In a non-limiting embodiment, the antigen-binding molecule of the present disclosure has resistance to protease cleavage. In certain embodiments, the resistance to protease cleavage of the antigen-binding molecule of the present disclosure is increased compared with a control antigen-binding molecule (for example, an antigen-binding molecule having a structure substantially similar to a naturally-occurring antibody structure) where the number of bonds between the antigen-binding domains is lesser by one or more compared to the antigen-binding molecule. In a further embodiment, the one less bond can be selected from bonds in which the amino acid residues from which the bonds between the antigen-binding domains originate are derived from mutated amino acid residues which are not present in a wild-type Fab or hinge region (for example, cysteine residues which are not present in the wild-type Fab or hinge region). If the proportion of the full-length molecule (for example, full-length IgG molecule) remaining after protease treatment is increased, or the proportion of a particular fragment (for example, Fab monomer) produced after protease treatment is reduced for an antigen-binding molecule compared to a control antigen-binding molecule, then it can be assessed that the resistance to protease cleavage is increased (protease resistance is improved).

In certain embodiments, the proportion of the full-length molecule remaining after protease treatment may be, relative to all antigen-binding molecules, for example, 0.5% or more, 1% or more, 1.5% or more, 2% or more, 2.5% or more, 3% or more, 3.5% or more, 4% or more, 4.5% or more, 5% or more, 7.5% or more, 10% or more, 12.5% or more, 15% or more, 20% or more, 25% or more, 30% or more, 35% or more, 40% or more, 45% or more, or 50% or more. In certain other embodiments, the proportion of a monomer of an antigen-binding domain (for example, Fab) produced after protease treatment may be, relative to all antigen-binding molecules, for example, 99% or less, 98% or less, 97% or less, 96% or less, 95% or less, 94% or less, 93% or less, 92% or less, 91% or less, 90% or less, 85% or less, 80% or less, 75% or less, 70% or less, 60% or less, 50% or less, 40% or less, 30% or less, 20% or less, or 10% or less. In certain other embodiments, the proportion of a dimer of an antigen-binding domain (for example, Fab) produced after protease treatment may be, relative to all antigen-binding molecules, for example, 0.5% or more, 1% or more, 1.5% or more, 2% or more, 2.5% or more, 3% or more, 3.5% or more, 4% or more, 4.5% or more, 5% or more, 7.5% or more, 10% or more, 12.5% or more, 15% or more, 20% or more, 25% or more, 30% or more, 35% or more, 40% or more, 45% or more, or 50% or more.

Examples of proteases include, but are not limited to, Lys-C, plasmin, human neutrophil elastase (HNE), and papain.

In a further aspect, an antigen-binding molecule according to any of the above embodiments may incorporate any of the features, singly or in combination, as described in Sections 1-7 below:

### 1. Antigen-Binding Molecule Affinity

In certain embodiments, an antigen-binding molecule provided herein has a dissociation constant (KD) of 1 micro M or less, 100 nM or less, 10 nM or less, 1 nM or less, 0.1 nM or less, 0.01 nM or less, or 0.001 nM or less (e.g., 10-8 M or less, e.g., from 10-8 M to 10-13 M, e.g., from 10-9 M to 10-13 M).

### 2. Antibody Fragments

In certain embodiments, an antigen-binding molecule provided herein is an antibody fragment. Antibody fragments include, but are not limited to, Fab, Fab′, Fab′-SH, F(ab′)₂, Fv, and scFv fragments, and other fragments described herein. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

### 3. Chimeric and Humanized Antibodies

In certain embodiments, an antigen-binding molecule provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

### 4. Human Antibodies

In certain embodiments, an antigen-binding molecule provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

### 5. Library-Derived Antigen-Binding Molecules

Antigen-binding molecules of the invention may be isolated by screening combinatorial libraries for antigen-binding molecules with the desired activity or activities. For example, a variety of methods are known in the art for generating phage display libraries and screening such libraries for antigen-binding molecules possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

### 6. Multispecific Antigen-Binding Molecules

In certain embodiments, an antigen-binding molecule provided herein is a multispecific antigen-binding molecule, e.g. a bispecific antigen-binding molecule. Multispecific antigen-binding molecules are monoclonal antigen-binding molecules that have binding specificities for at least two different sites. In certain embodiments, one of the binding specificities is for a particular antigen (e.g., CD3) and the other is for any other antigen (e.g., CD28 or cancer antigen). In certain embodiments, bispecific antigen-binding molecules may bind to two different epitopes on a single antigen. Bispecific antigen-binding molecules can be prepared as full-length antibodies or antibody fragments.

Techniques for making multispecific antigen-binding molecules include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” (also called “knobs-in-holes” or “KiH”) engineering (see, e.g., U.S. Pat. No. 5,731,168). Multi-specific antigen-binding molecules may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

### 7. Antigen-Binding Molecule Variants

In certain embodiments, amino acid sequence variants of the antigen-binding molecules provided herein are contemplated. For example, it may be desirable to improve the binding affinity and/or other biological properties of the antigen-binding molecule. Amino acid sequence variants of an antigen-binding molecule may be prepared by introducing appropriate modifications into the nucleotide sequence encoding the antigen-binding molecule, or by peptide synthesis. Such modifications include, for example, deletions from, and/or insertions into and/or substitutions of residues within the amino acid sequences of the antigen-binding molecule. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antigen-binding molecule variants having one or more amino acid substitutions are provided. Sites of interest for substitutional mutagenesis include the HVRs and FRs. Conservative substitutions are shown the table below under the heading of “preferred substitutions.” More substantial changes are provided in the table under the heading of “exemplary substitutions,” and as further described below in reference to amino acid side chain classes. Amino acid substitutions may be introduced into an antigen-binding molecule of interest and the products screened for a desired activity, e.g., retained/improved antigen binding, decreased immunogenicity, or improved ADCC or CDC.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antigen-binding molecule (e.g. a humanized or human antibody). Generally, the resulting variant(s) selected for further study will have modifications (e.g., improvements) in certain biological properties (e.g., increased affinity, reduced immunogenicity) relative to the parent antigen-binding molecule and/or will have substantially retained certain biological properties of the parent antigen-binding molecule. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antigen-binding molecule affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antigen-binding molecule variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antigen-binding molecule to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antigen-binding molecule that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antigen-binding molecule with antigen is affected. Further substitutions may be introduced at the amino acid locations demonstrating functional sensitivity to the initial substitutions. Alternatively, or additionally, a crystal structure of a complex of antigens and an antigen-binding molecule may be analyzed to identify contact points between the antigen-binding molecule and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding molecule with an N-terminal methionyl residue. Other insertional variants of the antigen-binding molecule include the fusion of an enzyme (e.g. for ADEPT) or a polypeptide which increases the plasma half-life of the antigen-binding molecule to the N- or C-terminus of the antigen-binding molecule.

### b) Glycosylation Variants

In certain embodiments, an antigen-binding molecule provided herein is altered to increase or decrease the extent to which the antigen-binding molecule is glycosylated. Addition or deletion of glycosylation sites to an antigen-binding molecule may be conveniently accomplished by altering the amino acid sequence such that one or more glycosylation sites is created or removed.

Where the antigen-binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antigen-binding molecule of the invention may be made in order to create antigen-binding molecule variants with certain improved properties.

In one embodiment, antigen-binding molecule variants are provided having a carbohydrate structure that lacks fucose attached (directly or indirectly) to an Fc region. For example, the amount of fucose in such antigen-binding molecule may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antigen-binding molecules. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antigen-binding molecule variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antigen-binding molecules include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antigen-binding molecule variants are further provided with bisected oligosaccharides, e.g., in which a biantennary oligosaccharide attached to the Fc region of the antigen-binding molecule is bisected by GlcNAc. Such antigen-binding molecule variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antigen-binding molecule variants with at least one galactose residue in the oligosaccharide attached to the Fc region are also provided. Such antigen-binding molecule variants may have improved CDC function. Such antigen-binding molecule variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antigen-binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antigen-binding molecule variant that possesses some but not all effector functions, which make it a desirable candidate for applications in which the half life of the antigen-binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antigen-binding molecule lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antigen-binding molecule is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antigen-binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antigen-binding molecule variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antigen-binding molecule variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antigen-Binding Molecule Variants

In certain embodiments, it may be desirable to create cysteine engineered antigen-binding molecules, e.g., “thioMAbs,” in which one or more residues of an antigen-binding molecule are substituted with cysteine residues. In particular embodiments, the substituted residues occur at accessible sites of the antigen-binding molecule. By substituting those residues with cysteine, reactive thiol groups are thereby positioned at accessible sites of the antigen-binding molecule and may be used to conjugate the antigen-binding molecule to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen-binding molecules may be generated as described, e.g., in U.S. Pat. No. 7,521,541.

### e) Antigen-Binding Molecule Derivatives

In certain embodiments, an antigen-binding molecule provided herein may be further modified to contain additional nonproteinaceous moieties that are known in the art and readily available. The moieties suitable for derivatization of the antigen-binding molecule include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antigen-binding molecule may vary, and if more than one polymer are attached, they can be the same or different molecules. In general, the number and/or type of polymers used for derivatization can be determined based on considerations including, but not limited to, the particular properties or functions of the antigen-binding molecule to be improved, whether the antigen-binding molecule derivative will be used in a therapy under defined conditions, etc.

In connection with an antigen-binding molecule in the present disclosure, examples of the desired property (activity) can include, but are not particularly limited to, binding activity, neutralizing activity, cytotoxic activity, agonist activity, antagonist activity, and enzymatic activity. The agonist activity is an activity of intracellularly transducing signals, for example, through the binding of an antibody to an antigen such as a receptor to induce change in some physiological activity. Examples of the physiological activity can include, but are not limited to, proliferative activity, survival activity, differentiation activity, transcriptional activity, membrane transport activity, binding activity, proteolytic activity, phosphorylating/dephosphorylating activity, redox activity, transfer activity, nucleolytic activity, dehydration activity, cell death-inducing activity, and apoptosis-inducing activity.

In another embodiment, conjugates of an antigen-binding molecule and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antigen binding molecule-nonproteinaceous moiety are killed.

### B. Recombinant Methods and Compositions

Antigen-binding molecules may be produced using recombinant methods and compositions, e.g., as described in U.S. Pat. No. 4,816,567. In one embodiment, isolated nucleic acid encoding an antigen-binding molecule in the present disclosure (a polypeptide comprising an antigen-binding domain described herein) is provided. Such nucleic acid may encode an amino acid sequence comprising the VL and/or an amino acid sequence comprising the VH of the antigen-binding molecule (e.g., the light and/or heavy chains of the antigen-binding molecule). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antigen-binding molecule and an amino acid sequence comprising the VH of the antigen-binding molecule, or (2) a first vector comprising a nucleic acid that encodes an amino acid sequence comprising the VL of the antigen-binding molecule and a second vector comprising a nucleic acid that encodes an amino acid sequence comprising the VH of the antigen-binding molecule. In one embodiment, the host cell is eukaryotic, e.g. a Chinese Hamster Ovary (CHO) cell or lymphoid cell (e.g., Y0, NS0, Sp2/0 cell). In one embodiment, a method of making an antigen-binding molecule in the present disclosure is provided, wherein the method comprises culturing a host cell comprising a nucleic acid encoding the antigen-binding molecule, as provided above, under conditions suitable for expression of the antigen-binding molecule, and optionally recovering the antigen-binding molecule from the host cell (or host cell culture medium).

For recombinant production of an antigen-binding molecule in the present disclosure, nucleic acid encoding an antigen-binding molecule, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antigen-binding molecule).

Suitable host cells for cloning or expression of antigen-binding molecule-encoding vectors include prokaryotic or eukaryotic cells described herein. For example, antigen-binding molecules may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antigen-binding molecule may be isolated from the bacterial cell paste in a soluble fraction and can be further purified.

In addition to prokaryotes, eukaryotic microbes such as filamentous fungi or yeast are suitable cloning or expression hosts for antigen-binding molecule-encoding vectors, including fungi and yeast strains whose glycosylation pathways have been “humanized,” resulting in the production of an antigen-binding molecule with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antigen-binding molecule are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding molecules in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antigen-binding molecule production, see, e.g., Yazaki and Wu, Methods in Molecular Biology, Vol. 248 (B. K. C. Lo, ed., Humana Press, Totowa, NJ), pp. 255-268 (2003).

### C. Assays

Antigen-binding molecules provided herein may be identified, screened for, or characterized for their physical/chemical properties and/or biological activities by various assays known in the art.

### 1. Binding Assays and Other Assays

In one aspect, an antigen-binding molecule in the present disclosure is tested for its antigen binding activity, e.g., by known methods such as ELISA, Western blot, etc.

### 2. Activity Assays

In one aspect, assays are provided for identifying antigen-binding molecules thereof having biological activity. Biological activity may include, e.g., activity of holding two antigen molecules at spatially close positions, activity of regulating interaction between two antigen molecules, activity of promoting activation of an receptor by a ligand, activity of promoting catalytic reaction of an enzyme with a substrate, promoting interaction between a cell expressing a first antigen and a cell expressing a second antigen, activity of promoting damage of a target cell by a cell with cytotoxic activity (e.g., a T cell, NK cell, monocyte, or macrophage), activity of regulating activation of two antigen molecules which are activated by association with each other, and resistance to protease cleavage. Antigen-binding molecules having such biological activity in vivo and/or in vitro are also provided.

Furthermore, an antigen-binding molecule in the present disclosure can exert various biological activities depending on the type of an antigen molecule to which the antigen-binding molecule binds. Examples of such antigen-binding molecules include an antigen-binding molecule which binds to a T cell receptor (TCR) complex (e.g., CD3) and has activity of inducing T cell activation (agonist activity); and an antigen-binding molecule which binds to a molecule of TNF receptor superfamily (e.g., OX40 or 4-1BB) or of other co-stimulatory molecules (e.g., CD28 or ICOS) and has activity of promoting the above-mentioned activation (agonist activity). In certain embodiments, such biological activity exerted through the binding to an antigen molecule is enhanced or diminished by the linking of two or more antigen-binding domains comprised in the antigen-binding molecule in the present disclosure. Without being limited by theory, in certain embodiments, such enhancement or diminishment may be achieved because the interaction between two or more antigen molecules is regulated through the binding to the antigen-binding molecule in the present disclosure (e.g., the association between two or more antigen molecules is promoted).

In certain embodiments, an antigen-binding molecule of the invention is tested for such biological activity. Whether two antigen molecules are held spatially close can be evaluated using techniques such as crystal structure analysis, electron microscopy, and electron tomography-based structural analysis of a complex composed of antigens and an antigen-binding molecule. Whether two antigen-binding domains are spatially close to each other or whether the mobility of two antigen-binding domains is reduced can also be evaluated by the above-mentioned techniques. In particular, as for techniques to analyze the three-dimensional structure of IgG molecules using electron tomography, see, for example, Zhang et al., Sci. Rep. 5:9803 (2015). In electron tomography, the frequency of occurrence of structures that a subject molecule may form can be shown by histograms, enabling distributional evaluation of structural changes such as reduced mobility of domains. For example, when the relationship between values that can be taken by structure-related parameters, such as distance and angle between two domains, and their frequency of occurrence is shown by histograms, one can determine that the mobility of the two domains is decreased if their areas of distribution are decreased. Activity exerted through interaction and such of two antigen molecules can be evaluated by selecting and using an appropriate activity measurement system from known ones according to the type of target antigen molecules. The effect on protease cleavage can be evaluated using methods known to those skilled in the art, or methods described in the Examples below.

### D. Pharmaceutical Formulations (Pharmaceutical Compositions)

Pharmaceutical formulations of an antigen-binding molecule as described herein are prepared by mixing such antigen-binding molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antigen-binding molecule formulations are described in U.S. Pat. No. 6,267,958. Aqueous antigen-binding molecule formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antigen-binding molecule, which matrices are in the form of shaped articles, e.g. films, or microcapsules.

The formulations to be used for in vivo administration are generally sterile. Sterility may be readily accomplished, e.g., by filtration through sterile filtration membranes.

### EXAMPLES

The following are examples of antigen-binding molecules and methods of the present disclosure. It will be understood that various other embodiments may be practiced, given the general description provided above.

### Example 1

Optimizing Methods for Producing, Purification and Assessment of Antibodies Having One or More Disulfide Bonds within Fab Region

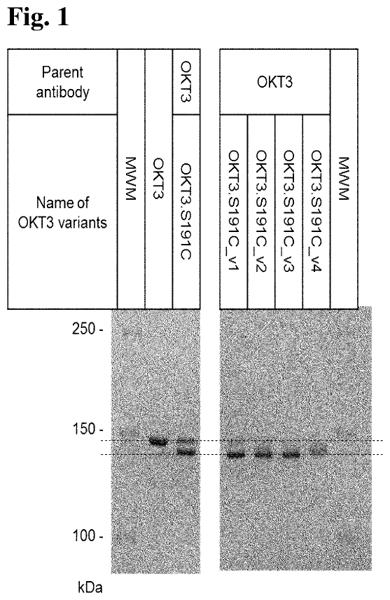

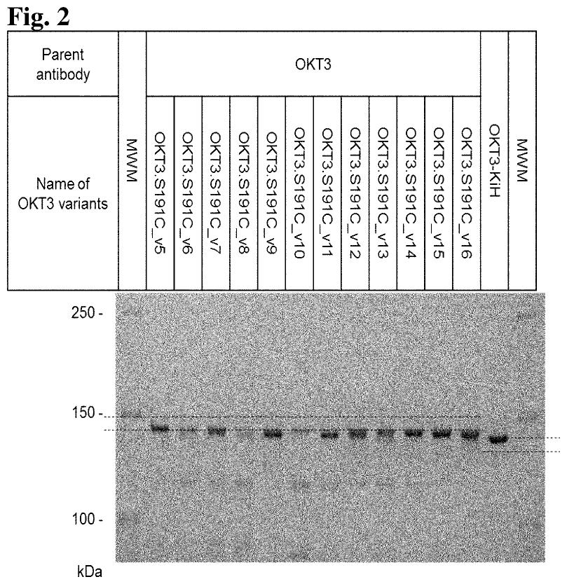

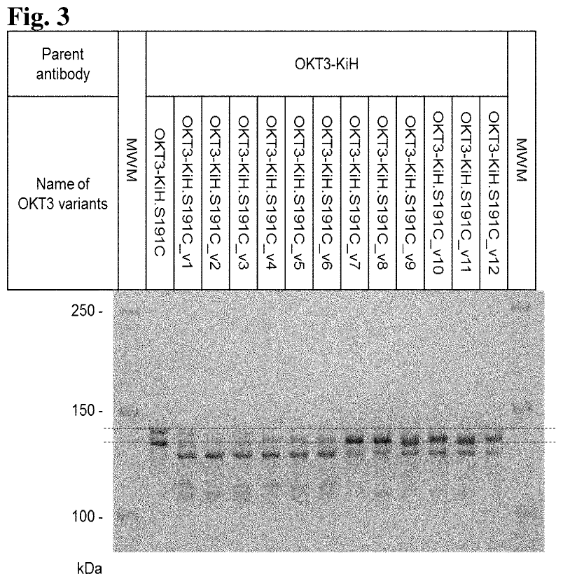

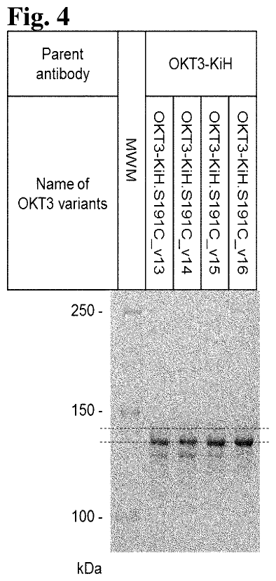

Preparation and assessment of antibodies having a single pair of cysteine substitution at various positions in the antibodies were described in Reference Examples 1-25. Based on the results of non-reducing SDS-PAGE (Reference Examples 8-2, 9-2, 10-2, and 11-2; see also FIGS. 1 to 4), it was found that some of the preparation of antibody having cysteine substitution comprises two or more structural variants/isoforms which differ in electrophoretic mobility, i.e. Double, Triple or Several bands as observed from the non-reducing SDS-PAGE gel images. For example, two bands were observed in the G1T4.S191C-IgG1 variant (cysteine substitution at the position 191 of the CH1 region) with about 66.3% percentage of the new band (corresponds to antibody preparation having one disulfide bond formed between two Fabs at position 191 of the CH1 region) relative to the band corresponding to that of the parent antibody. The results suggest that the antibody preparation of the G1T4.S191C-IgG1 variant comprises two or more structural isoforms which differ by one disulfide bond formed between the engineered cysteines, in particular isoform having the “paired cysteines” or isoform having the “free or unpaired cysteines”, can be generated during recombinant antibody production.

As described in further detail hereinbelow, the following non-limiting examples are directed to providing efficient and facile production, purification and analysis of the antibody having an engineered disulfide bond formed between the two Fabs of the antibody; methods for increasing structural homogeneity and relative abundance of the antibody in the “paired cysteines” form, i.e. having one or more engineered disulfide bond(s) formed between the two Fabs of the antibody; or methods for decreasing relative abundance of the antibody in the “free or unpaired cysteines” form, i.e. having no engineered disulfide bond formed between the two Fabs of the antibody.

### Example 1-1 Production of Antibodies Having Multiple Additional Disulfide Bonds within the Fab Region

To improve the percentage of antibody preparation of G1T4.S191C-IgG1 variant having an engineered disulfide bond formed at the position 191 of CH1 region of the antibody, additional one or two disulfide bonds were introduced into the heavy chain of an anti-human CD3 antibody, OKT3 (heavy chain: OKT3VH0000-G1T4 (SEQ ID NO: 1242), light chain: OKT3VL0000-KT0 (SEQ ID NO: 1243)) via cysteine substitution.

An amino acid residue structurally exposed to the surface of the OKT3 heavy chain constant region (G1T4, SEQ ID NO: 1244) was substituted with cysteine to produce the variant of OKT3 heavy chain constant region (G1T4.S191C, SEQ ID NO: 1245) shown in Table 1. In addition, other amino acid residues structurally exposed to the surface of G1T4.S191C were substituted with cysteine to produce the variants of G1T4.S191C shown in Table 2. These heavy chain constant regions were each linked with the OKT3 heavy chain variable region (OKT3VH0000, SEQ ID NO: 1246) to produce the OKT3 heavy chain variants, and expression vectors encoding the corresponding genes were produced by a method known in the art.

Similarly, amino acid residues structurally exposed to the surface of the OKT3 heavy chain constant region 1 (G1T4k, SEQ ID NO: 1263) and constant region 2 (G1T4h, SEQ ID NO: 1264) were substituted with cysteine to produce the variant of OKT3 heavy chain constant regions shown in Table 3, respectively. In addition, other amino acid residues structurally exposed to the surface of the variants shown in Table 3 were substituted with cysteine to produce the variants shown in Table 4. These heavy chain constant regions were each linked with the OKT3 heavy chain variable region (OKT3VH0000, SEQ ID NO: 1246) to produce the OKT3 heavy chain variants, and expression vectors encoding the corresponding genes were produced by a method known in the art. It is noted that the Knobs-into-Holes (KiH) mutations in the CH3 region are introduced into the heavy-chain constant regions 1 and 2 in this Example for promoting heterodimerization.

The OKT3 heavy chain variants produced as mentioned above were combined with the OKT3 light chain. The OKT3 variants shown in Table 5 and 6 were expressed by transient expression using Expi293 cells (Life technologies) by a method known to the person skilled in the art, and purified with Protein A by a method known to the person skilled in the art. In this Example, OKT3 and OKT3-KiH are called “parent antibodies”, OKT3.S191C and OKT3-KiH.S191C are called “S191C variants”, and their variants are called “additional variants”, respectively.

TABLE 1
G1T4 variant with single cysteine substitution

| | Position of cysteine | |
|---|---|---|
| Variant of heavy chain | substitution | SEQ |
| constant region | (EU numbering) | ID NO: |
| G1T4.S191C | 191 | 1245 |

TABLE 2
G1T4.S191C variants with additional cysteine substitution

| | Position of cysteine | |
|---|---|---|
| Variants of heavy chain | substitution | SEQ |
| constant region | (EU numbering) | ID NO: |
| G1T4.S191C.S131C.G194C | S131C/G194C | 1247 |
| G1T4.S191C.S132C.G194C | S132C/G194C | 1248 |
| G1T4.S191C.K133C.G194C | K133C/G194C | 1249 |
| G1T4.S191C.S134C.G194C | S134C/G194C | 1250 |
| G1T4.S191C.T135C.G194C | T135C/G194C | 1251 |
| G1T4.S191C.S136C.G194C | S136C/G194C | 1252 |
| G1T4.S191C.G137C.G194C | G137C/G194C | 1253 |
| G1T4.S191C.G138C.G194C | G138C/G194C | 1254 |
| G1T4.S191C.S131C.T195C | S131C/T195C | 1255 |
| G1T4.S191C.S132C.T195C | S132C/T195C | 1256 |
| G1T4.S191C.K133C.T195C | K133C/T195C | 1257 |
| G1T4.S191C.S134C.T195C | S134C/T195C | 1258 |
| G1T4.S191C.T135C.T195C | T135C/T195C | 1259 |
| G1T4.S191C.S136C.T195C | S136C/T195C | 1260 |
| G1T4.S191C.G137C.T195C | G137C/T195C | 1261 |
| G1T4.S191C.G138C.T195C | G138C/T195C | 1262 |

TABLE 3
G1T4k and G1T4h variants with single cysteine substitution

| | Position of cysteine | |
|---|---|---|
| Variants of heavy chain | substitution | SEQ |
| constant region | (EU numbering) | ID NO: |
| G1T4k.S191C | S191C | 1265 |
| G1T4h.S191C | S191C | 1266 |

TABLE 4
G1T4k.S191C and G1T4h.S191C variants
with additional cysteine substitution

| | Position of cysteine | |
|---|---|---|
| Variants of heavy chain | substitution | SEQ |
| constant region | (EU numbering) | ID NO: |
| G1T4k.S191C.S131C | S131C | 1267 |
| G1T4k.S191C.S132C | S132C | 1268 |
| G1T4k.S191C.K133C | K133C | 1269 |
| G1T4k.S191C.S134C | S134C | 1270 |
| G1T4k.S191C.T135C | T135C | 1271 |
| G1T4k.S191C.S136C | S136C | 1272 |
| G1T4k.S191C.G137C | G137C | 1273 |
| G1T4k.S191C.G138C | G138C | 1274 |
| G1T4k.S191C.S131C | S131C | 1275 |
| G1T4k.S191C.S132C | S132C | 1276 |
| G1T4k.S191C.K133C | K133C | 1277 |
| G1T4k.S191C.S134C | S134C | 1278 |
| G1T4k.S191C.T135C | T135C | 1279 |
| G1T4k.S191C.S136C | S136C | 1280 |
| G1T4k.S191C.G137C | G137C | 1281 |
| G1T4k.S191C.G138C | G138C | 1282 |
| G1T4h.S191C.G194C | G194C | 1283 |
| G1T4h.S191C.T195C | T195C | 1284 |

TABLE 5
OKT3 variants with cysteine substitution

| | | Heavy | Heavy | |
|---|---|---|---|---|
| | | chain | chain | |
| | | variable | constant | Light |
| | | region | region | chain |
| Name of OKT3 | Short | SEQ | SEQ | SEQ |
| variants | Name | ID NO: | ID NO: | ID NO: |
| OKT3 | OKT3 | 1246 | 1244 | 1243 |
| OKT3.S191C | OKT3.S191C | 1246 | 1245 | 1243 |
| OKT3.S191C.S131C.G194C | OKT3.S191C_v1 | 1246 | 1247 | 1243 |
| OKT3.S191C.S132C.G194C | OKT3.S191C_v2 | 1246 | 1248 | 1243 |
| OKT3.S191C.K133C.G194C | OKT3.S191C_v3 | 1246 | 1249 | 1243 |
| OKT3.S191C.S134C.G194C | OKT3.S191C_v4 | 1246 | 1250 | 1243 |
| OKT3.S191C.T135C.G194C | OKT3.S191C_v5 | 1246 | 1251 | 1243 |
| OKT3.S191C.S136C.G194C | OKT3.S191C_v6 | 1246 | 1252 | 1243 |
| OKT3.S191C.G137C.G194C | OKT3.S191C_v7 | 1246 | 1253 | 1243 |
| OKT3.S191C.G138C.G194C | OKT3.S191C_v8 | 1246 | 1254 | 1243 |
| OKT3.S191C.S131C.T195C | OKT3.S191C_v9 | 1246 | 1255 | 1243 |
| OKT3.S191C.S132C.T195C | OKT3.S191C_v10 | 1246 | 1256 | 1243 |
| OKT3.S191C.K133C.T195C | OKT3.S191C_v11 | 1246 | 1257 | 1243 |
| OKT3.S191C.S134C.T195C | OKT3.S191C_v12 | 1246 | 1258 | 1243 |
| OKT3.S191C.T135C.T195C | OKT3.S191C_v13 | 1246 | 1259 | 1243 |
| OKT3.S191C.S136C.T195C | OKT3.S191C_v14 | 1246 | 1260 | 1243 |
| OKT3.S191C.G137C.T195C | OKT3.S191C_v15 | 1246 | 1261 | 1243 |
| OKT3.S191C.G138C.T195C | OKT3.S191C_v16 | 1246 | 1262 | 1243 |

TABLE 6
OKT3-KiH variants with cysteine substitution

| | | Heavy | Heavy | Heavy | Heavy | |
|---|---|---|---|---|---|---|
| | | chain 1 | chain 1 | chain 2 | chain 2 | |
| | | variable | constant | variable | constant | Light |
| | | region | region | region | region | chain |
| | | SEQ ID | SEQ ID | SEQ ID | SEQ ID | SEQ ID |
| Name of OKT3 variants | Short Name | NO: | NO: | NO: | NO: | NO: |
| OKT3-KiH | OKT3-KiH | 1246 | 1263 | 1246 | 1264 | 1243 |
| OKT3-KiH.S191C | OKT3-KiH.S191C | 1246 | 1265 | 1246 | 1266 | 1243 |
| OKT3-KiH.S191C.S131C/ | OKT3-KiH.S191C_v1 | 1246 | 1267 | 1246 | 1283 | 1243 |
| S191C.G194C | | | | | | |
| OKT3-KiH.S191C.S132C/ | OKT3-KiH.S191C_v2 | 1246 | 1268 | 1246 | 1283 | 1243 |
| S191C.G194C | | | | | | |
| OKT3-KiH.S191C.K133C/ | OKT3-KiH.S191C_v3 | 1246 | 1269 | 1246 | 1283 | 1243 |
| S191C.G194C | | | | | | |
| OKT3-KiH.S191C.S134C/ | OKT3-KiH.S191C_v4 | 1246 | 1270 | 1246 | 1283 | 1243 |
| S191C.G194C | | | | | | |
| OKT3-KiH.S191C.T135C/ | OKT3-KiH.S191C_v5 | 1246 | 1271 | 1246 | 1283 | 1243 |
| S191C.G194C | | | | | | |
| OKT3-KiH.S191C.S136C/ | OKT3-KiH.S191C_v6 | 1246 | 1272 | 1246 | 1283 | 1243 |
| S191C.G194C | | | | | | |
| OKT3-KiH.S191C.G137C/ | OKT3-KiH.S191C_v7 | 1246 | 1273 | 1246 | 1283 | 1243 |
| S191C.G194C | | | | | | |
| OKT3-KiH.S191C.G138C/ | OKT3-KiH.S191C_v8 | 1246 | 1274 | 1246 | 1283 | 1243 |
| S191C.G194C | | | | | | |
| OKT3-KiH.S191C.S131C/ | OKT3-KiH.S191C_v9 | 1246 | 1267 | 1246 | 1284 | 1243 |
| S191C.T195C | | | | | | |
| OKT3-KiH.S191C.S132C/ | OKT3-KiH.S191C_v10 | 1246 | 1268 | 1246 | 1284 | 1243 |
| S191C.T195C | | | | | | |
| OKT3-KiH.S191C.K133C/ | OKT3-KiH.S191C_v11 | 1246 | 1269 | 1246 | 1284 | 1243 |
| S191C.T195C | | | | | | |
| OKT3-KiH.S191C.S134C/ | OKT3-KiH.S191C_v12 | 1246 | 1270 | 1246 | 1284 | 1243 |
| S191C.T195C | | | | | | |
| OKT3-KiH.S191C.T135C/ | OKT3-KiH.S191C_v13 | 1246 | 1271 | 1246 | 1284 | 1243 |
| S191C.T195C | | | | | | |
| OKT3-KiH.S191C.S136C/ | OKT3-KiH.S191C_v14 | 1246 | 1272 | 1246 | 1284 | 1243 |
| S191C.T195C | | | | | | |
| OKT3-KiH.S191C.G137C/ | OKT3-KiH.S191C_v15 | 1246 | 1273 | 1246 | 1284 | 1243 |
| S191C.T195C | | | | | | |
| OKT3-KiH.S191C.G138C/ | OKT3-KiH.S191C_v16 | 1246 | 1274 | 1246 | 1284 | 1243 |
| S191C.T195C | | | | | | |

### Example 1-2 Assessment of Electrophoretic Mobility in Polyacrylamide Gel of Antibodies Having Multiple Additional Disulfide Bonds within the Fab Region

It was examined whether the antibodies produced in Example 1-1 show a different electrophoretic mobility in polyacrylamide gel by non-reducing SDS-PAGE.

Sample Buffer Solution (2ME-) (×4) (Wako; 198-13282) was used for preparation of electrophoresis samples. The samples were treated for 10 minutes under the condition of specimen concentration 50 microgram/mL and 70 degrees C., and then subjected to non-reducing SDS-PAGE. In non-reducing SDS-PAGE, electrophoresis was carried out for 90 minutes at 125 V, using 4% SDS-PAGE mini 15 well 1.0 mm 15 well (TEFCO; Cat #01-052-6). Then, the gel was stained with CBB stain, the gel image was captured with ChemiDocTouchMP (BIORAD), and the bands were quantified with Image Lab (BIORAD).

The gel images are shown in FIGS. 1 to 4. In the gel images, two bands (“upper band” and “lower band”) were observed in the S191C variants, and the molecular weight of the upper bands correspond to that of the parent antibodies. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions. Among the antibody variant samples with additional cysteine substitutions, most of them showed a higher lower band to upper band ratio, compared to S191C variants. Thus, the results suggest that additional cysteine substitutions to the S191C variants as listed in Table 6 are likely to enhance/promote disulfide bond crosslinking of Fabs, and additional cysteine substitutions could be an effective way to improve or increase the percentage or structural homogeneity of antibody preparation of the S191C variants having an engineered disulfide bond formed at the position 191 of CH1 region of the antibody.

### Example 2 Assessment of Antibodies Having Additional Disulfide Bond and Charged Mutations within the Fab Region

### Example 2-1 Production of Antibodies Having Additional Disulfide Bond and Charged Mutations within the Fab Region

One disulfide bond and charge mutations were introduced into the heavy chain of an anti-human CD3 antibody, OKT3 (heavy chain: OKT3VH0000-G1T4 (SEQ ID NO: 1242), light chain: OKT3VL0000-KT0 (SEQ ID NO: 1243)).

An amino acid residue structurally exposed to the surface of the OKT3 heavy chain constant region (G1T4, SEQ ID NO: 1244) was substituted with cysteine to produce the variant of OKT3 heavy chain constant region (G1T4.S191C, SEQ ID NO: 1245) shown in Table 1. In addition, other amino acid residues structurally exposed to the surface of G1T4.S191C were substituted with charged amino acids to produce the variants of G1T4.S191C shown in Table 7. These heavy chain constant regions were each linked with the OKT3 heavy chain variable region (OKT3VH0000, SEQ ID NO: 1246) to produce the OKT3 heavy chain variants, and expression vectors encoding the corresponding genes were produced by a method known in the art.

TABLE 7
G1T4.S191C variants with additional
charged amino acid substitution

| Variants of | | SEQ |
|---|---|---|
| heavy chain | Charged mutations | ID |
| constant region | (EU numbering) | NO: |
| G1T4.S191C0004 | S136K/G137K/G138K/L193D/G194D/T195D | 1285 |
| G1T4.S191C0005 | S136K/G137K/G138K/L193E/G194E/T195E | 1286 |
| G1T4.S191C0006 | S136R/G137R/G138R/L193D/G194D/T195D | 1287 |
| G1T4.S191C0007 | S136R/G137R/G138R/L193E/G194E/T195E | 1288 |
| G1T4.S191C0019 | G137K/G138K/L193D/G194D/T195D | 1289 |
| G1T4.S191C0020 | S136K/G138K/L193D/G194D/T195D | 1290 |
| G1T4.S191C0021 | S136K/G137K/L193D/G194D/T195D | 1291 |
| G1T4.S191C0022 | S136K/G137K/G138K/G194D/T195D | 1292 |
| G1T4.S191C0023 | S136K/G137K/G138K/L193D/T195D | 1293 |
| G1T4.S191C0024 | S136K/G137K/G138K/L193D/G194D | 1294 |
| G1T4.S191C0025 | G137K/G138K/L193E/G194E/T195E | 1295 |
| G1T4.S191C0026 | S136K/G138K/L193E/G194E/T195E | 1296 |
| G1T4.S191C0027 | S136K/G137K/L193E/G194E/T195E | 1297 |
| G1T4.S191C0028 | S136K/G137K/G138K/G194E/T195E | 1298 |
| G1T4.S191C0029 | S136K/G137K/G138K/L193E/T195E | 1299 |
| G1T4.S191C0030 | S136K/G137K/G138K/L193E/G194E | 1300 |
| G1T4.S191C0031 | G137R/G138R/L193D/G194D/T195D | 1301 |
| G1T4.S191C0032 | S136R/G138R/L193D/G194D/T195D | 1302 |
| G1T4.S191C0033 | S136R/G137R/L193D/G194D/T195D | 1303 |
| G1T4.S191C0034 | S136R/G137R/G138R/G194D/T195D | 1304 |
| G1T4.S191C0035 | S136R/G137R/G138R/L193D/T195D | 1305 |
| G1T4.S191C0036 | S136R/G137R/G138R/L193D/G194D | 1306 |
| G1T4.S191C0052 | S136K/L193D/G194D/T195D | 1307 |
| G1T4.S191C0053 | G137K/L193D/G194D/T195D | 1308 |
| G1T4.S191C0054 | G138K/L193D/G194D/T195D | 1309 |
| G1T4.S191C0055 | S136K/L193E/G194E/T195E | 1310 |
| G1T4.S191C0056 | G137K/L193E/G194E/T195E | 1311 |
| G1T4.S191C0057 | G138K/L193E/G194E/T195E | 1312 |
| G1T4.S191C0058 | S136R/L193D/G194D/T195D | 1313 |
| G1T4.S191C0059 | G137R/L193D/G194D/T195D | 1314 |
| G1T4.S191C0060 | G138R/L193D/G194D/T195D | 1315 |
| G1T4.S191C0061 | S136R/L193E/G194E/T195E | 1316 |
| G1T4.S191C0062 | G137R/L193E/G194E/T195E | 1317 |
| G1T4.S191C0063 | G138R/L193E/G194E/T195E | 1318 |
| G1T4.S191C0078 | G138K/T195D | 1319 |
| G1T4.S191C0079 | G138K/T195E | 1320 |
| G1T4.S191C0080 | G138R/T195D | 1321 |
| G1T4.S191C0081 | G138R/T195E | 1322 |
| G1T4.S191C0082 | G138D/T195K | 1323 |
| G1T4.S191C0083 | G138D/T195R | 1324 |
| G1T4.S191C0084 | G138E/T195K | 1325 |
| G1T4.S191C0085 | G138E/T195R | 1326 |
| G1T4.S191C0086 | G138K/G194D | 1327 |
| G1T4.S191C0087 | G138K/G194E | 1328 |
| G1T4.S191C0088 | G138R/G194D | 1329 |
| G1T4.S191C0089 | G138R/G194E | 1330 |
| G1T4.S191C0090 | G138D/G194K | 1331 |
| G1T4.S191C0091 | G138D/G194R | 1332 |
| G1T4.S191C0092 | G138E/G194K | 1333 |
| G1T4.S191C0093 | G138E/G194R | 1334 |
| G1T4.S191C0094 | G137K/T195D | 1335 |
| G1T4.S191C0095 | G137K/T195E | 1336 |
| G1T4.S191C0096 | G137R/T195D | 1337 |
| G1T4.S191C0097 | G137R/T195E | 1338 |
| G1T4.S191C0098 | G137D/T195K | 1339 |
| G1T4.S191C0099 | G137D/T195R | 1340 |
| G1T4.S191C0100 | G137E/T195K | 1341 |
| G1T4.S191C0101 | G137E/T195R | 1342 |
| G1T4.S191C0102 | G137K/G194D | 1343 |
| G1T4.S191C0103 | G137K/G194E | 1344 |
| G1T4.S191C0104 | G137R/G194D | 1345 |
| G1T4.S191C0105 | G137R/G194E | 1346 |
| G1T4.S191C0106 | G137D/G194K | 1347 |
| G1T4.S191C0107 | G137D/G194R | 1348 |
| G1T4.S191C0108 | G137E/G194K | 1349 |
| G1T4.S191C0109 | G137E/G194R | 1350 |
| G1T4.S191C0110 | G137K/G138K/G194E/T195E | 1351 |
| G1T4.S191C0111 | G137K/G138K/L193E/T195E | 1352 |
| G1T4.S191C0112 | S136K/G138K/G194E/T195E | 1353 |
| G1T4.S191C0113 | S136K/G138K/L193E/T195E | 1354 |
| G1T4.S191C0114 | S136K/G137K/G194E/T195E | 1355 |
| G1T4.S191C0115 | S136K/G137K/L193E/T195E | 1356 |
| G1T4.S191C0116 | S136K/G194E/T195E | 1357 |
| G1T4.S191C0117 | S136K/L193E/T195E | 1358 |
| G1T4.S191C0118 | G137K/G194E/T195E | 1359 |
| G1T4.S191C0119 | G137K/L193E/T195E | 1360 |
| G1T4.S191C0120 | G138K/G194E/T195E | 1361 |
| G1T4.S191C0121 | G138K/L193E/T195E | 1362 |
| G1T4.S191C0122 | G137R/G138R/G194D/T195D | 1363 |
| G1T4.S191C0123 | G137R/G138R/L193D/T195D | 1364 |
| G1T4.S191C0124 | S136R/G138R/G194D/T195D | 1365 |
| G1T4.S191C0125 | S136R/G138R/L193D/T195D | 1366 |
| G1T4.S191C0126 | S136R/G137R/G194D/T195D | 1367 |
| G1T4.S191C0127 | S136R/G137R/L193D/T195D | 1368 |
| G1T4.S191C0128 | S136R/G194D/T195D | 1369 |
| G1T4.S191C0129 | S136R/L193D/T195D | 1370 |
| G1T4.S191C0130 | G137R/G194D/T195D | 1371 |
| G1T4.S191C0131 | G137R/L193D/T195D | 1372 |
| G1T4.S191C0132 | G138R/G194D/T195D | 1373 |
| G1T4.S191C0133 | G138R/L193D/T195D | 1374 |
| G1T4.S191C0134 | S136K/T195D | 1375 |
| G1T4.S191C0135 | S136K/T195E | 1376 |
| G1T4.S191C0136 | S136R/T195D | 1377 |
| G1T4.S191C0137 | S136R/T195E | 1378 |
| G1T4.S191C0138 | S136D/T195K | 1379 |
| G1T4.S191C0139 | S136D/T195R | 1380 |
| G1T4.S191C0140 | S136E/T195K | 1381 |
| G1T4.S191C0141 | S136E/T195R | 1382 |
| G1T4.S191C0142 | S136K/G194D | 1383 |
| G1T4.S191C0143 | S136K/G194E | 1384 |
| G1T4.S191C0144 | S136R/G194D | 1385 |
| G1T4.S191C0145 | S136R/G194E | 1386 |
| G1T4.S191C0146 | S136D/G194K | 1387 |
| G1T4.S191C0147 | S136D/G194R | 1388 |
| G1T4.S191C0148 | S136E/G194K | 1389 |
| G1T4.S191C0149 | S136E/G194R | 1390 |

The OKT3 heavy chain variants produced as mentioned above were combined with the OKT3 light chain. The OKT3 variants shown in Table 8 were expressed by transient expression using Expi293 cells (Life technologies) by a method known in the art, and purified with Protein A by a method known in the art. In this Example, OKT3 is called “parent antibody”, OKT3.S191C is called “S191C variant”, and its variants are called “charged variants”.

TABLE 8
OKT3 variants with cysteine and charged amino acid substitution

| | Heavy | Heavy | |
|---|---|---|---|
| | chain | chain | |
| | variable | constant | Light |
| | region | region | chain |
| Name of OKT3 | SEQ | SEQ | SEQ |
| variants | ID NO: | ID NO: | ID NO: |
| OKT3 | 1246 | 1244 | 1243 |
| OKT3.S191C | 1246 | 1245 | 1243 |
| OKT3.S191C0004 | 1246 | 1285 | 1243 |
| OKT3.S191C0005 | 1246 | 1286 | 1243 |
| OKT3.S191C0006 | 1246 | 1287 | 1243 |
| OKT3.S191C0007 | 1246 | 1288 | 1243 |
| OKT3.S191C0019 | 1246 | 1289 | 1243 |
| OKT3.S191C0020 | 1246 | 1290 | 1243 |
| OKT3.S191C0021 | 1246 | 1291 | 1243 |
| OKT3.S191C0022 | 1246 | 1292 | 1243 |
| OKT3.S191C0023 | 1246 | 1293 | 1243 |
| OKT3.S191C0024 | 1246 | 1294 | 1243 |
| OKT3.S191C0025 | 1246 | 1295 | 1243 |
| OKT3.S191C0026 | 1246 | 1296 | 1243 |
| OKT3.S191C0027 | 1246 | 1297 | 1243 |
| OKT3.S191C0028 | 1246 | 1298 | 1243 |
| OKT3.S191C0029 | 1246 | 1299 | 1243 |
| OKT3.S191C0030 | 1246 | 1300 | 1243 |
| OKT3.S191C0031 | 1246 | 1301 | 1243 |
| OKT3.S191C0032 | 1246 | 1302 | 1243 |
| OKT3.S191C0033 | 1246 | 1303 | 1243 |
| OKT3.S191C0034 | 1246 | 1304 | 1243 |
| OKT3.S191C0035 | 1246 | 1305 | 1243 |
| OKT3.S191C0036 | 1246 | 1306 | 1243 |
| OKT3.S191C0052 | 1246 | 1307 | 1243 |
| OKT3.S191C0053 | 1246 | 1308 | 1243 |
| OKT3.S191C0054 | 1246 | 1309 | 1243 |
| OKT3.S191C0055 | 1246 | 1310 | 1243 |
| OKT3.S191C0056 | 1246 | 1311 | 1243 |
| OKT3.S191C0057 | 1246 | 1312 | 1243 |
| OKT3.S191C0058 | 1246 | 1313 | 1243 |
| OKT3.S191C0059 | 1246 | 1314 | 1243 |
| OKT3.S191C0060 | 1246 | 1315 | 1243 |
| OKT3.S191C0061 | 1246 | 1316 | 1243 |
| OKT3.S191C0062 | 1246 | 1317 | 1243 |
| OKT3.S191C0063 | 1246 | 1318 | 1243 |
| OKT3.S191C0078 | 1246 | 1319 | 1243 |
| OKT3.S191C0079 | 1246 | 1320 | 1243 |
| OKT3.S191C0080 | 1246 | 1321 | 1243 |
| OKT3.S191C0081 | 1246 | 1322 | 1243 |
| OKT3.S191C0082 | 1246 | 1323 | 1243 |
| OKT3.S191C0083 | 1246 | 1324 | 1243 |
| OKT3.S191C0084 | 1246 | 1325 | 1243 |
| OKT3.S191C0085 | 1246 | 1326 | 1243 |
| OKT3.S191C0086 | 1246 | 1327 | 1243 |
| OKT3.S191C0087 | 1246 | 1328 | 1243 |
| OKT3.S191C0088 | 1246 | 1329 | 1243 |
| OKT3.S191C0089 | 1246 | 1330 | 1243 |
| OKT3.S191C0090 | 1246 | 1331 | 1243 |
| OKT3.S191C0091 | 1246 | 1332 | 1243 |
| OKT3.S191C0092 | 1246 | 1333 | 1243 |
| OKT3.S191C0093 | 1246 | 1334 | 1243 |
| OKT3.S191C0094 | 1246 | 1335 | 1243 |
| OKT3.S191C0095 | 1246 | 1336 | 1243 |
| OKT3.S191C0096 | 1246 | 1337 | 1243 |
| OKT3.S191C0097 | 1246 | 1338 | 1243 |
| OKT3.S191C0098 | 1246 | 1339 | 1243 |
| OKT3.S191C0099 | 1246 | 1340 | 1243 |
| OKT3.S191C0100 | 1246 | 1341 | 1243 |
| OKT3.S191C0101 | 1246 | 1342 | 1243 |
| OKT3.S191C0102 | 1246 | 1343 | 1243 |
| OKT3.S191C0103 | 1246 | 1344 | 1243 |
| OKT3.S191C0104 | 1246 | 1345 | 1243 |
| OKT3.S191C0105 | 1246 | 1346 | 1243 |
| OKT3.S191C0106 | 1246 | 1347 | 1243 |
| OKT3.S191C0107 | 1246 | 1348 | 1243 |
| OKT3.S191C0108 | 1246 | 1349 | 1243 |
| OKT3.S191C0109 | 1246 | 1350 | 1243 |
| OKT3.S191C0110 | 1246 | 1351 | 1243 |
| OKT3.S191C0111 | 1246 | 1352 | 1243 |
| OKT3.S191C0112 | 1246 | 1353 | 1243 |
| OKT3.S191C0113 | 1246 | 1354 | 1243 |
| OKT3.S191C0114 | 1246 | 1355 | 1243 |
| OKT3.S191C0115 | 1246 | 1356 | 1243 |
| OKT3.S191C0116 | 1246 | 1357 | 1243 |
| OKT3.S191C0117 | 1246 | 1358 | 1243 |
| OKT3.S191C0118 | 1246 | 1359 | 1243 |
| OKT3.S191C0119 | 1246 | 1360 | 1243 |
| OKT3.S191C0120 | 1246 | 1361 | 1243 |
| OKT3.S191C0121 | 1246 | 1362 | 1243 |
| OKT3.S191C0122 | 1246 | 1363 | 1243 |
| OKT3.S191C0123 | 1246 | 1364 | 1243 |
| OKT3.S191C0124 | 1246 | 1365 | 1243 |
| OKT3.S191C0125 | 1246 | 1366 | 1243 |
| OKT3.S191C0126 | 1246 | 1367 | 1243 |
| OKT3.S191C0127 | 1246 | 1368 | 1243 |
| OKT3.S191C0128 | 1246 | 1369 | 1243 |
| OKT3.S191C0129 | 1246 | 1370 | 1243 |
| OKT3.S191C0130 | 1246 | 1371 | 1243 |
| OKT3.S191C0131 | 1246 | 1372 | 1243 |
| OKT3.S191C0132 | 1246 | 1373 | 1243 |
| OKT3.S191C0133 | 1246 | 1374 | 1243 |
| OKT3.S191C0134 | 1246 | 1375 | 1243 |
| OKT3.S191C0135 | 1246 | 1376 | 1243 |
| OKT3.S191C0136 | 1246 | 1377 | 1243 |
| OKT3.S191C0137 | 1246 | 1378 | 1243 |
| OKT3.S191C0138 | 1246 | 1379 | 1243 |
| OKT3.S191C0139 | 1246 | 1380 | 1243 |
| OKT3.S191C0140 | 1246 | 1381 | 1243 |
| OKT3.S191C0141 | 1246 | 1382 | 1243 |
| OKT3.S191C0142 | 1246 | 1383 | 1243 |
| OKT3.S191C0143 | 1246 | 1384 | 1243 |
| OKT3.S191C0144 | 1246 | 1385 | 1243 |
| OKT3.S191C0145 | 1246 | 1386 | 1243 |
| OKT3.S191C0146 | 1246 | 1387 | 1243 |
| OKT3.S191C0147 | 1246 | 1388 | 1243 |
| OKT3.S191C0148 | 1246 | 1389 | 1243 |
| OKT3.S191C0149 | 1246 | 1390 | 1243 |

### Example 2-2 Assessment of Electrophoretic Mobility in Polyacrylamide Gel of Antibodies Having Additional Disulfide Bond and Charged Mutations within the Fab Region

Similarly to Example 1-2, non-reducing SDS-PAGE was carried out with the charged variants produced in Example 2-1, the gel image was captured, and intensities of bands were quantified.

In the gel images, two bands were observed in the S191C variant, and the molecular weight of the upper bands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions. The ratio of the lower band to upper band are shown in Table 9. Among charged variants, most of them showed a higher lower band to upper band ratio, compared to that of S191C variants. Thus, the results suggest that additional charged amino acid mutations to the S191C variants as listed in Table 7 are likely to enhance/promote disulfide bond crosslinking of Fabs, and additional charged amino acid mutations could be an effective way to improve or increase the percentage or structural homogeneity of antibody preparation of the S191C variants having an engineered disulfide bond formed at the position 191 of CH1 region of the antibody.

TABLE 9
The ratio of the lower band to upper band of OKT3 variants
with cysteine and charged amino acid substitution

| | Ratio of |
|---|---|
| Name of OKT3 | lower band to |
| variants | upper band (%) |
| OKT3 | 0 |
| OKT3.S191C | 65.4 |
| OKT3.S191C0004 | 86.3 |
| OKT3.S191C0005 | 87.2 |
| OKT3.S191C0006 | 87.4 |
| OKT3.S191C0007 | 83.8 |
| OKT3.S191C0019 | 85.1 |
| OKT3.S191C0020 | 82.6 |
| OKT3.S191C0021 | 83.7 |
| OKT3.S191C0022 | 77 |
| OKT3.S191C0023 | 78.1 |
| OKT3.S191C0024 | 75.2 |
| OKT3.S191C0025 | 84 |
| OKT3.S191C0026 | 85.9 |
| OKT3.S191C0027 | 85.6 |
| OKT3.S191C0028 | 70.3 |
| OKT3.S191C0029 | 77.4 |
| OKT3.S191C0030 | 79.4 |
| OKT3.S191C0031 | 87.9 |
| OKT3.S191C0032 | 84.4 |
| OKT3.S191C0033 | 85.4 |
| OKT3.S191C0034 | 79.9 |
| OKT3.S191C0035 | 82 |
| OKT3.S191C0036 | 82.3 |
| OKT3.S191C0052 | 75.5 |
| OKT3.S191C0053 | 85.7 |
| OKT3.S191C0054 | 77.7 |
| OKT3.S191C0055 | 79 |
| OKT3.S191C0056 | 87.2 |
| OKT3.S191C0057 | 83 |
| OKT3.S191C0058 | 84.7 |
| OKT3.S191C0059 | 78.6 |
| OKT3.S191C0060 | 74.7 |
| OKT3.S191C0061 | 88.4 |
| OKT3.S191C0062 | 87.2 |
| OKT3.S191C0063 | 85.5 |
| OKT3.S191C0078 | 74.6 |
| OKT3.S191C0079 | 69.7 |
| OKT3.S191C0080 | 76.5 |
| OKT3.S191C0081 | 72.9 |
| OKT3.S191C0082 | 56.2 |
| OKT3.S191C0083 | 69.7 |
| OKT3.S191C0084 | 46.5 |
| OKT3.S191C0085 | 67.1 |
| OKT3.S191C0086 | 58.2 |
| OKT3.S191C0087 | 49.7 |
| OKT3.S191C0088 | 63.7 |
| OKT3.S191C0089 | 65.5 |
| OKT3.S191C0090 | 43.9 |
| OKT3.S191C0091 | 56.9 |
| OKT3.S191C0092 | 43.9 |
| OKT3.S191C0093 | 53.5 |
| OKT3.S191C0094 | 79.2 |
| OKT3.S191C0095 | 77.6 |
| OKT3.S191C0096 | 79.3 |
| OKT3.S191C0097 | 71.1 |
| OKT3.S191C0098 | 45.3 |
| OKT3.S191C0099 | 60 |
| OKT3.S191C0100 | 45.6 |
| OKT3.S191C0101 | 55.9 |
| OKT3.S191C0102 | 72 |
| OKT3.S191C0103 | 73.2 |
| OKT3.S191C0104 | 76.3 |
| OKT3.S191C0105 | 74.3 |
| OKT3.S191C0106 | 42.1 |
| OKT3.S191C0107 | 52.1 |
| OKT3.S191C0108 | 38.1 |
| OKT3.S191C0109 | 44.8 |
| OKT3.S191C0110 | 72.1 |
| OKT3.S191C0111 | 79.2 |
| OKT3.S191C0112 | 72.5 |
| OKT3.S191C0113 | 78.8 |
| OKT3.S191C0114 | 71.5 |
| OKT3.S191C0115 | 78.5 |
| OKT3.S191C0116 | 66.4 |
| OKT3.S191C0117 | 78.2 |
| OKT3.S191C0118 | 86.3 |
| OKT3.S191C0119 | 83.4 |
| OKT3.S191C0120 | no data |
| OKT3.S191C0121 | 82.1 |
| OKT3.S191C0122 | 82.3 |
| OKT3.S191C0123 | 76.6 |
| OKT3.S191C0124 | 76.8 |
| OKT3.S191C0125 | 78.7 |
| OKT3.S191C0126 | 85.5 |
| OKT3.S191C0127 | 88.1 |
| OKT3.S191C0128 | 77.7 |
| OKT3.S191C0129 | 79.2 |
| OKT3.S191C0130 | 80 |
| OKT3.S191C0131 | 85.3 |
| OKT3.S191C0132 | 87.6 |
| OKT3.S191C0133 | 87.6 |
| OKT3.S191C0134 | 66.7 |
| OKT3.S191C0135 | 72.5 |
| OKT3.S191C0136 | 73.7 |
| OKT3.S191C0137 | 71.9 |
| OKT3.S191C0138 | 45.1 |
| OKT3.S191C0139 | 58.6 |
| OKT3.S191C0140 | 41.4 |
| OKT3.S191C0141 | 57 |
| OKT3.S191C0142 | 70.5 |
| OKT3.S191C0143 | 66 |
| OKT3.S191C0144 | 75.9 |
| OKT3.S191C0145 | 68.3 |
| OKT3.S191C0146 | 43.5 |
| OKT3.S191C0147 | 53.8 |
| OKT3.S191C0148 | 40 |
| OKT3.S191C0149 | 49.8 |

### Example 3 Assessment of Antibodies Having Additional Disulfide Bond and Hydrophobic Mutations within the Fab Region

### Example 3-1 Production of Antibodies Having Additional Disulfide Bond and Hydrophobic Mutations within Fab Region

One disulfide bond and charge mutations were introduced into the heavy chain of an anti-human CD3 antibody, OKT3 (heavy chain: OKT3VH0000-G1T4 (SEQ ID NO: 1242), light chain: OKT3VL0000-KT0 (SEQ ID NO: 1243)).

An amino acid residue structurally exposed to the surface of the OKT3 heavy chain constant region (G1T4, SEQ ID NO: 1244) was substituted with cysteine to produce the variant of OKT3 heavy chain constant region (G1T4.S191C, SEQ ID NO: 1245) shown in Table 1. In addition, other amino acid residues structurally exposed to the surface of G1T4.S191C were substituted with hydrophobic amino acids to produce the variants of G1T4.S191C shown in Table 10. These heavy chain constant regions were each linked with the OKT3 heavy chain variable region (OKT3VH0000, SEQ ID NO: 1246) to produce the OKT3 heavy chain variants, and expression vectors encoding the corresponding genes were produced by a method known in the art.

TABLE 10
G1T4.S191C variants with hydrophobic amino acid substitution

| Variants of | | |
|---|---|---|
| heavy chain | Hydrophobic mutations | SEQ |
| constant region | (EU numbering) | ID NO: |
| G1T4.S191C0001 | S136W/G137W/G138W/L193W/G194W/T195W | 1391 |
| G1T4.S191C0002 | S136L/G137L/G138L/G194L/T195L | 1392 |
| G1T4.S191C0003 | S136V/G137V/G138V/L193V/G194V/T195V | 1393 |
| G1T4.S191C0008 | S136A/G137A/G138A/L193A/G194A/T195A | 1394 |
| G1T4.S191C0009 | S136V/G137V/G138V/L193W/G194W/T195W | 1395 |
| G1T4.S191C0010 | S136W/G137W/G138W/L193V/G194V/T195V | 1396 |
| G1T4.S191C0011 | S136V/G137V/G138V/G194L/T195L | 1397 |
| G1T4.S191C0012 | S136L/G137L/G138L/L193V/G194V/T195V | 1398 |
| G1T4.S191C0013 | G137V/G138V/L193V/G194V/T195V | 1399 |
| G1T4.S191C0014 | S136V/G138V/L193V/G194V/T195V | 1400 |
| G1T4.S191C0015 | S136V/G137V/L193V/G194V/T195V | 1401 |
| G1T4.S191C0016 | S136V/G137V/G138V/G194V/T195V | 1402 |
| G1T4.S191C0017 | S136V/G137V/G138V/L193V/T195V | 1403 |
| G1T4.S191C0018 | S136V/G137V/G138V/L193V/G194V | 1404 |
| G1T4.S191C0037 | S136A/G137A/G138A/L193W/G194W/T195W | 1405 |
| G1T4.S191C0038 | S136A/G137A/G138A/G194L/T195L | 1406 |
| G1T4.S191C0039 | S136A/G137A/G138A/L193V/G194V/T195V | 1407 |
| G1T4.S191C0040 | S136W/G137W/G138W/L193A/G194A/T195A | 1408 |
| G1T4.S191C0041 | G137W/G138W/L193A/G194A/T195A | 1409 |
| G1T4.S191C0042 | S136W/G138W/L193A/G194A/T195A | 1410 |
| G1T4.S191C0043 | S136W/G137W/L193A/G194A/T195A | 1411 |
| G1T4.S191C0044 | S136W/L193A/G194A/T195A | 1412 |
| G1T4.S191C0045 | G137W/L193A/G194A/T195A | 1413 |
| G1T4.S191C0046 | G138W/L193A/G194A/T195A | 1414 |
| G1T4.S191C0047 | S136L/G137L/G138L/L193A/G194A/T195A | 1415 |
| G1T4.S191C0048 | S136V/G137V/G138V/L193A/G194A/T195A | 1416 |
| G1T4.S191C0049 | S136V/L193V/G194V/T195V | 1417 |
| G1T4.S191C0050 | G137V/L193V/G194V/T195V | 1418 |
| G1T4.S191C0051 | G138V/L193V/G194V/T195V | 1419 |
| G1T4.S191C0072 | S136W/L193S/G194V/T195A | 1420 |
| G1T4.S191C0073 | G137W/L193S/G194V/T195A | 1421 |
| G1T4.S191C0074 | G138W/L193S/G194V/T195A | 1422 |
| G1T4.S191C0075 | G137V/G138A/L193W | 1423 |
| G1T4.S191C0076 | G137V/G138A/G194W | 1424 |
| G1T4.S191C0077 | G137V/G138A/T195W | 1425 |

The OKT3 heavy chain variants produced as mentioned above were combined with the OKT3 light chain. The OKT3 variants shown in Table 11 were expressed by transient expression using Expi293 cells (Life technologies) by a method known in the art, and purified with Protein A by a method known in the art. In this Example, OKT3 is called “parent antibody”, OKT3.S191C is called “S191C variant”, and its variants are called “hydrophobic variants”.

TABLE 11
OKT3 variants with cysteine and hydrophobic
amino acid substitution

| | Heavy | Heavy | |
|---|---|---|---|
| | chain | chain | |
| | variable | constant | Light |
| | region | region | chain |
| Name of OKT3 | SEQ | SEQ | SEQ |
| variants | ID NO: | ID NO: | ID NO: |
| OKT3 | 1246 | 1244 | 1243 |
| OKT3.S191C | 1246 | 1245 | 1243 |
| OKT3.S191C0001 | 1246 | 1391 | 1243 |
| OKT3.S191C0002 | 1246 | 1392 | 1243 |
| OKT3.S191C0003 | 1246 | 1393 | 1243 |
| OKT3.S191C0008 | 1246 | 1394 | 1243 |
| OKT3.S191C0009 | 1246 | 1395 | 1243 |
| OKT3.S191C0010 | 1246 | 1396 | 1243 |
| OKT3.S191C0011 | 1246 | 1397 | 1243 |
| OKT3.S191C0012 | 1246 | 1398 | 1243 |
| OKT3.S191C0013 | 1246 | 1399 | 1243 |
| OKT3.S191C0014 | 1246 | 1400 | 1243 |
| OKT3.S191C0015 | 1246 | 1401 | 1243 |
| OKT3.S191C0016 | 1246 | 1402 | 1243 |
| OKT3.S191C0017 | 1246 | 1403 | 1243 |
| OKT3.S191C0018 | 1246 | 1404 | 1243 |
| OKT3.S191C0037 | 1246 | 1405 | 1243 |
| OKT3.S191C0038 | 1246 | 1406 | 1243 |
| OKT3.S191C0039 | 1246 | 1407 | 1243 |
| OKT3.S191C0040 | 1246 | 1408 | 1243 |
| OKT3.S191C0041 | 1246 | 1409 | 1243 |
| OKT3.S191C0042 | 1246 | 1410 | 1243 |
| OKT3.S191C0043 | 1246 | 1411 | 1243 |
| OKT3.S191C0044 | 1246 | 1412 | 1243 |
| OKT3.S191C0045 | 1246 | 1413 | 1243 |
| OKT3.S191C0046 | 1246 | 1414 | 1243 |
| OKT3.S191C0047 | 1246 | 1415 | 1243 |
| OKT3.S191C0048 | 1246 | 1416 | 1243 |
| OKT3.S191C0049 | 1246 | 1417 | 1243 |
| OKT3.S191C0050 | 1246 | 1418 | 1243 |
| OKT3.S191C0051 | 1246 | 1419 | 1243 |
| OKT3.S191C0072 | 1246 | 1420 | 1243 |
| OKT3.S191C0073 | 1246 | 1421 | 1243 |
| OKT3.S191C0074 | 1246 | 1422 | 1243 |
| OKT3.S191C0075 | 1246 | 1423 | 1243 |
| OKT3.S191C0076 | 1246 | 1424 | 1243 |
| OKT3.S191C0077 | 1246 | 1425 | 1243 |

### Example 3-2 Assessment of Electrophoretic Mobility in Polyacrylamide Gel of Antibodies Having Additional Disulfide Bond and Hydrophobic Mutations within the Fab Region

Similarly to Example 1-2, non-reducing SDS-PAGE was carried out with the hydrophobic variants produced in Example 3-1, the gel image was captured, and bands were quantified.

In the gel images, two bands were observed in S191C variant, and the molecular weight of the upper bands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions. The ratio of the lower bands to upper bands are shown in Table 12. Among hydrophobic variants, most of them showed a higher lower band to upper band ratio, compared to that of S191C variants. It is highly likely that structural changes such as crosslinking via disulfide bonds of Fabs were caused by cysteine substitution, which resulted in the change in electrophoretic mobility. Thus, the results suggest that additional hydrophobic amino acid mutations to the S191C variants as listed in Table 10 are likely to enhance/promote disulfide bond crosslinking of Fabs, and additional hydrophobic amino acid mutations could be an effective way to improve or increase the percentage or structural homogeneity of antibody preparation of the S191C variants having an engineered disulfide bond formed at the position 191 of CH1 region of the antibody.

TABLE 12
The ratio of the lower band to upper band of OKT3 variants
with cysteine and hydrophobic amino acid substitution

| | Ratio of |
|---|---|
| Name of OKT3 | lower band to |
| variants | upper band (%) |
| OKT3 | 0 |
| OKT3.S191C | 65.4 |
| OKT3.S191C0001 | no data |
| OKT3.S191C0002 | no data |
| OKT3.S191C0003 | no data |
| OKT3.S191C0008 | 75.7 |
| OKT3.S191C0009 | no data |
| OKT3.S191C0010 | no data |
| OKT3.S191C0011 | no data |
| OKT3.S191C0012 | 91.3 |
| OKT3.S191C0013 | no data |
| OKT3.S191C0014 | 83.5 |
| OKT3.S191C0015 | 82.7 |
| OKT3.S191C0016 | no data |
| OKT3.S191C0017 | 69.9 |
| OKT3.S191C0018 | 76.1 |
| OKT3.S191C0037 | 87.3 |
| OKT3.S191C0038 | 77.3 |
| OKT3.S191C0039 | 81 |
| OKT3.S191C0040 | 93.9 |
| OKT3.S191C0041 | 87.7 |
| OKT3.S191C0042 | 95.7 |
| OKT3.S191C0043 | 94.1 |
| OKT3.S191C0044 | 94.8 |
| OKT3.S191C0045 | 82 |
| OKT3.S191C0046 | 93.7 |
| OKT3.S191C0047 | 95.1 |
| OKT3.S191C0048 | 82.5 |
| OKT3.S191C0049 | 81.5 |
| OKT3.S191C0050 | 77.7 |
| OKT3.S191C0051 | no data |
| OKT3.S191C0072 | 83.9 |
| OKT3.S191C0073 | 85.5 |
| OKT3.S191C0074 | 87.6 |
| OKT3.S191C0075 | 63.2 |
| OKT3.S191C0076 | 88.3 |
| OKT3.S191C0077 | 84.1 |

### Example 4 Assessment of Effect of De-Cysteinylation by a Reducing Agent Such as 2-MEA to Promote Formation of Disulfide Bonds in Fabs

### Example 4-1 Production of Antibodies Having Cysteine Substitution in the Heavy Chain

Amino acid residue at position 191 (EU numbering) in the heavy chain of an anti-human IL6R neutralizing antibody, MRA, was substituted with cysteine (heavy chain: MRAH-G1T4.S191C (SEQ ID NO: 1426, light chain: MRAL-k0 (SEQ ID NO: 1427). Expression vectors encoding the corresponding genes were produced by a method known in the art.

This antibody was expressed by transient expression using Expi293 cells (Life technologies) by a method known in the art, and purified with Protein A by a method known in the art. It was concentrated to 24.1 mg/mL using Jumbosep Centrifugal Filter (PALL: OD030C65) for use in high concentrations.

### Example 4-2 Preparation of Antibody Samples Treated with 2-MEA

Using the antibody produced in Example 4-1, it was examined whether treatment/incubation with a reducing agent such as 2-MEA (2-Mercaptoethylamin) can promote formation of disulfide bonds in Fabs by inducing de-cysteinylation of capped-cysteine residues that do not form disulfide bond cross-linking. 2-MEA (Sigma-Aldrich: M6500) was dissolved in 25 mM NaCl, 20 mM Na-Phosphate buffer, pH7.0. The antibody and 2-MEA were mixed to the concentration shown in Table 13, and incubated in 5 mM NaCl, 20 mM Na-Phosphate buffer, pH7.0 at 37 degrees C. for 2 hours. To stop the reduction reaction, the buffer of the mixtures with 2-MEA was changed to the buffer without 2-MEA. Then, the samples were incubated at room temperature overnight for re-oxidation.

TABLE 13
Concentrations of antibody and 2-MEA in each sample

| | Antibody | Reagents | | |
|---|---|---|---|---|
| Sample | conc. | Conc. | FIG. | Lane |
| No. | (mM) | (mM) | No. | No. |
| 1 [Control] | 20 | 0 | 5, 6 | 3 |
| 2 | 20 | 0.01 | 5 | 4 |
| 3 | 20 | 0.05 | 5 | 5 |
| 4 | 20 | 0.1 | 5 | 6 |
| 5 | 20 | 0.25 | 6 | 4 |
| 6 | 20 | 0.5 | 6 | 5 |
| 7 | 20 | 1 | 6 | 6 |
| 8 | 20 | 2.5 | 6 | 7 |
| 9 | 20 | 5 | 6 | 8 |
| 10 | 20 | 10 | 6 | 9 |
| 11 | 20 | 25 | 6 | 10 |
| 12 | 20 | 50 | 6 | 11 |
| 13 | 20 | 100 | 6 | 12 |
| 14 [Control] | 1 | 0 | 7, 8 | 3 |
| 15 | 1 | 0.01 | 7 | 4 |
| 16 | 1 | 0.05 | 7 | 5 |
| 17 | 1 | 0.1 | 7 | 6 |
| 18 | 1 | 0.25 | 8 | 4 |
| 19 | 1 | 0.5 | 8 | 5 |
| 20 | 1 | 1 | 8 | 6 |
| 21 | 1 | 2.5 | 8 | 7 |
| 22 | 1 | 5 | 8 | 8 |
| 23 | 1 | 10 | 8 | 9 |
| 24 | 1 | 25 | 8 | 10 |
| 25 | 1 | 50 | 8 | 11 |
| 26 | 1 | 100 | 8 | 12 |

### Example 4-3 Assessment of Electrophoretic Mobility in Polyacrylamide Gel of the Samples in Each Concentration with Antibody and 2-MEA

It was examined whether the antibody samples treated with 2-MEA produced in Example 4-2 show a different electrophoretic mobility (i.e. different lower band to upper band ratio) in polyacrylamide gel by non-reducing SDS-PAGE.

Sample Buffer Solution (2ME-) (×4) (Wako; 198-13282) was used for preparation of electrophoresis samples. The samples were treated for 10 minutes under the condition of specimen concentration 100 microgram/mL and 70 degrees C., and then subjected to non-reducing SDS-PAGE. In non-reducing SDS-PAGE, electrophoresis was carried out for 90 minutes at 125 V, using 4% SDS-PAGE mini 15 well 1.0 mm 15 well (TEFCO; 01-052-6). Then, the gel was stained with CBB stain, the gel image was captured with ChemiDocTouchMP (BIORAD), and the bands were quantified with Image Lab (BIORAD).

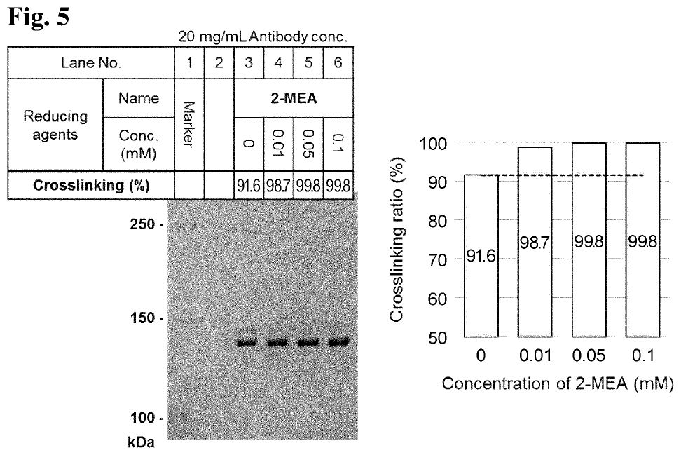

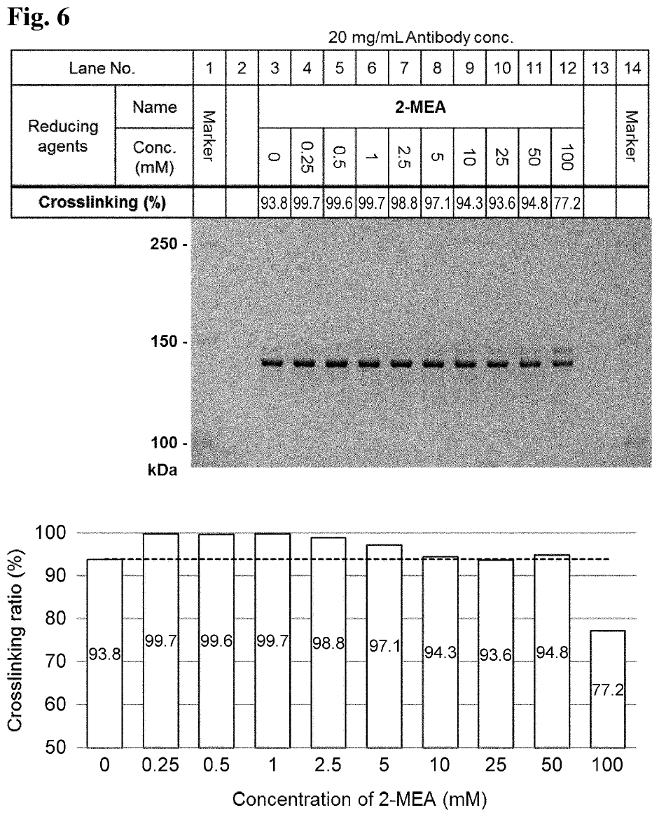

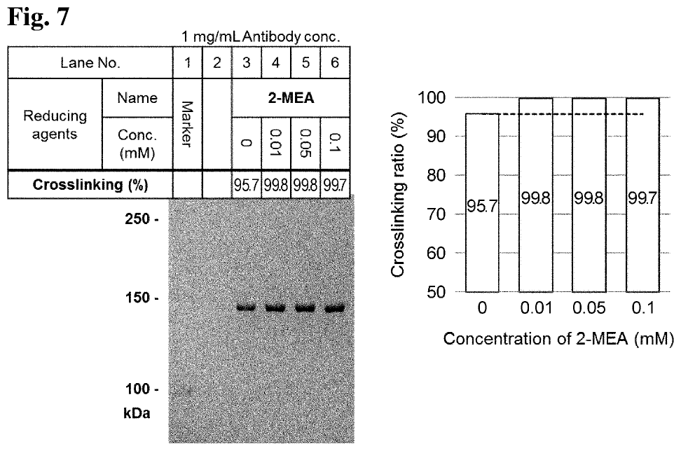

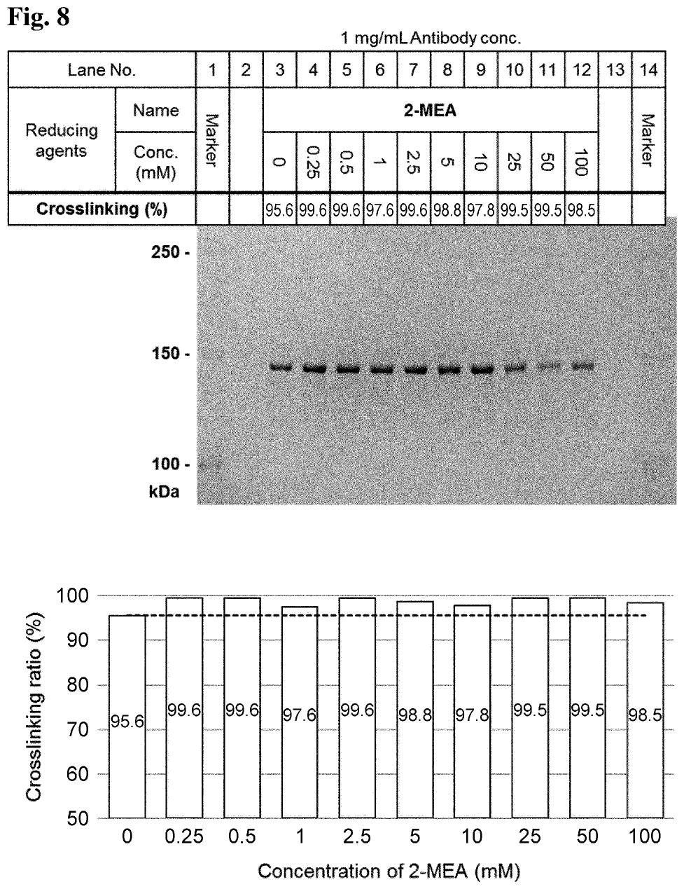

The gel images are shown in FIGS. 5 to 8. In the gel images, two bands were observed in the sample without (0 mM of) a reducing agent (control; lane 3 in each figure), and the molecular weights of the upper bands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions. The results show that most of antibody samples treated/incubated with 2-MEA showed a higher lower band to higher band ratio, compared to antibody samples without 2-MEA treatment. The results suggest that incubation of the antibody with a reducing agent such as 2-MEA could be an effective way to improve or increase the percentage or structural homogeneity of antibody preparation of the S191C variants having an engineered disulfide bond formed at the position 191 of the CH1 region of the antibody.

### Example 5 Assessment of Effect of De-Cysteinylation by a Reducing Agent Such as TCEP to Promote Formation of Disulfide Bonds in Fabs

### Example 5-1 Preparation of Antibody Samples Treated with TCEP

Using the antibody produced in Example 4-1, it was examined whether treatment/incubation with a reducing agent such as TCEP can promote formation of disulfide bonds in Fabs by inducing de-cysteinylation of capped-cysteine residues that do not form disulfide bond cross-linking.

TCEP (Sigma-Aldrich: C4706) was dissolved in ultra pure water and adjusted to pH 7 with NaOH. The antibody and TCEP were mixed to the concentration shown in Table 14, and incubated in 5 mM NaCl, 20 mM Na-Phosphate buffer, pH7.0 at 37 degrees C. for 2 hours. To stop the reduction reaction, the buffer of the mixtures with TCEP was changed to the buffer without TCEP. Then, the samples were incubated at room temperature (RT) overnight for re-oxidation.

TABLE 14
Concentrations of antibody and TCEP in each sample

| | Antibody | Reagents | | |
|---|---|---|---|---|
| Sample | conc. | Conc. | FIG. | Lane |
| No. | (mM) | (mM) | No. | No. |
| 1 [Control] | 20 | 0 | 9, 10 | 3 |
| 2 | 20 | 0.01 | 9 | 4 |
| 3 | 20 | 0.05 | 9 | 5 |
| 4 | 20 | 0.1 | 9 | 6 |
| 5 | 20 | 0.25 | 10 | 4 |
| 6 | 20 | 0.5 | 10 | 5 |
| 7 | 20 | 1 | 10 | 6 |
| 8 | 20 | 2.5 | 10 | 7 |
| 9 | 20 | 5 | 10 | 8 |
| 10 | 20 | 10 | 10 | 9 |
| 11 | 20 | 25 | 10 | 10 |
| 12 | 20 | 50 | 10 | 11 |
| 13 | 20 | 100 | 10 | 12 |
| 14 [Control] | 1 | 0 | 11 | 3 |
| 15 | 1 | 0.25 | 11 | 4 |
| 16 | 1 | 0.5 | 11 | 5 |
| 17 | 1 | 1 | 11 | 6 |
| 18 | 1 | 2.5 | 11 | 7 |
| 19 | 1 | 5 | 11 | 8 |
| 20 | 1 | 10 | 11 | 9 |
| 21 | 1 | 25 | 11 | 10 |
| 22 | 1 | 50 | 11 | 11 |
| 23 | 1 | 100 | 11 | 12 |

### Example 5-2 Assessment of Electrophoretic Mobility in Polyacrylamide Gel of the Samples in Each Concentration with Antibody and TCEP

Similarly to Example 4-3, non-reducing SDS-PAGE was carried out with the antibody samples treated with TCEP in Example 5-1, the gel image was captured, and bands were quantified.

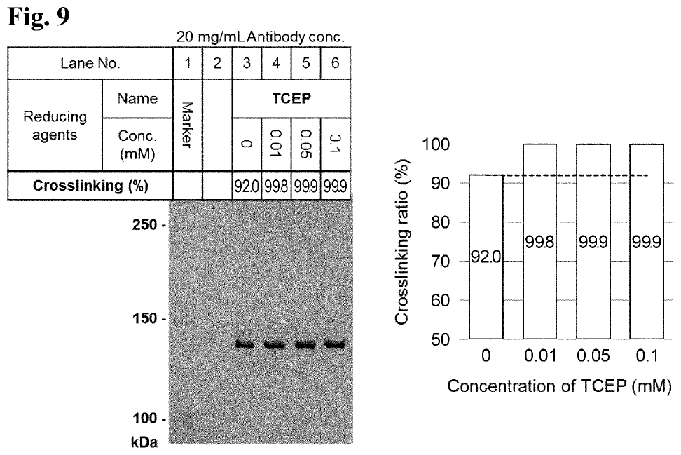

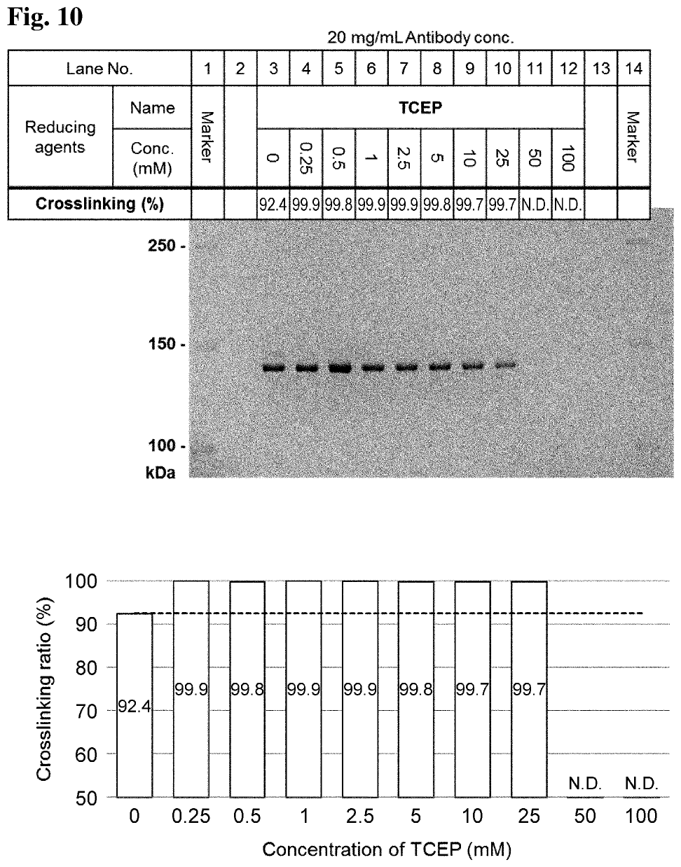

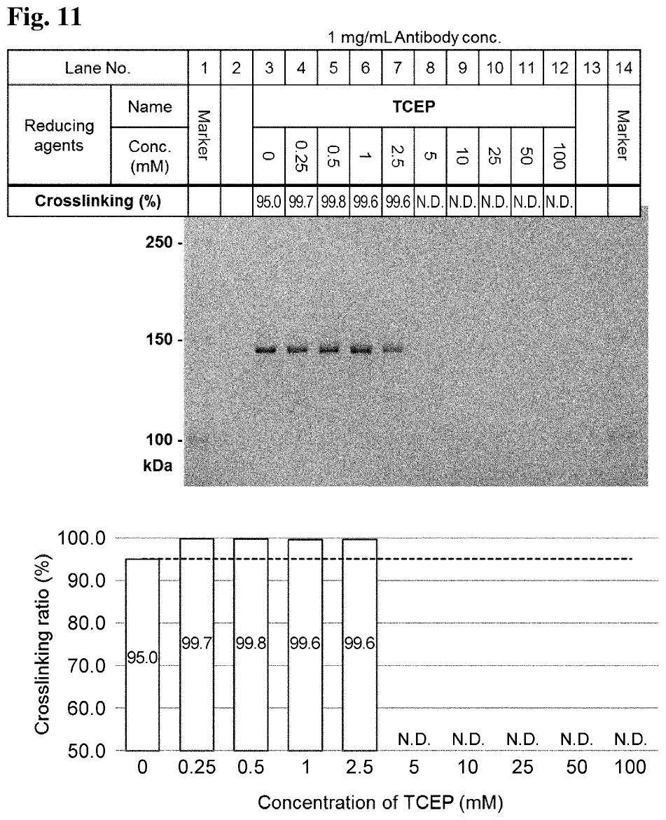

The gel images are shown in FIGS. 9 to 11. In the gel images, two bands were observed in the sample without (0 mM of) a reducing agent (control; lane 3 in each figure), and the molecular weights of the upper bands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions. The results show that most of samples incubated/treated with TCEP showed a higher lower band to upper band ratio, compared to that of an antibody sample without TCEP treatment. The results suggest that incubation of the antibody with a reducing agent such as TCEP could be an effective way to improve or increase the percentage or structural homogeneity of antibody preparation of the S191C variants having an engineered disulfide bond formed at the position 191 of the CH1 region of the antibody.

### Example 6 Assessment of Effect of De-Cysteinylation by Other Reducing Agents to Promote Formation of Disulfide Bonds in Fabs

### Example 6-1 Preparation of Reaction Samples Using 4 Reducing Agents

Using the antibody produced in Example 1-1, four different reducing agents, namely DTT, Cysteine, GSH, Na₂SO₃, were examined for whether they can promote formation of disulfide bonds in Fabs by inducing de-cysteinylation of capped-cysteine residues that do not form disulfide bond cross-linking.

DTT (Wako: 040-29223), L-Cysteine (Sigma-Aldrich: 168149), Glutathione (Wako: 077-02011) and Na₂SO₃ (Wako: 198-03412) were dissolved in 25 mM NaCl, 20 mM Na-Phosphate buffer, pH7.0. Na₂SO₃ was adjusted to pH 7 with HCl. The antibody and each reducing agent were mixed to the concentration shown in Table 15, and incubated in 5 mM NaCl, 20 mM Na-Phosphate buffer, pH7.0 at room temperature (RT) overnight. To stop the reduction reaction, the buffer of the mixtures with each reducing agent was changed to the buffer without the reducing agent. Then, the samples were incubated at room temperature overnight for re-oxidation.

TABLE 15
Concentrations of antibody and reducing reagents in each sample

| | Antibody | | Reagents | | |
|---|---|---|---|---|---|
| Sample | conc. | Reagents | Conc. | FIG. | Lane |
| No. | (mM) | name | (mM) | No. | No. |
| 1 [Control] | 20 | None | 0 | 12, 13 | 3 |
| 2 | 20 | DTT | 0.1 | 12 | 4 |
| 3 | 20 | DTT | 1 | 12 | 5 |
| 4 | 20 | DTT | 10 | 12 | 6 |
| 5 | 20 | DTT | 100 | 12 | 7 |
| 6 | 20 | Cysteine | 0.1 | 12 | 8 |
| 7 | 20 | Cysteine | 1 | 12 | 9 |
| 8 | 20 | Cysteine | 10 | 12 | 10 |
| 9 | 20 | Cysteine | 100 | 12 | 11 |
| 10 | 20 | GSH | 0.1 | 13 | 4 |
| 11 | 20 | GSH | 1 | 13 | 5 |
| 12 | 20 | GSH | 10 | 13 | 6 |
| 13 | 20 | GSH | 50 | 13 | 7 |
| 14 | 20 | Na₂SO₃ | 0.01 | 13 | 8 |
| 15 | 20 | Na₂SO₃ | 0.1 | 13 | 9 |
| 16 | 20 | Na₂SO₃ | 1 | 13 | 10 |
| 17 | 20 | Na₂SO₃ | 10 | 13 | 11 |

### Example 6-2 Assessment of Electrophoretic Mobility in Polyacrylamide Gel of the Samples in Each Concentration with Antibody and 4 Reducing Agents

Similarly to Example 4-3, non-reducing SDS-PAGE was carried out with the antibody samples produced in Example 6-1, the gel image was captured, and bands were quantified.

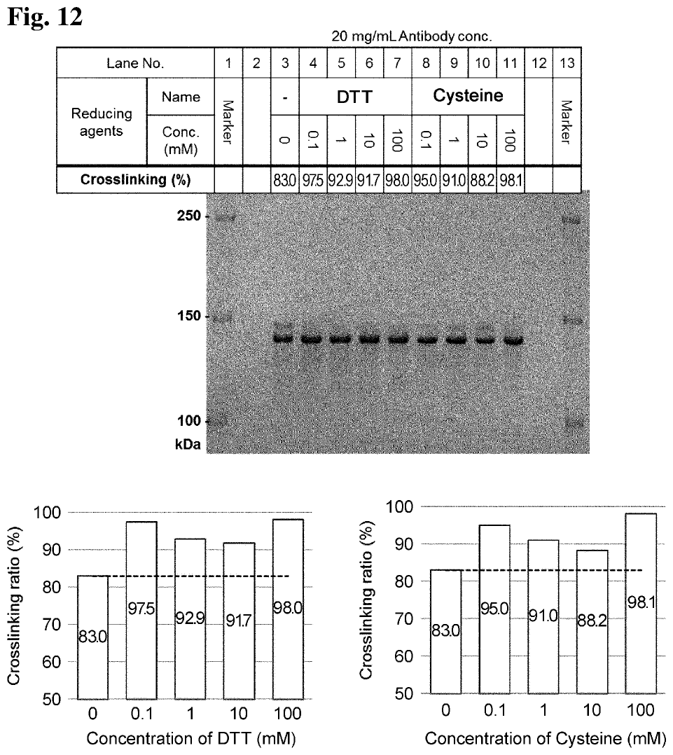

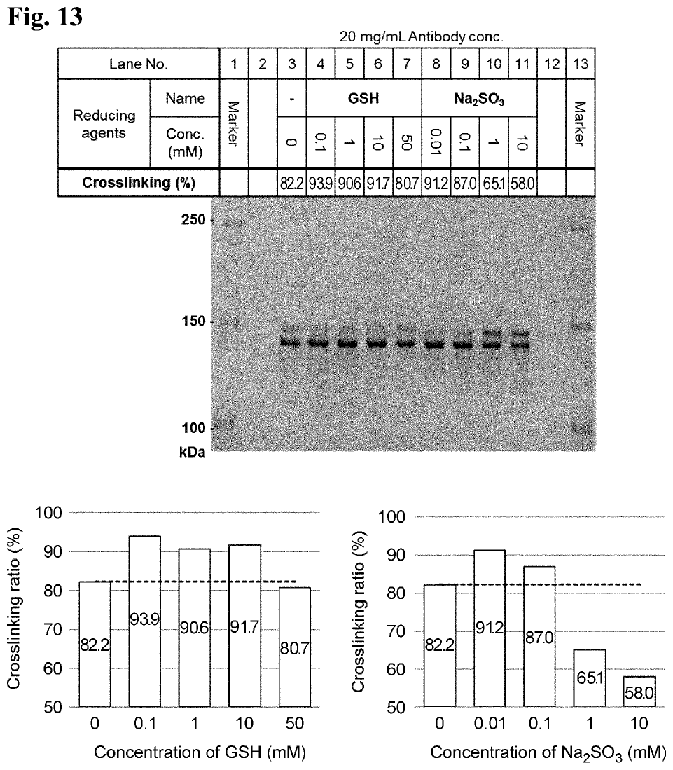

The gel images are shown in FIGS. 12 and 13. In the gel images, two bands were observed in the sample without (0 mM of) a reducing agent (control; lane 3 in each figure), and the molecular weights of the upper bands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions.

The results show that samples incubated/treated with the different reducing agents (DTT, Cysteine, GSH, and Na₂SO₃) all showed a higher lower band to upper band ratio, compared to that of an antibody sample without reducing agent treatment. The results suggest that incubation of the antibody with the reducing agent could be an effective way to improve or increase the percentage or structural homogeneity of antibody preparation of the S191C variants having an engineered disulfide bond formed at the position 191 of the CH1 region of the antibody.

### Example 7 Assessment of Effect of De-Cysteinylation by a Reducing Agent Such as 2-MEA and TCEP in Different pH Buffers

### Example 7-1 Preparation of Antibody Samples Treated with 2-MEA and TCEP

Using the antibody produced in Example 4-1, 2-MEA and TCEP were examined for whether they can promote formation of disulfide bonds in Fabs under various pH conditions.

2-MEA (Sigma-Aldrich: M6500) and TCEP (Sigma-Aldrich: C4706) were dissolved in 25 mM NaCl, 20 mM Na-Phosphate buffer, pH7.0. Especially, TCEP was adjusted to pH 7 with NaOH. 20 mg/mL of the antibody was mixed with 1 mM 2-MEA or 0.25 mM TCEP under each pH condition shown in Table 16. The composition of the pH buffer is as follows: 50 mM Acetic Acid pH3.1, 50 mM Acetic Acid adjust to pH4.0 with 1M Tris base, 50 mM Acetic Acid adjust to pH5.0 with 1M Tris base, 25 mM NaCl, 20 mM Na-Phosphate buffer pH6.0, 25 mM NaCl, 20 mM Na-Phosphate buffer pH7.0, 25 mM NaCl, 20 mM Na-Phosphate buffer pH8.0. Mixed samples were incubated in each pH buffer at 37 degrees C. for 2 hours. To stop the reduction reaction, the buffers of the mixtures with reducing agents were changed to the buffers without the reducing agents. Then, the samples were incubated at RT overnight for re-oxidation.

TABLE 16
pH of reaction buffers comprising the antibody
and reducing reagent in each sample

| | Antibody | | pH of | | |
|---|---|---|---|---|---|
| Sample | conc. | Reagents | reaction | FIG. | Lane |
| No. | (mM) | name | buffer | No. | No. |
| 1 [Control] | 20 | None | 3 | 14 | 3 |
| 2 | 20 | 2-MEA | 3 | 14 | 4 |
| 3 | 20 | TCEP | 3 | 14 | 5 |
| 4 [Control] | 20 | None | 4 | 14 | 6 |
| 5 | 20 | 2-MEA | 4 | 14 | 7 |
| 6 | 20 | TCEP | 4 | 14 | 8 |
| 7 [Control] | 20 | None | 5 | 14 | 9 |
| 8 | 20 | 2-MEA | 5 | 14 | 10 |
| 9 | 20 | TCEP | 5 | 14 | 11 |
| 10 [Control] | 20 | None | 6 | 15 | 3 |
| 11 | 20 | 2-MEA | 6 | 15 | 4 |
| 12 | 20 | TCEP | 6 | 15 | 5 |
| 13 [Control] | 20 | None | 7 | 15 | 6 |
| 8 | 20 | 2-MEA | 7 | 15 | 7 |
| 15 | 20 | TCEP | 7 | 15 | 8 |
| 16 [Control] | 20 | None | 8 | 15 | 9 |
| 17 | 20 | 2-MEA | 8 | 15 | 10 |
| 18 | 20 | TCEP | 8 | 15 | 11 |

### Example 7-2 Assessment of Electrophoretic Mobility in Polyacrylamide Gel of the Samples in Each pH Buffer

Similarly to Example 4-3, non-reducing SDS-PAGE was carried out with the reaction samples produced in Example 7-1, the gel image was captured, and bands were quantified.

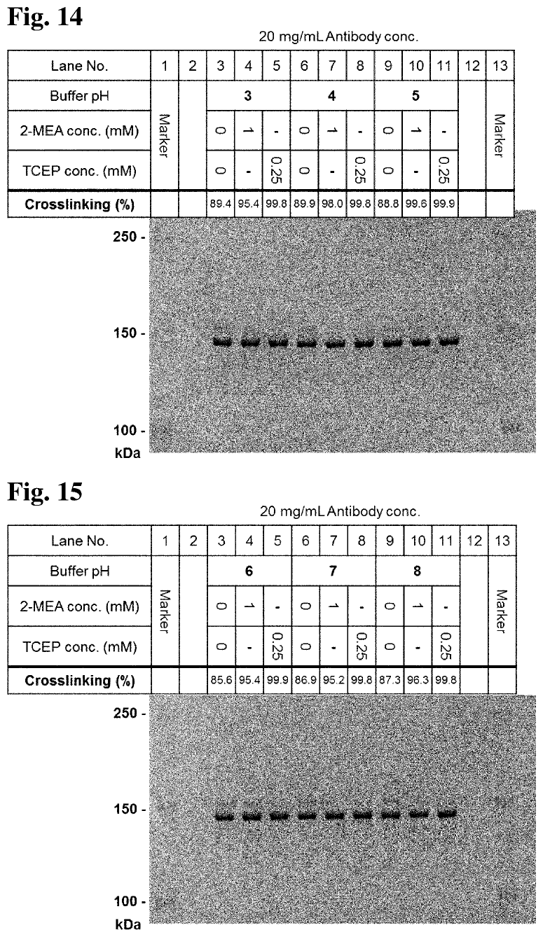

The gel images are shown in FIGS. 14 to 16. In the gel images, two bands were observed in the sample without (0 mM of) a reducing agent (control; lane 3, 6 and 9 in each figure), and the molecular weights of the upper hands correspond to that of the parent antibody. It is highly likely that structural changes such as crosslinking via disulfide bonds of Fabs were caused by cysteine substitution, which resulted in the change in electrophoretic mobility. Thus, the lower band can be considered to correspond to the antibody having one or more engineered disulfide bond(s) formed between the CH1 regions.

The results show that antibody samples incubated/treated with reducing agents at different pH conditions showed a higher lower band to upper band ratio, compared to that of an antibody sample without reducing agent treatment.

### Example 8 Separation of Crosslinked OKT3.S191C and its Variants by Cation Exchange Chromatography

### Example 8-1 Fractionation of OKT3.S191C by Cation Exchange Chromatography

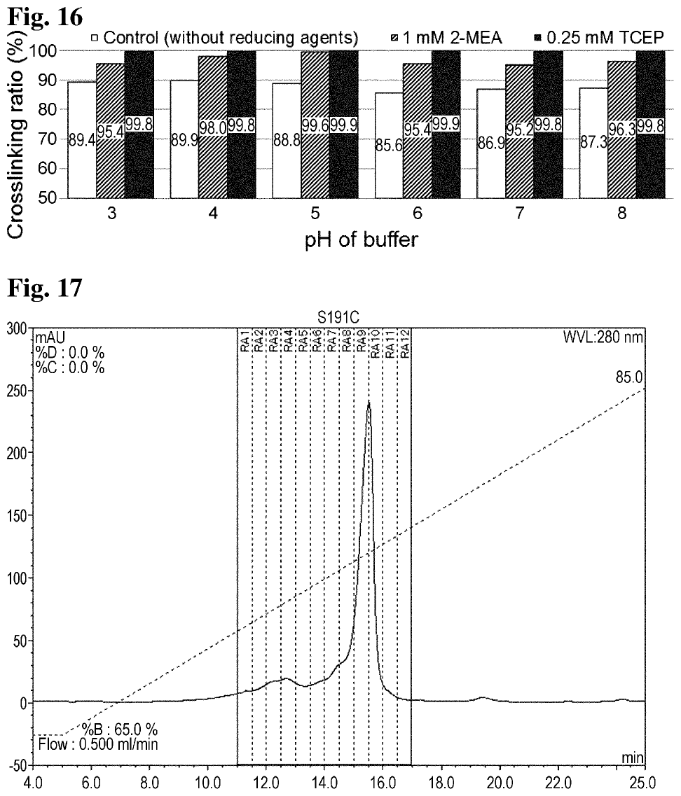

Cation exchange chromatography (CIEX) was conducted on a ProPac™ WCX-10 BioLC column, 4 mm×250 mm (Thermo) at a flow rate of 0.5 ml/min on an UltiMate 3000 UHPLC system (Thermo Scientific Dionex). The column temperature was set at 40 degrees C. Eighty microgram of OKT3.S191C (heavy chain: OKT3VH0000-G1T4.S191C (SEQ ID NO: 1428), light chain: OKT3VL0000-KT0 (SEQ ID NO: 1243)) were loaded after the column was equilibrated with 35% mobile phase A (CX-1 pH Gradient Buffer A, pH5.6, Thermo) mixed with 65% mobile phase B (CX-1 pH Gradient Buffer B, pH10.2, Thermo). Then the column was eluted with linear gradient from 65 to 85% mobile phase B for 20 min. Detection was done by UV detector (280 nm). Four times of injections were carried out and a total of 12 fractions were collected between 11 and 17 min, with samples taken at 30-sec intervals (FIG. 17). Each fraction was concentrated and evaluated using non-reducing SDS-PAGE (as described in Example 7-2). Chromatograms were analyzed using Chromeleon™ 6.8 (Thermo Scientific Dionex).

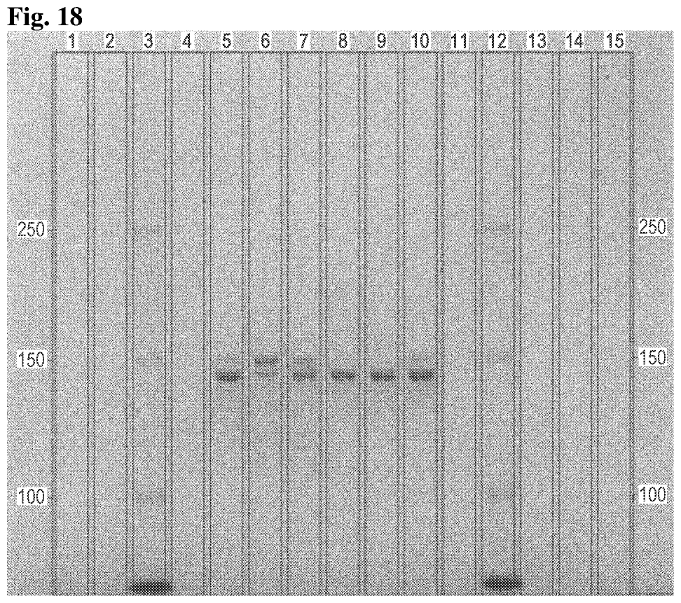

As shown in the non-reducing SDS-PAGE data (FIG. 18), the acidic peaks contained the non-crosslinked Fabs (upper band), whereas the main peak contained only crosslinked Fabs (lower band). This indicated that the non-crosslinked species were eluted faster (in fraction RA3-6) and the crosslinked Fab can be separated from them using cation exchange chromatography.

### Example 8-2 Fractionation of OKT3.S191C0110 by Cation Exchange Chromatography

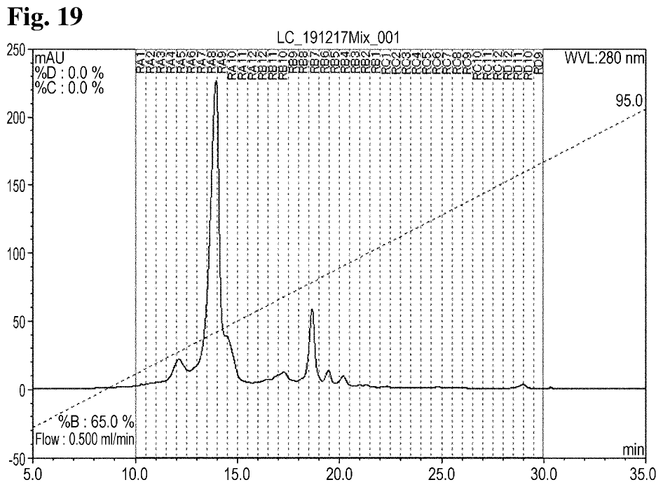

Cation exchange chromatography (CIEX) was conducted on a ProPac™ WCX-10 BioLC column, 4 mm×250 mm (Thermo) at a flow rate of 0.5 ml/min on an UltiMate 3000 UHPLC system (Thermo Scientific Dionex). The column temperature was set at 40 degrees C. Approximately 100 microgram of OKT3.S191C0110 (heavy chain: OKT3VH0000-G1T4.S191C0110 (SEQ ID NO: 1429), light chain: OKT3VL0000-KT0 (SEQ ID NO: 1243)) was loaded after the column was equilibrated with 35% mobile phase A (CX-1 pH Gradient Buffer A, pH5.6, Thermo) mixed with 65% mobile phase B (CX-1 pH Gradient Buffer B, pH10.2, Thermo). Then the column was eluted with linear gradient from 65 to 100% mobile phase B for 20 min. Detection was done by UV detector (280 nm). Three times of injections were carried out and a total of 40 fractions were collected between 10 and 30 min, with samples taken at 30-sec intervals (FIG. 19). Each fraction was concentrated and evaluated using non-reducing SDS-PAGE (described in Example 7-2). Chromatograms were analyzed using Chromeleon™ 6.8 (Thermo Scientific Dionex).

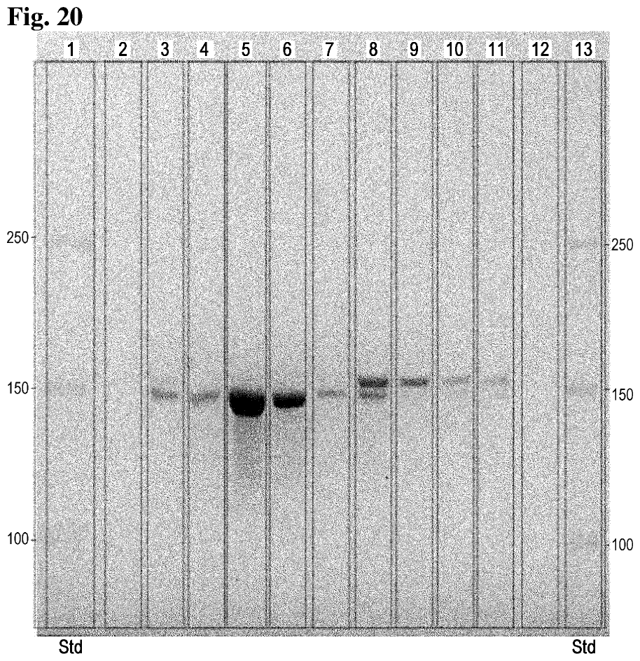

As shown in the SDS-PAGE data (FIG. 20), the antibody species with non-crosslinked Fabs (upper band) was observed in the acidic peaks and the basic peaks, whereas the main peak contained only the antibody species with crosslinked Fabs (lower band). This indicates that additional charge mutations affected the surface charge in the antibody species with non-crosslinked Fab. The cation exchange chromatography is a useful tool to purify the antibody with crosslinked Fabs.

### Example 9

Assessment of Antibodies Having Additional Disulfide Bond and Charged Mutations within Fab Region

### Example 9-1 Production of Antibodies Having Additional Disulfide Bond and Charged Mutations within Fab Region

One disulfide bond and charged mutations were introduced in the heavy chain of an anti-human CD3 antibody, OKT3 (heavy chain: OKT3VH0000-G1T4 (SEQ ID NO: 1242), light chain: OKT3VL0000-KT0 (SEQ ID NO: 1243)).

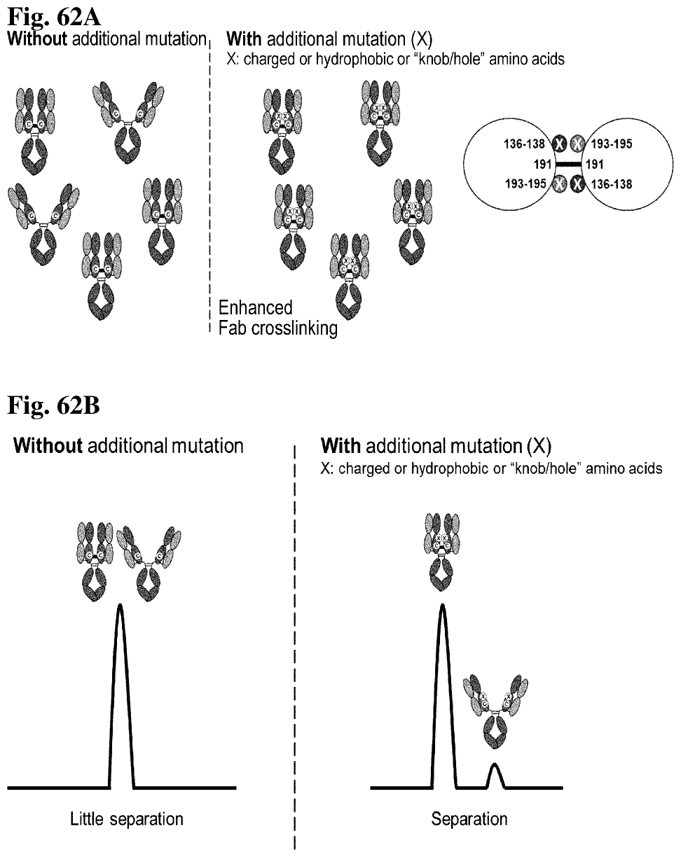

An amino acid residue structurally exposed to the surface of the OKT3 heavy chain constant region (G1T4, SEQ ID NO: 1244) was substituted with cysteine to produce the variant of OKT3 heavy chain constant region (G1T4.S191C, SEQ ID NO: 1245). In addition, CH1-CH1 interface amino acid residues structurally exposed to the surface of G1T4.S191C were substituted with charged amino acids (FIG. 62A) to produce the variants of G1T4.S191C shown in Table 82. These heavy chain constant regions were each linked with the OKT3 heavy chain variable region (OKT3VH0000, SEQ ID NO: 1246) to produce the OKT3 heavy chain variants, and expression vectors encoding the corresponding genes were produced by a method known in the art.

TABLE 82
G1T4.S191C variants with charged amino acid substitution

| Variants of | Charged mutations |
|---|---|
| heavy chain | made to G1T4.S191C |
| constant region | (EU numbering) |
| G1T4.S191C0150 | G137K/G138K/L193E |
| G1T4.S191C0151 | G137K/G138K/G194E |
| G1T4.S191C0152 | G137K/G138K/T195E |
| G1T4.S191C0153 | S136K/G138K/L193E |
| G1T4.S191C0154 | S136K/G138K/G194E |
| G1T4.S191C0155 | S136K/G138K/T195E |
| G1T4.S191C0156 | S136K/G137K/L193E |
| G1T4.S191C0157 | S136K/G137K/G194E |
| G1T4.S191C0158 | S136K/G137K/T195E |
| G1T4.S191C0159 | G137R/G138R/L193D |
| G1T4.S191C0160 | G137R/G138R/G194D |
| G1T4.S191C0161 | G137R/G138R/T195D |
| G1T4.S191C0162 | S136R/G138R/L193D |
| G1T4.S191C0163 | S136R/G138R/G194D |
| G1T4.S191C0164 | S136R/G138R/T195D |
| G1T4.S191C0165 | S136R/G137R/L193D |
| G1T4.S191C0166 | S136R/G137R/G194D |
| G1T4.S191C0167 | S136R/G137R/T195D |
| G1T4.S191C0168 | G137K/G138K/L193D |
| G1T4.S191C0169 | G137K/G138K/G194D |
| G1T4.S191C0170 | G137K/G138K/T195D |
| G1T4.S191C0171 | S136K/G138K/L193D |
| G1T4.S191C0172 | S136K/G138K/G194D |
| G1T4.S191C0173 | S136K/G138K/T195D |
| G1T4.S191C0174 | S136K/G137K/L193D |
| G1T4.S191C0175 | S136K/G137K/G194D |
| G1T4.S191C0176 | S136K/G137K/T195D |
| G1T4.S191C0177 | G137R/G138R/L193E |
| G1T4.S191C0178 | G137R/G138R/G194E |
| G1T4.S191C0179 | G137R/G138R/T195E |
| G1T4.S191C0180 | S136R/G138R/L193E |
| G1T4.S191C0181 | S136R/G138R/G194E |
| G1T4.S191C0182 | S136R/G138R/T195E |
| G1T4.S191C0183 | S136R/G137R/L193E |
| G1T4.S191C0184 | S136R/G137R/G194E |
| G1T4.S191C0185 | S136R/G137R/T195E |
| G1T4.S191C0186 | S136K/L193D |
| G1T4.S191C0187 | S136K/L193E |
| G1T4.S191C0188 | S136R/L193D |
| G1T4.S191C0189 | S136R/L193E |
| G1T4.S191C0190 | G137K/L193D |
| G1T4.S191C0191 | G137K/L193E |
| G1T4.S191C0192 | G137R/L193D |
| G1T4.S191C0193 | G137R/L193E |
| G1T4.S191C0194 | G138K/L193D |
| G1T4.S191C0195 | G138K/L193E |
| G1T4.S191C0196 | G138R/L193D |
| G1T4.S191C0197 | G138R/L193E |

The OKT3 heavy chain variants produced above were combined with the OKT3 light chain. The OKT3 variants shown in Table 83 were expressed by transient expression using Expi293 cells (Life technologies) by a method known in the art, and purified with Protein A by a method known in the art. In this Example, OKT3 is called “parent antibody”, OKT3.S191C is called “S191C variant”, and its variants are called “charged variants”.

TABLE 83
OKT3 variants with cysteine and charged amino acid substitution

| | Heavy | Heavy | |
|---|---|---|---|
| | chain | chain | |
| | variable | constant | Light |
| | region | region | T chain |
| Name of OKT3 | SEQ | (SEQ | SEQ |
| variants | ID NO: | ID NO) | ID NO: |
| OKT3 | 1246 | G1T4 | 1243 |
| | | (SEQ ID NO: 1244) | |
| OKT3.S191C | 1246 | G1T4.S191C | 1243 |
| | | (SEQ ID NO: 1245) | |
| OKT3.S191C0150 | 1246 | G1T4.S191C0150 | 1243 |
| OKT3.S191C0151 | 1246 | G1T4.S191C0151 | 1243 |
| OKT3.S191C0152 | 1246 | G1T4.S191C0152 | 1243 |
| OKT3.S191C0153 | 1246 | G1T4.S191C0153 | 1243 |
| OKT3.S191C0154 | 1246 | G1T4.S191C0154 | 1243 |
| OKT3.S191C0155 | 1246 | G1T4.S191C0155 | 1243 |
| OKT3.S191C0156 | 1246 | G1T4.S191C0156 | 1243 |
| OKT3.S191C0157 | 1246 | G1T4.S191C0157 | 1243 |
| OKT3.S191C0158 | 1246 | G1T4.S191C0158 | 1243 |
| OKT3.S191C0159 | 1246 | G1T4.S191C0159 | 1243 |
| OKT3.S191C0160 | 1246 | G1T4.S191C0160 | 1243 |
| OKT3.S191C0161 | 1246 | G1T4.S191C0161 | 1243 |
| OKT3.S191C0162 | 1246 | G1T4.S191C0162 | 1243 |
| OKT3.S191C0163 | 1246 | G1T4.S191C0163 | 1243 |
| OKT3.S191C0164 | 1246 | G1T4.S191C0164 | 1243 |
| OKT3.S191C0165 | 1246 | G1T4.S191C0165 | 1243 |
| OKT3.S191C0166 | 1246 | G1T4.S191C0166 | 1243 |
| OKT3.S191C0167 | 1246 | G1T4.S191C0167 | 1243 |
| OKT3.S191C0168 | 1246 | G1T4.S191C0168 | 1243 |
| OKT3.S191C0169 | 1246 | G1T4.S191C0169 | 1243 |
| OKT3.S191C0170 | 1246 | G1T4.S191C0170 | 1243 |
| OKT3.S191C0171 | 1246 | G1T4.S191C0171 | 1243 |
| OKT3.S191C0172 | 1246 | G1T4.S191C0172 | 1243 |
| OKT3.S191C0173 | 1246 | G1T4.S191C0173 | 1243 |
| OKT3.S191C0174 | 1246 | G1T4.S191C0174 | 1243 |
| OKT3.S191C0175 | 1246 | G1T4.S191C0175 | 1243 |
| OKT3.S191C0176 | 1246 | G1T4.S191C0176 | 1243 |
| OKT3.S191C0177 | 1246 | G1T4.S191C0177 | 1243 |
| OKT3.S191C0178 | 1246 | G1T4.S191C0178 | 1243 |
| OKT3.S191C0179 | 1246 | G1T4.S191C0179 | 1243 |
| OKT3.S191C0180 | 1246 | G1T4.S191C0180 | 1243 |
| OKT3.S191C0181 | 1246 | G1T4.S191C0181 | 1243 |
| OKT3.S191C0182 | 1246 | G1T4.S191C0182 | 1243 |
| OKT3.S191C0183 | 1246 | G1T4.S191C0183 | 1243 |
| OKT3.S191C0184 | 1246 | G1T4.S191C0184 | 1243 |
| OKT3.S191C0185 | 1246 | G1T4.S191C0185 | 1243 |
| OKT3.S191C0186 | 1246 | G1T4.S191C0186 | 1243 |
| OKT3.S191C0187 | 1246 | G1T4.S191C0187 | 1243 |
| OKT3.S191C0188 | 1246 | G1T4.S191C0188 | 1243 |
| OKT3.S191C0189 | 1246 | G1T4.S191C0189 | 1243 |
| OKT3.S191C0190 | 1246 | G1T4.S191C0190 | 1243 |
| OKT3.S191C0191 | 1246 | G1T4.S191C0191 | 1243 |
| OKT3.S191C0192 | 1246 | G1T4.S191C0192 | 1243 |
| OKT3.S191C0193 | 1246 | G1T4.S191C0193 | 1243 |
| OKT3.S191C0194 | 1246 | G1T4.S191C0194 | 1243 |
| OKT3.S191C0195 | 1246 | G1T4.S191C0195 | 1243 |
| OKT3.S191C0196 | 1246 | G1T4.S191C0196 | 1243 |
| OKT3.S191C0197 | 1246 | G1T4.S191C0197 | 1243 |

### Example 9-2 Assessment of Electrophoretic Mobility in Polyacrylamide Gel of Antibodies Having Additional Disulfide Bond and Charged Mutations within Fab Region

Similarly to Example 1-2, non-reducing SDS-PAGE was carried out with the charged variants produced in Example 9-1, the gel image was captured, and bands were quantified.

In the gel images, two bands were observed in S191C variant, and the molecular weight of the upper bands was similar to that of the parent antibody. The ratio of the lower bands to upper bands are shown in Table 84. Among charged variants, most of them showed higher ratio of lower bands to upper bands, compared to S191C variants. It is highly likely that structural changes such as crosslinking via disulfide bond of Fabs were caused by cysteine substitution, which resulted in the change in electrophoretic mobility. Thus, additional charged mutations to S191C variant are likely to enhance crosslinking of Fabs.

TABLE 84
The ratio of the lower bands to upper bands of OKT3 variants
with cysteine and charged amino acid substitution

| | Ratio of |
|---|---|
| Name of OKT3 | lower band to |
| variants | upper band (%) |
| OKT3 | 0 |
| OKT3.S191C | 65.4 |
| OKT3.S191C0150 | 70.0 |
| OKT3.S191C0151 | 58.5 |
| OKT3.S191C0152 | 66.5 |
| OKT3.S191C0153 | 70.7 |
| OKT3.S191C0154 | 56.7 |
| OKT3.S191C0155 | 65.9 |
| OKT3.S191C0156 | 70.2 |
| OKT3.S191C0157 | 71.6 |
| OKT3.S191C0158 | 68.5 |
| OKT3.S191C0159 | 68.9 |
| OKT3.S191C0160 | 68.1 |
| OKT3.S191C0161 | 79.1 |
| OKT3.S191C0162 | 69.6 |
| OKT3.S191C0163 | 63.3 |
| OKT3.S191C0164 | 70.8 |
| OKT3.S191C0165 | 76.7 |
| OKT3.S191C0166 | 75.6 |
| OKT3.S191C0167 | 68.0 |
| OKT3.S191C0168 | 66.2 |
| OKT3.S191C0169 | 58.8 |
| OKT3.S191C0170 | 74.5 |
| OKT3.S191C0171 | 72.9 |
| OKT3.S191C0172 | 64.0 |
| OKT3.S191C0173 | 69.1 |
| OKT3.S191C0174 | 62.1 |
| OKT3.S191C0175 | 70.4 |
| OKT3.S191C0176 | 89.8 |
| OKT3.S191C0177 | 76.7 |
| OKT3.S191C0178 | 86.3 |
| OKT3.S191C0179 | 82.2 |
| OKT3.S191C0180 | 80.5 |
| OKT3.S191C0181 | 76.0 |
| OKT3.S191C0182 | 78.8 |
| OKT3.S191C0183 | 78.5 |
| OKT3.S191C0184 | 80.6 |
| OKT3.S191C0185 | 83.5 |
| OKT3.S191C0186 | 75.7 |
| OKT3.S191C0187 | 81.6 |
| OKT3.S191C0188 | 68.5 |
| OKT3.S191C0189 | 83.4 |
| OKT3.S191C0190 | 78.0 |
| OKT3.S191C0191 | 72.5 |
| OKT3.S191C0192 | 80.6 |
| OKT3.S191C0193 | 76.3 |
| OKT3.S191C0194 | 84.8 |
| OKT3.S191C0195 | 79.3 |
| OKT3.S191C0196 | 82.7 |
| OKT3.S191C0197 | 85.5 |

### Example 9-3 Assessment of Peak Separation of Antibodies Having Additional Disulfide Bond and Charged Mutations within Fab Region by Cation Exchange Chromatography

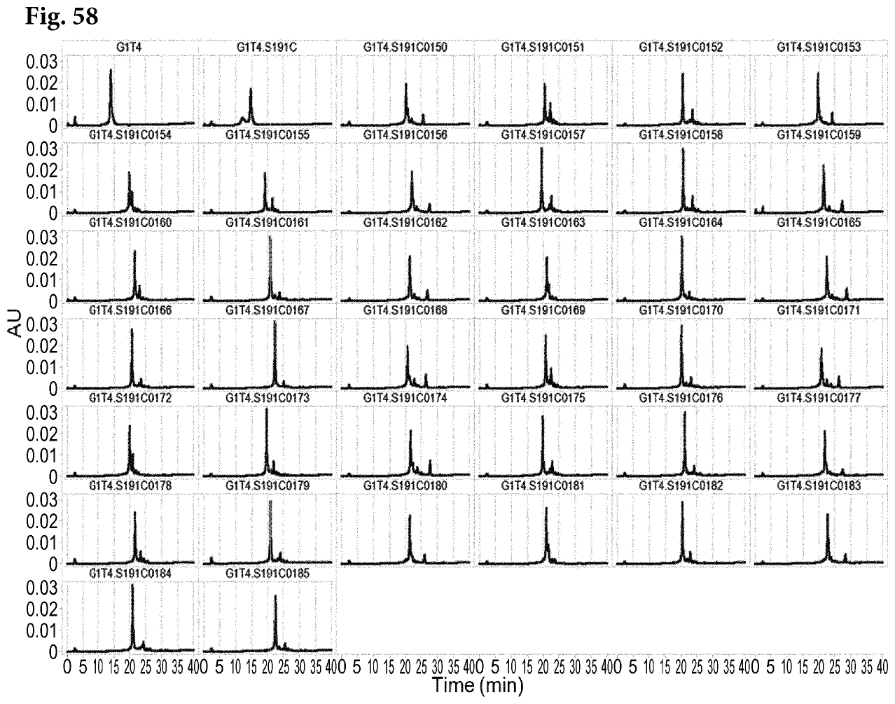

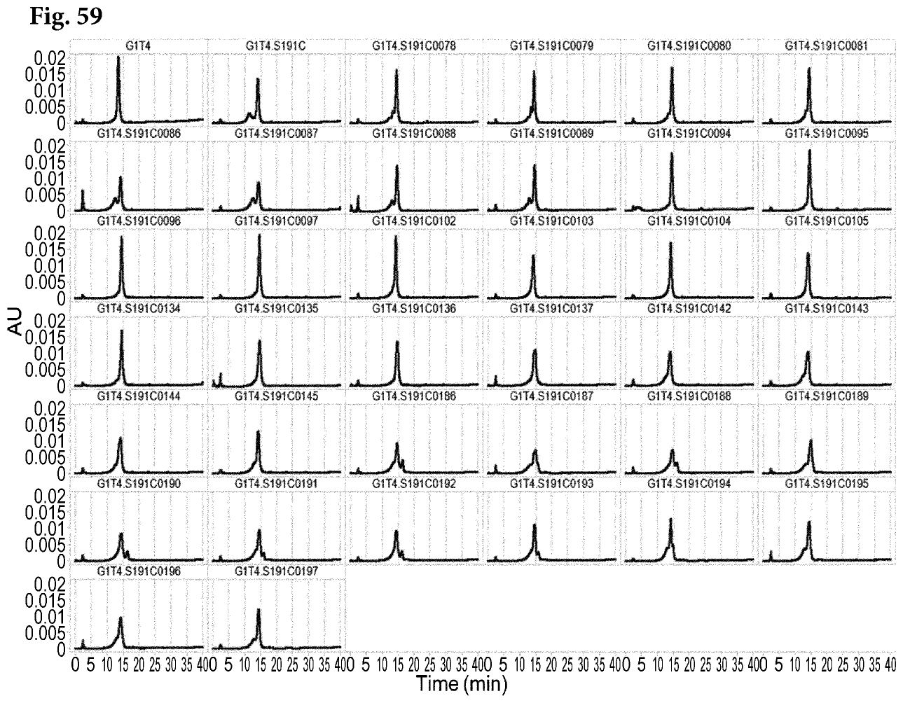

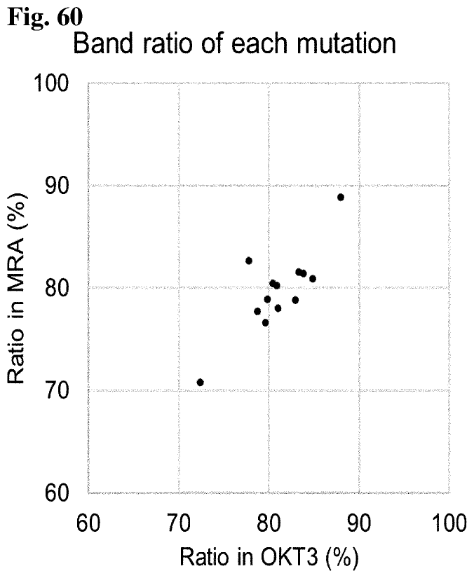

Cation exchange chromatography (CIEX) was conducted on a ProPac™ WCX-10 BioLC column, 4 mm×250 mm (Thermo) at a flow rate of 0.5 ml/min on an Alliance HPLC system (Waters). Column temperature was set at 40 degrees C. Eighty microgram of charged variants produced in Example 9-1 were loaded after column was equilibrated with 35% mobile phase A (CX-1 pH Gradient Buffer A, pH5.6, Thermo) mixed with 65% mobile phase B (CX-1 pH Gradient Buffer B, pH10.2, Thermo). Then the column was eluted with linear gradient from 65 to 100% mobile phase B for 35 min. Detection was done by UV detector (280 nm). Chromatograms of CIEX are shown in FIGS. 58 and 59.

In FIGS. 58 and 59, similar peak patterns to FIG. 19, which could separate crosslinked and non-crosslinked Fabs, were observed in some charged variants. It is highly likely that additional charged mutations to S191C variant can enhance not only crosslinking of Fabs but also separation between crosslinked and non-crosslinked Fabs by CIEX. See also FIG. 62B.

### Example 10

Assessment of Different Antibodies Having Additional Disulfide Bond and Charged Mutations within Fab Region

### Example 10-1 Production of Different Antibodies Having Additional Disulfide Bond and Charged Mutations within Fab Region

One disulfide bond and charged mutations were introduced in the heavy chain of an anti-human CD3 antibody, OKT3 (heavy chain: OKT3VH0000-G1T4 (SEQ ID NO: 1242), light chain: OKT3VL0000-KT0 (SEQ ID NO: 1243)). Similarly, one disulfide bond and charge mutations were introduced in the heavy chain of an anti-human IL-6R antibody, MRA (heavy chain: MRAH-G1T4 (SEQ ID NO: 15), light chain: MRAL-k0 (SEQ ID NO: 16)).

An amino acid residue structurally exposed to the surface of the OKT3 and MRA heavy chain constant region (G1T4, SEQ ID NO: 1244) was substituted with cysteine to produce the variant of OKT3 heavy chain constant region (G1T4.S191C, SEQ ID NO: 1245). In addition, CH1-CH1 interface amino acid residues structurally exposed to the surface of G1T4.S191C were substituted with charged amino acids (FIG. 62A) to produce the variants of G1T4.S191C shown in Table 85. These heavy chain constant regions were each linked with the OKT3 heavy chain variable region (OKT3VH0000, SEQ ID NO: 1246) and MRA heavy chain variable region (MRAH, SEQ ID NO: P17) respectively to produce the OKT3 and MRA heavy chain variants, and expression vectors encoding the corresponding genes were produced by a method known in the art.

TABLE 85
G1T4.S191C variants with charged amino acid substitution

| Variants of heavy | Charged mutations |
|---|---|
| chain constant | made to G1T4.S191C |
| region | (EU numbering) |
| G1T4.S191C0159 | G137R/G138R/L193D |
| G1T4.S191C0161 | G137R/G138R/T195D |
| G1T4.S191C0162 | S136R/G138R/L193D |
| G1T4.S191C0164 | S136R/G138R/T195D |
| G1T4.S191C0165 | S136R/G137R/L193D |
| G1T4.S191C0167 | S136R/G137R/T195D |
| G1T4.S191C0177 | G137R/G138R/L193E |
| G1T4.S191C0179 | G137R/G138R/T195E |
| G1T4.S191C0180 | S136R/G138R/L193E |
| G1T4.S191C0182 | S136R/G138R/T195E |
| G1T4.S191C0183 | S136R/G137R/L193E |
| G1T4.S191C0185 | S136R/G137R/T195E |

The OKT3 and MRA heavy chain variants produced above were combined with the OKT3 and MRA light chains respectively. The OKT3 and MRA variants shown in Table 86 were expressed by transient expression using Expi293 cells (Life technologies) by a method known in the art, and purified with Protein A by a method known in the art. In this Example, OKT3 and MRA are called “parent antibody”, OKT3.S191C and MRA.S191C are called “S191C variant”, and their variants are called “charged variants”.

TABLE 86
OKT3 and MRA variants with cysteine
and charged amino acid substitution

| | | Heavy | Heavy | |
|---|---|---|---|---|
| | | chain | chain | |
| | | variable | constant | Light |
| | | region | region | chain |
| Original | Name of OKT3 | SEQ | (SEQ | SEQ |
| antibody | variants | ID NO: | ID NO) | ID NO: |
| OKT3 | OKT3 | 1246 | G1T4 | 1243 |
| | | | (SEQ ID NO: 1244) | |
| OKT3 | OKT3.S191C | 1246 | G1T4.S191C | 1243 |
| | | | (SEQ ID NO: 1245) | |
| OKT3 | OKT3.S191C0159 | 1246 | G1T4.S191C0159 | 1243 |
| OKT3 | OKT3.S191C0161 | 1246 | G1T4.S191C0161 | 1243 |
| OKT3 | OKT3.S191C0162 | 1246 | G1T4.S191C0162 | 1243 |
| OKT3 | OKT3.S191C0164 | 1246 | G1T4.S191C0164 | 1243 |
| OKT3 | OKT3.S191C0165 | 1246 | G1T4.S191C0165 | 1243 |
| OKT3 | OKT3.S191C0167 | 1246 | G1T4.S191C0167 | 1243 |
| OKT3 | OKT3.S191C0177 | 1246 | G1T4.S191C0177 | 1243 |
| OKT3 | OKT3.S191C0179 | 1246 | G1T4.S191C0179 | 1243 |
| OKT3 | OKT3.S191C0180 | 1246 | G1T4.S191C0180 | 1243 |
| OKT3 | OKT3.S191C0182 | 1246 | G1T4.S191C0182 | 1243 |
| OKT3 | OKT3.S191C0183 | 1246 | G1T4.S191C0183 | 1243 |
| OKT3 | OKT3.S191C0185 | 1246 | G1T4.S191C0185 | 1243 |
| MRA | MRA | 17 | G1T4 | 16 |
| | | | (SEQ ID NO: 1244) | |
| MRA | MRA.S191C | 17 | G1T4.S191C | 16 |
| | | | (SEQ ID NO: 1245) | |
| MRA | MRA.S191C0159 | 17 | G1T4.S191C0159 | 16 |
| MRA | MRA.S191C0161 | 17 | G1T4.S191C0161 | 16 |
| MRA | MRA.S191C0162 | 17 | G1T4.S191C0162 | 16 |
| MRA | MRA.S191C0164 | 17 | G1T4.S191C0164 | 16 |
| MRA | MRA.S191C0165 | 17 | G1T4.S191C0165 | 16 |
| MRA | MRA.S191C0167 | 17 | G1T4.S191C0167 | 16 |
| MRA | MRA.S191C0177 | 17 | G1T4.S191C0177 | 16 |
| MRA | MRA.S191C0179 | 17 | G1T4.S191C0179 | 16 |
| MRA | MRA.S191C0180 | 17 | G1T4.S191C0180 | 16 |
| MRA | MRA.S191C0182 | 17 | G1T4.S191C0182 | 16 |
| MRA | MRA.S191C0183 | 17 | G1T4.S191C0183 | 16 |
| MRA | MRA.S191C0185 | 17 | G1T4.S191C0185 | 16 |

### Example 10-2 Assessment of Electrophoretic Mobility in Polyacrylamide Gel of Different Antibodies Having Additional Disulfide Bond and Charged Mutations within Fab Region

It was examined whether the antibodies produced in Example 10-1 show a different electrophoretic mobility in polyacrylamide gel by non-reducing SDS-PAGE.

Sample Buffer Solution (2ME-) (×4) (Wako; 198-13282) was used for preparation of electrophoresis samples. The samples were treated for 10 minutes under the condition of specimen concentration 75 microgram/mL and 70 degrees C., and then subjected to non-reducing SDS-PAGE. In non-reducing SDS-PAGE, electrophoresis was carried out for 90 minutes at 126 V, using 4% SDS-PAGE mini 15 well 1.0 mm 15 well (TEFCO; Cat #01-052-6). Then, the gel was stained with CBB stain, the gel image was captured with ChemiDocTouchMP (BIORAD), and the bands were quantified with Image Lab (BIORAD).

In the gel images, two bands were observed in S191C variant, and the molecular weight of the upper bands was similar to that of the parent antibody. The ratio of the lower bands to upper bands are shown in Table 87 and plotted in a scatter diagram shown in FIG. 60. Good correlation between the ratio of lower bands to upper bands in OKT3 and MRA was observed. Thus, additional charged mutations to S191C variant are likely to enhance crosslinking of Fabs of not only OKT3 but also other antibodies binding to other antigen such as MRA.

TABLE 87
The ratio of the lower bands to upper bands of OKT3 and MRA
variants with cysteine and charged amino acid substitution

| | | Ratio of |
|---|---|---|
| Original | Name of OKT3 | lower band to |
| antibody | variants | upper band (%) |
| OKT3 | OKT3 | 0 |
| OKT3 | OKT3.S191C | 72.4 |
| OKT3 | OKT3.S191C0159 | 80.9 |
| OKT3 | OKT3.S191C0161 | 83.0 |
| OKT3 | OKT3.S191C0162 | 83.3 |
| OKT3 | OKT3.S191C0164 | 78.7 |
| OKT3 | OKT3.S191C0165 | 81.0 |
| OKT3 | OKT3.S191C0167 | 79.6 |
| OKT3 | OKT3.S191C0177 | 80.5 |
| OKT3 | OKT3.S191C0179 | 79.9 |
| OKT3 | OKT3.S191C0180 | 83.9 |
| OKT3 | OKT3.S191C0182 | 77.8 |
| OKT3 | OKT3.S191C0183 | 88.0 |
| OKT3 | OKT3.S191C0185 | 84.8 |
| MRA | MRA | 0 |
| MRA | MRA.S191C | 70.8 |
| MRA | MRA.S191C0159 | 80.3 |
| MRA | MRA.S191C0161 | 78.8 |
| MRA | MRA.S191C0162 | 81.6 |
| MRA | MRA.S191C0164 | 77.7 |
| MRA | MRA.S191C0165 | 78.0 |
| MRA | MRA.S191C0167 | 76.7 |
| MRA | MRA.S191C0177 | 80.5 |
| MRA | MRA.S191C0179 | 78.9 |
| MRA | MRA.S191C0180 | 81.4 |
| MRA | MRA.S191C0182 | 82.7 |
| MRA | MRA.S191C0183 | 88.9 |
| MRA | MRA.S191C0185 | 80.9 |

### Example 10-3 Assessment of Peak Separation of Different Antibodies Having Additional Disulfide Bond and Charged Mutations within Fab Region by Cation Exchange Chromatography

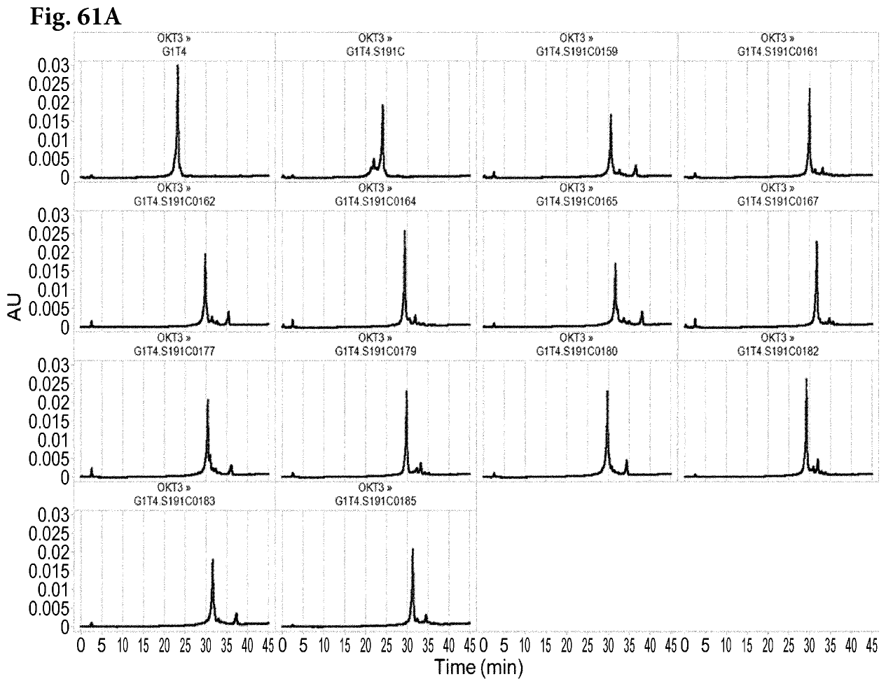

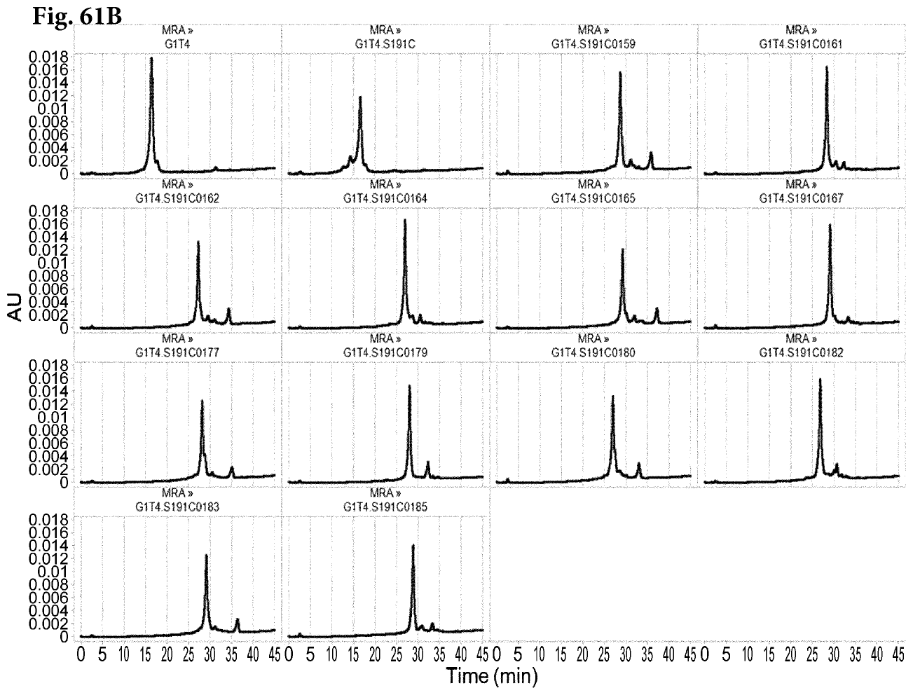

Cation exchange chromatography (CIEX) was conducted on a ProPac™ WCX-10 BioLC column, 4 mm×250 mm (Thermo) at a flow rate of 0.5 ml/min on an Alliance HPLC system (Waters). Column temperature was set at 40 degrees C. Eighty microgram of charged variants produced in Example 10-1 were loaded after column was equilibrated with 45% mobile phase A (CX-1 pH Gradient Buffer A, pH5.6, Thermo) mixed with 55% mobile phase B (CX-1 pH Gradient Buffer B, pH10.2, Thermo). Then the column was eluted with linear gradient from 55 to 95% mobile phase B for 40 min. Detection was done by UV detector (280 nm). Chromatograms of CIEX are shown in FIGS. 61A and 61B.

In FIGS. 61A and 61B, similar peak patterns between OKT3 and MRA variants were observed. Thus, additional charged mutations to S191C variant are likely to enhance separation between crosslinked and non-crosslinked Fabs of not only OKT3 but also other antibodies binding to other antigen such as MRA by CIEX.

### Reference Example 1 Concept of Fab-Crosslinked Antibody

Agonist antibodies are superior in properties such as stability, pharmacokinetics, and production methods compared to natural ligands and their fusion proteins, and their pharmaceutical development is under way. However, in general, agonist antibodies with strong activity are more difficult to obtain than mere binding or neutralizing antibodies. A solution to this problem is therefore being wanted.

Properties needed for an agonist antibody may depend on the type of the ligand. For agonist antibodies against the TNF receptor superfamily, typified by Death receptor (DR), OX40, 4-1BB, CD40, and such, it has been reported that multimerization of antibody or ligand contributes to the activation. As techniques for increasing this effect, use of natural ligands, crosslinking by anti-Fc antibodies, crosslinking via Fc gamma Rs, multimerization of antibody binding domains, multimerization via antibody Fc, and such have been reported to enhance the agonist activity. It is also known that, for certain types of antigens, adjustment of the distance of antigen-binding sites using antibody Fab structure or scFv leads to enhancement of the agonist activity regardless of multimerization.

As another technique, an agonist antibody against a cytokine receptor which is a bispecific antibody capable of binding to different epitopes within the same antigen has been reported. Moreover, a method of improving agonist activity by using chemical conjugation to crosslink two different Fabs in a similar manner has been reported.

More methods besides those mentioned above for improving the activity of agonist antibodies are wanted. However, no simple method to achieve this has been reported. Thus, the inventors developed a method for crosslinking Fabs with each other through introducing minimum mutations, and demonstrated that this actually enhanced the agonist activity, thereby completing the invention. An exemplifying embodiment is shown in FIG. 21.

### Reference Example 2 Production of Expression Vectors for Modified Antibodies, and Expression and Purification of Modified Antibodies

An antibody gene inserted in an expression vector for animal cells was subjected to amino acid residue sequence substitution by a method known to the person skilled in the art using PCR, the In-Fusion Advantage PCR cloning kit (TAKARA), or such, to construct an expression vector for a modified antibody. The nucleotide sequence of the resulting expression vector was determined by a method known to the person skilled in the art. The produced expression vector was transiently introduced into FreeStyle293 (registered trademark) or Expi293 (registered trademark) cells (Invitrogen) and the cells were allowed to express the modified antibody into culture supernatant. The modified antibody was purified from the obtained culture supernatant by a method known to the person skilled in the art using rProtein A Sepharose (registered trademark) Fast Flow (GE Healthcare). Absorbance at 280 nm was measured using a spectrophotometer. An absorption coefficient was calculated from the measured value using the PACE method and used to calculate the antibody concentration (Protein Science 1995; 4:2411-2423).

The amount of aggregates of the modified antibody was analyzed by a method known to the person skilled in the art using Agilent 1260 Infinity (registered trademark) (Agilent Technologies) for HPLC and G3000SW_{XL} (TOSOH) as a gel filtration chromatography column. The concentration of the purified antibody was 0.1 mg/mL, and 10 microliter of the antibody was injected.

Antibodies prepared by this method (anti-CD3 epsilon antibodies, anti-CD28 antibodies, and anti-CD3 epsilon×anti-CD28 bispecific antibodies) are shown in Table 17.

TABLE 17
Antibody names, SEQ ID NOs

| | SEQ ID NO: | | | |
|---|---|---|---|---|
| Antibody | Heavy | Light | Heavy | Light |
| name | chain 1 | chain 1 | chain 2 | chain 2 |
| CD3-G4s | 1 | 10 | — | — |
| CD3-G4sHH | 2 | 10 | — | — |
| CD3-G4sLL | 1 | 11 | — | — |
| CD3-G1s | 4 | 10 | — | — |
| OKT3-G1s | 5 | 12 | — | — |
| CD28-G1 | 6 | 13 | — | — |
| CD3-G1sLL | 4 | 11 | — | — |
| CD3-G1sHH | 7 | 10 | — | — |
| CD3//CD28-G1s | 4 | 10 | 6 | 13 |
| CD3//CD28-G1sLL | 4 | 11 | 6 | 14 |
| CD3//CD28-G1sHH | 7 | 10 | 9 | 13 |
| CD3//CD28-G1sLH | 4 | 11 | 9 | 13 |
| CD3//CD28-G1sHL | 7 | 10 | 6 | 14 |
| OKT3//CD28-G1s | 5 | 12 | 6 | 13 |
| OKT3//CD28-G1sHH | 8 | 12 | 9 | 13 |
| OKT3//CD28-G1sHL | 8 | 12 | 6 | 14 |

HH: position 191 (EU numbering) was altered to Cys in the two H chain constant regions
LL: position 126 (EU numbering) was altered to Cys in the two L chain constant regions
HL, LH: position 191 (EU numbering) was altered to Cys in one H chain constant region, and position 126 (EU numbering) was altered to Cys in one L chain constant region

### Reference Example 3 Preparation of Bispecific Antibodies

The purified antibody was dialyzed into TBS (WAKO) buffer and its concentration was adjusted to 1 mg/mL. As a 10× reaction buffer, 250 mM 2-MEA (SIGMA) was prepared. Two different homodimeric antibodies prepared in Reference Example 2 were mixed in equal amount. To this mixture, a 1/10 volume of the 10× reaction buffer was added and mixed. The mixture was allowed to stand at 37 degrees C. for 90 minutes. After the reaction, the mixture was dialyzed into TBS to obtain a solution of a bispecific antibody in which the above two different antibodies were heterodimerized. The antibody concentration was measured by the above-mentioned method, and the antibody was subjected to subsequent experiments.

### Reference Example 4 Assessment of Agonist Activity

### Reference Example 4-1 Preparation of Jurkat Cell Suspension

Jurkat cells (TCR/CD3 Effector Cells (NFAT), Promega) were collected from flasks. The cells were washed with Assay Buffer (RPMI 1640 medium (Gibco), 10% FBS (HyClone), 1% MEM Non Essential Amino Acids (Invitrogen), and 1 mM Sodium Pyruvate (Invitrogen)), and then suspended at 3×10⁶ cells/mL in Assay Buffer. This suspension of Jurkat cells was subjected to subsequent experiments.

### Reference Example 4-2 Preparation of Luminescence Reagent Solution

100 mL of Bio-Glo Luciferase Assay Buffer (Promega) was added to the bottle of Bio-Glo Luciferase Assay Substrate (Promega), and mixed by inversion. The bottle was protected from light and frozen at −20 degrees C. This luminescence reagent solution was subjected to subsequent experiments.

### Reference Example 4-3 T Cell Activation Assay

T cell activation by agonist signaling was assessed based on the fold change of luciferase luminescence. The aforementioned Jurkat cells are cells transformed with a luciferase reporter gene having a NFAT responsive sequence. When the cells are stimulated by an anti-TCR/CD3 antibody, the NFAT pathway is activated via intracellular signaling, thereby inducing luciferase expression. The Jurkat cells suspension prepared as described above was added to a 384-well flat-bottomed white plate at 10 microliter per well (3×10⁴ cells/well). Next, the antibody solution prepared at each concentration (150, 15, 1.5, 0.15, 0.015, 0.0015, 0.00015, 0.000015 nM) was added at 20 microliter per well. This plate was allowed to stand in a 5% CO₂ incubator at 37 degrees C. for 24 hours. After the incubation, the luminescence reagent solution was thawed, and 30 microliter of the solution was added to each well. The plate was then allowed to stand at room temperature for 10 minutes. Luciferase luminescence in each well of the plate was measured using a luminometer.

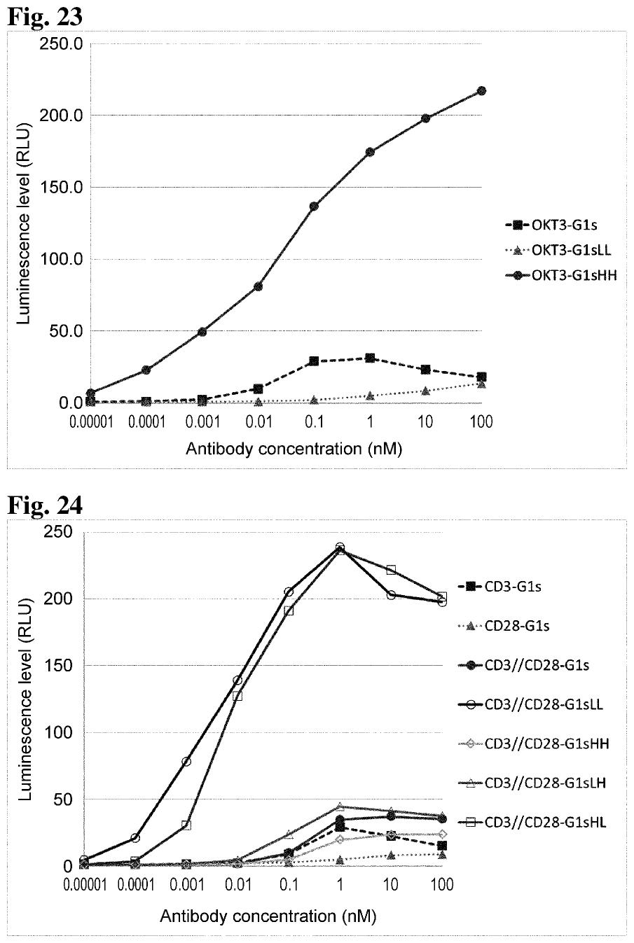

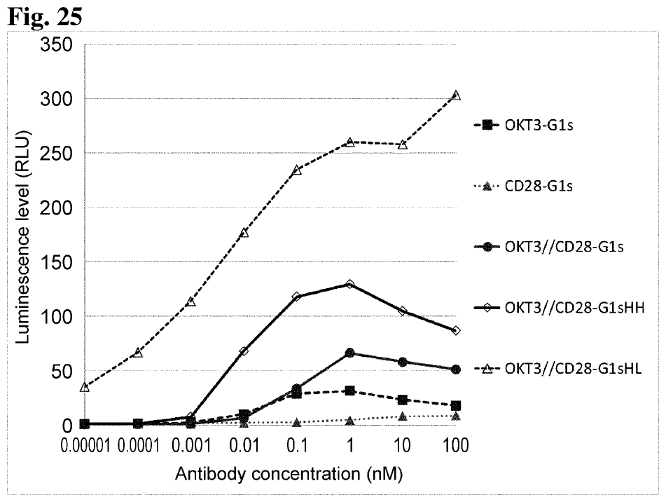

As a result, modified molecules with an additional disulfide bond linking the Fab-Fab of anti-CD3 epsilon antibody showed varied CD3-mediated signaling compared to the wild-type molecule (unmodified molecule) as shown in FIGS. 22 and 23. Furthermore, as shown in FIGS. 24 and 25, modified molecules of a bispecific antibody composed of an anti-CD3 epsilon antibody and an anti-CD28 antibody with an additional disulfide bond linking the Fab-Fab also showed largely varied CD3- and/or CD28-mediated signaling compared to the wild-type molecule. These results suggest that introducing modifications of the present invention can enhance or diminish agonist activity possessed by antigen-binding molecules such as antibodies.

### Reference Example 5 Assessment of Antibodies Having Cysteine Substitution at Various Positions in the Heavy Chain

### Reference Example 5-1 Assessment of Antibodies Having Cysteine Substitution at Various Positions in the Heavy Chain

The heavy chain variable region and constant region of an anti-human IL6R neutralizing antibody, MRA (heavy chain: MRAH-G1T4 (SEQ ID NO: 15), light chain: MRAL-k0 (SEQ ID NO: 16)) were subjected to a study in which an arbitrary amino acid residue structurally exposed to the surface was substituted with cysteine.

Amino acid residues within the heavy chain variable region of MRA (MRAH, SEQ ID NO: 17) were substituted with cysteine to produce variants of the heavy chain variable region of MRA shown in Table 18. These variants of the heavy chain variable region of MRA were each linked with the heavy chain constant region of MRA (G1T4, SEQ ID NO: 18) to produce variants of the heavy chain of MRA, and expression vectors encoding the corresponding genes were produced by a method known to the person skilled in the art.

In addition, amino acid residues within the heavy chain constant region of MRA (G1T4, SEQ ID NO: 18) were substituted with cysteine to produce variants of the heavy chain constant region of MRA shown in Table 19. These variants of the heavy chain constant region of MRA were each linked with the heavy chain variable region of MRA (MRAH, SEQ ID NO: 17) to produce variants of the heavy chain of MRA, and expression vectors encoding the corresponding genes were produced by a method known to the person skilled in the art.

The MRA heavy chain variants produced above were combined with the MRA light chain. The resultant MRA variants shown in Table 20 were expressed by transient expression using FreeStyle293 cells (Invitrogen) or Expi293 cells (Life technologies) by a method known to the person skilled in the art, and purified with Protein A by a method known to the person skilled in the art.

TABLE 18
Variants of MRA heavy chain variable region
and position of cysteine substitution

| Variant of MRA | Position of cysteine | |
|---|---|---|
| heavy chain | substitution | SEQ |
| variable region | (Kabat numbering) | ID NO: |
| MRAH.Q5C | 5 | 21 |
| MRAH.E6C | 6 | 22 |
| MRAH.S7C | 7 | 23 |
| MRAH.G8C | 8 | 24 |
| MRAH.P9C | 9 | 25 |
| MRAH.G10C | 10 | 26 |
| MRAH.L11C | 11 | 27 |
| MRAH.V12C | 12 | 28 |
| MRAH.R13C | 13 | 29 |
| MRAH.P14C | 14 | 30 |
| MRAH.S15C | 15 | 31 |
| MRAH.Q16C | 16 | 32 |
| MRAH.T17C | 17 | 33 |
| MRAH.L18C | 18 | 34 |
| MRAH.S19C | 19 | 35 |
| MRAH.L20C | 20 | 36 |
| MRAH.T21C | 21 | 37 |
| MRAH.T23C | 23 | 38 |
| MRAH.S25C | 25 | 39 |
| MRAH.G26C | 26 | 40 |
| MRAH.S28C | 28 | 41 |
| MRAH.T30C | 30 | 42 |
| MRAH.R66C | 66 | 43 |
| MRAH.V67C | 67 | 44 |
| MRAH.T68C | 68 | 45 |
| MRAH.L70C | 70 | 46 |
| MRAH.D72C | 72 | 47 |
| MRAH.T73C | 73 | 48 |
| MRAH.S74C | 74 | 49 |
| MRAH.K75C | 75 | 50 |
| MRAH.N76C | 76 | 51 |
| MRAH.Q77C | 77 | 52 |
| MRAH.S79C | 79 | 53 |
| MRAH.L80C | 80 | 54 |
| MRAH.R81C | 81 | 55 |
| MRAH.L82C | 82 | 56 |
| MRAH.S82aC | 82a | 57 |
| MRAH.S82bC | 82b | 58 |
| MRAH.V82cC | 82c | 59 |
| MRAH.S112C | 112 | 60 |
| MRAH.S113C | 113 | 61 |
| MRAH.S31C | 31 | 62 |
| MRAH.W35C | 35 | 63 |
| MRAH.S35aC | 35a | 64 |
| MRAH.Y50C | 50 | 65 |
| MRAH.I51C | 51 | 66 |
| MRAH.S52C | 52 | 67 |
| MRAH.S62C | 62 | 68 |
| MRAH.L63C | 63 | 69 |
| MRAH.K64C | 64 | 70 |
| MRAH.S65C | 65 | 71 |
| MRAH.D101C | 101 | 72 |
| MRAH.Y102C | 102 | 73 |

TABLE 19
Variants of MRA heavy chain constant region
and position of cysteine substitution

| Variant of MRA | Position of cysteine | |
|---|---|---|
| heavy chain | substitution | SEQ |
| constant region | (EU numbering) | ID NO: |
| G1T4.A118C | 118 | 74 |
| G1T4.S119C | 119 | 75 |
| G1T4.T120C | 120 | 76 |
| G1T4.K121C | 121 | 77 |
| G1T4.G122C | 122 | 78 |
| G1T4.P123C | 123 | 79 |
| G1T4.S124C | 124 | 80 |
| G1T4.V125C | 125 | 81 |
| G1T4.F126C | 126 | 82 |
| G1T4.P127C | 127 | 83 |
| G1T4.S131C | 131 | 84 |
| G1T4.S132C | 132 | 85 |
| G1T4.K133C | 133 | 86 |
| G1T4.S134C | 134 | 87 |
| G1T4.T135C | 135 | 88 |
| G1T4.S136C | 136 | 89 |
| G1T4.G137C | 137 | 90 |
| G1T4.G138C | 138 | 91 |
| G1T4.T139C | 139 | 92 |
| G1T4.A140C | 140 | 93 |
| G1T4.A141C | 141 | 94 |
| G1T4.D148C | 148 | 95 |
| G1T4.Y149C | 149 | 96 |
| G1T4.F150C | 150 | 97 |
| G1T4.P151C | 151 | 98 |
| G1T4.E152C | 152 | 99 |
| G1T4.P153C | 153 | 100 |
| G1T4.V154C | 154 | 101 |
| G1T4.T155C | 155 | 102 |
| G1T4.V156C | 156 | 103 |
| G1T4.S157C | 157 | 104 |
| G1T4.W158C | 158 | 105 |
| G1T4.N159C | 159 | 106 |
| G1T4.S160C | 160 | 107 |
| G1T4.G161C | 161 | 108 |
| G1T4.A162C | 162 | 109 |
| G1T4.L163C | 163 | 110 |
| G1T4.T164C | 164 | 111 |
| G1T4.S165C | 165 | 112 |
| G1T4.G166C | 166 | 113 |
| G1T4.V167C | 167 | 114 |
| G1T4.V173C | 173 | 115 |
| G1T4.L174C | 174 | 116 |
| G1T4.Q175C | 175 | 117 |
| G1T4.S176C | 176 | 118 |
| G1T4.S177C | 177 | 119 |
| G1T4.G178C | 178 | 120 |
| G1T4.L179C | 179 | 121 |
| G1T4.Y180C | 180 | 122 |
| G1T4.V186C | 186 | 123 |
| G1T4.T187C | 187 | 124 |
| G1T4.V188C | 188 | 125 |
| G1T4.P189C | 189 | 126 |
| G1T4.S190C | 190 | 127 |
| G1T4.S191C | 191 | 128 |
| G1T4.S192C | 192 | 129 |
| G1T4.L193C | 193 | 130 |
| G1T4.G194C | 194 | 131 |
| G1T4.T195C | 195 | 132 |
| G1T4.Q196C | 196 | 133 |
| G1T4.T197C | 197 | 134 |
| G1T4.Y198C | 198 | 135 |
| G1T4.I199C | 199 | 136 |
| G1T4.N201C | 201 | 137 |
| G1T4.V202C | 202 | 138 |
| G1T4.N203C | 203 | 139 |
| G1T4.H204C | 204 | 140 |
| G1T4.K205C | 205 | 141 |
| G1T4.P206C | 206 | 142 |
| G1T4.S207C | 207 | 143 |
| G1T4.N208C | 208 | 144 |
| G1T4.T209C | 209 | 145 |
| G1T4.K210C | 210 | 146 |
| G1T4.V211C | 211 | 147 |
| G1T4.D212C | 212 | 148 |
| G1T4.K213C | 213 | 149 |
| G1T4.R214C | 214 | 150 |
| G1T4.V215C | 215 | 151 |
| G1T4.E216C | 216 | 152 |
| G1T4.P217C | 217 | 153 |
| G1T4.K218C | 218 | 154 |
| G1T4.S219C | 219 | 155 |

TABLE 20
MRA variants

| | SEQ ID NO: | | | |
|---|---|---|---|---|
| | Heavy | Heavy | Light | Light |
| | chain | chain | chain | chain |
| Antibody | variable | constant | variable | constant |
| name | region | region | region | region |
| MRAH.Q5C-G1T4 | 21 | 18 | 19 | 20 |
| MRAH.E6C-G1T4 | 22 | 18 | 19 | 20 |
| MRAH.S7C-G1T4 | 23 | 18 | 19 | 20 |
| MRAH.G8C-G1T4 | 24 | 18 | 19 | 20 |
| MRAH.P9C-G1T4 | 25 | 18 | 19 | 20 |
| MRAH.G10C-G1T4 | 26 | 18 | 19 | 20 |
| MRAH.L11C-G1T4 | 27 | 18 | 19 | 20 |
| MRAH.V12C-G1T4 | 28 | 18 | 19 | 20 |
| MRAH.R13C-G1T4 | 29 | 18 | 19 | 20 |
| MRAH.P14C-G1T4 | 30 | 18 | 19 | 20 |
| MRAH.S15C-G1T4 | 31 | 18 | 19 | 20 |
| MRAH.Q16C-G1T4 | 32 | 18 | 19 | 20 |
| MRAH.T17C-G1T4 | 33 | 18 | 19 | 20 |
| MRAH.L18C-G1T4 | 34 | 18 | 19 | 20 |
| MRAH.S19C-G1T4 | 35 | 18 | 19 | 20 |
| MRAH.L20C-G1T4 | 36 | 18 | 19 | 20 |
| MRAH.T21C-G1T4 | 37 | 18 | 19 | 20 |
| MRAH.T23C-G1T4 | 38 | 18 | 19 | 20 |
| MRAH.S25C-G1T4 | 39 | 18 | 19 | 20 |
| MRAH.G26C-G1T4 | 40 | 18 | 19 | 20 |
| MRAH.S28C-G1T4 | 41 | 18 | 19 | 20 |
| MRAH.T30C-G1T4 | 42 | 18 | 19 | 20 |
| MRAH.R66C-G1T4 | 43 | 18 | 19 | 20 |
| MRAH.V67C-G1T4 | 44 | 18 | 19 | 20 |
| MRAH.T68C-G1T4 | 45 | 18 | 19 | 20 |
| MRAH.L70C-G1T4 | 46 | 18 | 19 | 20 |
| MRAH.D72C-G1T4 | 47 | 18 | 19 | 20 |
| MRAH.T73C-G1T4 | 48 | 18 | 19 | 20 |
| MRAH.S74C-G1T4 | 49 | 18 | 19 | 20 |
| MRAH.K75C-G1T4 | 50 | 18 | 19 | 20 |
| MRAH.N76C-G1T4 | 51 | 18 | 19 | 20 |
| MRAH.Q77C-G1T4 | 52 | 18 | 19 | 20 |
| MRAH.S79C-G1T4 | 53 | 18 | 19 | 20 |
| MRAH.L80C-G1T4 | 54 | 18 | 19 | 20 |
| MRAH.R81C-G1T4 | 55 | 18 | 19 | 20 |
| MRAH.L82C-G1T4 | 56 | 18 | 19 | 20 |
| MRAH.S82aC-G1T4 | 57 | 18 | 19 | 20 |
| MRAH.S82bC-G1T4 | 58 | 18 | 19 | 20 |
| MRAH.V82cC-G1T4 | 59 | 18 | 19 | 20 |
| MRAH.S112C-G1T4 | 60 | 18 | 19 | 20 |
| MRAH.S113C-G1T4 | 61 | 18 | 19 | 20 |
| MRAH.S31C-G1T4 | 62 | 18 | 19 | 20 |
| MRAH.W35C-G1T4 | 63 | 18 | 19 | 20 |
| MRAH.S35aC-G1T4 | 64 | 18 | 19 | 20 |
| MRAH.Y50C-G1T4 | 65 | 18 | 19 | 20 |
| MRAH.I51C-G1T4 | 66 | 18 | 19 | 20 |
| MRAH.S52C-G1T4 | 67 | 18 | 19 | 20 |
| MRAH.S62C-G1T4 | 68 | 18 | 19 | 20 |
| MRAH.L63C-G1T4 | 69 | 18 | 19 | 20 |
| MRAH.K64C-G1T4 | 70 | 18 | 19 | 20 |
| MRAH.S65C-G1T4 | 71 | 18 | 19 | 20 |
| MRAH.D101C-G1T4 | 72 | 18 | 19 | 20 |
| MRAH.Y102C-G1T4 | 73 | 18 | 19 | 20 |
| MRAH-G1T4.A118C | 17 | 74 | 19 | 20 |
| MRAH-G1T4.S119C | 17 | 75 | 19 | 20 |
| MRAH-G1T4.T120C | 17 | 76 | 19 | 20 |
| MRAH-G1T4.K121C | 17 | 77 | 19 | 20 |
| MRAH-G1T4.G122C | 17 | 78 | 19 | 20 |
| MRAH-G1T4.P123C | 17 | 79 | 19 | 20 |
| MRAH-G1T4.S124C | 17 | 80 | 19 | 20 |
| MRAH-G1T4.V125C | 17 | 81 | 19 | 20 |
| MRAH-G1T4.F126C | 17 | 82 | 19 | 20 |
| MRAH-G1T4.P127C | 17 | 83 | 19 | 20 |
| MRAH-G1T4.S131C | 17 | 84 | 19 | 20 |
| MRAH-G1T4.S132C | 17 | 85 | 19 | 20 |
| MRAH-G1T4.K133C | 17 | 86 | 19 | 20 |
| MRAH-G1T4.S134C | 17 | 87 | 19 | 20 |
| MRAH-G1T4.T135C | 17 | 88 | 19 | 20 |
| MRAH-G1T4.S136C | 17 | 89 | 19 | 20 |
| MRAH-G1T4.G137C | 17 | 90 | 19 | 20 |
| MRAH-G1T4.G138C | 17 | 91 | 19 | 20 |
| MRAH-G1T4.T139C | 17 | 92 | 19 | 20 |
| MRAH-G1T4.A140C | 17 | 93 | 19 | 20 |
| MRAH-G1T4.A141C | 17 | 94 | 19 | 20 |
| MRAH-G1T4.D148C | 17 | 95 | 19 | 20 |
| MRAH-G1T4.Y149C | 17 | 96 | 19 | 20 |
| MRAH-G1T4.F150C | 17 | 97 | 19 | 20 |
| MRAH-G1T4.P151C | 17 | 98 | 19 | 20 |
| MRAH-G1T4.E152C | 17 | 99 | 19 | 20 |
| MRAH-G1T4.P153C | 17 | 100 | 19 | 20 |
| MRAH-G1T4.V154C | 17 | 101 | 19 | 20 |
| MRAH-G1T4.T155C | 17 | 102 | 19 | 20 |
| MRAH-G1T4.V156C | 17 | 103 | 19 | 20 |
| MRAH-G1T4.S157C | 17 | 104 | 19 | 20 |
| MRAH-G1T4.W158C | 17 | 105 | 19 | 20 |
| MRAH-G1T4.N159C | 17 | 106 | 19 | 20 |
| MRAH-G1T4.S160C | 17 | 107 | 19 | 20 |
| MRAH-G1T4.G161C | 17 | 108 | 19 | 20 |
| MRAH-G1T4.A162C | 17 | 109 | 19 | 20 |
| MRAH-G1T4.L163C | 17 | 110 | 19 | 20 |
| MRAH-G1T4.T164C | 17 | 111 | 19 | 20 |
| MRAH-G1T4.S165C | 17 | 112 | 19 | 20 |
| MRAH-G1T4.G166C | 17 | 113 | 19 | 20 |
| MRAH-G1T4.V167C | 17 | 114 | 19 | 20 |
| MRAH-G1T4.V173C | 17 | 115 | 19 | 20 |
| MRAH-G1T4.L174C | 17 | 116 | 19 | 20 |
| MRAH-G1T4.Q175C | 17 | 117 | 19 | 20 |
| MRAH-G1T4.S176C | 17 | 118 | 19 | 20 |
| MRAH-G1T4.S177C | 17 | 119 | 19 | 20 |
| MRAH-G1T4.G178C | 17 | 120 | 19 | 20 |
| MRAH-G1T4.L179C | 17 | 121 | 19 | 20 |
| MRAH-G1T4.Y180C | 17 | 122 | 19 | 20 |
| MRAH-G1T4.V186C | 17 | 123 | 19 | 20 |
| MRAH-G1T4.T187C | 17 | 124 | 19 | 20 |
| MRAH-G1T4.V188C | 17 | 125 | 19 | 20 |
| MRAH-G1T4.P189C | 17 | 126 | 19 | 20 |
| MRAH-G1T4.S190C | 17 | 127 | 19 | 20 |
| MRAH-G1T4.S191C | 17 | 128 | 19 | 20 |
| MRAH-G1T4.S192C | 17 | 129 | 19 | 20 |
| MRAH-G1T4.L193C | 17 | 130 | 19 | 20 |
| MRAH-G1T4.G194C | 17 | 131 | 19 | 20 |
| MRAH-G1T4.T195C | 17 | 132 | 19 | 20 |
| MRAH-G1T4.Q196C | 17 | 133 | 19 | 20 |
| MRAH-G1T4.T197C | 17 | 134 | 19 | 20 |
| MRAH-G1T4.Y198C | 17 | 135 | 19 | 20 |
| MRAH-G1T4.I199C | 17 | 136 | 19 | 20 |
| MRAH-G1T4.N201C | 17 | 137 | 19 | 20 |
| MRAH-G1T4.V202C | 17 | 138 | 19 | 20 |
| MRAH-G1T4.N203C | 17 | 139 | 19 | 20 |
| MRAH-G1T4.H204C | 17 | 140 | 19 | 20 |
| MRAH-G1T4.K205C | 17 | 141 | 19 | 20 |
| MRAH-G1T4.P206C | 17 | 142 | 19 | 20 |
| MRAH-G1T4.S207C | 17 | 143 | 19 | 20 |
| MRAH-G1T4.N208C | 17 | 144 | 19 | 20 |
| MRAH-G1T4.T209C | 17 | 145 | 19 | 20 |
| MRAH-G1T4.K210C | 17 | 146 | 19 | 20 |
| MRAH-G1T4.V211C | 17 | 147 | 19 | 20 |
| MRAH-G1T4.D212C | 17 | 148 | 19 | 20 |
| MRAH-G1T4.K213C | 17 | 149 | 19 | 20 |
| MRAH-G1T4.R214C | 17 | 150 | 19 | 20 |
| MRAH-G1T4.V215C | 17 | 151 | 19 | 20 |
| MRAH-G1T4.E216C | 17 | 152 | 19 | 20 |
| MRAH-G1T4.P217C | 17 | 153 | 19 | 20 |
| MRAH-G1T4.K218C | 17 | 154 | 19 | 20 |
| MRAH-G1T4.S219C | 17 | 155 | 19 | 20 |

### Reference Example 5-2 Assessment of Protease-Mediated Fab Fragmentation of Antibodies Having Cysteine Substitution at Various Positions in the Heavy Chain

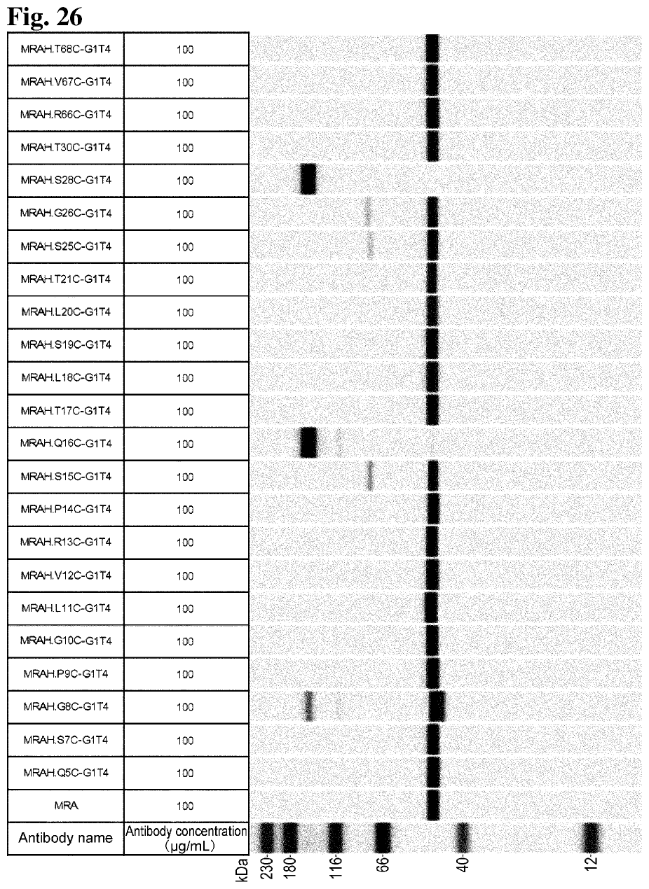

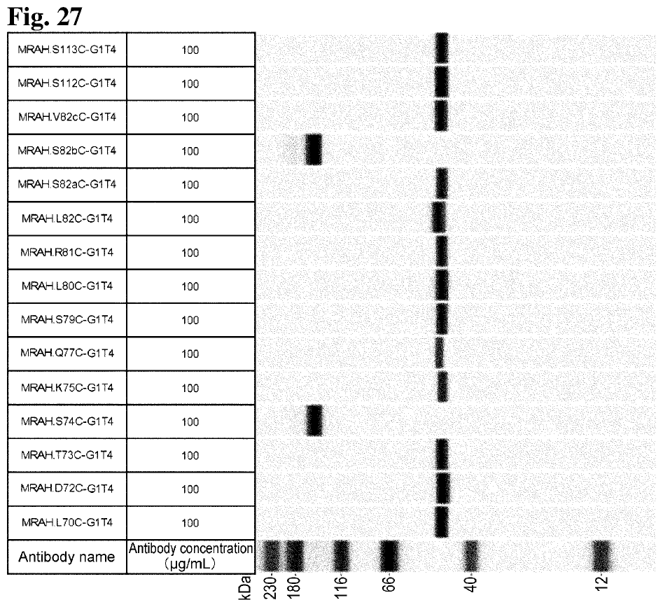

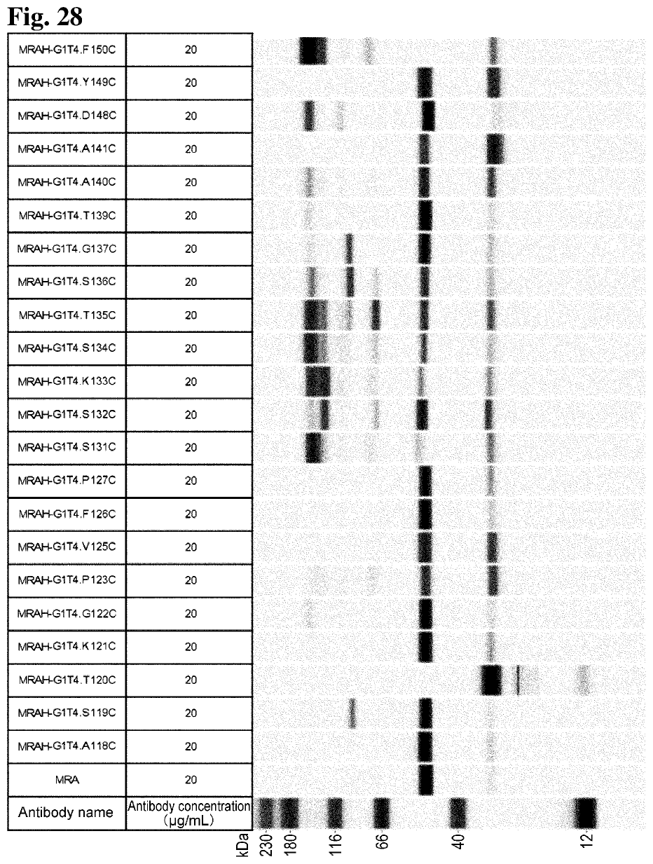

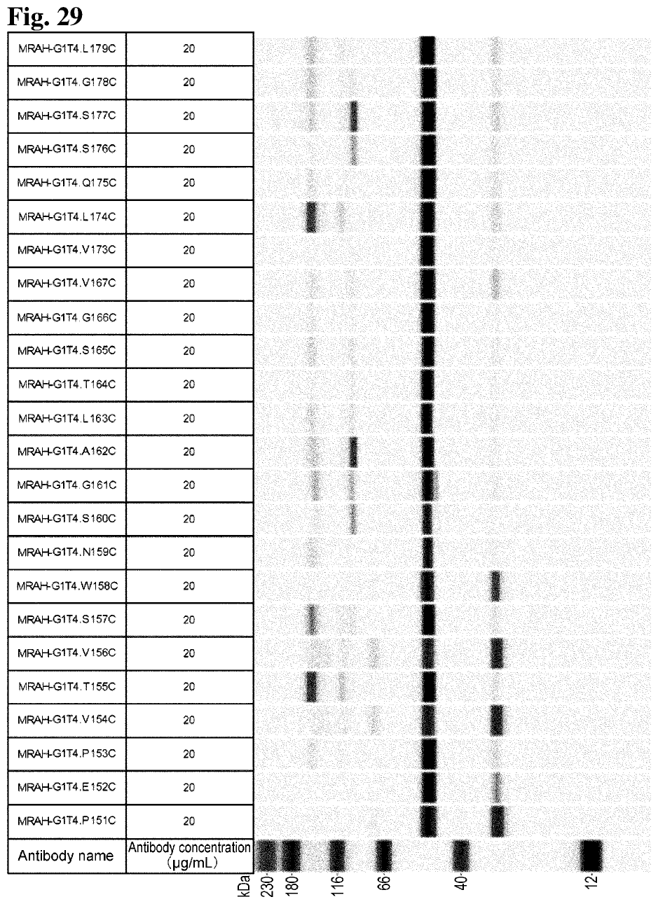

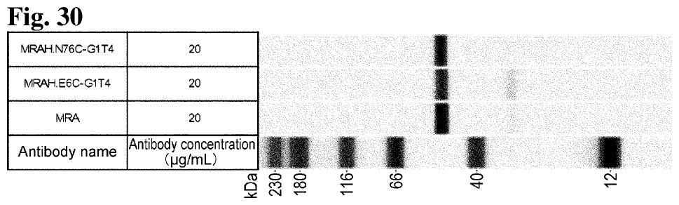

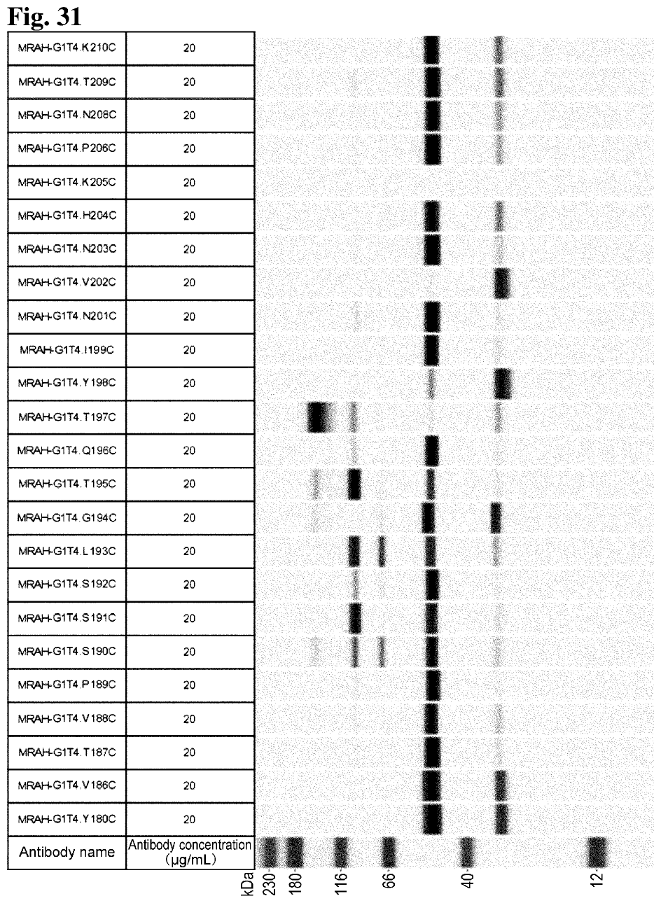

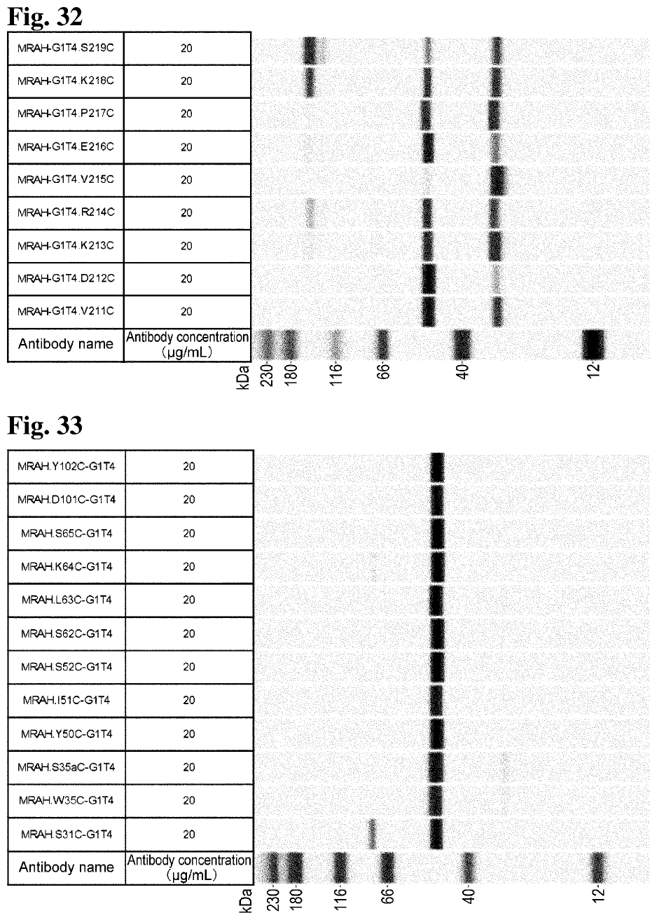

Using a protease that cleaves the heavy chain hinge region of antibody to cause Fab fragmentation, the MRA variants produced in Reference Example 5-1 were examined for whether they acquired protease resistance so that their fragmentation would be inhibited. The protease used was Lys-C (Endoproteinase Lys-C Sequencing Grade) (SIGMA; 11047825001). Reaction was performed under the conditions of 2 ng/microliter protease, 100 microgram/mL antibody, 80% 25 mM Tris-HCl pH 8.0, 20% PBS, and 35 degrees C. for two hours, or under the conditions of 2 ng/microliter protease, 20 microgram/mL antibody, 80% 25 mM Tris-HCl pH 8.0, 20% PBS, and 35 degrees C. for one hour. The sample was then subjected to non-reducing capillary electrophoresis. Wes (Protein Simple) was used for capillary electrophoresis, and an HRP-labeled anti-kappa chain antibody (abcam; ab46527) was used for detection. The results are shown in FIGS. 26 to 33. Lys-C treatment of MRA caused cleavage of the heavy chain hinge region, resulting in disappearance of the band of IgG at around 150 kDa and appearance of the band of Fab at around 50 kDa. For the MRA variants produced in Reference Example 5-1, some showed the band of Fab dimer appearing at around 96 kDa and some showed the band of undigested IgG detected at around 150 kDa after the protease treatment. The area of each band obtained after the protease treatment was outputted using software dedicated for Wes (Compass for SW; Protein Simple) to calculate the percentage of the band areas of undigested IgG, Fab dimer, etc. The calculated percentage of each band is shown in Table 21.

TABLE 21

| | | | | Heavy | Light |
|---|---|---|---|---|---|
| | | | | chain | chain |
| Antibody | IgG | Fab-Fab | Fab | SEQ | SEQ |
| name | (%) | (%) | (%) | ID NO: | ID NO: |
| MRAH.Q5C-G1T4 | 0.2 | 1.5 | 97.6 | 21 | 16 |
| MRAH.E6C-G1T4 | 0 | 0.3 | 80.7 | 22 | 16 |
| MRAH.S7C-G1T4 | 0.4 | 1.9 | 96.9 | 23 | 16 |
| MRAH.G8C-G1T4 | 16.6 | 1.1 | 76.7 | 24 | 16 |
| MRAH.P9C-G1T4 | 0.2 | 1.5 | 97.2 | 25 | 16 |
| MRAH.G10C-G1T4 | 0.6 | 1.9 | 96.9 | 26 | 16 |
| MRAH.L11C-G1T4 | 0 | 1.2 | 98.3 | 27 | 16 |
| MRAH.V12C-G1T4 | 0.2 | 1 | 97.6 | 28 | 16 |
| MRAH.R13C-G1T4 | 0.6 | 1.9 | 96.6 | 29 | 16 |
| MRAH.P14C-G1T4 | 0.3 | 1.7 | 97.7 | 30 | 16 |
| MRAH.S15C-G1T4 | 0.9 | 1.3 | 81.4 | 31 | 16 |
| MRAH.Q16C-G1T4 | 92.5 | 0 | 2 | 32 | 16 |
| MRAH.T17C-G1T4 | 0.4 | 1.4 | 97.8 | 33 | 16 |
| MRAH.L18C-G1T4 | 0.3 | 0.6 | 96.1 | 34 | 16 |
| MRAH.S19C-G1T4 | 0.3 | 1.2 | 98.1 | 35 | 16 |
| MRAH.L20C-G1T4 | 1 | 0.3 | 93.3 | 36 | 16 |
| MRAH.T21C-G1T4 | 0.5 | 1 | 98.3 | 37 | 16 |
| MRAH.T23C-G1T4 | no data | no data | no data | 38 | 16 |
| MRAH.S25C-G1T4 | 0.3 | 2.8 | 87 | 39 | 16 |
| MRAH.G26C-G1T4 | 0.4 | 1.7 | 85.5 | 40 | 16 |
| MRAH.S28C-G1T4 | 98.6 | 0 | 0.2 | 41 | 16 |
| MRAH.T30C-G1T4 | 0.5 | 0.7 | 97.8 | 42 | 16 |
| MRAH.R66C-G1T4 | 0.2 | 1.2 | 97.9 | 43 | 16 |
| MRAH.V67C-G1T4 | 0.3 | 0.4 | 97.8 | 44 | 16 |
| MRAH.T68C-G1T4 | 0.2 | 1.4 | 97.7 | 45 | 16 |
| MRAH.L70C-G1T4 | 0.2 | 0.9 | 98 | 46 | 16 |
| MRAH.D72C-G1T4 | 0.3 | 0.8 | 97.6 | 47 | 16 |
| MRAH.T73C-G1T4 | 0.5 | 0.9 | 97.7 | 48 | 16 |
| MRAH.S74C-G1T4 | 97.1 | 0 | 0.3 | 49 | 16 |
| MRAH.K75C-G1T4 | 0.1 | 1.5 | 97 | 50 | 16 |
| MRAH.N76C-G1T4 | 0.4 | 0.4 | 93.1 | 51 | 16 |
| MRAH.Q77C-G1T4 | 0.1 | 0.2 | 99.6 | 52 | 16 |
| MRAH.S79C-G1T4 | 0.1 | 1.6 | 96.7 | 53 | 16 |
| MRAH.L80C-G1T4 | 0.2 | 0 | 96.5 | 54 | 16 |
| MRAH.R81C-G1T4 | 0 | 1.4 | 98 | 55 | 16 |
| MRAH.L82C-G1T4 | 0 | 0 | 96.8 | 56 | 16 |
| MRAH.S82aC-G1T4 | 0.6 | 1 | 96.7 | 57 | 16 |
| MRAH.S82bC-G1T4 | 97.5 | 0 | 0.3 | 58 | 16 |
| MRAH.V82cC-G1T4 | 0.1 | 0.3 | 95.6 | 59 | 16 |
| MRAH.S112C-G1T4 | 0.1 | 1.1 | 97.6 | 60 | 16 |
| MRAH.S113C-G1T4 | 0.1 | 2.8 | 95.9 | 61 | 16 |
| MRAH.S31C-G1T4 | 0.5 | 2 | 75.7 | 62 | 16 |
| MRAH.W35C-G1T4 | 0.1 | 0.3 | 91.1 | 63 | 16 |
| MRAH.S35aC-G1T4 | 0 | 0.6 | 90.7 | 64 | 16 |
| MRAH.Y50C-G1T4 | 0.2 | 1.5 | 95.8 | 65 | 16 |
| MRAH.I51C-G1T4 | 0.2 | 0.8 | 94.4 | 66 | 16 |
| MRAH.S52C-G1T4 | 0.3 | 1.7 | 96.4 | 67 | 16 |
| MRAH.S62C-G1T4 | 0.2 | 1.1 | 97.6 | 68 | 16 |
| MRAH.L63C-G1T4 | 0.4 | 1.4 | 94.2 | 69 | 16 |
| MRAH.K64C-G1T4 | 0 | 1.6 | 91.7 | 70 | 16 |
| MRAH.S65C-G1T4 | 0.3 | 1.7 | 95.6 | 71 | 16 |
| MRAH.D101C-G1T4 | 0 | 1.2 | 97 | 72 | 16 |
| MRAH.Y102C-G1T4 | 0.2 | 1.3 | 96.8 | 73 | 16 |
| MRAH-G1T4.A118C | 1.2 | 1 | 89 | 74 | 16 |
| MRAH-G1T4.S119C | 2.3 | 14 | 77.7 | 75 | 16 |
| MRAH-G1T4.T120C | 0 | 0.1 | 0.1 | 76 | 16 |
| MRAH-G1T4.K121C | 2.4 | 1.1 | 82.2 | 77 | 16 |
| MRAH-G1T4.G122C | 8 | 1.4 | 79.8 | 78 | 16 |
| MRAH-G1T4.P123C | 7.1 | 0 | 45.7 | 79 | 16 |
| MRAH-G1T4.S124C | 0.8 | 1.7 | 94.5 | 80 | 16 |
| MRAH-G1T4.V125C | 2.3 | 0 | 62 | 81 | 16 |
| MRAH-G1T4.F126C | 2.1 | 1 | 85.5 | 82 | 16 |
| MRAH-G1T4.P127C | 2.9 | 1.4 | 77.4 | 83 | 16 |
| MRAH-G1T4.S131C | 68.4 | 0 | 0 | 84 | 16 |
| MRAH-G1T4.S132C | 13.9 | 0.8 | 54.6 | 85 | 16 |
| MRAH-G1T4.K133C | 66.8 | 0 | 0 | 86 | 16 |
| MRAH-G1T4.S134C | 63.5 | 0 | 21.9 | 87 | 16 |
| MRAH-G1T4.T135C | 44.7 | 13.2 | 23.6 | 88 | 16 |
| MRAH-G1T4.S136C | 22.9 | 27.3 | 35.1 | 89 | 16 |
| MRAH-G1T4.G137C | 8.4 | 18.1 | 62.1 | 90 | 16 |
| MRAH-G1T4.G138C | no data | no data | no data | 91 | 16 |
| MRAH-G1T4.T139C | 7.4 | 1.4 | 82.1 | 92 | 16 |
| MRAH-G1T4.A140C | 20.2 | 0 | 47.2 | 93 | 16 |
| MRAH-G1T4.A141C | 0.3 | 0 | 31.9 | 94 | 16 |
| MRAH-G1T4.D148C | 21 | 0 | 64.8 | 95 | 16 |
| MRAH-G1T4.Y149C | 0.5 | 0 | 58.1 | 96 | 16 |
| MRAH-G1T4.F150C | 79.2 | 0 | 0.4 | 97 | 16 |
| MRAH-G1T4.P151C | 2 | 0 | 56.1 | 98 | 16 |
| MRAH-G1T4.E152C | 0.9 | 0.3 | 84.8 | 99 | 16 |
| MRAH-G1T4.P153C | 4.4 | 0.8 | 86.6 | 100 | 16 |
| MRAH-G1T4.V154C | 4 | 0 | 45.7 | 101 | 16 |
| MRAH-G1T4.T155C | 20.2 | 1.4 | 67.6 | 102 | 16 |
| MRAH-G1T4.V156C | 7 | 0 | 39.2 | 103 | 16 |
| MRAH-G1T4.S157C | 13.5 | 3.2 | 75.9 | 104 | 16 |
| MRAH-G1T4.W158C | 4.2 | 0 | 66.1 | 105 | 16 |
| MRAH-G1T4.N159C | 13.9 | 1.9 | 76.1 | 106 | 16 |
| MRAH-G1T4.S160C | 7.7 | 20.9 | 66.2 | 107 | 16 |
| MRAH-G1T4.G161C | 14.1 | 12 | 68.6 | 108 | 16 |
| MRAH-G1T4.A162C | 9.6 | 17.9 | 65.8 | 109 | 16 |
| MRAH-G1T4.L163C | 10.2 | 6.1 | 75.9 | 110 | 16 |
| MRAH-G1T4.T164C | 3.8 | 3.2 | 88.7 | 111 | 16 |
| MRAH-G1T4.S165C | 7.8 | 4.1 | 81.5 | 112 | 16 |
| MRAH-G1T4.G166C | 4.5 | 2.2 | 89.4 | 113 | 16 |
| MRAH-G1T4.V167C | 5.5 | 2.5 | 81.2 | 114 | 16 |
| MRAH-G1T4.V173C | 2.1 | 1.6 | 92.2 | 115 | 16 |
| MRAH-G1T4.L174C | 19.8 | 0 | 67.1 | 116 | 16 |
| MRAH-G1T4.Q175C | 4.4 | 1.1 | 86.6 | 117 | 16 |
| MRAH-G1T4.S176C | 2.3 | 7.7 | 85.5 | 118 | 16 |
| MRAH-G1T4.S177C | 7.1 | 12.4 | 71.6 | 119 | 16 |
| MRAH-G1T4.G178C | 6.2 | 2.4 | 85.5 | 120 | 16 |
| MRAH-G1T4.L179C | 0.2 | 0 | 0 | 121 | 16 |
| MRAH-G1T4.Y180C | 0 | 0 | 72.7 | 122 | 16 |
| MRAH-G1T4.V186C | 0 | 0 | 73.3 | 123 | 16 |
| MRAH-G1T4.T187C | 0.8 | 2.5 | 90.3 | 124 | 16 |
| MRAH-G1T4.V188C | 0.3 | 4 | 82.7 | 125 | 16 |
| MRAH-G1T4.P189C | 0.9 | 4.7 | 89.6 | 126 | 16 |
| MRAH-G1T4.S190C | 10.9 | 0 | 74.4 | 127 | 16 |
| MRAH-G1T4.S191C | 2.3 | 46.4 | 45.1 | 128 | 16 |
| MRAH-G1T4.S192C | 1.3 | 11 | 83 | 129 | 16 |
| MRAH-G1T4.L193C | 3.6 | 0 | 70.5 | 130 | 16 |
| MRAH-G1T4.G194C | 13.8 | 0 | 0 | 131 | 16 |
| MRAH-G1T4.T195C | 29.6 | 0 | 57.3 | 132 | 16 |
| MRAH-G1T4.Q196C | 1.5 | 0 | 92.6 | 133 | 16 |
| MRAH-G1T4.T197C | 81.5 | 0 | 4.5 | 134 | 16 |
| MRAH-G1T4.Y198C | 0.1 | 0.3 | 17.1 | 135 | 16 |
| MRAH-G1T4.I199C | 1 | 1.7 | 91.6 | 136 | 16 |
| MRAH-G1T4.N201C | 0.7 | 4 | 90.3 | 137 | 16 |
| MRAH-G1T4.V202C | 0 | 0.1 | 6.6 | 138 | 16 |
| MRAH-G1T4.N203C | 0.6 | 2.4 | 89.8 | 139 | 16 |
| MRAH-G1T4.H204C | 0.4 | 2.2 | 77.7 | 140 | 16 |
| MRAH-G1T4.K205C | 0.2 | 2.3 | 85.5 | 141 | 16 |
| MRAH-G1T4.P206C | 0.4 | 2.1 | 86.9 | 142 | 16 |
| MRAH-G1T4.S207C | no data | no data | no data | 143 | 16 |
| MRAH-G1T4.N208C | 0.4 | 0 | 86.2 | 144 | 16 |
| MRAH-G1T4.T209C | 0.7 | 0 | 83.1 | 145 | 16 |
| MRAH-G1T4.K210C | 0.6 | 0 | 81.7 | 146 | 16 |
| MRAH-G1T4.V211C | 0.3 | 1 | 67.6 | 147 | 16 |
| MRAH-G1T4.D212C | 1.1 | 1.8 | 80.9 | 148 | 16 |
| MRAH-G1T4.K213C | 6.5 | 0 | 41.9 | 149 | 16 |
| MRAH-G1T4.R214C | 18.6 | 0 | 42.7 | 150 | 16 |
| MRAH-G1T4.V215C | 0 | 0 | 11.8 | 151 | 16 |
| MRAH-G1T4.E216C | 7.4 | 0 | 64.8 | 152 | 16 |
| MRAH-G1T4.P217C | 4.5 | 0.2 | 43.3 | 153 | 16 |
| MRAH-G1T4.K218C | 30.8 | 0 | 29.5 | 154 | 16 |
| MRAH-G1T4.S219C | 46.9 | 0.1 | 18 | 155 | 16 |

From this result, it was found that cysteine substitution in the heavy chain variable region or heavy chain constant region improved the protease resistance of the heavy chain hinge region in the MRA variants shown in Table 22. Alternatively, the result suggested that a Fab dimer was formed by a covalent bond between the Fab-Fab.

TABLE 22
MRA variants

| | SEQ ID NO: | | | |
|---|---|---|---|---|
| | Heavy | Heavy | Light | Light |
| | chain | chain | chain | chain |
| Antibody | variable | constant | variable | constant |
| name | region | region | region | region |
| MRAH.G8C-G1T4 | 24 | 18 | 19 | 20 |
| MRAH.Q16C-G1T4 | 32 | 18 | 19 | 20 |
| MRAH.S28C-G1T4 | 41 | 18 | 19 | 20 |
| MRAH.S74C-G1T4 | 49 | 18 | 19 | 20 |
| MRAH.S82bC-G1T4 | 58 | 18 | 19 | 20 |
| MRAH-G1T4.S119C | 17 | 75 | 19 | 20 |
| MRAH-G1T4.G122C | 17 | 78 | 19 | 20 |
| MRAH-G1T4.P123C | 17 | 79 | 19 | 20 |
| MRAH-G1T4.S131C | 17 | 84 | 19 | 20 |
| MRAH-G1T4.S132C | 17 | 85 | 19 | 20 |
| MRAH-G1T4.K133C | 17 | 86 | 19 | 20 |
| MRAH-G1T4.S134C | 17 | 87 | 19 | 20 |
| MRAH-G1T4.T135C | 17 | 88 | 19 | 20 |
| MRAH-G1T4.S136C | 17 | 89 | 19 | 20 |
| MRAH-G1T4.G137C | 17 | 90 | 19 | 20 |
| MRAH-G1T4.T139C | 17 | 92 | 19 | 20 |
| MRAH-G1T4.A140C | 17 | 93 | 19 | 20 |
| MRAH-G1T4.D148C | 17 | 95 | 19 | 20 |
| MRAH-G1T4.F150C | 17 | 97 | 19 | 20 |
| MRAH-G1T4.T155C | 17 | 102 | 19 | 20 |
| MRAH-G1T4.V156C | 17 | 103 | 19 | 20 |
| MRAH-G1T4.S157C | 17 | 104 | 19 | 20 |
| MRAH-G1T4.N159C | 17 | 106 | 19 | 20 |
| MRAH-G1T4.S160C | 17 | 107 | 19 | 20 |
| MRAH-G1T4.G161C | 17 | 108 | 19 | 20 |
| MRAH-G1T4.A162C | 17 | 109 | 19 | 20 |
| MRAH-G1T4.L163C | 17 | 110 | 19 | 20 |
| MRAH-G1T4.S165C | 17 | 112 | 19 | 20 |
| MRAH-G1T4.V167C | 17 | 114 | 19 | 20 |
| MRAH-G1T4.L174C | 17 | 116 | 19 | 20 |
| MRAH-G1T4.S176C | 17 | 118 | 19 | 20 |
| MRAH-G1T4.S177C | 17 | 119 | 19 | 20 |
| MRAH-G1T4.G178C | 17 | 120 | 19 | 20 |
| MRAH-G1T4.S190C | 17 | 127 | 19 | 20 |
| MRAH-G1T4.S191C | 17 | 128 | 19 | 20 |
| MRAH-G1T4.S192C | 17 | 129 | 19 | 20 |
| MRAH-G1T4.G194C | 17 | 131 | 19 | 20 |
| MRAH-G1T4.T195C | 17 | 132 | 19 | 20 |
| MRAH-G1T4.T197C | 17 | 134 | 19 | 20 |
| MRAH-G1T4.K213C | 17 | 149 | 19 | 20 |
| MRAH-G1T4.R214C | 17 | 150 | 19 | 20 |
| MRAH-G1T4.E216C | 17 | 152 | 19 | 20 |
| MRAH-G1T4.K218C | 17 | 154 | 19 | 20 |
| MRAH-G1T4.S219C | 17 | 155 | 19 | 20 |

### Reference Example 6 Assessment of Antibodies Having Cysteine Substitution at Various Positions in the Light Chain

### Reference Example 6-1 Assessment of Antibodies Having Cysteine Substitution at Various Positions in the Light Chain

The light chain variable region and constant region of an anti-human IL6R neutralizing antibody, MRA (heavy chain: MRAH-G1T4 (SEQ ID NO: 15), light chain: MRAL-k0 (SEQ ID NO: 16)) were subjected to a study in which an arbitrary amino acid residue structurally exposed to the surface was substituted with cysteine. Amino acid residues within the light chain variable region of MRA (MRAL, SEQ ID NO: 19) were substituted with cysteine to produce variants of the light chain variable region of MRA shown in Table 23. These variants of the light chain variable region of MRA were each linked with the light chain constant region of MRA (k0, SEQ ID NO: 20) to produce variants of the light chain of MRA, and expression vectors encoding the corresponding genes were produced by a method known to the person skilled in the art.

In addition, amino acid residues within the light chain constant region of MRA (k0, SEQ ID NO: 20) were substituted with cysteine to produce variants of the light chain constant region of MRA shown in Table 24. These variants of the light chain constant region of MRA were each linked with the light chain variable region of MRA (MRAL, SEQ ID NO: 19) to produce variants of the light chain of MRA, and expression vectors encoding the corresponding genes were produced by a method known to the person skilled in the art.

The MRA light chain variants produced above were combined with the MRA heavy chain. The resultant MRA variants shown in Table 25 were expressed by transient expression using FreeStyle293 cells (Invitrogen) or Expi293 cells (Life technologies) by a method known to the person skilled in the art, and purified with Protein A by a method known to the person skilled in the art.

TABLE 23
Variants of MRA light chain variable region
and position of cysteine substitution

| Variant of MRA | Position of cysteine | |
|---|---|---|
| light chain | substitution | SEQ |
| variable region | (Kabat numbering) | ID NO: |
| MRAL.T5C | 5 | 156 |
| MRAL.Q6C | 6 | 157 |
| MRAL.S7C | 7 | 158 |
| MRAL.P8C | 8 | 159 |
| MRAL.S9C | 9 | 160 |
| MRAL.S10C | 10 | 161 |
| MRAL.L11C | 11 | 162 |
| MRAL.S12C | 12 | 163 |
| MRAL.A13C | 13 | 164 |
| MRAL.S14C | 14 | 165 |
| MRAL.V15C | 15 | 166 |
| MRAL.G16C | 16 | 167 |
| MRAL.D17C | 17 | 168 |
| MRAL.R18C | 18 | 169 |
| MRAL.V19C | 19 | 170 |
| MRAL.T20C | 20 | 171 |
| MRAL.I21C | 21 | 172 |
| MRAL.T22C | 22 | 173 |
| MRAL.G57C | 57 | 174 |
| MRAL.V58C | 58 | 175 |
| MRAL.P59C | 59 | 176 |
| MRAL.S60C | 60 | 177 |
| MRAL.R61C | 61 | 178 |
| MRAL.F62C | 62 | 179 |
| MRAL.S63C | 63 | 180 |
| MRAL.S65C | 65 | 181 |
| MRAL.S67C | 67 | 182 |
| MRAL.G68C | 68 | 183 |
| MRAL.T69C | 69 | 184 |
| MRAL.D70C | 70 | 185 |
| MRAL.T72C | 72 | 186 |
| MRAL.F73C | 73 | 187 |
| MRAL.T74C | 74 | 188 |
| MRAL.I75C | 75 | 189 |
| MRAL.S76C | 76 | 190 |
| MRAL.S77C | 77 | 191 |
| MRAL.L78C | 78 | 192 |
| MRAL.Q79C | 79 | 193 |
| MRAL.F98C | 98 | 194 |
| MRAL.G99C | 99 | 195 |
| MRAL.Q100C | 100 | 196 |
| MRAL.G101C | 101 | 197 |
| MRAL.T102C | 102 | 198 |
| MRAL.K103C | 103 | 199 |
| MRAL.V104C | 104 | 200 |
| MRAL.E105C | 105 | 201 |
| MRAL.I106C | 106 | 202 |
| MRAL.K107C | 107 | 203 |
| MRAL.A25C | 25 | 204 |
| MRAL.S26C | 26 | 205 |
| MRAL.Q27C | 27 | 206 |
| MRAL.Y32C | 32 | 207 |
| MRAL.L33C | 33 | 208 |
| MRAL.N34C | 34 | 209 |
| MRAL.Y50C | 50 | 210 |
| MRAL.T51C | 51 | 211 |
| MRAL.H55C | 55 | 212 |
| MRAL.S56C | 56 | 213 |
| MRAL.Y96C | 96 | 214 |
| MRAL.T97C | 97 | 215 |

TABLE 24
Variants of MRA light chain constant region
and position of cysteine substitution

| Variant of MRA | Position of cysteine | |
|---|---|---|
| light chain constant | substitution | SEQ ID |
| region | (EU numbering) | NO: |
| k0.R108C | 108 | 216 |
| k0.T109C | 109 | 217 |
| k0.V110C | 110 | 218 |
| k0.A111C | 111 | 219 |
| k0.A112C | 112 | 220 |
| k0.P113C | 113 | 221 |
| k0.S114C | 114 | 222 |
| k0.V115C | 115 | 223 |
| k0.F116C | 116 | 224 |
| k0.P120C | 120 | 225 |
| k0.S121C | 121 | 226 |
| k0.D122C | 122 | 227 |
| k0.E123C | 123 | 228 |
| k0.Q124C | 124 | 229 |
| k0.L125C | 125 | 230 |
| k0.K126C | 126 | 231 |
| k0.S127C | 127 | 232 |
| k0.G128C | 128 | 233 |
| k0.T129C | 129 | 234 |
| k0.A130C | 130 | 235 |
| k0.S131C | 131 | 236 |
| k0.L136C | 136 | 237 |
| k0.N137C | 137 | 238 |
| k0.N138C | 138 | 239 |
| k0.F139C | 139 | 240 |
| k0.Y140C | 140 | 241 |
| k0.P141C | 141 | 242 |
| k0.R142C | 142 | 243 |
| k0.E143C | 143 | 244 |
| k0.A144C | 144 | 245 |
| k0.K145C | 145 | 246 |
| k0.V146C | 146 | 247 |
| k0.Q147C | 147 | 248 |
| k0.W148C | 148 | 249 |
| k0.K149C | 149 | 250 |
| k0.V150C | 150 | 251 |
| k0.D151C | 151 | 252 |
| k0.N152C | 152 | 253 |
| k0.A153C | 153 | 254 |
| k0.L154C | 154 | 255 |
| k0.Q155C | 155 | 256 |
| k0.S156C | 156 | 257 |
| k0.G157C | 157 | 258 |
| k0.N158C | 158 | 259 |
| k0.S159C | 159 | 260 |
| k0.Q160C | 160 | 261 |
| k0.E161C | 161 | 262 |
| k0.S162C | 162 | 263 |
| k0.V163C | 163 | 264 |
| k0.T164C | 164 | 265 |
| k0.E165C | 165 | 266 |
| k0.Q166C | 166 | 267 |
| k0.D167C | 167 | 268 |
| k0.S168C | 168 | 269 |
| k0.K169C | 169 | 270 |
| k0.D170C | 170 | 271 |
| k0.S171C | 171 | 272 |
| k0.T172C | 172 | 273 |
| k0.Y173C | 173 | 274 |
| k0.S174C | 174 | 275 |
| k0.L175C | 175 | 276 |
| k0.T180C | 180 | 277 |
| k0.L181C | 181 | 278 |
| k0.S182C | 182 | 279 |
| k0.K183C | 183 | 280 |
| k0.A184C | 184 | 281 |
| k0.D185C | 185 | 282 |
| k0.Y186C | 186 | 283 |
| k0.E187C | 187 | 284 |
| k0.K188C | 188 | 285 |
| k0.H189C | 189 | 286 |
| k0.K190C | 190 | 287 |
| k0.V191C | 191 | 288 |
| k0.Y192C | 192 | 289 |
| k0.A193C | 193 | 290 |
| k0.E195C | 195 | 291 |
| k0.V196C | 196 | 292 |
| k0.T197C | 197 | 293 |
| k0.H198C | 198 | 294 |
| k0.Q199C | 199 | 295 |
| k0.G200C | 200 | 296 |
| k0.L201C | 201 | 297 |
| k0.S202C | 202 | 298 |
| k0.S203C | 203 | 299 |
| k0.P204C | 204 | 300 |
| k0.V205C | 205 | 301 |
| k0.T206C | 206 | 302 |
| k0.K207C | 207 | 303 |
| k0.S208C | 208 | 304 |
| k0.F209C | 209 | 305 |
| k0.N210C | 210 | 306 |
| k0.R211C | 211 | 307 |
| k0.G212C | 212 | 308 |
| k0.E213C | 213 | 309 |

TABLE 25
MRA variants

SEQ ID NO:

| | Heavy | Heavy | Light | Light |
|---|---|---|---|---|
| | chain | chain | chain | chain |
| | variable | constant | variable | constant |
| Antibody name | region | region | region | region |
| MRAL.T5C-k0 | 17 | 18 | 156 | 20 |
| MRAL.Q6C-k0 | 17 | 18 | 157 | 20 |
| MRAL.S7C-k0 | 17 | 18 | 158 | 20 |
| MRAL.P8C-k0 | 17 | 18 | 159 | 20 |
| MRAL.S9C-k0 | 17 | 18 | 160 | 20 |
| MRAL.S10C-k0 | 17 | 18 | 161 | 20 |
| MRAL.L11C-k0 | 17 | 18 | 162 | 20 |
| MRAL.S12C-k0 | 17 | 18 | 163 | 20 |
| MRAL.A13C-k0 | 17 | 18 | 164 | 20 |
| MRAL.S14C-k0 | 17 | 18 | 165 | 20 |
| MRAL.V15C-k0 | 17 | 18 | 166 | 20 |
| MRAL.G16C-k0 | 17 | 18 | 167 | 20 |
| MRAL.D17C-k0 | 17 | 18 | 168 | 20 |
| MRAL.R18C-k0 | 17 | 18 | 169 | 20 |
| MRAL.V19C-k0 | 17 | 18 | 170 | 20 |
| MRAL.T20C-k0 | 17 | 18 | 171 | 20 |
| MRAL.I21C-k0 | 17 | 18 | 172 | 20 |
| MRAL.T22C-k0 | 17 | 18 | 173 | 20 |
| MRAL.G57C-k0 | 17 | 18 | 174 | 20 |
| MRAL.V58C-k0 | 17 | 18 | 175 | 20 |
| MRAL.P59C-k0 | 17 | 18 | 176 | 20 |
| MRAL.S60C-k0 | 17 | 18 | 177 | 20 |
| MRAL.R61C-k0 | 17 | 18 | 178 | 20 |
| MRAL.F62C-k0 | 17 | 18 | 179 | 20 |
| MRAL.S63C-k0 | 17 | 18 | 180 | 20 |
| MRAL.S65C-k0 | 17 | 18 | 181 | 20 |
| MRAL.S67C-k0 | 17 | 18 | 182 | 20 |
| MRAL.G68C-k0 | 17 | 18 | 183 | 20 |
| MRAL.T69C-k0 | 17 | 18 | 184 | 20 |
| MRAL.D70C-k0 | 17 | 18 | 185 | 20 |
| MRAL.T72C-k0 | 17 | 18 | 186 | 20 |
| MRAL.F73C-k0 | 17 | 18 | 187 | 20 |
| MRAL.T74C-k0 | 17 | 18 | 188 | 20 |
| MRAL.I75C-k0 | 17 | 18 | 189 | 20 |
| MRAL.S76C-k0 | 17 | 18 | 190 | 20 |
| MRAL.S77C-k0 | 17 | 18 | 191 | 20 |
| MRAL.L78C-k0 | 17 | 18 | 192 | 20 |
| MRAL.Q79C-k0 | 17 | 18 | 193 | 20 |
| MRAL.F98C-k0 | 17 | 18 | 194 | 20 |
| MRAL.G99C-k0 | 17 | 18 | 195 | 20 |
| MRAL.Q100C-k0 | 17 | 18 | 196 | 20 |
| MRAL.G101C-k0 | 17 | 18 | 197 | 20 |
| MRAL.T102C-k0 | 17 | 18 | 198 | 20 |
| MRAL.K103C-k0 | 17 | 18 | 199 | 20 |
| MRAL.V104C-k0 | 17 | 18 | 200 | 20 |
| MRAL.E105C-k0 | 17 | 18 | 201 | 20 |
| MRAL.I106C-k0 | 17 | 18 | 202 | 20 |
| MRAL.K107C-k0 | 17 | 18 | 203 | 20 |
| MRAL.A25C-k0 | 17 | 18 | 204 | 20 |
| MRAL.S26C-k0 | 17 | 18 | 205 | 20 |
| MRAL.Q27C-k0 | 17 | 18 | 206 | 20 |
| MRAL.Y32C-k0 | 17 | 18 | 207 | 20 |
| MRAL.L33C-k0 | 17 | 18 | 208 | 20 |
| MRAL.N34C-k0 | 17 | 18 | 209 | 20 |
| MRAL.Y50C-k0 | 17 | 18 | 210 | 20 |
| MRAL.T51C-k0 | 17 | 18 | 211 | 20 |
| MRAL.H55C-k0 | 17 | 18 | 212 | 20 |
| MRAL.S56C-k0 | 17 | 18 | 213 | 20 |
| MRAL.Y96C-k0 | 17 | 18 | 214 | 20 |
| MRAL.T97C-k0 | 17 | 18 | 215 | 20 |
| MRAL-k0.R108C | 17 | 18 | 19 | 216 |
| MRAL-k0.T109C | 17 | 18 | 19 | 217 |
| MRAL-k0.V110C | 17 | 18 | 19 | 218 |
| MRAL-k0.A111C | 17 | 18 | 19 | 219 |
| MRAL-k0.A112C | 17 | 18 | 19 | 220 |
| MRAL-k0.P113C | 17 | 18 | 19 | 221 |
| MRAL-k0.S114C | 17 | 18 | 19 | 222 |
| MRAL-k0.V115C | 17 | 18 | 19 | 223 |
| MRAL-k0.F116C | 17 | 18 | 19 | 224 |
| MRAL-k0.P120C | 17 | 18 | 19 | 225 |
| MRAL-k0.S121C | 17 | 18 | 19 | 226 |
| MRAL-k0.D122C | 17 | 18 | 19 | 227 |
| MRAL-k0.E123C | 17 | 18 | 19 | 228 |
| MRAL-k0.Q124C | 17 | 18 | 19 | 229 |
| MRAL-k0.L125C | 17 | 18 | 19 | 230 |
| MRAL-k0.K126C | 17 | 18 | 19 | 231 |
| MRAL-k0.S127C | 17 | 18 | 19 | 232 |
| MRAL-k0.G128C | 17 | 18 | 19 | 233 |
| MRAL-k0.T129C | 17 | 18 | 19 | 234 |
| MRAL-k0.A130C | 17 | 18 | 19 | 235 |
| MRAL-k0.S131C | 17 | 18 | 19 | 236 |
| MRAL-k0.L136C | 17 | 18 | 19 | 237 |
| MRAL-k0.N137C | 17 | 18 | 19 | 238 |
| MRAL-k0.N138C | 17 | 18 | 19 | 239 |
| MRAL-k0.F139C | 17 | 18 | 19 | 240 |
| MRAL-k0.Y140C | 17 | 18 | 19 | 241 |
| MRAL-k0.P141C | 17 | 18 | 19 | 242 |
| MRAL-k0.R142C | 17 | 18 | 19 | 243 |
| MRAL-k0.E143C | 17 | 18 | 19 | 244 |
| MRAL-k0.A144C | 17 | 18 | 19 | 245 |
| MRAL-k0.K145C | 17 | 18 | 19 | 246 |
| MRAL-k0.V146C | 17 | 18 | 19 | 247 |
| MRAL-k0.Q147C | 17 | 18 | 19 | 248 |
| MRAL-k0.W148C | 17 | 18 | 19 | 249 |
| MRAL-k0.K149C | 17 | 18 | 19 | 250 |
| MRAL-k0.V150C | 17 | 18 | 19 | 251 |
| MRAL-k0.D151C | 17 | 18 | 19 | 252 |
| MRAL-k0.N152C | 17 | 18 | 19 | 253 |
| MRAL-k0.A153C | 17 | 18 | 19 | 254 |
| MRAL-k0.L154C | 17 | 18 | 19 | 255 |
| MRAL-k0.Q155C | 17 | 18 | 19 | 256 |
| MRAL-k0.S156C | 17 | 18 | 19 | 257 |
| MRAL-k0.G157C | 17 | 18 | 19 | 258 |
| MRAL-k0.N158C | 17 | 18 | 19 | 259 |
| MRAL-k0.S159C | 17 | 18 | 19 | 260 |
| MRAL-k0.Q160C | 17 | 18 | 19 | 261 |
| MRAL-k0.E161C | 17 | 18 | 19 | 262 |
| MRAL-k0.S162C | 17 | 18 | 19 | 263 |
| MRAL-k0.V163C | 17 | 18 | 19 | 264 |
| MRAL-k0.T164C | 17 | 18 | 19 | 265 |
| MRAL-k0.E165C | 17 | 18 | 19 | 266 |
| MRAL-k0.Q166C | 17 | 18 | 19 | 267 |
| MRAL-k0.D167C | 17 | 18 | 19 | 268 |
| MRAL-k0.S168C | 17 | 18 | 19 | 269 |
| MRAL-k0.K169C | 17 | 18 | 19 | 270 |
| MRAL-k0.D170C | 17 | 18 | 19 | 271 |
| MRAL-k0.S171C | 17 | 18 | 19 | 272 |
| MRAL-k0.T172C | 17 | 18 | 19 | 273 |
| MRAL-k0.Y173C | 17 | 18 | 19 | 274 |
| MRAL-k0.S174C | 17 | 18 | 19 | 275 |
| MRAL-k0.L175C | 17 | 18 | 19 | 276 |
| MRAL-k0.T180C | 17 | 18 | 19 | 277 |
| MRAL-k0.L181C | 17 | 18 | 19 | 278 |
| MRAL-k0.S182C | 17 | 18 | 19 | 279 |
| MRAL-k0.K183C | 17 | 18 | 19 | 280 |
| MRAL-k0.A184C | 17 | 18 | 19 | 281 |
| MRAL-k0.D185C | 17 | 18 | 19 | 282 |
| MRAL-k0.Y186C | 17 | 18 | 19 | 283 |
| MRAL-k0.E187C | 17 | 18 | 19 | 284 |
| MRAL-k0.K188C | 17 | 18 | 19 | 285 |
| MRAL-k0.H189C | 17 | 18 | 19 | 286 |
| MRAL-k0.K190C | 17 | 18 | 19 | 287 |
| MRAL-k0.V191C | 17 | 18 | 19 | 288 |
| MRAL-k0.Y192C | 17 | 18 | 19 | 289 |
| MRAL-k0.A193C | 17 | 18 | 19 | 290 |
| MRAL-k0.E195C | 17 | 18 | 19 | 291 |
| MRAL-k0.V196C | 17 | 18 | 19 | 292 |
| MRAL-k0.T197C | 17 | 18 | 19 | 293 |
| MRAL-k0.H198C | 17 | 18 | 19 | 294 |
| MRAL-k0.Q199C | 17 | 18 | 19 | 295 |
| MRAL-k0.G200C | 17 | 18 | 19 | 296 |
| MRAL-k0.L201C | 17 | 18 | 19 | 297 |
| MRAL-k0.S202C | 17 | 18 | 19 | 298 |
| MRAL-k0.S203C | 17 | 18 | 19 | 299 |
| MRAL-k0.P204C | 17 | 18 | 19 | 300 |
| MRAL-k0.V205C | 17 | 18 | 19 | 301 |
| MRAL-k0.T206C | 17 | 18 | 19 | 302 |
| MRAL-k0.K207C | 17 | 18 | 19 | 303 |
| MRAL-k0.S208C | 17 | 18 | 19 | 304 |
| MRAL-k0.F209C | 17 | 18 | 19 | 305 |
| MRAL-k0.N210C | 17 | 18 | 19 | 306 |
| MRAL-k0.R211C | 17 | 18 | 19 | 307 |
| MRAL-k0.G212C | 17 | 18 | 19 | 308 |
| MRAL-k0.E213C | 17 | 18 | 19 | 309 |

### Reference Example 6-2 Assessment of Protease-Mediated Fab Fragmentation of Antibodies Having Cysteine Substitution at Various Positions in the Light Chain

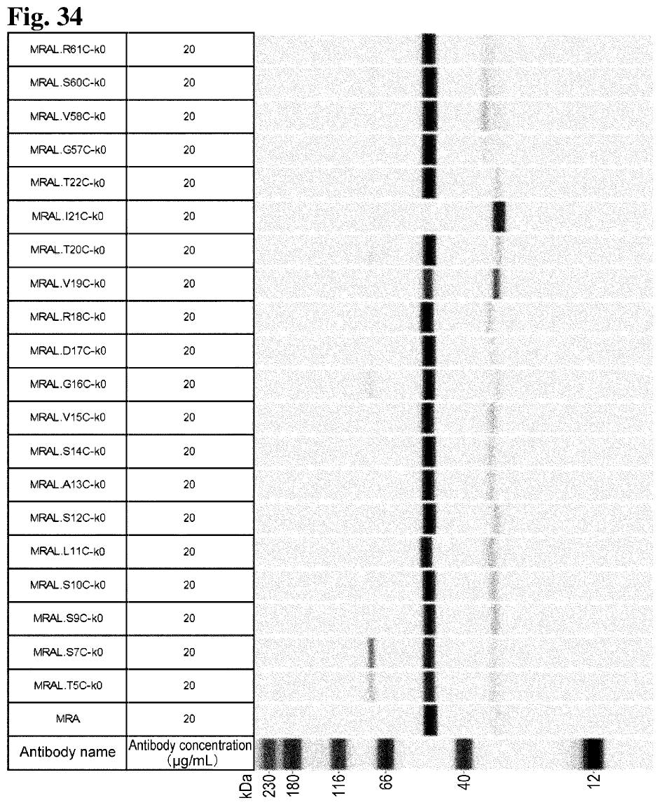

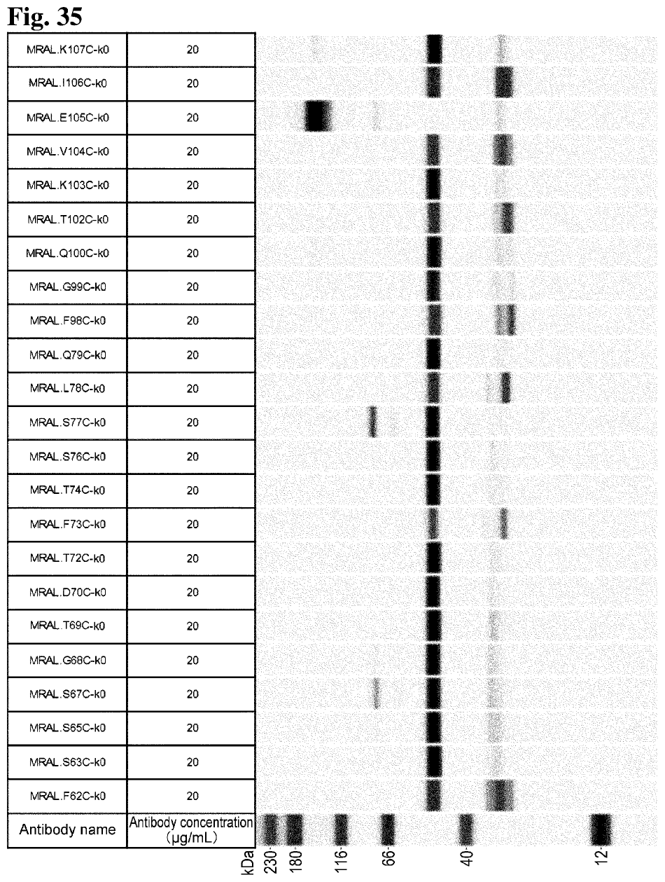

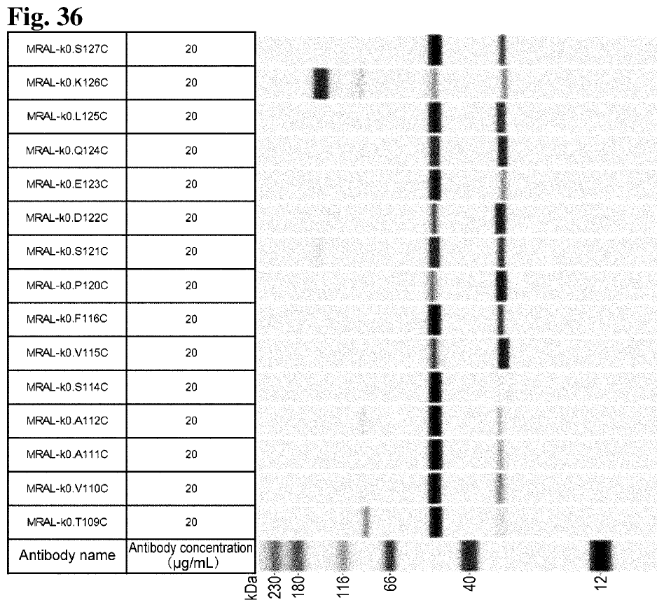

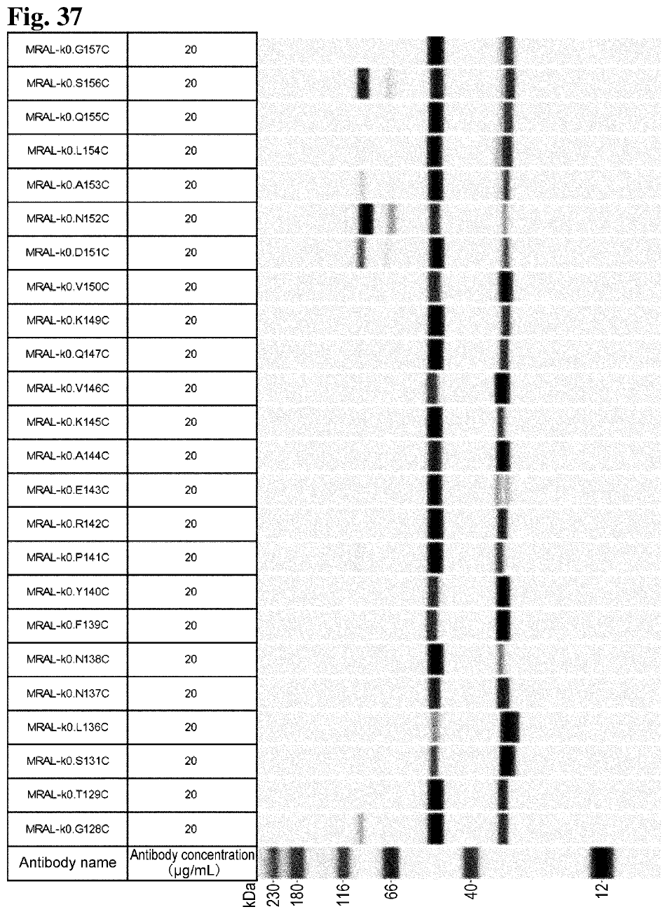

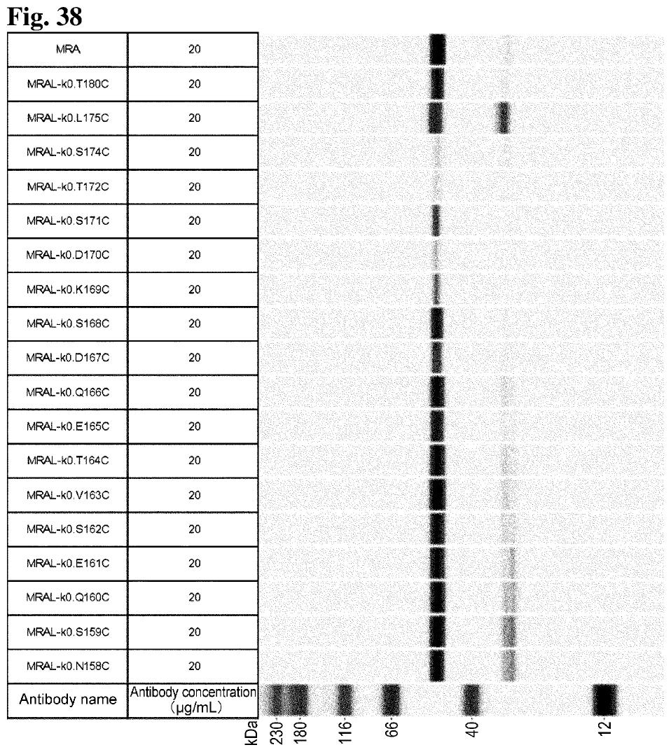

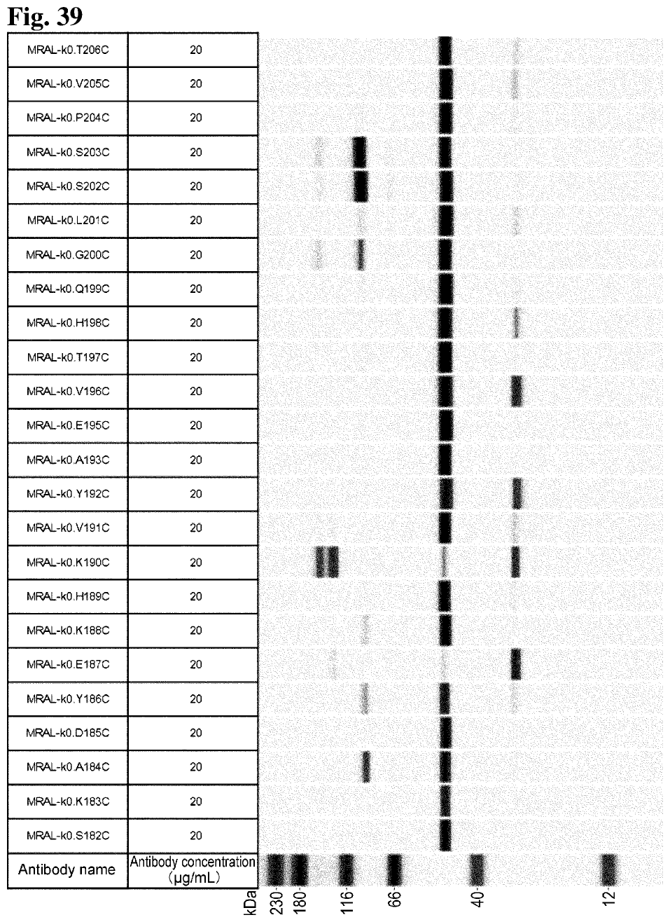

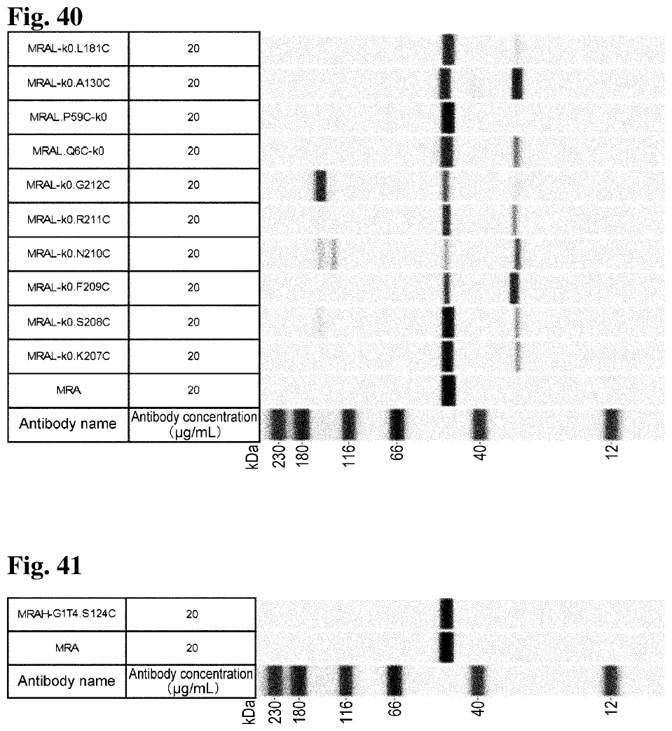

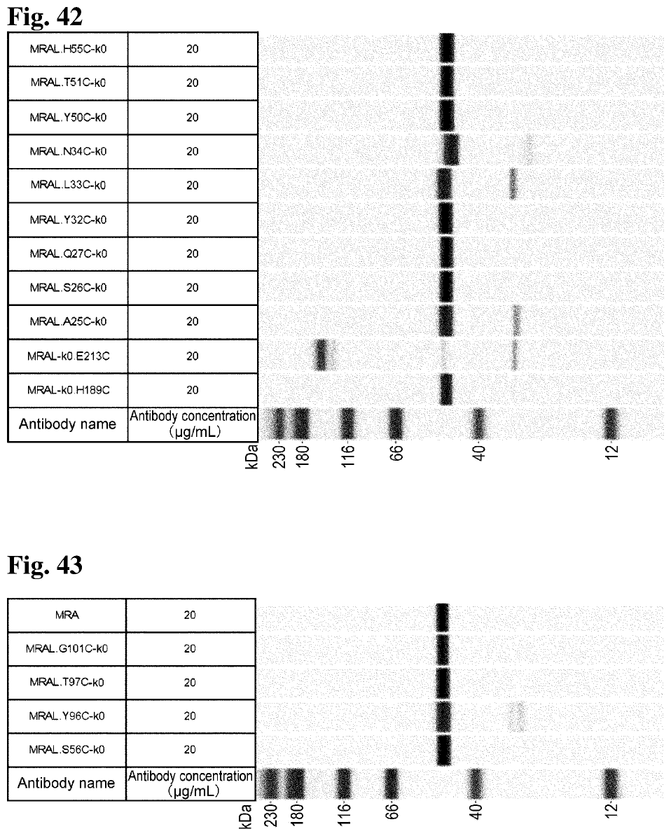

Using a protease that cleaves the heavy chain hinge region of antibody to cause Fab fragmentation, the MRA variants produced in Reference Example 6-1 were examined for whether they acquired protease resistance so that their fragmentation would be inhibited. The protease used was Lys-C (Endoproteinase Lys-C Sequencing Grade) (SIGMA; 11047825001). Reaction was performed under the conditions of 2 ng/microliter protease, 100 microgram/mL antibody, 80% 25 mM Tris-HCl pH 8.0, 20% PBS, and 35 degrees C. for two hours, or under the conditions of 2 ng/microliter protease, 20 microgram/mL antibody, 80% 25 mM Tris-HCl pH 8.0, 20% PBS, and 35 degrees C. for one hour. The sample was then subjected to non-reducing capillary electrophoresis. Wes (Protein Simple) was used for capillary electrophoresis, and an HRP-labeled anti-kappa chain antibody (abcam; ab46527) was used for detection. The results are shown in FIGS. 24 to 43. Lys-C treatment of MRA caused cleavage of the heavy chain hinge region, resulting in disappearance of the band of IgG at around 150 kDa and appearance of the band of Fab at around 50 kDa. For the MRA variants produced in Reference Example 6-1, some showed the band of Fab dimer appearing at around 96 kDa and some showed the band of undigested IgG detected at around 150 kDa after the protease treatment. The area of each band obtained after the protease treatment was outputted using software dedicated for Wes (Compass for SW; Protein Simple) to calculate the percentage of the band areas of undigested IgG, Fab dimer, etc. The calculated percentage of each band is shown in Table 26.

TABLE 26

| | | | | Heavy | Light |
|---|---|---|---|---|---|
| | | | | chain | chain |
| | IgG | Fab-Fab | Fab | SEQ ID | SEQ ID |
| Antibody name | (%) | (%) | (%) | NO: | NO: |
| MRAL.T5C-k0 | 0.1 | 0 | 71.1 | 15 | 156 |
| MRAL.Q6C-k0 | 0.1 | 0 | 74.5 | 15 | 157 |
| MRAL.S7C-k0 | 0.2 | 0 | 68.8 | 15 | 158 |
| MRAL.P8C-k0 | no data | no data | no data | 15 | 159 |
| MRAL.S9C-k0 | 0.3 | 0.4 | 82.9 | 15 | 160 |
| MRAL.S10C-k0 | 0.2 | 0.4 | 85.8 | 15 | 161 |
| MRAL.L11C-k0 | 0 | 0 | 83.4 | 15 | 162 |
| MRAL.S12C-k0 | 0.9 | 0.4 | 87.2 | 15 | 163 |
| MRAL.A13C-k0 | 0.1 | 0 | 88.6 | 15 | 164 |
| MRAL.S14C-k0 | 0.3 | 0.6 | 85.9 | 15 | 165 |
| MRAL.V15C-k0 | 0.2 | 0 | 84.8 | 15 | 166 |
| MRAL.G16C-k0 | 0.8 | 0 | 82.3 | 15 | 167 |
| MRAL.D17C-k0 | 0 | 0 | 92.3 | 15 | 168 |
| MRAL.R18C-k0 | 0.2 | 0.4 | 87.1 | 15 | 169 |
| MRAL.V19C-k0 | 0 | 0 | 63.3 | 15 | 170 |
| MRAL.T20C-k0 | 0.5 | 0.6 | 83.6 | 15 | 171 |
| MRAL.I21C-k0 | 0 | 0 | 5 | 15 | 172 |
| MRAL.T22C-k0 | 0 | 0.3 | 89.5 | 15 | 173 |
| MRAL.G57C-k0 | 0.2 | 0 | 91.7 | 15 | 174 |
| MRAL.V58C-k0 | 0.4 | 0.7 | 88 | 15 | 175 |
| MRAL.P59C-k0 | 0.7 | 1.5 | 94.6 | 15 | 176 |
| MRAL.S60C-k0 | 0.1 | 0 | 86.9 | 15 | 177 |
| MRAL.R61C-k0 | 0 | 0.3 | 86.9 | 15 | 178 |
| MRAL.F62C-k0 | 0.2 | 0 | 60 | 15 | 179 |
| MRAL.S63C-k0 | 0.5 | 0.6 | 88.1 | 15 | 180 |
| MRAL.S65C-k0 | 0.4 | 0.8 | 83.3 | 15 | 181 |
| MRAL.S67C-k0 | 1.5 | 0 | 72.8 | 15 | 182 |
| MRAL.G68C-k0 | 0.7 | 0.9 | 83.9 | 15 | 183 |
| MRAL.T69C-k0 | 1.1 | 0.6 | 86.4 | 15 | 184 |
| MRAL.D70C-k0 | 0.8 | 0.9 | 88.2 | 15 | 185 |
| MRAL.T72C-k0 | 0.6 | 0.7 | 90.1 | 15 | 186 |
| MRAL.F73C-k0 | 0.3 | 0 | 59.5 | 15 | 187 |
| MRAL.T74C-k0 | 0.2 | 0.6 | 95.6 | 15 | 188 |
| MRAL.I75C-k0 | no data | no data | no data | 15 | 189 |
| MRAL.S76C-k0 | 0.6 | 0.8 | 90.4 | 15 | 190 |
| MRAL.S77C-k0 | 1.1 | 0 | 74.2 | 15 | 191 |
| MRAL.L78C-k0 | 4.9 | 0 | 54.7 | 15 | 192 |
| MRAL.Q79C-k0 | 1.2 | 0.6 | 93.1 | 15 | 193 |
| MRAL.F98C-k0 | 0.6 | 0.8 | 71.8 | 15 | 194 |
| MRAL.G99C-k0 | 0.6 | 0.4 | 88.2 | 15 | 195 |
| MRAL.Q100C-k0 | 5 | 0.8 | 85 | 15 | 196 |
| MRAL.G101C-k0 | 0.3 | 0.4 | 98.1 | 15 | 197 |
| MRAL.T102C-k0 | 0.3 | 0 | 52.8 | 15 | 198 |
| MRAL.K103C-k0 | 1.1 | 0.4 | 89.2 | 15 | 199 |
| MRAL.V104C-k0 | 0.2 | 0.6 | 48.2 | 15 | 200 |
| MRAL.E105C-k0 | 90.8 | 0 | 1.2 | 15 | 201 |
| MRAL.I106C-k0 | 1.8 | 0 | 47.3 | 15 | 202 |
| MRAL.K107C-k0 | 5.4 | 0 | 82.6 | 15 | 203 |
| MRAL.A25C-k0 | 0.1 | 0.5 | 80 | 15 | 204 |
| MRAL.S26C-k0 | 0.3 | 1.4 | 94 | 15 | 205 |
| MRAL.Q27C-k0 | 0.3 | 1.3 | 94.6 | 15 | 206 |
| MRAL.Y32C-k0 | 0 | 1.2 | 95.7 | 15 | 207 |
| MRAL.L33C-k0 | 0 | 0 | 79.2 | 15 | 208 |
| MRAL.N34C-k0 | 0.3 | 0.4 | 95.7 | 15 | 209 |
| MRAL.Y50C-k0 | 0.4 | 1.3 | 97 | 15 | 210 |
| MRAL.T51C-k0 | 0.2 | 1.2 | 96.9 | 15 | 211 |
| MRAL.H55C-k0 | 0.2 | 1.5 | 95.7 | 15 | 212 |
| MRAL.S56C-k0 | 0.1 | 0.8 | 97 | 15 | 213 |
| MRAL.Y96C-k0 | 0.1 | 0.2 | 91.3 | 15 | 214 |
| MRAL.T97C-k0 | 0.3 | 0.9 | 97.5 | 15 | 215 |
| MRAL-k0.R108C | no data | no data | no data | 15 | 216 |
| MRAL-k0.T109C | 0.5 | 16 | 74.5 | 15 | 217 |
| MRAL-k0.V110C | 1.2 | 4 | 75 | 15 | 218 |
| MRAL-k0.A111C | 0.2 | 0.7 | 85.9 | 15 | 219 |
| MRAL-k0.A112C | 3.3 | 6.1 | 80.3 | 15 | 220 |
| MRAL-k0.P113C | no data | no data | no data | 15 | 221 |
| MRAL-k0.S114C | 0.3 | 0.7 | 94 | 15 | 222 |
| MRAL-k0.V115C | 0 | 0.1 | 34.9 | 15 | 223 |
| MRAL-k0.F116C | 0.3 | 0.3 | 77.3 | 15 | 224 |
| MRAL-k0.P120C | 0 | 0 | 28.8 | 15 | 225 |
| MRAL-k0.S121C | 8.6 | 0 | 57.4 | 15 | 226 |
| MRAL-k0.D122C | 1.8 | 0.1 | 30.3 | 15 | 227 |
| MRAL-k0.E123C | 2.3 | 1.6 | 75.9 | 15 | 228 |
| MRAL-k0.Q124C | 1.3 | 0.9 | 50.4 | 15 | 229 |
| MRAL-k0.L125C | 0.4 | 0.1 | 66.6 | 15 | 230 |
| MRAL-k0.K126C | 59.3 | 9.9 | 16.5 | 15 | 231 |
| MRAL-k0.S127C | 0.3 | 0.9 | 79 | 15 | 232 |
| MRAL-k0.G128C | 0.2 | 7 | 71.5 | 15 | 233 |
| MRAL-k0.T129C | 0 | 0.4 | 76.2 | 15 | 234 |
| MRAL-k0.A130C | 0 | 0 | 49.9 | 15 | 235 |
| MRAL-k0.S131C | 0 | 0 | 16.7 | 15 | 236 |
| MRAL-k0.L136C | 0 | 0 | 15 | 15 | 237 |
| MRAL-k0.N137C | 0 | 0 | 47.5 | 15 | 238 |
| MRAL-k0.N138C | 0 | 0.5 | 86.8 | 15 | 239 |
| MRAL-k0.F139C | 0 | 0 | 0 | 15 | 240 |
| MRAL-k0.Y140C | 0 | 0 | 29.9 | 15 | 241 |
| MRAL-k0.P141C | 0.1 | 2.7 | 79.8 | 15 | 242 |
| MRAL-k0.R142C | 0 | 0.6 | 74.1 | 15 | 243 |
| MRAL-k0.E143C | 0 | 0.5 | 88.4 | 15 | 244 |
| MRAL-k0.A144C | 0 | 0.1 | 42.1 | 15 | 245 |
| MRAL-k0.K145C | 0 | 0.9 | 82.8 | 15 | 246 |
| MRAL-k0.V146C | 0 | 0 | 26.5 | 15 | 247 |
| MRAL-k0.Q147C | 0 | 1.8 | 78.5 | 15 | 248 |
| MRAL-k0.W148C | no data | no data | no data | 15 | 249 |
| MRAL-k0.K149C | 0 | 0.6 | 79.5 | 15 | 250 |
| MRAL-k0.V150C | 0 | 0 | 34.8 | 15 | 251 |
| MRAL-k0.D151C | 2.7 | 14.9 | 66.5 | 15 | 252 |
| MRAL-k0.N152C | 1.2 | 58.4 | 26.8 | 15 | 253 |
| MRAL-k0.A153C | 0 | 7.1 | 71.8 | 15 | 254 |
| MRAL-k0.L154C | 0 | 2.3 | 66.5 | 15 | 255 |
| MRAL-k0.Q155C | 0 | 0.6 | 73.3 | 15 | 256 |
| MRAL-k0.S156C | 0.3 | 32.3 | 40.5 | 15 | 257 |
| MRAL-k0.G157C | 0 | 1.4 | 71.8 | 15 | 258 |
| MRAL-k0.N158C | 0 | 0.7 | 76.2 | 15 | 259 |
| MRAL-k0.S159C | 0 | 1.1 | 74.7 | 15 | 260 |
| MRAL-k0.Q160C | 0 | 1.5 | 78.5 | 15 | 261 |
| MRAL-k0.E161C | 0 | 1 | 79.8 | 15 | 262 |
| MRAL-k0.S162C | 0.6 | 1.6 | 86.7 | 15 | 263 |
| MRAL-k0.V163C | 0 | 1.7 | 87.1 | 15 | 264 |
| MRAL-k0.T164C | 0 | 2.6 | 84.3 | 15 | 265 |
| MRAL-k0.E165C | 0 | 0.6 | 89.5 | 15 | 266 |
| MRAL-k0.Q166C | 0 | 2 | 86.2 | 15 | 267 |
| MRAL-k0.D167C | 0 | 0.5 | 90.5 | 15 | 268 |
| MRAL-k0.S168C | 0 | 0.8 | 94.1 | 15 | 269 |
| MRAL-k0.K169C | 0 | 0.4 | 95.3 | 15 | 270 |
| MRAL-k0.D170C | 0.2 | 0.1 | 96 | 15 | 271 |
| MRAL-k0.S171C | 0 | 0.1 | 93.8 | 15 | 272 |
| MRAL-k0.T172C | 0 | 0 | 77.4 | 15 | 273 |
| MRAL-k0.Y173C | no data | no data | no data | 15 | 274 |
| MRAL-k0.S174C | 0 | 0 | 65.8 | 15 | 275 |
| MRAL-k0.L175C | 0 | 0.2 | 59.3 | 15 | 276 |
| MRAL-k0.T180C | 0 | 0.3 | 93.3 | 15 | 277 |
| MRAL-k0.L181C | 1.3 | 0.6 | 86.4 | 15 | 278 |
| MRAL-k0.S182C | 0.9 | 1.9 | 95 | 15 | 279 |
| MRAL-k0.K183C | 4.4 | 0.9 | 90.7 | 15 | 280 |
| MRAL-k0.A184C | 1.6 | 27.9 | 67.7 | 15 | 281 |
| MRAL-k0.D185C | 0.5 | 1.1 | 96.5 | 15 | 282 |
| MRAL-k0.Y186C | 2.4 | 18.9 | 67.4 | 15 | 283 |
| MRAL-k0.E187C | 2.3 | 0 | 11.2 | 15 | 284 |
| MRAL-k0.K188C | 1.8 | 8.6 | 85.8 | 15 | 285 |
| MRAL-k0.H189C | 1 | 0.8 | 93 | 15 | 286 |
| MRAL-k0.K190C | 25.5 | 0.2 | 11.4 | 15 | 287 |
| MRAL-k0.V191C | 2.8 | 1.6 | 84 | 15 | 288 |
| MRAL-k0.Y192C | 0.4 | 1.1 | 67.5 | 15 | 289 |
| MRAL-k0.A193C | 1.7 | 1.4 | 94.5 | 15 | 290 |
| MRAL-k0.E195C | 0.9 | 1.7 | 95.5 | 15 | 291 |
| MRAL-k0.V196C | 1 | 1.1 | 67.5 | 15 | 292 |
| MRAL-k0.T197C | 0.8 | 1.5 | 94.8 | 15 | 293 |
| MRAL-k0.H198C | 0.7 | 1.3 | 85 | 15 | 294 |
| MRAL-k0.Q199C | 1.4 | 2.5 | 92.9 | 15 | 295 |
| MRAL-k0.G200C | 7.3 | 14.8 | 75.6 | 15 | 296 |
| MRAL-k0.L201C | 1.7 | 5 | 88 | 15 | 297 |
| MRAL-k0.S202C | 2.8 | 46.4 | 49.4 | 15 | 298 |
| MRAL-k0.S203C | 9.1 | 0 | 87.1 | 15 | 299 |
| MRAL-k0.P204C | 1 | 0 | 95.8 | 15 | 300 |
| MRAL-k0.V205C | 1.7 | 1 | 88.4 | 15 | 301 |
| MRAL-k0.T206C | 1.4 | 0.7 | 90.1 | 15 | 302 |
| MRAL-k0.K207C | 3.2 | 0.5 | 79.8 | 15 | 303 |
| MRAL-k0.S208C | 7.7 | 0.8 | 77.8 | 15 | 304 |
| MRAL-k0.F209C | 0 | 0 | 37.2 | 15 | 305 |
| MRAL-k0.N210C | 22.8 | 0 | 20.2 | 15 | 306 |
| MRAL-k0.R211C | 9.2 | 0 | 59.7 | 15 | 307 |
| MRAL-k0.G212C | 58.9 | 0 | 28.7 | 15 | 308 |
| MRAL-k0.E213C | 55.1 | 0 | 12.1 | 15 | 309 |

From this result, it was found that cysteine substitution in the light chain variable region or light chain constant region improved the protease resistance of the heavy chain hinge region in the MRA variants shown in Table 27. Alternatively, the result suggested that a Fab dimer was formed by a covalent bond between the Fab-Fab.

TABLE 27
MRA variants

SEQ ID NO:

| | Heavy | Heavy | Light | Light |
|---|---|---|---|---|
| | chain | chain | chain | chain |
| | variable | constant | variable | constant |
| Antibody name | region | region | region | region |
| MRAL.Q100C-k0 | 17 | 18 | 196 | 20 |
| MRAL.E105C-k0 | 17 | 18 | 201 | 20 |
| MRAL.K107C-k0 | 17 | 18 | 203 | 20 |
| MRAL-k0.T109C | 17 | 18 | 19 | 217 |
| MRAL-k0.A112C | 17 | 18 | 19 | 220 |
| MRAL-k0.S121C | 17 | 18 | 19 | 226 |
| MRAL-k0.K126C | 17 | 18 | 19 | 231 |
| MRAL-k0.G128C | 17 | 18 | 19 | 233 |
| MRAL-k0.D151C | 17 | 18 | 19 | 252 |
| MRAL-k0.N152C | 17 | 18 | 19 | 253 |
| MRAL-k0.A153C | 17 | 18 | 19 | 254 |
| MRAL-k0.S156C | 17 | 18 | 19 | 257 |
| MRAL-k0.A184C | 17 | 18 | 19 | 281 |
| MRAL-k0.Y186C | 17 | 18 | 19 | 283 |
| MRAL-k0.K188C | 17 | 18 | 19 | 285 |
| MRAL-k0.K190C | 17 | 18 | 19 | 287 |
| MRAL-k0.G200C | 17 | 18 | 19 | 296 |
| MRAL-k0.L201C | 17 | 18 | 19 | 297 |
| MRAL-k0.S202C | 17 | 18 | 19 | 298 |
| MRAL-k0.S203C | 17 | 18 | 19 | 299 |
| MRAL-k0.S208C | 17 | 18 | 19 | 304 |
| MRAL-k0.N210C | 17 | 18 | 19 | 306 |
| MRAL-k0.R211C | 17 | 18 | 19 | 307 |
| MRAL-k0.G212C | 17 | 18 | 19 | 308 |
| MRAL-k0.E213C | 17 | 18 | 19 | 309 |

### Reference Example 7 Study of Methods for Assessing Antibodies Having Cysteine Substitution

### Reference Example 7-1 Production of Antibodies Having Cysteine Substitution in the Light Chain

The amino acid residue at position 126 according to Kabat numbering in the light chain constant region (k0, SEQ ID NO: 20) of MRA, an anti-human IL6R neutralizing antibody (heavy chain: MRAH-G1T4 (SEQ ID NO: 15), light chain: MRAL-k0 (SEQ ID NO: 16)), was substituted with cysteine to produce a variant of the light chain constant region of MRA, k0.K126C (SEQ ID No: 231). This variant of the light chain constant region of MRA was linked with the MRA light chain variable region (MRAL, SEQ ID NO: 19) to produce a variant of the light chain of MRA, and an expression vector encoding the corresponding gene was produced by a method known to the person skilled in the art.

The MRA light chain variant produced above was combined with the MRA heavy chain. The resultant MRA variant MRAL-k0.K126C (heavy chain: MRAH-G1T4 (SEQ ID NO: 15), light chain variable region: MRAL (SEQ ID NO: 19), light chain constant region: k0.K126C (SEQ ID NO: 231)) was expressed by transient expression using FreeStyle293 cells (Invitrogen) or Expi293 cells (Life technologies) by a method known to the person skilled in the art, and purified with Protein A by a method known to the person skilled in the art.

### Reference Example 7-2 Assessment of Protease-Mediated Capillary Electrophoresis of Antibodies Having Cysteine Substitution in the Light Chain

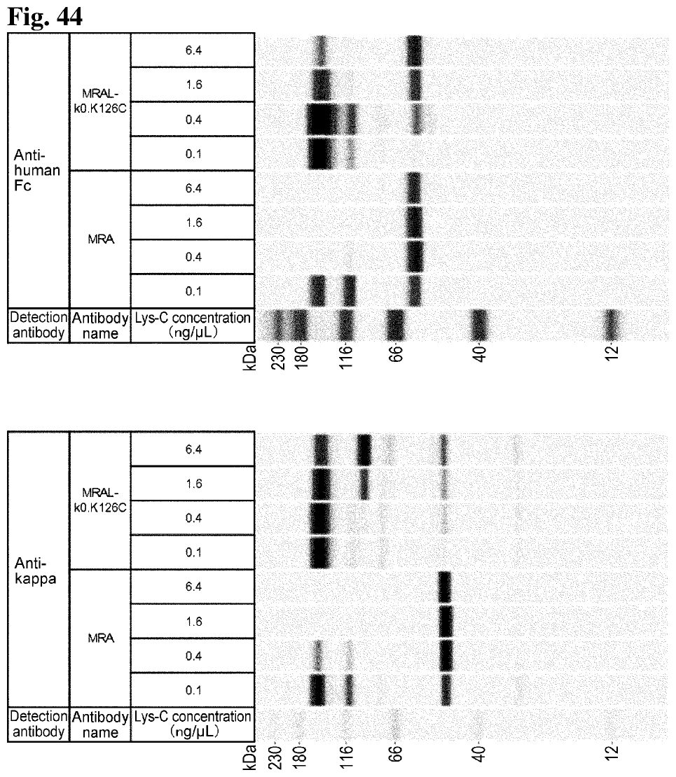

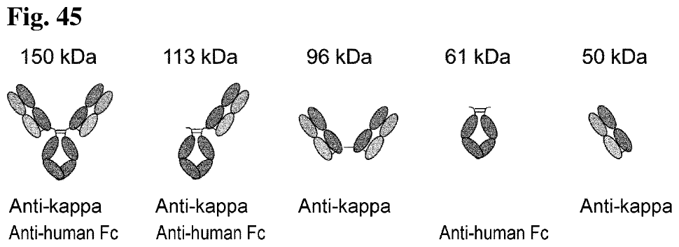

Using a protease that cleaves the heavy chain hinge region of antibody to cause Fab fragmentation, the MRA light chain variant produced in Reference Example 7-1 was examined for whether it acquired protease resistance so that its fragmentation would be inhibited. The protease used was Lys-C (Endoproteinase Lys-C Sequencing Grade) (SIGMA; 11047825001). Reaction was performed under the conditions of 0.1, 0.4, 1.6, or 6.4 ng/microliter protease, 100 microgram/mL antibody, 80% 25 mM Tris-HCl pH 8.0, 20% PBS, and 35 degrees C. for two hours. The sample was then subjected to non-reducing capillary electrophoresis. Wes (Protein Simple) was used for capillary electrophoresis, and an HRP-labeled anti-kappa chain antibody (abcam; ab46527) or an HRP-labeled anti-human Fc antibody (Protein Simple; 043-491) was used for detection. The result is shown in FIG. 44. For MRA treated with Lys-C, detection with the anti-kappa chain antibody showed disappearance of the band at around 150 kDa and appearance of a new band at around 50 kDa, and, at low Lys-C concentrations, also showed appearance of a slight band at 113 kDa. Detection with the anti-human Fc antibody showed disappearance of the band at around 150 kDa and appearance of a new band at around 61 kDa, and, at low Lys-C concentrations, also showed appearance of a slight band at 113 kDa. For the MRA variant produced in Reference Example 7-1, on the other hand, the band at around 150 kDa hardly disappeared, and a new band appeared at around 96 kDa. Detection with the anti-human Fc antibody showed that the band at around 150 kDa hardly disappeared and a new band appeared at around 61 kDa, and, at low Lys-C concentrations, a slight band also appeared at 113 kDa. The above results suggested that, as shown in FIG. 45, the band at around 150 kDa was IgG, the band at around 113 kDa was a one-arm form in which the heavy chain hinge was cleaved once, the band at around 96 kDa was a Fab dimer, the band at around 61 kDa was Fc, and the band at around 50 kDa was Fab.

### Reference Example 8 Assessment of Antibodies Having Cysteine Substitution at Various Positions of IgG1

### Reference Example 8-1 Production of Antibodies Having Cysteine Substitution at Various Positions of IgG1

The heavy chain and light chain of an anti-human IL6R neutralizing antibody, MRA-IgG1 (heavy chain: MRAH-G1T4 (SEQ ID NO: 15), light chain: MRAL-k0 (SEQ ID NO: 16)), were subjected to a study in which an arbitrary amino acid residue structurally exposed to the surface was substituted with cysteine.

Amino acid residues within the MRA-IgG1 heavy chain variable region (MRAH, SEQ ID NO: 17) were substituted with cysteine to produce variants of the MRA-IgG1 heavy chain variable region shown in Table 28. These variants of the MRA-IgG1 heavy chain variable region were each linked with the MRA-IgG1 heavy chain constant region (G1T4, SEQ ID NO: 18) to produce MRA-IgG1 heavy chain variants, and expression vectors encoding the corresponding genes were produced by a method known to the person skilled in the art. In addition, amino acid residues within the MRA-IgG1 heavy chain constant region (G1T4, SEQ ID NO: 18) were substituted with cysteine to produce variants of the MRA-IgG1 heavy chain constant region shown in Table 29. These variants of the MRA-IgG1 heavy chain constant region were each linked with the MRA-IgG1 heavy chain variable region (MRAH, SEQ ID NO: 17) to produce MRA-IgG1 heavy chain variants, and expression vectors encoding the corresponding genes were produced by a method known to the person skilled in the art.

TABLE 28

| Variant of MRA-IgG1 | Position of cysteine | |
|---|---|---|
| heavy chain variable | substitution | SEQ ID |
| region | (Kabat numbering) | NO: |
| MRAH.Q5C | 5 | 322 |
| MRAH.E6C | 6 | 323 |
| MRAH.S7C | 7 | 324 |
| MRAH.G8C | 8 | 325 |
| MRAH.P9C | 9 | 326 |
| MRAH.G10C | 10 | 327 |
| MRAH.L11C | 11 | 328 |
| MRAH.V12C | 12 | 329 |
| MRAH.R13C | 13 | 330 |
| MRAH.P14C | 14 | 331 |
| MRAH.S15C | 15 | 332 |
| MRAH.Q16C | 16 | 333 |
| MRAH.T17C | 17 | 334 |
| MRAH.L18C | 18 | 335 |
| MRAH.S19C | 19 | 336 |
| MRAH.L20C | 20 | 337 |
| MRAH.T21C | 21 | 338 |
| MRAH.T23C | 23 | 339 |
| MRAH.S25C | 25 | 340 |
| MRAH.G26C | 26 | 341 |
| MRAH.S28C | 28 | 342 |
| MRAH.T30C | 30 | 343 |
| MRAH.S31C | 31 | 344 |
| MRAH.W35C | 35 | 345 |
| MRAH.S35aC | 35a | 346 |
| MRAH.Y50C | 50 | 347 |
| MRAH.I51C | 51 | 348 |
| MRAH.S52C | 52 | 349 |
| MRAH.S62C | 62 | 350 |
| MRAH.L63C | 63 | 351 |
| MRAH.K64C | 64 | 352 |
| MRAH.S65C | 65 | 353 |
| MRAH.R66C | 66 | 354 |
| MRAH.V67C | 67 | 355 |
| MRAH.T68C | 68 | 356 |
| MRAH.L70C | 70 | 357 |
| MRAH.D72C | 72 | 358 |
| MRAH.T73C | 73 | 359 |
| MRAH.S74C | 74 | 360 |
| MRAH.K75C | 75 | 361 |
| MRAH.N76C | 76 | 362 |
| MRAH.Q77C | 77 | 363 |
| MRAH.S79C | 79 | 364 |
| MRAH.L80C | 80 | 365 |
| MRAH.R81C | 81 | 366 |
| MRAH.L82C | 82 | 367 |
| MRAH.S82aC | 82a | 368 |
| MRAH.S82bC | 82b | 369 |
| MRAH.V82cC | 82c | 370 |
| MRAH.D101C | 101 | 371 |
| MRAH.Y102C | 102 | 372 |
| MRAH.S112C | 112 | 373 |
| MRAH.S113C | 113 | 374 |

TABLE 29

| Variant of MRA-IgG1 | Position of cysteine | |
|---|---|---|
| heavy chain constant | substitution | SEQ ID |
| region | (EU numbering) | NO: |
| G1T4.A118C | 118 | 375 |
| G1T4.S119C | 119 | 376 |
| G1T4.T120C | 120 | 377 |
| G1T4.K121C | 121 | 378 |
| G1T4.G122C | 122 | 379 |
| G1T4.P123C | 123 | 380 |
| G1T4.S124C | 124 | 381 |
| G1T4.V125C | 125 | 382 |
| G1T4.F126C | 126 | 383 |
| G1T4.P127C | 127 | 384 |
| G1T4.S131C | 131 | 385 |
| G1T4.S132C | 132 | 386 |
| G1T4.K133C | 133 | 387 |
| G1T4.S134C | 134 | 388 |
| G1T4.T135C | 135 | 389 |
| G1T4.S136C | 136 | 390 |
| G1T4.G137C | 137 | 391 |
| G1T4.G138C | 138 | 392 |
| G1T4.T139C | 139 | 393 |
| G1T4.A140C | 140 | 394 |
| G1T4.A141C | 141 | 395 |
| G1T4.D148C | 148 | 396 |
| G1T4.Y149C | 149 | 397 |
| G1T4.F150C | 150 | 398 |
| G1T4.P151C | 151 | 399 |
| G1T4.E152C | 152 | 400 |
| G1T4.P153C | 153 | 401 |
| G1T4.V154C | 154 | 402 |
| G1T4.T155C | 155 | 403 |
| G1T4.V156C | 156 | 404 |
| G1T4.S157C | 157 | 405 |
| G1T4.W158C | 158 | 406 |
| G1T4.N159C | 159 | 407 |
| G1T4.S160C | 160 | 408 |
| G1T4.G161C | 161 | 409 |
| G1T4.A162C | 162 | 410 |
| G1T4.L163C | 163 | 411 |
| G1T4.T164C | 164 | 412 |
| G1T4.S165C | 165 | 413 |
| G1T4.G166C | 166 | 414 |
| G1T4.V167C | 167 | 415 |
| G1T4.V173C | 173 | 416 |
| G1T4.L174C | 174 | 417 |
| G1T4.Q175C | 175 | 418 |
| G1T4.S176C | 176 | 419 |
| G1T4.S177C | 177 | 420 |
| G1T4.G178C | 178 | 421 |
| G1T4.L179C | 179 | 422 |
| G1T4.Y180C | 180 | 423 |
| G1T4.V186C | 186 | 424 |
| G1T4.T187C | 187 | 425 |
| G1T4.V188C | 188 | 426 |
| G1T4.P189C | 189 | 427 |
| G1T4.S190C | 190 | 428 |
| G1T4.S191C | 191 | 429 |
| G1T4.S192C | 192 | 430 |
| G1T4.L193C | 193 | 431 |
| G1T4.G194C | 194 | 432 |
| G1T4.T195C | 195 | 433 |
| G1T4.Q196C | 196 | 434 |
| G1T4.T197C | 197 | 435 |
| G1T4.Y198C | 198 | 436 |
| G1T4.I199C | 199 | 437 |
| G1T4.N201C | 201 | 438 |
| G1T4.V202C | 202 | 439 |
| G1T4.N203C | 203 | 440 |
| G1T4.H204C | 204 | 441 |
| G1T4.K205C | 205 | 442 |
| G1T4.P206C | 206 | 443 |
| G1T4.S207C | 207 | 444 |
| G1T4.N208C | 208 | 445 |
| G1T4.T209C | 209 | 446 |
| G1T4.K210C | 210 | 447 |
| G1T4.V211C | 211 | 448 |
| G1T4.D212C | 212 | 449 |
| G1T4.K213C | 213 | 450 |
| G1T4.R214C | 214 | 451 |
| G1T4.V215C | 215 | 452 |
| G1T4.E216C | 216 | 453 |
| G1T4.P217C | 217 | 454 |
| G1T4.K218C | 218 | 455 |
| G1T4.S219C | 219 | 456 |

Similarly, amino acid residues within the MRA-IgG1 light chain variable region (MRAL, SEQ ID NO: 19) were substituted with cysteine to produce variants of the MRA-IgG1 light chain variable region shown in Table 30. These variants of the MRA-IgG1 light chain variable region were each linked with the MRA-IgG1 light chain constant region (k0, SEQ ID NO: 20) to produce MRA-IgG1 light chain variants, and expression vectors encoding the corresponding genes were produced by a method known to the person skilled in the art. In addition, amino acid residues within the MRA-IgG1 light chain constant region (k0, SEQ ID NO: 20) were substituted with cysteine to produce variants of the MRA-IgG1 light chain constant region shown in Table 31. These variants of the MRA-IgG1 heavy chain constant region were each linked with the MRA-IgG1 light chain variable region (MRAL, SEQ ID NO: 19) to produce MRA-IgG1 light chain variants, and expression vectors encoding the corresponding genes were produced by a method known to the person skilled in the art.

TABLE 30

| Variant of MRA-IgG1 | Position of cysteine | |
|---|---|---|
| light chain variable | substitution | |
| region | (Kabat numbering) | SEQ ID NO: |
| MRAL.T5C | 5 | 457 |
| MRAL.Q6C | 6 | 458 |
| MRAL.S7C | 7 | 459 |
| MRAL.P8C | 8 | 460 |
| MRAL.S9C | 9 | 461 |
| MRAL.S10C | 10 | 462 |
| MRAL.L11C | 11 | 463 |
| MRAL.S12C | 12 | 464 |
| MRAL.A13C | 13 | 465 |
| MRAL.S14C | 14 | 466 |
| MRAL.V15C | 15 | 467 |
| MRAL.G16C | 16 | 468 |
| MRAL.D17C | 17 | 469 |
| MRAL.R18C | 18 | 470 |
| MRAL.V19C | 19 | 471 |
| MRAL.T20C | 20 | 472 |
| MRAL.I21C | 21 | 473 |
| MRAL.T22C | 22 | 474 |
| MRAL.A25C | 25 | 475 |
| MRAL.S26C | 26 | 476 |
| MRAL.Q27C | 27 | 477 |
| MRAL.Y32C | 32 | 478 |
| MRAL.L33C | 33 | 479 |
| MRAL.N34C | 34 | 480 |
| MRAL.Y50C | 50 | 481 |
| MRAL.T51C | 51 | 482 |
| MRAL.H55C | 55 | 483 |
| MRAL.S56C | 56 | 484 |
| MRAL.G57C | 57 | 485 |
| MRAL.V58C | 58 | 486 |
| MRAL.P59C | 59 | 487 |
| MRAL.S60C | 60 | 488 |
| MRAL.R61C | 61 | 489 |
| MRAL.F62C | 62 | 490 |
| MRAL.S63C | 63 | 491 |
| MRAL.S65C | 65 | 492 |
| MRAL.S67C | 67 | 493 |
| MRAL.G68C | 68 | 494 |
| MRAL.T69C | 69 | 495 |
| MRAL.D70C | 70 | 496 |
| MRAL.T72C | 72 | 497 |
| MRAL.F73C | 73 | 498 |
| MRAL.T74C | 74 | 499 |
| MRAL.I75C | 75 | 500 |
| MRAL.S76C | 76 | 501 |
| MRAL.S77C | 77 | 502 |
| MRAL.L78C | 78 | 503 |
| MRAL.Q79C | 79 | 504 |
| MRAL.Y96C | 96 | 505 |
| MRAL.T97C | 97 | 506 |
| MRAL.F98C | 98 | 507 |
| MRAL.G99C | 99 | 508 |
| MRAL.Q100C | 100 | 509 |
| MRAL.G101C | 101 | 510 |
| MRAL.T102C | 102 | 511 |
| MRAL.K103C | 103 | 512 |
| MRAL.V104C | 104 | 513 |
| MRAL.E105C | 105 | 514 |
| MRAL.I106C | 106 | 515 |
| MRAL.K107C | 107 | 516 |

TABLE 31

| Variant of MRA-IgG1 | Position of cysteine | |
|---|---|---|
| light chain constant | substitution | |
| region | (Kabat numbering) | SEQ ID NO: |
| k0.R108C | 108 | 517 |
| k0.T109C | 109 | 518 |
| k0.V110C | 110 | 519 |
| k0.A111C | 111 | 520 |
| k0.A112C | 112 | 521 |
| k0.P113C | 113 | 522 |
| k0.S114C | 114 | 523 |
| k0.V115C | 115 | 524 |
| k0.F116C | 116 | 525 |
| k0.P120C | 120 | 526 |
| k0.S121C | 121 | 527 |
| k0.D122C | 122 | 528 |
| k0.E123C | 123 | 529 |
| k0.Q124C | 124 | 530 |
| k0.L125C | 125 | 531 |
| k0.K126C | 126 | 532 |
| k0.S127C | 127 | 533 |
| k0.G128C | 128 | 534 |
| k0.T129C | 129 | 535 |
| k0.A130C | 130 | 536 |
| k0.S131C | 131 | 537 |
| k0.L136C | 136 | 538 |
| k0.N137C | 137 | 539 |
| k0.N138C | 138 | 540 |
| k0.F139C | 139 | 541 |
| k0.Y140C | 140 | 542 |
| k0.P141C | 141 | 543 |
| k0.R142C | 142 | 544 |
| k0.E143C | 143 | 545 |
| k0.A144C | 144 | 546 |
| k0.K145C | 145 | 547 |
| k0.V146C | 146 | 548 |
| k0.Q147C | 147 | 549 |
| k0.W148C | 148 | 550 |
| k0.K149C | 149 | 551 |
| k0.V15OC | 150 | 552 |
| k0.D151C | 151 | 553 |
| k0.N152C | 152 | 554 |
| k0.A153C | 153 | 555 |
| k0.L154C | 154 | 556 |
| k0.Q155C | 155 | 557 |
| k0.S156C | 156 | 558 |
| k0.G157C | 157 | 559 |
| k0.N158C | 158 | 560 |
| k0.S159C | 159 | 561 |
| k0.Q160C | 160 | 562 |
| k0.E161C | 161 | 563 |
| k0.S162C | 162 | 564 |
| k0.V163C | 163 | 565 |
| k0.T164C | 164 | 566 |
| k0.E165C | 165 | 567 |
| k0.Q166C | 166 | 568 |
| k0.D167C | 167 | 569 |
| k0.S168C | 168 | 570 |
| k0.K169C | 169 | 571 |
| k0.D170C | 170 | 572 |
| k0.S171C | 171 | 573 |
| k0.T172C | 172 | 574 |
| k0.Y173C | 173 | 575 |
| k0.S174C | 174 | 576 |
| k0.L175C | 175 | 577 |
| k0.T180C | 180 | 578 |
| k0.L181C | 181 | 579 |
| k0.S182C | 182 | 580 |
| k0.K183C | 183 | 581 |
| k0.A184C | 184 | 582 |
| k0.D185C | 185 | 583 |
| k0.Y186C | 186 | 584 |
| k0.E187C | 187 | 585 |
| k0.K188C | 188 | 586 |
| k0.H189C | 189 | 587 |
| k0.K190C | 190 | 588 |
| k0.V191C | 191 | 589 |
| k0.Y192C | 192 | 590 |
| k0.A193C | 193 | 591 |
| k0.E195C | 195 | 592 |
| k0.V196C | 196 | 593 |
| k0.T197C | 197 | 594 |
| k0.H198C | 198 | 595 |
| k0.Q199C | 199 | 596 |
| k0.G200C | 200 | 597 |
| k0.L201C | 201 | 598 |
| k0.S202C | 202 | 599 |
| k0.S203C | 203 | 600 |
| k0.P204C | 204 | 601 |
| k0.V205C | 205 | 602 |
| k0.T206C | 206 | 603 |
| k0.K207C | 207 | 604 |
| k0.S208C | 208 | 605 |
| k0.F209C | 209 | 606 |
| k0.N210C | 210 | 607 |
| k0.R211C | 211 | 608 |
| k0.G212C | 212 | 609 |
| k0.E213C | 213 | 610 |

The MRA-IgG1 heavy chain variants produced above were combined with the MRA-IgG1 light chain, or the MRA-IgG1 heavy chain was combined with the MRA-IgG1 light chain variants. The resultant MRA-IgG1 heavy chain variants and MRA-IgG1 light chain variants shown in Tables 32 and 33 were expressed by transient expression using FreeStyle293 cells (Invitrogen) or Expi293 cells (Life technologies) by a method known to the person skilled in the art, and purified with Protein A by a method known to the person skilled in the art.

TABLE 32

| | Heavy | Heavy | Light | Light |
|---|---|---|---|---|
| | chain | chain | chain | chain |
| | variable | constant | variable | constant |
| | region | region | region | region |
| MRA-IgG1 heavy chain | SEQ ID | SEQ ID | SEQ ID | SEQ ID |
| variant name | NO: | NO: | NO: | NO: |
| MRAH.Q5C-IgG1 | 322 | 18 | 19 | 20 |
| MRAH.E6C-IgG1 | 323 | 18 | 19 | 20 |
| MRAH.S7C-IgG1 | 324 | 18 | 19 | 20 |
| MRAH.G8C-IgG1 | 325 | 18 | 19 | 20 |
| MRAH.P9C-IgG1 | 326 | 18 | 19 | 20 |
| MRAH.G10C-IgG1 | 327 | 18 | 19 | 20 |
| MRAH.L11C-IgG1 | 328 | 18 | 19 | 20 |
| MRAH.V12C-IgG1 | 329 | 18 | 19 | 20 |
| MRAH.R13C-IgG1 | 330 | 18 | 19 | 20 |
| MRAH.P14C-IgG1 | 331 | 18 | 19 | 20 |
| MRAH.S15C-IgG1 | 332 | 18 | 19 | 20 |
| MRAH.Q16C-IgG1 | 333 | 18 | 19 | 20 |
| MRAH.T17C-IgG1 | 334 | 18 | 19 | 20 |
| MRAH.L18C-IgG1 | 335 | 18 | 19 | 20 |
| MRAH.S19C-IgG1 | 336 | 18 | 19 | 20 |
| MRAH.L20C-IgG1 | 337 | 18 | 19 | 20 |
| MRAH.T21C-IgG1 | 338 | 18 | 19 | 20 |
| MRAH.T23C-IgG1 | 339 | 18 | 19 | 20 |
| MRAH.S25C-IgG1 | 340 | 18 | 19 | 20 |
| MRAH.G26C-IgG1 | 341 | 18 | 19 | 20 |
| MRAH.S28C-IgG1 | 342 | 18 | 19 | 20 |
| MRAH.T30C-IgG1 | 343 | 18 | 19 | 20 |
| MRAH.S31C-IgG1 | 344 | 18 | 19 | 20 |
| MRAH.W35C-IgG1 | 345 | 18 | 19 | 20 |
| MRAH.S35aC-IgG1 | 346 | 18 | 19 | 20 |
| MRAH.Y50C-IgG1 | 347 | 18 | 19 | 20 |
| MRAH.I51C-IgG1 | 348 | 18 | 19 | 20 |
| MRAH.S52C-IgG1 | 349 | 18 | 19 | 20 |
| MRAH.S62C-IgG1 | 350 | 18 | 19 | 20 |
| MRAH.L63C-IgG1 | 351 | 18 | 19 | 20 |
| MRAH.K64C-IgG1 | 352 | 18 | 19 | 20 |
| MRAH.S65C-IgG1 | 353 | 18 | 19 | 20 |
| MRAH.R66C-IgG1 | 354 | 18 | 19 | 20 |
| MRAH.V67C-IgG1 | 355 | 18 | 19 | 20 |
| MRAH.T68C-IgG1 | 356 | 18 | 19 | 20 |
| MRAH.L70C-IgG1 | 357 | 18 | 19 | 20 |
| MRAH.D72C-IgG1 | 358 | 18 | 19 | 20 |
| MRAH.T73C-IgG1 | 359 | 18 | 19 | 20 |
| MRAH.S74C-IgG1 | 360 | 18 | 19 | 20 |
| MRAH.K75C-IgG1 | 361 | 18 | 19 | 20 |
| MRAH.N76C-IgG1 | 362 | 18 | 19 | 20 |
| MRAH.Q77C-IgG1 | 363 | 18 | 19 | 20 |
| MRAH.S79C-IgG1 | 364 | 18 | 19 | 20 |
| MRAH.L80C-IgG1 | 365 | 18 | 19 | 20 |
| MRAH.R81C-IgG1 | 366 | 18 | 19 | 20 |
| MRAH.L82C-IgG1 | 367 | 18 | 19 | 20 |
| MRAH.S82aC-IgG1 | 368 | 18 | 19 | 20 |
| MRAH.S82bC-IgG1 | 369 | 18 | 19 | 20 |
| MRAH.V82cC-IgG1 | 370 | 18 | 19 | 20 |
| MRAH.D101C-IgG1 | 371 | 18 | 19 | 20 |
| MRAH.Y102C-IgG1 | 372 | 18 | 19 | 20 |
| MRAH.S112C-IgG1 | 373 | 18 | 19 | 20 |
| MRAH.S113C-IgG1 | 374 | 18 | 19 | 20 |
| G1T4.A118C-IgG1 | 17 | 375 | 19 | 20 |
| G1T4.S119C-IgG1 | 17 | 376 | 19 | 20 |
| G1T4.T120C-IgG1 | 17 | 377 | 19 | 20 |
| G1T4.K121C-IgG1 | 17 | 378 | 19 | 20 |
| G1T4.G122C-IgG1 | 17 | 379 | 19 | 20 |
| G1T4.P123C-IgG1 | 17 | 380 | 19 | 20 |
| G1T4.S124C-IgG1 | 17 | 381 | 19 | 20 |
| G1T4.V125C-IgG1 | 17 | 382 | 19 | 20 |
| G1T4.F126C-IgG1 | 17 | 383 | 19 | 20 |
| G1T4.P127C-IgG1 | 17 | 384 | 19 | 20 |
| G1T4.S131C-IgG1 | 17 | 385 | 19 | 20 |
| G1T4.S132C-IgG1 | 17 | 386 | 19 | 20 |
| G1T4.K133C-IgG1 | 17 | 387 | 19 | 20 |
| G1T4.S134C-IgG1 | 17 | 388 | 19 | 20 |
| G1T4.T135C-IgG1 | 17 | 389 | 19 | 20 |
| G1T4.S136C-IgG1 | 17 | 390 | 19 | 20 |
| G1T4.G137C-IgG1 | 17 | 391 | 19 | 20 |
| G1T4.G138C-IgG1 | 17 | 392 | 19 | 20 |
| G1T4.T139C-IgG1 | 17 | 393 | 19 | 20 |
| G1T4.A140C-IgG1 | 17 | 394 | 19 | 20 |
| G1T4.A141C-IgG1 | 17 | 395 | 19 | 20 |
| G1T4.D148C-IgG1 | 17 | 396 | 19 | 20 |
| G1T4.Y149C-IgG1 | 17 | 397 | 19 | 20 |
| G1T4.F150C-IgG1 | 17 | 398 | 19 | 20 |
| G1T4.P151C-IgG1 | 17 | 399 | 19 | 20 |
| G1T4.E152C-IgG1 | 17 | 400 | 19 | 20 |
| G1T4.P153C-IgG1 | 17 | 401 | 19 | 20 |
| G1T4.V154C-IgG1 | 17 | 402 | 19 | 20 |
| G1T4.T155C-IgG1 | 17 | 403 | 19 | 20 |
| G1T4.V156C-IgG1 | 17 | 404 | 19 | 20 |
| G1T4.S157C-IgG1 | 17 | 405 | 19 | 20 |
| G1T4.W158C-IgG1 | 17 | 406 | 19 | 20 |
| G1T4.N159C-IgG1 | 17 | 407 | 19 | 20 |
| G1T4.S160C-IgG1 | 17 | 408 | 19 | 20 |
| G1T4.G161C-IgG1 | 17 | 409 | 19 | 20 |
| G1T4.A162C-IgG1 | 17 | 410 | 19 | 20 |
| G1T4.L163C-IgG1 | 17 | 411 | 19 | 20 |
| G1T4.T164C-IgG1 | 17 | 412 | 19 | 20 |
| G1T4.S165C-IgG1 | 17 | 413 | 19 | 20 |
| G1T4.G166C-IgG1 | 17 | 414 | 19 | 20 |
| G1T4.V167C-IgG1 | 17 | 415 | 19 | 20 |
| G1T4.V173C-IgG1 | 17 | 416 | 19 | 20 |
| G1T4.L174C-IgG1 | 17 | 417 | 19 | 20 |
| G1T4.Q175C-IgG1 | 17 | 418 | 19 | 20 |
| G1T4.S176C-IgG1 | 17 | 419 | 19 | 20 |
| G1T4.S177C-IgG1 | 17 | 420 | 19 | 20 |
| G1T4.G178C-IgG1 | 17 | 421 | 19 | 20 |
| G1T4.L179C-IgG1 | 17 | 422 | 19 | 20 |
| G1T4.Y180C-IgG1 | 17 | 423 | 19 | 20 |
| G1T4.V186C-IgG1 | 17 | 424 | 19 | 20 |
| G1T4.T187C-IgG1 | 17 | 425 | 19 | 20 |
| G1T4.V188C-IgG1 | 17 | 426 | 19 | 20 |
| G1T4.P189C-IgG1 | 17 | 427 | 19 | 20 |
| G1T4.S190C-IgG1 | 17 | 428 | 19 | 20 |
| G1T4.S191C-IgG1 | 17 | 429 | 19 | 20 |
| G1T4.S192C-IgG1 | 17 | 430 | 19 | 20 |
| G1T4.L193C-IgG1 | 17 | 431 | 19 | 20 |
| G1T4.G194C-IgG1 | 17 | 432 | 19 | 20 |
| G1T4.T195C-IgG1 | 17 | 433 | 19 | 20 |
| G1T4.Q196C-IgG1 | 17 | 434 | 19 | 20 |
| G1T4.T197C-IgG1 | 17 | 435 | 19 | 20 |
| G1T4.Y198C-IgG1 | 17 | 436 | 19 | 20 |
| G1T4.I199C-IgG1 | 17 | 437 | 19 | 20 |
| G1T4.N201C-IgG1 | 17 | 438 | 19 | 20 |
| G1T4.V202C-IgG1 | 17 | 439 | 19 | 20 |
| G1T4.N203C-IgG1 | 17 | 440 | 19 | 20 |
| G1T4.H204C-IgG1 | 17 | 441 | 19 | 20 |
| G1T4.K205C-IgG1 | 17 | 442 | 19 | 20 |
| G1T4.P206C-IgG1 | 17 | 443 | 19 | 20 |
| G1T4.S207C-IgG1 | 17 | 444 | 19 | 20 |
| G1T4.N208C-IgG1 | 17 | 445 | 19 | 20 |
| G1T4.T209C-IgG1 | 17 | 446 | 19 | 20 |
| G1T4.K210C-IgG1 | 17 | 447 | 19 | 20 |
| G1T4.V211C-IgG1 | 17 | 448 | 19 | 20 |
| G1T4.D212C-IgG1 | 17 | 449 | 19 | 20 |
| G1T4.K213C-IgG1 | 17 | 450 | 19 | 20 |
| G1T4.R214C-IgG1 | 17 | 451 | 19 | 20 |
| G1T4.V215C-IgG1 | 17 | 452 | 19 | 20 |
| G1T4.E216C-IgG1 | 17 | 453 | 19 | 20 |
| G1T4.P217C-IgG1 | 17 | 454 | 19 | 20 |
| G1T4.K218C-IgG1 | 17 | 455 | 19 | 20 |
| G1T4.S219C-IgG1 | 17 | 456 | 19 | 20 |

TABLE 33

| | Heavy | Heavy | Light | Light |
|---|---|---|---|---|
| | chain | chain | chain | chain |
| | variable | constant | variable | constant |
| | region | region | region | region |
| MRA-IgG1 light chain | SEQ ID | SEQ ID | SEQ ID | SEQ ID |
| variant name | NO: | NO: | NO: | NO: |
| MRAL.T5C-IgG1 | 17 | 18 | 457 | 20 |
| MRAL.Q6C-IgG1 | 17 | 18 | 458 | 20 |
| MRAL.S7C-IgG1 | 17 | 18 | 459 | 20 |
| MRAL.P8C-IgG1 | 17 | 18 | 460 | 20 |
| MRAL.S9C-IgG1 | 17 | 18 | 461 | 20 |
| MRAL.S10C-IgG1 | 17 | 18 | 462 | 20 |
| MRAL.L11C-IgG1 | 17 | 18 | 463 | 20 |
| MRAL.S12C-IgG1 | 17 | 18 | 464 | 20 |
| MRAL.A13C-IgG1 | 17 | 18 | 465 | 20 |
| MRAL.S14C-IgG1 | 17 | 18 | 466 | 20 |
| MRAL.V15C-IgG1 | 17 | 18 | 467 | 20 |
| MRAL.G16C-IgG1 | 17 | 18 | 468 | 20 |
| MRAL.D17C-IgG1 | 17 | 18 | 469 | 20 |
| MRAL.R18C-IgG1 | 17 | 18 | 470 | 20 |
| MRAL.V19C-IgG1 | 17 | 18 | 471 | 20 |
| MRAL.T20C-IgG1 | 17 | 18 | 472 | 20 |
| MRAL.I21C-IgG1 | 17 | 18 | 473 | 20 |
| MRAL.T22C-IgG1 | 17 | 18 | 474 | 20 |
| MRAL.A25C-IgG1 | 17 | 18 | 475 | 20 |
| MRAL.S26C-IgG1 | 17 | 18 | 476 | 20 |
| MRAL.Q27C-IgG1 | 17 | 18 | 477 | 20 |
| MRAL.Y32C-IgG1 | 17 | 18 | 478 | 20 |
| MRAL.L33C-IgG1 | 17 | 18 | 479 | 20 |
| MRAL.N34C-IgG1 | 17 | 18 | 480 | 20 |
| MRAL.Y50C-IgG1 | 17 | 18 | 481 | 20 |
| MRAL.T51C-IgG1 | 17 | 18 | 482 | 20 |
| MRAL.H55C-IgG1 | 17 | 18 | 483 | 20 |
| MRAL.S56C-IgG1 | 17 | 18 | 484 | 20 |
| MRAL.G57C-IgG1 | 17 | 18 | 485 | 20 |
| MRAL.V58C-IgG1 | 17 | 18 | 486 | 20 |
| MRAL.P59C-IgG1 | 17 | 18 | 487 | 20 |
| MRAL.S60C-IgG1 | 17 | 18 | 488 | 20 |
| MRAL.R61C-IgG1 | 17 | 18 | 489 | 20 |
| MRAL.F62C-IgG1 | 17 | 18 | 490 | 20 |
| MRAL.S63C-IgG1 | 17 | 18 | 491 | 20 |
| MRAL.S65C-IgG1 | 17 | 18 | 492 | 20 |
| MRAL.S67C-IgG1 | 17 | 18 | 493 | 20 |
| MRAL.G68C-IgG1 | 17 | 18 | 494 | 20 |
| MRAL.T69C-IgG1 | 17 | 18 | 495 | 20 |
| MRAL.D70C-IgG1 | 17 | 18 | 496 | 20 |
| MRAL.T72C-IgG1 | 17 | 18 | 497 | 20 |
| MRAL.F73C-IgG1 | 17 | 18 | 498 | 20 |
| MRAL.T74C-IgG1 | 17 | 18 | 499 | 20 |
| MRAL.I75C-IgG1 | 17 | 18 | 500 | 20 |
| MRAL.S76C-IgG1 | 17 | 18 | 501 | 20 |
| MRAL.S77C-IgG1 | 17 | 18 | 502 | 20 |
| MRAL.L78C-IgG1 | 17 | 18 | 503 | 20 |
| MRAL.Q79C-IgG1 | 17 | 18 | 504 | 20 |
| MRAL.Y96C-IgG1 | 17 | 18 | 505 | 20 |
| MRAL.T97C-IgG1 | 17 | 18 | 506 | 20 |
| MRAL.F98C-IgG1 | 17 | 18 | 507 | 20 |
| MRAL.G99C-IgG1 | 17 | 18 | 508 | 20 |
| MRAL.Q100C-IgG1 | 17 | 18 | 509 | 20 |
| MRAL.G101C-IgG1 | 17 | 18 | 510 | 20 |
| MRAL.T102C-IgG1 | 17 | 18 | 511 | 20 |
| MRAL.K103C-IgG1 | 17 | 18 | 512 | 20 |
| MRAL.V104C-IgG1 | 17 | 18 | 513 | 20 |
| MRAL.E105C-IgG1 | 17 | 18 | 514 | 20 |
| MRAL.I106C-IgG1 | 17 | 18 | 515 | 20 |
| MRAL.K107C-IgG1 | 17 | 18 | 516 | 20 |
| k0.R108C-IgG1 | 17 | 18 | 19 | 517 |
| k0.T109C-IgG1 | 17 | 18 | 19 | 518 |
| k0.V110C-IgG1 | 17 | 18 | 19 | 519 |
| k0.A111C-IgG1 | 17 | 18 | 19 | 520 |
| k0.A112C-IgG1 | 17 | 18 | 19 | 521 |
| k0.P113C-IgG1 | 17 | 18 | 19 | 522 |
| k0.S114C-IgG1 | 17 | 18 | 19 | 523 |
| k0.V115C-IgG1 | 17 | 18 | 19 | 524 |
| k0.F116C-IgG1 | 17 | 18 | 19 | 525 |
| k0.P120C-IgG1 | 17 | 18 | 19 | 526 |
| k0.S121C-IgG1 | 17 | 18 | 19 | 527 |
| k0.D122C-IgG1 | 17 | 18 | 19 | 528 |
| k0.E123C-IgG1 | 17 | 18 | 19 | 529 |
| k0.Q124C-IgG1 | 17 | 18 | 19 | 530 |
| k0.L125C-IgG1 | 17 | 18 | 19 | 531 |
| k0.K126C-IgG1 | 17 | 18 | 19 | 532 |
| k0.S127C-IgG1 | 17 | 18 | 19 | 533 |
| k0.G128C-IgG1 | 17 | 18 | 19 | 534 |
| k0.T129C-IgG1 | 17 | 18 | 19 | 535 |
| k0.A130C-IgG1 | 17 | 18 | 19 | 536 |
| k0.S131C-IgG1 | 17 | 18 | 19 | 537 |
| k0.L136C-IgG1 | 17 | 18 | 19 | 538 |
| k0.N137C-IgG1 | 17 | 18 | 19 | 539 |
| k0.N138C-IgG1 | 17 | 18 | 19 | 540 |
| k0.F139C-IgG1 | 17 | 18 | 19 | 541 |
| k0.Y140C-IgG1 | 17 | 18 | 19 | 542 |
| k0.P141C-IgG1 | 17 | 18 | 19 | 543 |
| k0.R142C-IgG1 | 17 | 18 | 19 | 544 |
| k0.E143C-IgG1 | 17 | 18 | 19 | 545 |
| k0.A144C-IgG1 | 17 | 18 | 19 | 546 |
| k0.K145C-IgG1 | 17 | 18 | 19 | 547 |
| k0.V146C-IgG1 | 17 | 18 | 19 | 548 |
| k0.Q147C-IgG1 | 17 | 18 | 19 | 549 |
| k0.W148C-IgG1 | 17 | 18 | 19 | 550 |
| k0.K149C-IgG1 | 17 | 18 | 19 | 551 |
| k0.V150C-IgG1 | 17 | 18 | 19 | 552 |
| k0.D151C-IgG1 | 17 | 18 | 19 | 553 |
| k0.N152C-IgG1 | 17 | 18 | 19 | 554 |
| k0.A153C-IgG1 | 17 | 18 | 19 | 555 |
| k0.L154C-IgG1 | 17 | 18 | 19 | 556 |
| k0.Q155C-IgG1 | 17 | 18 | 19 | 557 |
| k0.S156C-IgG1 | 17 | 18 | 19 | 558 |
| k0.G157C-IgG1 | 17 | 18 | 19 | 559 |
| k0.N158C-IgG1 | 17 | 18 | 19 | 560 |
| k0.S159C-IgG1 | 17 | 18 | 19 | 561 |
| k0.Q160C-IgG1 | 17 | 18 | 19 | 562 |
| k0.E161C-IgG1 | 17 | 18 | 19 | 563 |
| k0.S162C-IgG1 | 17 | 18 | 19 | 564 |
| k0.V163C-IgG1 | 17 | 18 | 19 | 565 |
| k0.T164C-IgG1 | 17 | 18 | 19 | 566 |
| k0.E165C-IgG1 | 17 | 18 | 19 | 567 |
| k0.Q166C-IgG1 | 17 | 18 | 19 | 568 |
| k0.D167C-IgG1 | 17 | 18 | 19 | 569 |
| k0.S168C-IgG1 | 17 | 18 | 19 | 570 |
| k0.K169C-IgG1 | 17 | 18 | 19 | 571 |
| k0.D170C-IgG1 | 17 | 18 | 19 | 572 |
| k0.S171C-IgG1 | 17 | 18 | 19 | 573 |
| k0.T172C-IgG1 | 17 | 18 | 19 | 574 |
| k0.Y173C-IgG1 | 17 | 18 | 19 | 575 |
| k0.S174C-IgG1 | 17 | 18 | 19 | 576 |
| k0.L175C-IgG1 | 17 | 18 | 19 | 577 |
| k0.T180C-IgG1 | 17 | 18 | 19 | 578 |
| k0.L181C-IgG1 | 17 | 18 | 19 | 579 |
| k0.S182C-IgG1 | 17 | 18 | 19 | 580 |
| k0.K183C-IgG1 | 17 | 18 | 19 | 581 |
| k0.A184C-IgG1 | 17 | 18 | 19 | 582 |
| k0.D185C-IgG1 | 17 | 18 | 19 | 583 |
| k0.Y186C-IgG1 | 17 | 18 | 19 | 584 |
| k0.E187C-IgG1 | 17 | 18 | 19 | 585 |
| k0.K188C-IgG1 | 17 | 18 | 19 | 586 |
| k0.H189C-IgG1 | 17 | 18 | 19 | 587 |
| k0.K190C-IgG1 | 17 | 18 | 19 | 588 |
| k0.V191C-IgG1 | 17 | 18 | 19 | 589 |
| k0.Y192C-IgG1 | 17 | 18 | 19 | 590 |
| k0.A193C-IgG1 | 17 | 18 | 19 | 591 |
| k0.E195C-IgG1 | 17 | 18 | 19 | 592 |
| k0.V196C-IgG1 | 17 | 18 | 19 | 593 |
| k0.T197C-IgG1 | 17 | 18 | 19 | 594 |
| k0.H198C-IgG1 | 17 | 18 | 19 | 595 |
| k0.Q199C-IgG1 | 17 | 18 | 19 | 596 |
| k0.G200C-IgG1 | 17 | 18 | 19 | 597 |
| k0.L201C-IgG1 | 17 | 18 | 19 | 598 |
| k0.S202C-IgG1 | 17 | 18 | 19 | 599 |
| k0.S203C-IgG1 | 17 | 18 | 19 | 600 |
| k0.P204C-IgG1 | 17 | 18 | 19 | 601 |
| k0.V205C-IgG1 | 17 | 18 | 19 | 602 |
| k0.T206C-IgG1 | 17 | 18 | 19 | 603 |
| k0.K207C-IgG1 | 17 | 18 | 19 | 604 |
| k0.S208C-IgG1 | 17 | 18 | 19 | 605 |
| k0.F209C-IgG1 | 17 | 18 | 19 | 606 |
| k0.N210C-IgG1 | 17 | 18 | 19 | 607 |
| k0.R211C-IgG1 | 17 | 18 | 19 | 608 |
| k0.G212C-IgG1 | 17 | 18 | 19 | 609 |
| k0.E213C-IgG1 | 17 | 18 | 19 | 610 |

### Reference Example 8-2 Assessment of Electrophoretic Mobility in Polyacrylamide Gel of Antibodies Having Cysteine Substitution at Various Positions of IgG1

It was examined with non-reducing SDS-PAGE whether the MRA-IgG1 variants produced in Reference Example 8-1 show a different electrophoretic mobility to MRA-IgG1. Sample Buffer Solution (2ME-) (×4) (Wako; 198-13282) was used for preparing electrophoresis samples, the samples were treated for 10 minutes under the condition of specimen concentration 50 microgram/mL and 70 degrees C., and then subjected to non-reducing SDS-PAGE. In non-reducing SDS-PAGE, electrophoresis was carried out for 90 minutes at 125 V, using 4% SDS-PAGE mini 15 well 1.0 mm 15 well (TEFCO; Cat #01-052-6). Then, the gel was stained with CBB stain, the gel image was captured with ChemiDocTouchMP (BIORAD), and the bands were quantified with Image Lab (BIORAD).

From the obtained gel image, the variants were classified into 7 groups according to the band pattern of each of the MRA-IgG1 variants: Single (one band at a molecular weight region similar to that of MRA-IgG1), Double (two bands at a molecular weight region similar to that of MRA-IgG1), Triple (three bands at a molecular weight region similar to that of MRA-IgG1), Several (four or more bands at a molecular weight region similar to that of MRA-IgG1), LMW (band(s) at a molecular weight region lower than that of MRA-IgG1), HMW (band(s) at a molecular weight region higher than that of MRA-IgG1), and Faint (band(s) blurry and difficult to determine). Regarding the MRA-IgG1 variants classified as “Double”, one of the two bands showed the same electrophoretic mobility as MRA-IgG1 while the other band showed slightly faster or slower mobility. Thus, for the MRA-IgG1 variants classified as “Double”, the percentage of the bands showing different mobility to MRA-IgG1 (percentage of new band (%)) was also calculated. Grouping of the band patterns for MRA-IgG1 heavy chain variants and MRA-IgG1 light chain variants, and the calculation results of the band percentage are respectively shown in Tables 34 and 35. From Tables 34 and 35, variants classified into the Double and Triple groups are shown in Table 36. In these variants, it is highly likely that cysteine substitution caused structural changes such as crosslinkage of Fabs, which resulted in the change in electrophoretic mobility. It is noted that while Table 35 indicates “no data” for MRAL.K107C-IgG1, position 107 (Kabat numbering), which is the position of cysteine substitution in this variant, is a position where the residue structurally exposed to the surface is present in the hinge region. Thus, in this variant also, it is highly likely that cysteine substitution causes structural changes such as crosslinkage of Fabs, and results in the change in electrophoretic mobility.

TABLE 34

| MRA-IgG1 heavy chain | | Percentage of new |
|---|---|---|
| variant name | Group | band (%) |
| MRAH.Q5C-IgG1 | Single | — |
| MRAH.E6C-IgG1 | Double | 30.7 |
| MRAH.S7C-IgG1 | Single | — |
| MRAH.G8C-IgG1 | Single | — |
| MRAH.P9C-IgG1 | Single | — |
| MRAH.G10C-IgG1 | Single | — |
| MRAH.L11C-IgG1 | Single | — |
| MRAH.V12C-IgG1 | Single | — |
| MRAH.R13C-IgG1 | Single | — |
| MRAH.P14C-IgG1 | Single | — |
| MRAH.S15C-IgG1 | Single | — |
| MRAH.Q16C-IgG1 | Single | — |
| MRAH.T17C-IgG1 | Single | — |
| MRAH.L18C-IgG1 | Faint | — |
| MRAH.S19C-IgG1 | Single | — |
| MRAH.L20C-IgG1 | Faint | — |
| MRAH.T21C-IgG1 | Single | — |
| MRAH.T23C-IgG1 | no data | — |
| MRAH.S25C-IgG1 | Double | 20.2 |
| MRAH.G26C-IgG1 | Double | 14.5 |
| MRAH.S28C-IgG1 | Single | — |
| MRAH.T30C-IgG1 | Single | — |
| MRAH.S31C-IgG1 | Single | — |
| MRAH.W35C-IgG1 | Faint | — |
| MRAH.S35aC-IgG1 | Faint | — |
| MRAH.Y50C-IgG1 | Single | — |
| MRAH.I51C-IgG1 | Faint | — |
| MRAH.S52C-IgG1 | Single | — |
| MRAH.S62C-IgG1 | Single | — |
| MRAH.L63C-IgG1 | Single | — |
| MRAH.K64C-IgG1 | Single | — |
| MRAH.S65C-IgG1 | Single | — |
| MRAH.R66C-IgG1 | Single | — |
| MRAH.V67C-IgG1 | Single | — |
| MRAH.T68C-IgG1 | Single | — |
| MRAH.L70C-IgG1 | no data | — |
| MRAH.D72C-IgG1 | Single | — |
| MRAH.T73C-IgG1 | Single | — |
| MRAH.S74C-IgG1 | Single | — |
| MRAH.K75C-IgG1 | Single | — |
| MRAH.N76C-IgG1 | Single | — |
| MRAH.Q77C-IgG1 | Single | — |
| MRAH.S79C-IgG1 | Single | — |
| MRAH.L80C-IgG1 | Faint | — |
| MRAH.R81C-IgG1 | Single | — |
| MRAH.L82C-IgG1 | Faint | — |
| MRAH.S82aC-IgG1 | Single | — |
| MRAH.S82bC-IgG1 | Single | — |
| MRAH.V82cC-IgG1 | Faint | — |
| MRAH.D101C-IgG1 | Single | — |
| MRAH.Y102C-IgG1 | Single | — |
| MRAH.S112C-IgG1 | Single | — |
| MRAH.S113C-IgG1 | Single | — |
| G1T4.A118C-IgG1 | Single | — |
| G1T4.S119C-IgG1 | Double | 18.4 |
| G1T4.T120C-IgG1 | Single | — |
| G1T4.K121C-IgG1 | Single | — |
| G1T4.G122C-IgG1 | Single | — |
| G1T4.P123C-IgG1 | LMW | — |
| G1T4.S124C-IgG1 | Single | — |
| G1T4.V125C-IgG1 | LMW | — |
| G1T4.F126C-IgG1 | Single | — |
| G1T4.P127C-IgG1 | LMW | — |
| G1T4.S131C-IgG1 | Triple | — |
| G1T4.S132C-IgG1 | Triple | — |
| G1T4.K133C-IgG1 | Triple | — |
| G1T4.S134C-IgG1 | Triple | — |
| G1T4.T135C-IgG1 | Triple | — |
| G1T4.S136C-IgG1 | Triple | — |
| G1T4.G137C-IgG1 | Triple | — |
| G1T4.G138C-IgG1 | Double | 56.7 |
| G1T4.T139C-IgG1 | Single | — |
| G1T4.A140C-IgG1 | Single | — |
| G1T4.A141C-IgG1 | Faint | — |
| G1T4.D148C-IgG1 | Single | — |
| G1T4.Y149C-IgG1 | Faint | — |
| G1T4.F150C-IgG1 | Single | — |
| G1T4.P151C-IgG1 | Faint | — |
| G1T4.E152C-IgG1 | Single | — |
| G1T4.P153C-IgG1 | Single | — |
| G1T4.V154C-IgG1 | LMW | — |
| G1T4.T155C-IgG1 | Single | — |
| G1T4.V156C-IgG1 | LMW | — |
| G1T4.S157C-IgG1 | Single | — |
| G1T4.W158C-IgG1 | LMW | — |
| G1T4.N159C-IgG1 | Double | 24 |
| G1T4.S160C-IgG1 | Double | 35.7 |
| G1T4.G161C-IgG1 | Double | 27.2 |
| G1T4.A162C-IgG1 | Double | 27.8 |
| G1T4.L163C-IgG1 | Double | 16.7 |
| G1T4.T164C-IgG1 | Double | 13.8 |
| G1T4.S165C-IgG1 | Single | — |
| G1T4.G166C-IgG1 | Single | — |
| G1T4.V167C-IgG1 | Single | — |
| G1T4.V173C-IgG1 | Single | — |
| G1T4.L174C-IgG1 | Single | — |
| G1T4.Q175C-IgG1 | Single | — |
| G1T4.S176C-IgG1 | Single | — |
| G1T4.S177C-IgG1 | Single | — |
| G1T4.G178C-IgG1 | Single | — |
| G1T4.L179C-IgG1 | Single | — |
| G1T4.Y180C-IgG1 | LMW | — |
| G1T4.V186C-IgG1 | LMW | — |
| G1T4.T187C-IgG1 | Single | — |
| G1T4.V188C-IgG1 | LMW | — |
| G1T4.P189C-IgG1 | no data | — |
| G1T4.S190C-IgG1 | Double | 31.8 |
| G1T4.S191C-IgG1 | Double | 66.3 |
| G1T4.S192C-IgG1 | Double | 26.8 |
| G1T4.L193C-IgG1 | LMW | — |
| G1T4.G194C-IgG1 | Faint | — |
| G1T4.T195C-IgG1 | Double | 78.1 |
| G1T4.Q196C-IgG1 | Double | 27.4 |
| G1T4.T197C-IgG1 | Double | 84.4 |
| G1T4.Y198C-IgG1 | Faint | — |
| G1T4.I199C-IgG1 | Single | — |
| G1T4.N201C-IgG1 | Double | 17.5 |
| G1T4.V202C-IgG1 | LMW | — |
| G1T4.N203C-IgG1 | Double | 17.2 |
| G1T4.H204C-IgG1 | Faint | — |
| G1T4.K205C-IgG1 | Double | 18.4 |
| G1T4.P206C-IgG1 | Double | 14.4 |
| G1T4.S207C-IgG1 | Double | 21.5 |
| G1T4.N208C-IgG1 | Double | 16.1 |
| G1T4.T209C-IgG1 | Single | — |
| G1T4.K210C-IgG1 | Single | — |
| G1T4.V211C-IgG1 | Double | 27.2 |
| G1T4.D212C-IgG1 | Double | 28.2 |
| G1T4.K213C-IgG1 | LMW | — |
| G1T4.R214C-IgG1 | LMW | — |
| G1T4.V215C-IgG1 | LMW | — |
| G1T4.E216C-IgG1 | LMW | — |
| G1T4.P217C-IgG1 | LMW | — |
| G1T4.K218C-IgG1 | Double | 39.3 |
| G1T4.S219C-IgG1 | Double | 68.7 |

TABLE 35

| MRA-IgG1 light chain | | Percentage of new |
|---|---|---|
| variant name | Group | band (%) |
| MRAL.T5C-IgG1 | Single | — |
| MRAL.Q6C-IgG1 | LMW | — |
| MRAL.S7C-IgG1 | Single | — |
| MRAL.P8C-IgG1 | no data | — |
| MRAL.S9C-IgG1 | Single | — |
| MRAL.S10C-IgG1 | Single | — |
| MRAL.L11C-IgG1 | Single | — |
| MRAL.S12C-IgG1 | Single | — |
| MRAL.A13C-IgG1 | Single | — |
| MRAL.S14C-IgG1 | Single | — |
| MRAL.V15C-IgG1 | Single | — |
| MRAL.G16C-IgG1 | Single | — |
| MRAL.D17C-IgG1 | Single | — |
| MRAL.R18C-IgG1 | Single | — |
| MRAL.V19C-IgG1 | LMW | — |
| MRAL.T20C-IgG1 | Single | — |
| MRAL.I21C-IgG1 | Double | 68.9 |
| MRAL.T22C-IgG1 | Single | — |
| MRAL.A25C-IgG1 | no data | — |
| MRAL.S26C-IgG1 | no data | — |
| MRAL.Q27C-IgG1 | Triple | — |
| MRAL.Y32C-IgG1 | Single | — |
| MRAL.L33C-IgG1 | LMW | — |
| MRAL.N34C-IgG1 | LMW | — |
| MRAL.Y50C-IgG1 | Single | — |
| MRAL.T51C-IgG1 | Single | — |
| MRAL.H55C-IgG1 | Single | — |
| MRAL.S56C-IgG1 | Single | — |
| MRAL.G57C-IgG1 | Single | — |
| MRAL.V58C-IgG1 | Double | 17.4 |
| MRAL.P59C-IgG1 | Single | — |
| MRAL.S60C-IgG1 | Single | — |
| MRAL.R61C-IgG1 | Single | — |
| MRAL.F62C-IgG1 | LMW | — |
| MRAL.S63C-IgG1 | Single | — |
| MRAL.S65C-IgG1 | Single | — |
| MRAL.S67C-IgG1 | Single | — |
| MRAL.G68C-IgG1 | Single | — |
| MRAL.T69C-IgG1 | Single | — |
| MRAL.D70C-IgG1 | Single | — |
| MRAL.T72C-IgG1 | Single | — |
| MRAL.F73C-IgG1 | LMW | — |
| MRAL.T74C-IgG1 | Single | — |
| MRAL.I75C-IgG1 | no data | — |
| MRAL.S76C-IgG1 | Single | — |
| MRAL.S77C-IgG1 | Double | 18.1 |
| MRAL.L78C-IgG1 | LMW | — |
| MRAL.Q79C-IgG1 | Single | — |
| MRAL.Y96C-IgG1 | LMW | — |
| MRAL.T97C-IgG1 | Single | — |
| MRAL.F98C-IgG1 | LMW | — |
| MRAL.G99C-IgG1 | no data | — |
| MRAL.Q100C-IgG1 | Single | — |
| MRAL.G101C-IgG1 | Single | — |
| MRAL.T102C-IgG1 | LMW | — |
| MRAL.K103C-IgG1 | Single | — |
| MRAL.V104C-IgG1 | LMW | — |
| MRAL.E105C-IgG1 | no data | — |
| MRAL.I106C-IgG1 | Single | — |
| MRAL.K107C-IgG1 | no data | — |
| k0.R108C-IgG1 | Single | — |
| k0.T109C-IgG1 | Double | 23.1 |
| k0.V110C-IgG1 | Single | — |
| k0.A111C-IgG1 | Single | — |
| k0.A112C-IgG1 | Double | 21.6 |
| k0.P113C-IgG1 | Single | — |
| k0.S114C-IgG1 | Single | — |
| k0.V115C-IgG1 | LMW | — |
| k0.F116C-IgG1 | Single | — |
| k0.P120C-IgG1 | LMW | — |
| k0.S121C-IgG1 | Several | — |
| k0.D122C-IgG1 | LMW | — |
| k0.E123C-IgG1 | Double | 18.1 |
| k0.Q124C-IgG1 | LMW | — |
| k0.L125C-IgG1 | LMW | — |
| k0.K126C-IgG1 | Triple | — |
| k0.S127C-IgG1 | Single | — |
| k0.G128C-IgG1 | Double | 19.4 |
| k0.T129C-IgG1 | Single | — |
| k0.A130C-IgG1 | LMW | — |
| k0.S131C-IgG1 | LMW | — |
| k0.L136C-IgG1 | LMW | — |
| k0.N137C-IgG1 | LMW | — |
| k0.N138C-IgG1 | Single | — |
| k0.F139C-IgG1 | LMW | — |
| k0.Y140C-IgG1 | LMW | — |
| k0.P141C-IgG1 | Single | — |
| k0.R142C-IgG1 | Single | — |
| k0.E143C-IgG1 | Single | — |
| k0.A144C-IgG1 | LMW | — |
| k0.K145C-IgG1 | Single | — |
| k0.V146C-IgG1 | LMW | — |
| k0.Q147C-IgG1 | Single | — |
| k0.W148C-IgG1 | LMW | — |
| k0.K149C-IgG1 | Single | — |
| k0.V150C-IgG1 | LMW | — |
| k0.D151C-IgG1 | Single | — |
| k0.N152C-IgG1 | Double | 62.4 |
| k0.A153C-IgG1 | Single | — |
| k0.L154C-IgG1 | Single | — |
| k0.Q155C-IgG1 | Single | — |
| k0.S156C-IgG1 | HMW | — |
| k0.G157C-IgG1 | Single | — |
| k0.N158C-IgG1 | Single | — |
| k0.S159C-IgG1 | Single | — |
| k0.Q160C-IgG1 | Single | — |
| k0.E161C-IgG1 | Single | — |
| k0.S162C-IgG1 | Single | — |
| k0.V163C-IgG1 | Single | — |
| k0.T164C-IgG1 | Single | — |
| k0.E165C-IgG1 | Single | — |
| k0.Q166C-IgG1 | Single | — |
| k0.D167C-IgG1 | Single | — |
| k0.S168C-IgG1 | Single | — |
| k0.K169C-IgG1 | Single | — |
| k0.D170C-IgG1 | Single | — |
| k0.S171C-IgG1 | Single | — |
| k0.T172C-IgG1 | LMW | — |
| k0.Y173C-IgG1 | LMW | — |
| k0.S174C-IgG1 | Single | — |
| k0.L175C-IgG1 | LMW | — |
| k0.T180C-IgG1 | Single | — |
| k0.L181C-IgG1 | Single | — |
| k0.S182C-IgG1 | Single | — |
| k0.K183C-IgG1 | Single | — |
| k0.A184C-IgG1 | Single | — |
| k0.D185C-IgG1 | Single | — |
| k0.Y186C-IgG1 | Double | 26.3 |
| k0.E187C-IgG1 | LMW | — |
| k0.K188C-IgG1 | Single | — |
| k0.H189C-IgG1 | Triple | — |
| k0.K190C-IgG1 | LMW | — |
| k0.V191C-IgG1 | LMW | — |
| k0.Y192C-IgG1 | Single | — |
| k0.A193C-IgG1 | Single | — |
| k0.E195C-IgG1 | Single | — |
| k0.V196C-IgG1 | Single | — |
| k0.T197C-IgG1 | Single | — |
| k0.H198C-IgG1 | Faint | — |
| k0.Q199C-IgG1 | Single | — |
| k0.G200C-IgG1 | Double | 18.7 |
| k0.L201C-IgG1 | Single | — |
| k0.S202C-IgG1 | Double | 42.3 |
| k0.S203C-IgG1 | Double | 45.5 |
| k0.P204C-IgG1 | Single | — |
| k0.V205C-IgG1 | Single | — |
| k0.T206C-IgG1 | Single | — |
| k0.K207C-IgG1 | Single | — |
| k0.S208C-IgG1 | Single | — |
| k0.F209C-IgG1 | LMW | — |
| k0.N210C-IgG1 | LMW | — |
| k0.R211C-IgG1 | Single | — |
| k0.G212C-IgG1 | Double | 68.5 |
| k0.E213C-IgG1 | LMW | — |

TABLE 36

| MRA-IgG1 variant | | Percentage of new |
|---|---|---|
| name | Group | band (%) |
| MRAH.E6C-IgG1 | Double | 30.7 |
| MRAH.S25C-IgG1 | Double | 20.2 |
| MRAH.G26C-IgG1 | Double | 14.5 |
| G1T4.S119C-IgG1 | Double | 18.4 |
| G1T4.S131C-IgG1 | Triple | — |
| G1T4.S132C-IgG1 | Triple | — |
| G1T4.K133C-IgG1 | Triple | — |
| G1T4.S134C-IgG1 | Triple | — |
| G1T4.T135C-IgG1 | Triple | — |
| G1T4.S136C-IgG1 | Triple | — |
| G1T4.G137C-IgG1 | Triple | — |
| G1T4.G138C-IgG1 | Double | 56.7 |
| G1T4.N159C-IgG1 | Double | 24 |
| G1T4.S160C-IgG1 | Double | 35.7 |
| G1T4.G161C-IgG1 | Double | 27.2 |
| G1T4.A162C-IgG1 | Double | 27.8 |
| G1T4.L163C-IgG1 | Double | 16.7 |
| G1T4.T164C-IgG1 | Double | 13.8 |
| G1T4.S190C-IgG1 | Double | 31.8 |
| G1T4.S191C-IgG1 | Double | 66.3 |
| G1T4.S192C-IgG1 | Double | 26.8 |
| G1T4.T195C-IgG1 | Double | 78.1 |
| G1T4.Q196C-IgG1 | Double | 27.4 |
| G1T4.T197C-IgG1 | Double | 84.4 |
| G1T4.N201C-IgG1 | Double | 17.5 |
| G1T4.N203C-IgG1 | Double | 17.2 |
| G1T4.K205C-IgG1 | Double | 18.4 |
| G1T4.P206C-IgG1 | Double | 14.4 |
| G1T4.S207C-IgG1 | Double | 21.5 |
| G1T4.N208C-IgG1 | Double | 16.1 |
| G1T4.V211C-IgG1 | Double | 27.2 |
| G1T4.D212C-IgG1 | Double | 28.2 |
| G1T4.K218C-IgG1 | Double | 39.3 |
| G1T4.S219C-IgG1 | Double | 68.7 |
| MRAL.I21C-IgG1 | Double | 68.9 |
| MRAL.Q27C-IgG1 | Triple | — |
| MRAL.V58C-IgG1 | Double | 17.4 |
| MRAL.S77C-IgG1 | Double | 18.1 |
| k0.T109C-IgG1 | Double | 23.1 |
| k0.A112C-IgG1 | Double | 21.6 |
| k0.E123C-IgG1 | Double | 18.1 |
| k0.K126C-IgG1 | Triple | — |
| k0.G128C-IgG1 | Double | 19.4 |
| k0.N152C-IgG1 | Double | 62.4 |
| k0.Y186C-IgG1 | Double | 26.3 |
| k0.H189C-IgG1 | Triple | — |
| k0.G200C-IgG1 | Double | 18.7 |
| k0.S202C-IgG1 | Double | 42.3 |
| k0.S203C-IgG1 | Double | 45.5 |
| k0.G212C-IgG1 | Double | 68.5 |

### Reference Example 9 Assessment of Antibodies Having Cysteine Substitution at Various Positions of IgG4

### Reference Example 9-1 Production of Antibodies Having Cysteine Substitution at Various Positions of IgG4

The heavy chain and light chain of an anti-human IL6R neutralizing antibody, MRA-IgG4 (heavy chain: MRAH-G4T1 (SEQ ID NO: 310), light chain: MRAL-k0 (SEQ ID NO: 16)), were subjected to a study in which an arbitrary amino acid residue structurally exposed to the surface was substituted with cysteine.

Amino acid residues within the MRA-IgG4 heavy chain variable region (MRAH, SEQ ID NO: 17) were substituted with cysteine to produce variants of the MRA-IgG4 heavy chain variable region shown in Table 37. These variants of the MRA-IgG4 heavy chain variable region were each linked with the MRA-IgG4 heavy chain constant region (G4T1, SEQ ID NO: 311) to produce MRA-IgG4 heavy chain variants, and expression vectors encoding the corresponding genes were produced by a method known to the person skilled in the art. In addition, amino acid residues within the MRA-IgG4 heavy chain constant region (G4T1, SEQ ID NO: 311) were substituted with cysteine to produce variants of the MRA-IgG4 heavy chain constant region shown in Table 38. These variants of the MRA-IgG4 heavy chain constant region were each linked with the MRA-IgG4 heavy chain variable region (MRAH, SEQ ID NO: 17) to produce MRA-IgG4 heavy chain variants, and expression vectors encoding the corresponding genes were produced by a method known to the person skilled in the art.

TABLE 37

| Variant of MRA-IgG4 | Position of cysteine | |
|---|---|---|
| heavy chain variable | substitution | |
| region | (Rabat numbering) | SEQ ID NO: |
| MRAH.Q5C | 5 | 322 |
| MRAH.E6C | 6 | 323 |
| MRAH.S7C | 7 | 324 |
| MRAH.G8C | 8 | 325 |
| MRAH.P9C | 9 | 326 |
| MRAH.G10C | 10 | 327 |
| MRAH.L11C | 11 | 328 |
| MRAH.V12C | 12 | 329 |
| MRAH.R13C | 13 | 330 |
| MRAH.P14C | 14 | 331 |
| MRAH.S15C | 15 | 332 |
| MRAH.Q16C | 16 | 333 |
| MRAH.T17C | 17 | 334 |
| MRAH.L18C | 18 | 335 |
| MRAH.S19C | 19 | 336 |
| MRAH.L20C | 20 | 337 |
| MRAH.T21C | 21 | 338 |
| MRAH.T23C | 23 | 339 |
| MRAH.S25C | 25 | 340 |
| MRAH.G26C | 26 | 341 |
| MRAH.S28C | 28 | 342 |
| MRAH.T30C | 30 | 343 |
| MRAH.S31C | 31 | 344 |
| MRAH.W35C | 35 | 345 |
| MRAH.S35aC | 35a | 346 |
| MRAH.Y50C | 50 | 347 |
| MRAH.I51C | 51 | 348 |
| MRAH.S52C | 52 | 349 |
| MRAH.S62C | 62 | 350 |
| MRAH.L63C | 63 | 351 |
| MRAH.K64C | 64 | 352 |
| MRAH.S65C | 65 | 353 |
| MRAH.R66C | 66 | 354 |
| MRAH.V67C | 67 | 355 |
| MRAH.T68C | 68 | 356 |
| MRAH.L70C | 70 | 357 |
| MRAH.D72C | 72 | 358 |
| MRAH.T73C | 73 | 359 |
| MRAH.S74C | 74 | 360 |
| MRAH.K75C | 75 | 361 |
| MRAH.N76C | 76 | 362 |
| MRAH.Q77C | 77 | 363 |
| MRAH.S79C | 79 | 364 |
| MRAH.L80C | 80 | 365 |
| MRAH.R81C | 81 | 366 |
| MRAH.L82C | 82 | 367 |
| MRAH.S82aC | 82a | 368 |
| MRAH.S82bC | 82b | 369 |
| MRAH.V82cC | 82c | 370 |
| MRAH.D101C | 101 | 371 |
| MRAH.Y102C | 102 | 372 |
| MRAH.S112C | 112 | 373 |
| MRAH.S113C | 113 | 374 |

TABLE 38

| Variant of MRA-IgG4 | Position of cysteine | |
|---|---|---|
| heavy chain constant | substitution | |
| region | (EU numbering) | SEQ ID NO: |
| G4T1.A118C | 118 | 611 |
| G4T1.S119C | 119 | 612 |
| G4T1.T120C | 120 | 613 |
| G4T1.K121C | 121 | 614 |
| G4T1.G122C | 122 | 615 |
| G4T1.P123C | 123 | 616 |
| G4T1.S124C | 124 | 617 |
| G4T1.V125C | 125 | 618 |
| G4T1.F126C | 126 | 619 |
| G4T1.P127C | 127 | 620 |
| G4T1.S132C | 132 | 621 |
| G4T1.R133C | 133 | 622 |
| G4T1.S134C | 134 | 623 |
| G4T1.T135C | 135 | 624 |
| G4T1.S136C | 136 | 625 |
| G4T1.E137C | 137 | 626 |
| G4T1.S138C | 138 | 627 |
| G4T1.T139C | 139 | 628 |
| G4T1.A140C | 140 | 629 |
| G4T1.A141C | 141 | 630 |
| G4T1.D148C | 148 | 631 |
| G4T1.Y149C | 149 | 632 |
| G4T1.F150C | 150 | 633 |
| G4T1.P151C | 151 | 634 |
| G4T1.E152C | 152 | 635 |
| G4T1.P153C | 153 | 636 |
| G4T1.V154C | 154 | 637 |
| G4T1.T155C | 155 | 638 |
| G4T1.V156C | 156 | 639 |
| G4T1.S157C | 157 | 640 |
| G4T1.W158C | 158 | 641 |
| G4T1.N159C | 159 | 642 |
| G4T1.S160C | 160 | 643 |
| G4T1.G161C | 161 | 644 |
| G4T1.A162C | 162 | 645 |
| G4T1.L163C | 163 | 646 |
| G4T1.T164C | 164 | 647 |
| G4T1.S165C | 165 | 648 |
| G4T1.G166C | 166 | 649 |
| G4T1.V167C | 167 | 650 |
| G4T1.V173C | 173 | 651 |
| G4T1.L174C | 174 | 652 |
| G4T1.Q175C | 175 | 653 |
| G4T1.S176C | 176 | 654 |
| G4T1.S177C | 177 | 655 |
| G4T1.G178C | 178 | 656 |
| G4T1.L179C | 179 | 657 |
| G4T1.Y180C | 180 | 658 |
| G4T1.V186C | 186 | 659 |
| G4T1.T187C | 187 | 660 |
| G4T1.V188C | 188 | 661 |
| G4T1.P189C | 189 | 662 |
| G4T1.S190C | 190 | 663 |
| G4T1.S191C | 191 | 664 |
| G4T1.S192C | 192 | 665 |
| G4T1.L193C | 193 | 666 |
| G4T1.G194C | 194 | 667 |
| G4T1.T195C | 195 | 668 |
| G4T1.Q196C | 196 | 669 |
| G4T1.T197C | 197 | 670 |
| G4T1.Y198C | 198 | 671 |
| G4T1.T199C | 199 | 672 |
| G4T1.N201C | 201 | 673 |
| G4T1.V202C | 202 | 674 |
| G4T1.D203C | 203 | 675 |
| G4T1.H204C | 204 | 676 |
| G4T1.K205C | 205 | 677 |
| G4T1.P206C | 206 | 678 |
| G4T1.S207C | 207 | 679 |
| G4T1.N208C | 208 | 680 |
| G4T1.T209C | 209 | 681 |
| G4T1.K210C | 210 | 682 |
| G4T1.V211C | 211 | 683 |
| G4T1.D212C | 212 | 684 |
| G4T1.K213C | 213 | 685 |
| G4T1.R214C | 214 | 686 |
| G4T1.V215C | 215 | 687 |
| G4T1.E216C | 216 | 688 |
| G4T1.S217C | 217 | 689 |
| G4T1.K218C | 218 | 690 |

The MRA-IgG4 heavy chain variants produced above were combined with the MRA-IgG4 light chain, or the MRA-IgG4 heavy chain was combined with the MRA-IgG4 light chain variants produced in Reference Example 8-1. The resultant MRA-IgG4 heavy chain variants and MRA-IgG4 light chain variants shown in Tables 39 and 40 were expressed by transient expression using FreeStyle293 cells (Invitrogen) or Expi293 cells (Life technologies) by a method known to the person skilled in the art, and purified with Protein A by a method known to the person skilled in the art.

TABLE 39

| | Heavy | Heavy | Light | Light |
|---|---|---|---|---|
| | chain | chain | chain | chain |
| | variable | constant | variable | constant |
| | region | region | region | region |
| MRA-IgG4 heavy chain | SEQ ID | SEQ ID | SEQ ID | SEQ ID |
| variant name | NO: | NO: | NO: | NO: |
| MRAH.Q5C-IgG4 | 322 | 311 | 19 | 20 |
| MRAH.E6C-IgG4 | 323 | 311 | 19 | 20 |
| MRAH.S7C-IgG4 | 324 | 311 | 19 | 20 |
| MRAH.G8C-IgG4 | 325 | 311 | 19 | 20 |
| MRAH.P9C-IgG4 | 326 | 311 | 19 | 20 |
| MRAH.G10C-IgG4 | 327 | 311 | 19 | 20 |
| MRAH.L11C-IgG4 | 328 | 311 | 19 | 20 |
| MRAH.V12C-IgG4 | 329 | 311 | 19 | 20 |
| MRAH.R13C-IgG4 | 330 | 311 | 19 | 20 |
| MRAH.P14C-IgG4 | 331 | 311 | 19 | 20 |
| MRAH.S15C-IgG4 | 332 | 311 | 19 | 20 |
| MRAH.Q16C-IgG4 | 333 | 311 | 19 | 20 |
| MRAH.T17C-IgG4 | 334 | 311 | 19 | 20 |
| MRAH.L18C-IgG4 | 335 | 311 | 19 | 20 |
| MRAH.S19C-IgG4 | 336 | 311 | 19 | 20 |
| MRAH.L20C-IgG4 | 337 | 311 | 19 | 20 |
| MRAH.T21C-IgG4 | 338 | 311 | 19 | 20 |
| MRAH.T23C-IgG4 | 339 | 311 | 19 | 20 |
| MRAH.S25C-IgG4 | 340 | 311 | 19 | 20 |
| MRAH.G26C-IgG4 | 341 | 311 | 19 | 20 |
| MRAH.S28C-IgG4 | 342 | 311 | 19 | 20 |
| MRAH.T30C-IgG4 | 343 | 311 | 19 | 20 |
| MRAH.S31C-IgG4 | 344 | 311 | 19 | 20 |
| MRAH.W35C-IgG4 | 345 | 311 | 19 | 20 |
| MRAH.S35aC-IgG4 | 346 | 311 | 19 | 20 |
| MRAH.Y50C-IgG4 | 347 | 311 | 19 | 20 |
| MRAH.I51C-IgG4 | 348 | 311 | 19 | 20 |
| MRAH.S52C-IgG4 | 349 | 311 | 19 | 20 |
| MRAH.S62C-IgG4 | 350 | 311 | 19 | 20 |
| MRAH.L63C-IgG4 | 351 | 311 | 19 | 20 |
| MRAH.K64C-IgG4 | 352 | 311 | 19 | 20 |
| MRAH.S65C-IgG4 | 353 | 311 | 19 | 20 |
| MRAH.R66C-IgG4 | 354 | 311 | 19 | 20 |
| MRAH.V67C-IgG4 | 355 | 311 | 19 | 20 |
| MRAH.T68C-IgG4 | 356 | 311 | 19 | 20 |
| MRAH.L70C-IgG4 | 357 | 311 | 19 | 20 |
| MRAH.D72C-IgG4 | 358 | 311 | 19 | 20 |
| MRAH.T73C-IgG4 | 359 | 311 | 19 | 20 |
| MRAH.S74C-IgG4 | 360 | 311 | 19 | 20 |
| MRAH.K75C-IgG4 | 361 | 311 | 19 | 20 |
| MRAH.N76C-IgG4 | 362 | 311 | 19 | 20 |
| MRAH.Q77C-IgG4 | 363 | 311 | 19 | 20 |
| MRAH.S79C-IgG4 | 364 | 311 | 19 | 20 |
| MRAH.L80C-IgG4 | 365 | 311 | 19 | 20 |
| MRAH.R81C-IgG4 | 366 | 311 | 19 | 20 |
| MRAH.L82C-IgG4 | 367 | 311 | 19 | 20 |
| MRAH.S82aC-IgG4 | 368 | 311 | 19 | 20 |
| MRAH.S82bC-IgG4 | 369 | 311 | 19 | 20 |
| MRAH.V82cC-IgG4 | 370 | 311 | 19 | 20 |
| MRAH.D101C-IgG4 | 371 | 311 | 19 | 20 |
| MRAH.Y102C-IgG4 | 372 | 311 | 19 | 20 |
| MRAH.S112C-IgG4 | 373 | 311 | 19 | 20 |
| MRAH.S113C-IgG4 | 374 | 311 | 19 | 20 |
| G4T1.A118C-IgG4 | 17 | 611 | 19 | 20 |
| G4T1.S119C-IgG4 | 17 | 612 | 19 | 20 |
| G4T1.T120C-IgG4 | 17 | 613 | 19 | 20 |
| G4T1.K121C-IgG4 | 17 | 614 | 19 | 20 |
| G4T1.G122C-IgG4 | 17 | 615 | 19 | 20 |
| G4T1.P123C-IgG4 | 17 | 616 | 19 | 20 |
| G4T1.S124C-IgG4 | 17 | 617 | 19 | 20 |
| G4T1.V125C-IgG4 | 17 | 618 | 19 | 20 |
| G4T1.F126C-IgG4 | 17 | 619 | 19 | 20 |
| G4T1.P127C-IgG4 | 17 | 620 | 19 | 20 |
| G4T1.S132C-IgG4 | 17 | 621 | 19 | 20 |
| G4T1.R133C-IgG4 | 17 | 622 | 19 | 20 |
| G4T1.S134C-IgG4 | 17 | 623 | 19 | 20 |
| G4T1.T135C-IgG4 | 17 | 624 | 19 | 20 |
| G4T1.S136C-IgG4 | 17 | 625 | 19 | 20 |
| G4T1.E137C-IgG4 | 17 | 626 | 19 | 20 |
| G4T1.S138C-IgG4 | 17 | 627 | 19 | 20 |
| G4T1.T139C-IgG4 | 17 | 628 | 19 | 20 |
| G4T1.A140C-IgG4 | 17 | 629 | 19 | 20 |
| G4T1.A141C-IgG4 | 17 | 630 | 19 | 20 |
| G4T1.D148C-IgG4 | 17 | 631 | 19 | 20 |
| G4T1.Y149C-IgG4 | 17 | 632 | 19 | 20 |
| G4T1.F150C-IgG4 | 17 | 633 | 19 | 20 |
| G4T1.P151C-IgG4 | 17 | 634 | 19 | 20 |
| G4T1.E152C-IgG4 | 17 | 635 | 19 | 20 |
| G4T1.P153C-IgG4 | 17 | 636 | 19 | 20 |
| G4T1.V154C-IgG4 | 17 | 637 | 19 | 20 |
| G4T1.T155C-IgG4 | 17 | 638 | 19 | 20 |
| G4T1.V156C-IgG4 | 17 | 639 | 19 | 20 |
| G4T1.S157C-IgG4 | 17 | 640 | 19 | 20 |
| G4T1.W158C-IgG4 | 17 | 641 | 19 | 20 |
| G4T1.N159C-IgG4 | 17 | 642 | 19 | 20 |
| G4T1.S160C-IgG4 | 17 | 643 | 19 | 20 |
| G4T1.G161C-IgG4 | 17 | 644 | 19 | 20 |
| G4T1.A162C-IgG4 | 17 | 645 | 19 | 20 |
| G4T1.L163C-IgG4 | 17 | 646 | 19 | 20 |
| G4T1.T164C-IgG4 | 17 | 647 | 19 | 20 |
| G4T1.S165C-IgG4 | 17 | 648 | 19 | 20 |
| G4T1.G166C-IgG4 | 17 | 649 | 19 | 20 |
| G4T1.V167C-IgG4 | 17 | 650 | 19 | 20 |
| G4T1.V173C-IgG4 | 17 | 651 | 19 | 20 |
| G4T1.L174C-IgG4 | 17 | 652 | 19 | 20 |
| G4T1.Q175C-IgG4 | 17 | 653 | 19 | 20 |
| G4T1.S176C-IgG4 | 17 | 654 | 19 | 20 |
| G4T1.S177C-IgG4 | 17 | 655 | 19 | 20 |
| G4T1.G178C-IgG4 | 17 | 656 | 19 | 20 |
| G4T1.L179C-IgG4 | 17 | 657 | 19 | 20 |
| G4T1.Y180C-IgG4 | 17 | 658 | 19 | 20 |
| G4T1.V186C-IgG4 | 17 | 659 | 19 | 20 |
| G4T1.T187C-IgG4 | 17 | 660 | 19 | 20 |
| G4T1.V188C-IgG4 | 17 | 661 | 19 | 20 |
| G4T1.P189C-IgG4 | 17 | 662 | 19 | 20 |
| G4T1.S190C-IgG4 | 17 | 663 | 19 | 20 |
| G4T1.S191C-IgG4 | 17 | 664 | 19 | 20 |
| G4T1.S192C-IgG4 | 17 | 665 | 19 | 20 |
| G4T1.L193C-IgG4 | 17 | 666 | 19 | 20 |
| G4T1.G194C-IgG4 | 17 | 667 | 19 | 20 |
| G4T1.T195C-IgG4 | 17 | 668 | 19 | 20 |
| G4T1.Q196C-IgG4 | 17 | 669 | 19 | 20 |
| G4T1.T197C-IgG4 | 17 | 670 | 19 | 20 |
| G4T1.Y198C-IgG4 | 17 | 671 | 19 | 20 |
| G4T1.T199C-IgG4 | 17 | 672 | 19 | 20 |
| G4T1.N201C-IgG4 | 17 | 673 | 19 | 20 |
| G4T1.V202C-IgG4 | 17 | 674 | 19 | 20 |
| G4T1.D203C-IgG4 | 17 | 675 | 19 | 20 |
| G4T1.H204C-IgG4 | 17 | 676 | 19 | 20 |
| G4T1.K205C-IgG4 | 17 | 677 | 19 | 20 |
| G4T1.P206C-IgG4 | 17 | 678 | 19 | 20 |
| G4T1.S207C-IgG4 | 17 | 679 | 19 | 20 |
| G4T1.N208C-IgG4 | 17 | 680 | 19 | 20 |
| G4T1.T209C-IgG4 | 17 | 681 | 19 | 20 |
| G4T1.K210C-IgG4 | 17 | 682 | 19 | 20 |
| G4T1.V211C-IgG4 | 17 | 683 | 19 | 20 |
| G4T1.D212C-IgG4 | 17 | 684 | 19 | 20 |
| G4T1.K213C-IgG4 | 17 | 685 | 19 | 20 |
| G4T1.R214C-IgG4 | 17 | 686 | 19 | 20 |
| G4T1.V215C-IgG4 | 17 | 687 | 19 | 20 |
| G4T1.E216C-IgG4 | 17 | 688 | 19 | 20 |
| G4T1.S217C-IgG4 | 17 | 689 | 19 | 20 |
| G4T1.K218C-IgG4 | 17 | 690 | 19 | 20 |

TABLE 40

| | Heavy | Heavy | Light | Light |
|---|---|---|---|---|
| | chain | chain | chain | chain |
| | variable | constant | variable | constant |
| | region | region | region | region |
| MRA-IgG4 light chain | SEQ ID | SEQ ID | SEQ ID | SEQ ID |
| variant name | NO: | NO: | NO: | NO: |
| MRAL.T5C-IgG4 | 17 | 311 | 457 | 20 |
| MRAL.Q6C-IgG4 | 17 | 311 | 458 | 20 |
| MRAL.S7C-IgG4 | 17 | 311 | 459 | 20 |
| MRAL.P8C-IgG4 | 17 | 311 | 460 | 20 |
| MRAL.S9C-IgG4 | 17 | 311 | 461 | 20 |
| MRAL.S10C-IgG4 | 17 | 311 | 462 | 20 |
| MRAL.L11C-IgG4 | 17 | 311 | 463 | 20 |
| MRAL.S12C-IgG4 | 17 | 311 | 464 | 20 |
| MRAL.A13C-IgG4 | 17 | 311 | 465 | 20 |
| MRAL.S14C-IgG4 | 17 | 311 | 466 | 20 |
| MRAL.V15C-IgG4 | 17 | 311 | 467 | 20 |
| MRAL.G16C-IgG4 | 17 | 311 | 468 | 20 |
| MRAL.D17C-IgG4 | 17 | 311 | 469 | 20 |
| MRAL.R18C-IgG4 | 17 | 311 | 470 | 20 |
| MRAL.V19C-IgG4 | 17 | 311 | 471 | 20 |
| MRAL.T20C-IgG4 | 17 | 311 | 472 | 20 |
| MRAL.I21C-IgG4 | 17 | 311 | 473 | 20 |
| MRAL.T22C-IgG4 | 17 | 311 | 474 | 20 |
| MRAL.A25C-IgG4 | 17 | 311 | 475 | 20 |
| MRAL.S26C-IgG4 | 17 | 311 | 476 | 20 |
| MRAL.Q27C-IgG4 | 17 | 311 | 477 | 20 |
| MRAL.Y32C-IgG4 | 17 | 311 | 478 | 20 |
| MRAL.L33C-IgG4 | 17 | 311 | 479 | 20 |
| MRAL.N34C-IgG4 | 17 | 311 | 480 | 20 |
| MRAL.Y50C-IgG4 | 17 | 311 | 481 | 20 |
| MRAL.T51C-IgG4 | 17 | 311 | 482 | 20 |
| MRAL.H55C-IgG4 | 17 | 311 | 483 | 20 |
| MRAL.S56C-IgG4 | 17 | 311 | 484 | 20 |
| MRAL.G57C-IgG4 | 17 | 311 | 485 | 20 |
| MRAL.V58C-IgG4 | 17 | 311 | 486 | 20 |
| MRAL.P59C-IgG4 | 17 | 311 | 487 | 20 |
| MRAL.S60C-IgG4 | 17 | 311 | 488 | 20 |
| MRAL.R61C-IgG4 | 17 | 311 | 489 | 20 |
| MRAL.F62C-IgG4 | 17 | 311 | 490 | 20 |
| MRAL.S63C-IgG4 | 17 | 311 | 491 | 20 |
| MRAL.S65C-IgG4 | 17 | 311 | 492 | 20 |
| MRAL.S67C-IgG4 | 17 | 311 | 493 | 20 |
| MRAL.G68C-IgG4 | 17 | 311 | 494 | 20 |
| MRAL.T69C-IgG4 | 17 | 311 | 495 | 20 |
| MRAL.D70C-IgG4 | 17 | 311 | 496 | 20 |
| MRAL.T72C-IgG4 | 17 | 311 | 497 | 20 |
| MRAL.F73C-IgG4 | 17 | 311 | 498 | 20 |
| MRAL.T74C-IgG4 | 17 | 311 | 499 | 20 |
| MRAL.I75C-IgG4 | 17 | 311 | 500 | 20 |
| MRAL.S76C-IgG4 | 17 | 311 | 501 | 20 |
| MRAL.S77C-IgG4 | 17 | 311 | 502 | 20 |
| MRAL.L78C-IgG4 | 17 | 311 | 503 | 20 |
| MRAL.Q79C-IgG4 | 17 | 311 | 504 | 20 |
| MRAL.Y96C-IgG4 | 17 | 311 | 505 | 20 |
| MRAL.T97C-IgG4 | 17 | 311 | 506 | 20 |
| MRAL.F98C-IgG4 | 17 | 311 | 507 | 20 |
| MRAL.G99C-IgG4 | 17 | 311 | 508 | 20 |
| MRAL.Q100C-IgG4 | 17 | 311 | 509 | 20 |
| MRAL.G101C-IgG4 | 17 | 311 | 510 | 20 |
| MRAL.T102C-IgG4 | 17 | 311 | 511 | 20 |
| MRAL.K103C-IgG4 | 17 | 311 | 512 | 20 |
| MRAL.V104C-IgG4 | 17 | 311 | 513 | 20 |
| MRAL.E105C-IgG4 | 17 | 311 | 514 | 20 |
| MRAL.I106C-IgG4 | 17 | 311 | 515 | 20 |
| MRAL.K107C-IgG4 | 17 | 311 | 516 | 20 |
| k0.R108C-IgG4 | 17 | 311 | 19 | 517 |
| k0.T109C-IgG4 | 17 | 311 | 19 | 518 |
| k0.V110C-IgG4 | 17 | 311 | 19 | 519 |
| k0.A111C-IgG4 | 17 | 311 | 19 | 520 |
| k0.A112C-IgG4 | 17 | 311 | 19 | 521 |
| k0.P113C-IgG4 | 17 | 311 | 19 | 522 |
| k0.S114C-IgG4 | 17 | 311 | 19 | 523 |
| k0.V115C-IgG4 | 17 | 311 | 19 | 524 |
| k0.F116C-IgG4 | 17 | 311 | 19 | 525 |
| k0.P120C-IgG4 | 17 | 311 | 19 | 526 |
| k0.S121C-IgG4 | 17 | 311 | 19 | 527 |
| k0.D122C-IgG4 | 17 | 311 | 19 | 528 |
| k0.E123C-IgG4 | 17 | 311 | 19 | 529 |
| k0.Q124C-IgG4 | 17 | 311 | 19 | 530 |
| k0.L125C-IgG4 | 17 | 311 | 19 | 531 |
| k0.K126C-IgG4 | 17 | 311 | 19 | 532 |
| k0.S127C-IgG4 | 17 | 311 | 19 | 533 |
| k0.G128C-IgG4 | 17 | 311 | 19 | 534 |
| k0.T129C-IgG4 | 17 | 311 | 19 | 535 |
| k0.A130C-IgG4 | 17 | 311 | 19 | 536 |
| k0.S131C-IgG4 | 17 | 311 | 19 | 537 |
| k0.L136C-IgG4 | 17 | 311 | 19 | 538 |
| k0.N137C-IgG4 | 17 | 311 | 19 | 539 |
| k0.N138C-IgG4 | 17 | 311 | 19 | 540 |
| k0.F139C-IgG4 | 17 | 311 | 19 | 541 |
| k0.Y140C-IgG4 | 17 | 311 | 19 | 542 |
| k0.P141C-IgG4 | 17 | 311 | 19 | 543 |
| k0.R142C-IgG4 | 17 | 311 | 19 | 544 |
| k0.E143C-IgG4 | 17 | 311 | 19 | 545 |
| k0.A144C-IgG4 | 17 | 311 | 19 | 546 |
| k0.K145C-IgG4 | 17 | 311 | 19 | 547 |
| k0.V146C-IgG4 | 17 | 311 | 19 | 548 |
| k0.Q147C-IgG4 | 17 | 311 | 19 | 549 |
| k0.W148C-IgG4 | 17 | 311 | 19 | 550 |
| k0.K149C-IgG4 | 17 | 311 | 19 | 551 |
| k0.V150C-IgG4 | 17 | 311 | 19 | 552 |
| k0.D151C-IgG4 | 17 | 311 | 19 | 553 |
| k0.N152C-IgG4 | 17 | 311 | 19 | 554 |
| k0.A153C-IgG4 | 17 | 311 | 19 | 555 |
| k0.L154C-IgG4 | 17 | 311 | 19 | 556 |
| k0.Q155C-IgG4 | 17 | 311 | 19 | 557 |
| k0.S156C-IgG4 | 17 | 311 | 19 | 558 |
| k0.G157C-IgG4 | 17 | 311 | 19 | 559 |
| k0.N158C-IgG4 | 17 | 311 | 19 | 560 |
| k0.S159C-IgG4 | 17 | 311 | 19 | 561 |
| k0.Q160C-IgG4 | 17 | 311 | 19 | 562 |
| k0.E161C-IgG4 | 17 | 311 | 19 | 563 |
| k0.S162C-IgG4 | 17 | 311 | 19 | 564 |
| k0.V163C-IgG4 | 17 | 311 | 19 | 565 |
| k0.T164C-IgG4 | 17 | 311 | 19 | 566 |
| k0.E165C-IgG4 | 17 | 311 | 19 | 567 |
| k0.Q166C-IgG4 | 17 | 311 | 19 | 568 |
| k0.D167C-IgG4 | 17 | 311 | 19 | 569 |
| k0.S168C-IgG4 | 17 | 311 | 19 | 570 |
| k0.K169C-IgG4 | 17 | 311 | 19 | 571 |
| k0.D170C-IgG4 | 17 | 311 | 19 | 572 |
| k0.S171C-IgG4 | 17 | 311 | 19 | 573 |
| k0.T172C-IgG4 | 17 | 311 | 19 | 574 |
| k0.Y173C-IgG4 | 17 | 311 | 19 | 575 |
| k0.S174C-IgG4 | 17 | 311 | 19 | 576 |
| k0.L175C-IgG4 | 17 | 311 | 19 | 577 |
| k0.T180C-IgG4 | 17 | 311 | 19 | 578 |
| k0.L181C-IgG4 | 17 | 311 | 19 | 579 |
| k0.S182C-IgG4 | 17 | 311 | 19 | 580 |
| k0.K183C-IgG4 | 17 | 311 | 19 | 581 |
| k0.A184C-IgG4 | 17 | 311 | 19 | 582 |
| k0.D185C-IgG4 | 17 | 311 | 19 | 583 |
| k0.Y186C-IgG4 | 17 | 311 | 19 | 584 |
| k0.E187C-IgG4 | 17 | 311 | 19 | 585 |
| k0.K188C-IgG4 | 17 | 311 | 19 | 586 |
| k0.H189C-IgG4 | 17 | 311 | 19 | 587 |
| k0.K190C-IgG4 | 17 | 311 | 19 | 588 |
| k0.V191C-IgG4 | 17 | 311 | 19 | 589 |
| k0.Y192C-IgG4 | 17 | 311 | 19 | 590 |
| k0.A193C-IgG4 | 17 | 311 | 19 | 591 |
| k0.E195C-IgG4 | 17 | 311 | 19 | 592 |
| k0.V196C-IgG4 | 17 | 311 | 19 | 593 |
| k0.T197C-IgG4 | 17 | 311 | 19 | 594 |
| k0.H198C-IgG4 | 17 | 311 | 19 | 595 |
| k0.Q199C-IgG4 | 17 | 311 | 19 | 596 |
| k0.G200C-IgG4 | 17 | 311 | 19 | 597 |
| k0.L201C-IgG4 | 17 | 311 | 19 | 598 |
| k0.S202C-IgG4 | 17 | 311 | 19 | 599 |
| k0.S203C-IgG4 | 17 | 311 | 19 | 600 |
| k0.P204C-IgG4 | 17 | 311 | 19 | 601 |
| k0.V205C-IgG4 | 17 | 311 | 19 | 602 |
| k0.T206C-IgG4 | 17 | 311 | 19 | 603 |
| k0.K207C-IgG4 | 17 | 311 | 19 | 604 |
| k0.S208C-IgG4 | 17 | 311 | 19 | 605 |
| k0.F209C-IgG4 | 17 | 311 | 19 | 606 |
| k0.N210C-IgG4 | 17 | 311 | 19 | 607 |
| k0.R211C-IgG4 | 17 | 311 | 19 | 608 |
| k0.G212C-IgG4 | 17 | 311 | 19 | 609 |
| k0.E213C-IgG4 | 17 | 311 | 19 | 610 |

### Reference Example 9-2 Assessment of Electrophoretic Mobility in Polyacrylamide Gel of Antibodies Having Cysteine Substitution at Various Positions of IgG4

Similarly to Reference Example 8-2, non-reducing SDS-PAGE was carried out with the MRA-IgG4 variants produced in Reference Example 9-1, the gel image was captured, and bands were quantified.

From the obtained gel image, the variants were classified into 7 groups according to the band pattern of each of the MRA-IgG4 variants: Single (one band at a molecular weight region similar to that of MRA-IgG4), Double (two bands at a molecular weight region similar to that of MRA-IgG4), Triple (three bands at a molecular weight region similar to that of MRA-IgG4), Several (four or more bands at a molecular weight region similar to that of MRA-IgG4), LMW (band(s) at a molecular weight region lower than that of MRA-IgG4), HMW (band(s) at a molecular weight region higher than that of MRA-IgG4), and Faint (band(s) blurry and difficult to determine). Regarding the MRA-IgG4 variants classified as “Double”, one of the two bands showed the same electrophoretic mobility as MRA-IgG4 while the other band showed slightly faster or slower mobility. Thus, for the MRA-IgG4 variants classified as “Double”, the percentage of the bands showing different mobility to MRA-IgG4 (percentage of new band (%)) was also calculated. Grouping of the band patterns for MRA-IgG4 heavy chain variants and MRA-IgG4 light chain variants, and the calculation results of the band percentage are respectively shown in Tables 41 and 42. From Tables 41 and 42, variants classified into the Double and Triple groups are shown in Table 43. In these variants, it is highly likely that cysteine substitution caused structural changes such as crosslinkage of Fabs, which resulted in the change in electrophoretic mobility. It is noted that while Table 26 indicates “no data” for MRAL.K107C-IgG4, position 107 (Kabat numbering), which is the position of cysteine substitution in this variant, is a position where the residue structurally exposed to the surface is present in the hinge region. Thus, in this variant also, it is highly likely that cysteine substitution causes structural changes such as crosslinkage of Fabs, and results in the change in electrophoretic mobility.

TABLE 41

| MRA-IgG4 heavy chain | | Percentage of new |
|---|---|---|
| variant name | Group | band (%) |
| MRAH.Q5C-IgG4 | Single | — |
| MRAH.E6C-IgG4 | Double | 5.8 |
| MRAH.S7C-IgG4 | Single | — |
| MRAH.G8C-IgG4 | Single | — |
| MRAH.P9C-IgG4 | Single | — |
| MRAH.G10C-IgG4 | Single | — |
| MRAH.L11C-IgG4 | Single | — |
| MRAH.V12C-IgG4 | Faint | — |
| MRAH.R13C-IgG4 | Single | — |
| MRAH.P14C-IgG4 | Single | — |
| MRAH.S15C-IgG4 | Single | — |
| MRAH.Q16C-IgG4 | Single | — |
| MRAH.T17C-IgG4 | Single | — |
| MRAH.L18C-IgG4 | LMW | — |
| MRAH.S19C-IgG4 | Single | — |
| MRAH.L20C-IgG4 | LMW | — |
| MRAH.T21C-IgG4 | Single | — |
| MRAH.T23C-IgG4 | Single | — |
| MRAH.S25C-IgG4 | Double | 62.1 |
| MRAH.G26C-IgG4 | Double | 9.4 |
| MRAH.S28C-IgG4 | Single | — |
| MRAH.T30C-IgG4 | Single | — |
| MRAH.S31C-IgG4 | Single | — |
| MRAH.W35C-IgG4 | LMW | — |
| MRAH.S35aC-IgG4 | LMW | — |
| MRAH.Y50C-IgG4 | Single | — |
| MRAH.I51C-IgG4 | LMW | — |
| MRAH.S52C-IgG4 | Single | — |
| MRAH.S62C-IgG4 | Single | — |
| MRAH.L63C-IgG4 | Single | — |
| MRAH.K64C-IgG4 | Single | — |
| MRAH.S65C-IgG4 | Single | — |
| MRAH.R66C-IgG4 | Single | — |
| MRAH.V67C-IgG4 | LMW | — |
| MRAH.T68C-IgG4 | Single | — |
| MRAH.L70C-IgG4 | Single | — |
| MRAH.D72C-IgG4 | Single | — |
| MRAH.T73C-IgG4 | Single | — |
| MRAH.S74C-IgG4 | Double | 5.3 |
| MRAH.K75C-IgG4 | Single | — |
| MRAH.N76C-IgG4 | Single | — |
| MRAH.Q77C-IgG4 | Single | — |
| MRAH.S79C-IgG4 | Single | — |
| MRAH.L80C-IgG4 | LMW | — |
| MRAH.R81C-IgG4 | Single | — |
| MRAH.L82C-IgG4 | LMW | — |
| MRAH.S82aC-IgG4 | Single | — |
| MRAH.S82bC-IgG4 | Single | — |
| MRAH.V82cC-IgG4 | LMW | — |
| MRAH.D101C-IgG4 | Single | — |
| MRAH.Y102C-IgG4 | Single | — |
| MRAH.S112C-IgG4 | Single | — |
| MRAH.S113C-IgG4 | Single | — |
| G4T1.A118C-IgG4 | Single | — |
| G4T1.S119C-IgG4 | Double | 11 |
| G4T1.T120C-IgG4 | Single | — |
| G4T1.K121C-IgG4 | Single | — |
| G4T1.G122C-IgG4 | Single | — |
| G4T1.P123C-IgG4 | LMW | — |
| G4T1.S124C-IgG4 | Single | — |
| G4T1.V125C-IgG4 | LMW | — |
| G4T1.F126C-IgG4 | LMW | — |
| G4T1.P127C-IgG4 | LMW | — |
| G4T1.S132C-IgG4 | Triple | — |
| G4T1.R133C-IgG4 | Double | 82.9 |
| G4T1.S134C-IgG4 | Double | 80.4 |
| G4T1.T135C-IgG4 | Double | 88.6 |
| G4T1.S136C-IgG4 | Double | 82.4 |
| G4T1.E137C-IgG4 | Double | 44.7 |
| G4T1.S138C-IgG4 | Double | 52.6 |
| G4T1.T139C-IgG4 | Single | — |
| G4T1.A140C-IgG4 | Triple | — |
| G4T1.A141C-IgG4 | Single | — |
| G4T1.D148C-IgG4 | Single | — |
| G4T1.Y149C-IgG4 | Faint | — |
| G4T1.F150C-IgG4 | Single | — |
| G4T1.P151C-IgG4 | LMW | — |
| G4T1.E152C-IgG4 | Single | — |
| G4T1.P153C-IgG4 | Single | — |
| G4T1.V154C-IgG4 | LMW | — |
| G4T1.T155C-IgG4 | Single | — |
| G4T1.V156C-IgG4 | LMW | — |
| G4T1.S157C-IgG4 | Single | — |
| G4T1.W158C-IgG4 | LMW | — |
| G4T1.N159C-IgG4 | Double | 19.9 |
| G4T1.S160C-IgG4 | Double | 29.5 |
| G4T1.G161C-IgG4 | Double | 21.4 |
| G4T1.A162C-IgG4 | Double | 35.6 |
| G4T1.L163C-IgG4 | Double | 21.1 |
| G4T1.T164C-IgG4 | Double | 12.8 |
| G4T1.S165C-IgG4 | Double | 17 |
| G4T1.G166C-IgG4 | Double | 13 |
| G4T1.V167C-IgG4 | Double | 20.4 |
| G4T1.V173C-IgG4 | Double | 15.6 |
| G4T1.L174C-IgG4 | Double | 18.6 |
| G4T1.Q175C-IgG4 | Single | — |
| G4T1.S176C-IgG4 | Double | 20.3 |
| G4T1.S177C-IgG4 | Single | — |
| G4T1.G178C-IgG4 | Double | 22.5 |
| G4T1.L179C-IgG4 | Double | 26.1 |
| G4T1.Y180C-IgG4 | LMW | — |
| G4T1.V186C-IgG4 | LMW | — |
| G4T1.T187C-IgG4 | Double | 23.3 |
| G4T1.V188C-IgG4 | Double | 25.5 |
| G4T1.P189C-IgG4 | Double | 30.4 |
| G4T1.S190C-IgG4 | Double | 54.7 |
| G4T1.S191C-IgG4 | Double | 78.3 |
| G4T1.S192C-IgG4 | Double | 46.9 |
| G4T1.L193C-IgG4 | Double | 89.5 |
| G4T1.G194C-IgG4 | Double | 89.2 |
| G4T1.T195C-IgG4 | Double | 90.3 |
| G4T1.Q196C-IgG4 | Double | 63.4 |
| G4T1.T197C-IgG4 | Double | 79.8 |
| G4T1.Y198C-IgG4 | LMW | — |
| G4T1.T199C-IgG4 | LMW | — |
| G4T1.N201C-IgG4 | LMW | — |
| G4T1.V202C-IgG4 | LMW | — |
| G4T1.D203C-IgG4 | LMW | — |
| G4T1.H204C-IgG4 | LMW | — |
| G4T1.K205C-IgG4 | LMW | — |
| G4T1.P206C-IgG4 | LMW | — |
| G4T1.S207C-IgG4 | LMW | — |
| G4T1.N208C-IgG4 | LMW | — |
| G4T1.T209C-IgG4 | LMW | — |
| G4T1.K210C-IgG4 | Single | — |
| G4T1.V211C-IgG4 | Single | — |
| G4T1.D212C-IgG4 | Single | — |
| G4T1.K213C-IgG4 | Triple | — |
| G4T1.R214C-IgG4 | Single | — |
| G4T1.V215C-IgG4 | Double | 57.3 |
| G4T1.E216C-IgG4 | Single | — |
| G4T1.S217C-IgG4 | Single | — |
| G4T1.K218C-IgG4 | Single | — |

TABLE 42

| MRA-IgG4 light chain | | Percentage of new |
|---|---|---|
| variant name | Group | band (%) |
| MRAL.T5C-IgG4 | HMW | — |
| MRAL.Q6C-IgG4 | Faint | — |
| MRAL.S7C-IgG4 | Single | — |
| MRAL.P8C-IgG4 | no data | — |
| MRAL.S9C-IgG4 | Single | — |
| MRAL.S10C-IgG4 | Single | — |
| MRAL.L11C-IgG4 | Single | — |
| MRAL.S12C-IgG4 | Single | — |
| MRAL.A13C-IgG4 | Single | — |
| MRAL.S14C-IgG4 | Single | — |
| MRAL.V15C-IgG4 | Single | — |
| MRAL.G16C-IgG4 | Single | — |
| MRAL.D17C-IgG4 | Single | — |
| MRAL.R18C-IgG4 | Single | — |
| MRAL.V19C-IgG4 | Double | 29.2 |
| MRAL.T20C-IgG4 | Single | — |
| MRAL.I21C-IgG4 | Faint | — |
| MRAL.T22C-IgG4 | Single | — |
| MRAL.A25C-IgG4 | Faint | — |
| MRAL.S26C-IgG4 | Single | — |
| MRAL.Q27C-IgG4 | Single | — |
| MRAL.Y32C-IgG4 | Single | — |
| MRAL.L33C-IgG4 | Faint | — |
| MRAL.N34C-IgG4 | Faint | — |
| MRAL.Y50C-IgG4 | Single | — |
| MRAL.T51C-IgG4 | Single | — |
| MRAL.H55C-IgG4 | Single | — |
| MRAL.S56C-IgG4 | Double | 12.2 |
| MRAL.G57C-IgG4 | Double | 13.5 |
| MRAL.V58C-IgG4 | Double | 12.3 |
| MRAL.P59C-IgG4 | Double | 3.4 |
| MRAL.S60C-IgG4 | Double | 17.9 |
| MRAL.R61C-IgG4 | Single | — |
| MRAL.F62C-IgG4 | Double | 39.1 |
| MRAL.S63C-IgG4 | Single | — |
| MRAL.S65C-IgG4 | Single | — |
| MRAL.S67C-IgG4 | Single | — |
| MRAL.G68C-IgG4 | Single | — |
| MRAL.T69C-IgG4 | Single | — |
| MRAL.D70C-IgG4 | Single | — |
| MRAL.T72C-IgG4 | Single | — |
| MRAL.F73C-IgG4 | Double | 36.9 |
| MRAL.T74C-IgG4 | Single | — |
| MRAL.175C-IgG4 | no data | — |
| MRAL.S76C-IgG4 | Single | — |
| MRAL.S77C-IgG4 | Double | 51.2 |
| MRAL.L78C-IgG4 | Faint | — |
| MRAL.Q79C-IgG4 | Single | — |
| MRAL.Y96C-IgG4 | Faint | — |
| MRAL.T97C-IgG4 | Single | — |
| MRAL.F98C-IgG4 | Faint | — |
| MRAL.G99C-IgG4 | Double | 26.7 |
| MRAL.Q100C-IgG4 | Single | — |
| MRAL.G101C-IgG4 | Single | — |
| MRAL.T102C-IgG4 | Faint | — |
| MRAL.K103C-IgG4 | Single | — |
| MRAL.V104C-IgG4 | Faint | — |
| MRAL.E105C-IgG4 | Single | — |
| MRAL.I106C-IgG4 | Faint | — |
| MRAL.K107C-IgG4 | no data | — |
| k0.R108C-IgG4 | Single | — |
| k0.T109C-IgG4 | Double | 14.5 |
| k0.V110C-IgG4 | Double | 13.2 |
| k0.A111C-IgG4 | Single | — |
| k0.A112C-IgG4 | Double | 12 |
| k0.P113C-IgG4 | Single | — |
| k0.S114C-IgG4 | Single | — |
| k0.V115C-IgG4 | Faint | — |
| k0.F116C-IgG4 | Triple | — |
| k0.P120C-IgG4 | Faint | — |
| k0.S121C-IgG4 | Single | — |
| k0.D122C-IgG4 | LMW | — |
| k0.E123C-IgG4 | Single | — |
| k0.Q124C-IgG4 | Faint | — |
| k0.L125C-IgG4 | Single | — |
| k0.K126C-IgG4 | Double | 86.3 |
| k0.S127C-IgG4 | Single | — |
| k0.G128C-IgG4 | Single | — |
| k0.T129C-IgG4 | Single | — |
| k0.A130C-IgG4 | Faint | — |
| k0.S131C-IgG4 | LMW | — |
| k0.L136C-IgG4 | LMW | — |
| k0.N137C-IgG4 | Triple | — |
| k0.N138C-IgG4 | Single | — |
| k0.F139C-IgG4 | LMW | — |
| k0.Y140C-IgG4 | LMW | — |
| k0.P141C-IgG4 | Single | — |
| k0.R142C-IgG4 | Single | — |
| k0.E143C-IgG4 | Single | — |
| k0.A144C-IgG4 | LMW | — |
| k0.K145C-IgG4 | Single | — |
| k0.V146C-IgG4 | LMW | — |
| k0.Q147C-IgG4 | Single | — |
| k0.W148C-IgG4 | LMW | — |
| k0.K149C-IgG4 | Single | — |
| k0.V150C-IgG4 | LMW | — |
| k0.D151C-IgG4 | Double | 21.9 |
| k0.N152C-IgG4 | Double | 68.7 |
| k0.A153C-IgG4 | Single | — |
| k0.L154C-IgG4 | Single | — |
| k0.Q155C-IgG4 | Single | — |
| k0.S156C-IgG4 | HMW | — |
| k0.G157C-IgG4 | Single | — |
| k0.N158C-IgG4 | Single | — |
| k0.S159C-IgG4 | Single | — |
| k0.Q160C-IgG4 | Single | — |
| k0.E161C-IgG4 | Single | — |
| k0.S162C-IgG4 | Single | — |
| k0.V163C-IgG4 | Single | — |
| k0.T164C-IgG4 | Single | — |
| k0.E165C-IgG4 | Single | — |
| k0.Q166C-IgG4 | Single | — |
| k0.D167C-IgG4 | Single | — |
| k0.S168C-IgG4 | Single | — |
| k0.K169C-IgG4 | Single | — |
| k0.D170C-IgG4 | Single | — |
| k0.S171C-IgG4 | Single | — |
| k0.T172C-IgG4 | Faint | — |
| k0.Y173C-IgG4 | Faint | — |
| k0.S174C-IgG4 | Faint | — |
| k0.L175C-IgG4 | Faint | — |
| k0.T180C-IgG4 | Single | — |
| k0.L181C-IgG4 | Faint | — |
| k0.S182C-IgG4 | Single | — |
| k0.K183C-IgG4 | Single | — |
| k0.A184C-IgG4 | Double | 11.8 |
| k0.D185C-IgG4 | Single | — |
| k0.Y186C-IgG4 | Double | 31.7 |
| k0.E187C-IgG4 | LMW | — |
| k0.K188C-IgG4 | Single | — |
| k0.H189C-IgG4 | Faint | — |
| k0.K190C-IgG4 | LMW | — |
| k0.V191C-IgG4 | LMW | — |
| k0.Y192C-IgG4 | Faint | — |
| k0.A193C-IgG4 | Single | — |
| k0.E195C-IgG4 | Single | — |
| k0.V196C-IgG4 | Faint | — |
| k0.T197C-IgG4 | Single | — |
| k0.H198C-IgG4 | Faint | — |
| k0.Q199C-IgG4 | Single | — |
| k0.G200C-IgG4 | Double | 21.7 |
| k0.L201C-IgG4 | Double | 3.7 |
| k0.S202C-IgG4 | Double | 61.5 |
| k0.S203C-IgG4 | Double | 39 |
| k0.P204C-IgG4 | Single | — |
| k0.V205C-IgG4 | Single | — |
| k0.T206C-IgG4 | Single | — |
| k0.K207C-IgG4 | Single | — |
| k0.S208C-IgG4 | Single | — |
| k0.F209C-IgG4 | Double | 82.2 |
| k0.N210C-IgG4 | LMW | — |
| k0.R211C-IgG4 | Double | 12.1 |
| k0.G212C-IgG4 | Double | 25.6 |
| k0.E213C-IgG4 | Double | 90.9 |

TABLE 43

| MRA-IgG4 variant | | Percentage of new |
|---|---|---|
| name | Group | band (%) |
| MRAH.E6C-IgG4 | Double | 5.8 |
| MRAH.S25C-IgG4 | Double | 62.1 |
| MRAH.G26C-IgG4 | Double | 9.4 |
| MRAH.S74C-IgG4 | Double | 5.3 |
| G4T1.S119C-IgG4 | Double | 11 |
| G4T1.S132C-IgG4 | Triple | — |
| G4T1.R133C-IgG4 | Double | 82.9 |
| G4T1.S134C-IgG4 | Double | 80.4 |
| G4T1.T135C-IgG4 | Double | 88.6 |
| G4T1.S136C-IgG4 | Double | 82.4 |
| G4T1.E137C-IgG4 | Double | 44.7 |
| G4T1.S138C-IgG4 | Double | 52.6 |
| G4T1.A140C-IgG4 | Triple | — |
| G4T1.N159C-IgG4 | Double | 19.9 |
| G4T1.S160C-IgG4 | Double | 29.5 |
| G4T1.G161C-IgG4 | Double | 21.4 |
| G4T1.A162C-IgG4 | Double | 35.6 |
| G4T1.L163C-IgG4 | Double | 21.1 |
| G4T1.T164C-IgG4 | Double | 12.8 |
| G4T1.S165C-IgG4 | Double | 17 |
| G4T1.G166C-IgG4 | Double | 13 |
| G4T1.V167C-IgG4 | Double | 20.4 |
| G4T1.V173C-IgG4 | Double | 15.6 |
| G4T1.L174C-IgG4 | Double | 18.6 |
| G4T1.S176C-IgG4 | Double | 20.3 |
| G4T1.G178C-IgG4 | Double | 22.5 |
| G4T1.L179C-IgG4 | Double | 26.1 |
| G4T1.T187C-IgG4 | Double | 23.3 |
| G4T1.V188C-IgG4 | Double | 25.5 |
| G4T1.P189C-IgG4 | Double | 30.4 |
| G4T1.S190C-IgG4 | Double | 54.7 |
| G4T1.S191C-IgG4 | Double | 78.3 |
| G4T1.S192C-IgG4 | Double | 46.9 |
| G4T1.L193C-IgG4 | Double | 89.5 |
| G4T1.G194C-IgG4 | Double | 89.2 |
| G4T1.T195C-IgG4 | Double | 90.3 |
| G4T1.Q196C-IgG4 | Double | 63.4 |
| G4T1.T197C-IgG4 | Double | 79.8 |
| G4T1.K213C-IgG4 | Triple | — |
| G4T1.V215C-IgG4 | Double | 57.3 |
| MRAL.V19C-IgG4 | Double | 29.2 |
| MRAL.S56C-IgG4 | Double | 12.2 |
| MRAL.G57C-IgG4 | Double | 13.5 |
| MRAL.V58C-IgG4 | Double | 12.3 |
| MRAL.P59C-IgG4 | Double | 3.4 |
| MRAL.S60C-IgG4 | Double | 17.9 |
| MRAL.F62C-IgG4 | Double | 39.1 |
| MRAL.F73C-IgG4 | Double | 36.9 |
| MRAL.S77C-IgG4 | Double | 51.2 |
| MRAL.G99C-IgG4 | Double | 26.7 |
| k0.T109C-IgG4 | Double | 14.5 |
| k0.V110C-IgG4 | Double | 13.2 |
| k0.A112C-IgG4 | Double | 12 |
| k0.F116C-IgG4 | Triple | — |
| k0.K126C-IgG4 | Double | 86.3 |
| k0.N137C-IgG4 | Triple | — |
| k0.D151C-IgG4 | Double | 21.9 |
| k0.N152C-IgG4 | Double | 68.7 |
| k0.A184C-IgG4 | Double | 11.8 |
| k0.Y186C-IgG4 | Double | 31.7 |
| k0.G200C-IgG4 | Double | 21.7 |
| k0.L201C-IgG4 | Double | 3.7 |
| k0.S202C-IgG4 | Double | 61.5 |
| k0.S203C-IgG4 | Double | 39 |
| k0.F209C-IgG4 | Double | 82.2 |
| k0.R211C-IgG4 | Double | 12.1 |
| k0.G212C-IgG4 | Double | 25.6 |
| k0.E213C-IgG4 | Double | 90.9 |

### Reference Example 10 Assessment of Antibodies Having Cysteine Substitution at Various Positions of IgG2

### Reference Example 10-1 Production of Antibodies Having Cysteine Substitution at Various Positions of IgG2

The heavy chain and light chain of an anti-human IL6R neutralizing antibody, MRA-IgG2 (heavy chain: MRAH-G2d (SEQ ID NO: 312), light chain: MRAL-k0 (SEQ ID NO: 16)), were subjected to a study in which an arbitrary amino acid residue structurally exposed to the surface was substituted with cysteine.

Amino acid residues within the MRA-IgG2 heavy chain variable region (MRAH, SEQ ID NO: 17) were substituted with cysteine to produce variants of the MRA-IgG2 heavy chain variable region shown in Table 44. These variants of the MRA-IgG2 heavy chain variable region were each linked with the MRA-IgG2 heavy chain constant region (G2d, SEQ ID NO: 313) to produce MRA-IgG2 heavy chain variants, and expression vectors encoding the corresponding genes were produced by a method known to the person skilled in the art. In addition, amino acid residues within the MRA-IgG2 heavy chain constant region (G2d, SEQ ID NO: 313) were substituted with cysteine to produce variants of the MRA-IgG2 heavy chain constant region shown in Table 45. These variants of the MRA-IgG2 heavy chain constant region were each linked with the MRA-IgG2 heavy chain variable region (MRAH, SEQ ID NO: 17) to produce MRA-IgG2 heavy chain variants, and expression vectors encoding the corresponding genes were produced by a method known to the person skilled in the art.

TABLE 44

| Variant of MRA-IgG2 | Position of cysteine | |
|---|---|---|
| heavy chain variable | substitution | SEQ ID |
| region | (Kabat numbering) | NO: |
| MRAH.Q5C | 5 | 322 |
| MRAH.E6C | 6 | 323 |
| MRAH.S7C | 7 | 324 |
| MRAH.G8C | 8 | 325 |
| MRAH.P9C | 9 | 326 |
| MRAH.G10C | 10 | 327 |
| MRAH.L11C | 11 | 328 |
| MRAH.V12C | 12 | 329 |
| MRAH.R13C | 13 | 330 |
| MRAH.P14C | 14 | 331 |
| MRAH.S15C | 15 | 332 |
| MRAH.Q16C | 16 | 333 |
| MRAH.T17C | 17 | 334 |
| MRAH.L18C | 18 | 335 |
| MRAH.S19C | 19 | 336 |
| MRAH.L20C | 20 | 337 |
| MRAH.T21C | 21 | 338 |
| MRAH.T23C | 23 | 339 |
| MRAH.S25C | 25 | 340 |
| MRAH.G26C | 26 | 341 |
| MRAH.S28C | 28 | 342 |
| MRAH.T30C | 30 | 343 |
| MRAH.S31C | 31 | 344 |
| MRAH.W35C | 35 | 345 |
| MRAH.S35aC | 35a | 346 |
| MRAH.Y50C | 50 | 347 |
| MRAH.I51C | 51 | 348 |
| MRAH.S52C | 52 | 349 |
| MRAH.S62C | 62 | 350 |
| MRAH.L63C | 63 | 351 |
| MRAH.K64C | 64 | 352 |
| MRAH.S65C | 65 | 353 |
| MRAH.R66C | 66 | 354 |
| MRAH.V67C | 67 | 355 |
| MRAH.T68C | 68 | 356 |
| MRAH.L70C | 70 | 357 |
| MRAH.D72C | 72 | 358 |
| MRAH.T73C | 73 | 359 |
| MRAH.S74C | 74 | 360 |
| MRAH.K75C | 75 | 361 |
| MRAH.N76C | 76 | 362 |
| MRAH.Q77C | 77 | 363 |
| MRAH.S79C | 79 | 364 |
| MRAH.L80C | 80 | 365 |
| MRAH.R81C | 81 | 366 |
| MRAH.L82C | 82 | 367 |
| MRAH.S82aC | 82a | 368 |
| MRAH.S82bC | 82b | 369 |
| MRAH.V82cC | 82c | 370 |
| MRAH.D101C | 101 | 371 |
| MRAH.Y102C | 102 | 372 |
| MRAH.S112C | 112 | 373 |
| MRAH.S113C | 113 | 374 |

TABLE 45

| Variant of MRA-IgG2 | Position of cysteine | |
|---|---|---|
| heavy chain constant | substitution | SEQ ID |
| region | (EU numbering) | NO: |
| G2d.A118C | 118 | 691 |
| G2d.S119C | 119 | 692 |
| G2d.T120C | 120 | 693 |
| G2d.K121C | 121 | 694 |
| G2d.G122C | 122 | 695 |
| G2d.P123C | 123 | 696 |
| G2d.S124C | 124 | 697 |
| G2d.V125C | 125 | 698 |
| G2d.F126C | 126 | 699 |
| G2d.P127C | 127 | 700 |
| G2d.S132C | 132 | 701 |
| G2d.R133C | 133 | 702 |
| G2d.S134C | 134 | 703 |
| G2d.T135C | 135 | 704 |
| G2d.S136C | 136 | 705 |
| G2d.E137C | 137 | 706 |
| G2d.S138C | 138 | 707 |
| G2d.T139C | 139 | 708 |
| G2d.A140C | 140 | 709 |
| G2d.A141C | 141 | 710 |
| G2d.D148C | 148 | 711 |
| G2d.Y149C | 149 | 712 |
| G2d.F150C | 150 | 713 |
| G2d.P151C | 151 | 714 |
| G2d.E152C | 152 | 715 |
| G2d.P153C | 153 | 716 |
| G2d.V154C | 154 | 717 |
| G2d.T155C | 155 | 718 |
| G2d.V156C | 156 | 719 |
| G2d.S157C | 157 | 720 |
| G2d.W158C | 158 | 721 |
| G2d.N159C | 159 | 722 |
| G2d.S160C | 160 | 723 |
| G2d.G161C | 161 | 724 |
| G2d.A162C | 162 | 725 |
| G2d.L163C | 163 | 726 |
| G2d.T164C | 164 | 727 |
| G2d.S165C | 165 | 728 |
| G2d.G166C | 166 | 729 |
| G2d.V167C | 167 | 730 |
| G2d.V173C | 173 | 731 |
| G2d.L174C | 174 | 732 |
| G2d.Q175C | 175 | 733 |
| G2d.S176C | 176 | 734 |
| G2d.S177C | 177 | 735 |
| G2d.G178C | 178 | 736 |
| G2d.L179C | 179 | 737 |
| G2d.Y180C | 180 | 738 |
| G2d.V186C | 186 | 739 |
| G2d.T187C | 187 | 740 |
| G2d.V188C | 188 | 741 |
| G2d.P189C | 189 | 742 |
| G2d.S190C | 190 | 743 |
| G2d.S191C | 191 | 744 |
| G2d.N192C | 192 | 745 |
| G2d.F193C | 193 | 746 |
| G2d.G194C | 194 | 747 |
| G2d.T195C | 195 | 748 |
| G2d.Q196C | 196 | 749 |
| G2d.T197C | 197 | 750 |
| G2d.Y198C | 198 | 751 |
| G2d.T199C | 199 | 752 |
| G2d.N201C | 201 | 753 |
| G2d.V202C | 202 | 754 |
| G2d.D203C | 203 | 755 |
| G2d.H204C | 204 | 756 |
| G2d.K205C | 205 | 757 |
| G2d.P206C | 206 | 758 |
| G2d.S207C | 207 | 759 |
| G2d.N208C | 208 | 760 |
| G2d.T209C | 209 | 761 |
| G2d.K210C | 210 | 762 |
| G2d.V211C | 211 | 763 |
| G2d.D212C | 212 | 764 |
| G2d.K213C | 213 | 765 |
| G2d.T214C | 214 | 766 |
| G2d.V215C | 215 | 767 |
| G2d.E216C | 216 | 768 |
| G2d.R217C | 217 | 769 |
| G2d.K218C | 218 | 770 |

The MRA-IgG2 heavy chain variants produced above were combined with the MRA-IgG2 light chain, or the MRA-IgG2 heavy chain was combined with the MRA-IgG2 light chain variants produced in Reference Example 8-1. The resultant MRA-IgG2 heavy chain variants and MRA-IgG2 light chain variants shown in Tables 46 and 47 were expressed by transient expression using FreeStyle293 cells (Invitrogen) or Expi293 cells (Life technologies) by a method known to the person skilled in the art, and purified with Protein A by a method known to the person skilled in the art.

TABLE 46

| | Heavy | Heavy | Light | Light |
|---|---|---|---|---|
| | chain | chain | chain | chain |
| | variable | constant | variable | constant |
| | region | region | region | region |
| MRA-IgG2 heavy chain | SEQ | SEQ | SEQ | SEQ |
| variant name | ID NO: | ID NO: | ID NO: | ID NO: |
| MRAH.Q5C-IgG2 | 322 | 313 | 19 | 20 |
| MRAH.E6C-IgG2 | 323 | 313 | 19 | 20 |
| MRAH.S7C-IgG2 | 324 | 313 | 19 | 20 |
| MRAH.G8C-IgG2 | 325 | 313 | 19 | 20 |
| MRAH.P9C-IgG2 | 326 | 313 | 19 | 20 |
| MRAH.G10C-IgG2 | 327 | 313 | 19 | 20 |
| MRAH.L11C-IgG2 | 328 | 313 | 19 | 20 |
| MRAH.V12C-IgG2 | 329 | 313 | 19 | 20 |
| MRAH.R13C-IgG2 | 330 | 313 | 19 | 20 |
| MRAH.P14C-IgG2 | 331 | 313 | 19 | 20 |
| MRAH.S15C-IgG2 | 332 | 313 | 19 | 20 |
| MRAH.Q16C-IgG2 | 333 | 313 | 19 | 20 |
| MRAH.T17C-IgG2 | 334 | 313 | 19 | 20 |
| MRAH.L18C-IgG2 | 335 | 313 | 19 | 20 |
| MRAH.S19C-IgG2 | 336 | 313 | 19 | 20 |
| MRAH.L20C-IgG2 | 337 | 313 | 19 | 20 |
| MRAH.T21C-IgG2 | 338 | 313 | 19 | 20 |
| MRAH.T23C-IgG2 | 339 | 313 | 19 | 20 |
| MRAH.S25C-IgG2 | 340 | 313 | 19 | 20 |
| MRAH.G26C-IgG2 | 341 | 313 | 19 | 20 |
| MRAH.S28C-IgG2 | 342 | 313 | 19 | 20 |
| MRAH.T30C-IgG2 | 343 | 313 | 19 | 20 |
| MRAH.S31C-IgG2 | 344 | 313 | 19 | 20 |
| MRAH.W35C-IgG2 | 345 | 313 | 19 | 20 |
| MRAH.S35aC-IgG2 | 346 | 313 | 19 | 20 |
| MRAH.Y50C-IgG2 | 347 | 313 | 19 | 20 |
| MRAH.I51C-IgG2 | 348 | 313 | 19 | 20 |
| MRAH.S52C-IgG2 | 349 | 313 | 19 | 20 |
| MRAH.S62C-IgG2 | 350 | 313 | 19 | 20 |
| MRAH.L63C-IgG2 | 351 | 313 | 19 | 20 |
| MRAH.K64C-IgG2 | 352 | 313 | 19 | 20 |
| MRAH.S65C-IgG2 | 353 | 313 | 19 | 20 |
| MRAH.R66C-IgG2 | 354 | 313 | 19 | 20 |
| MRAH.V67C-IgG2 | 355 | 313 | 19 | 20 |
| MRAH.T68C-IgG2 | 356 | 313 | 19 | 20 |
| MRAH.L70C-IgG2 | 357 | 313 | 19 | 20 |
| MRAH.D72C-IgG2 | 358 | 313 | 19 | 20 |
| MRAH.T73C-IgG2 | 359 | 313 | 19 | 20 |
| MRAH.S74C-IgG2 | 360 | 313 | 19 | 20 |
| MRAH.K75C-IgG2 | 361 | 313 | 19 | 20 |
| MRAH.N76C-IgG2 | 362 | 313 | 19 | 20 |
| MRAH.Q77C-IgG2 | 363 | 313 | 19 | 20 |
| MRAH.S79C-IgG2 | 364 | 313 | 19 | 20 |
| MRAH.L80C-IgG2 | 365 | 313 | 19 | 20 |
| MRAH.R81C-IgG2 | 366 | 313 | 19 | 20 |
| MRAH.L82C-IgG2 | 367 | 313 | 19 | 20 |
| MRAH.S82aC-IgG2 | 368 | 313 | 19 | 20 |
| MRAH.S82bC-IgG2 | 369 | 313 | 19 | 20 |
| MRAH.V82cC-IgG2 | 370 | 313 | 19 | 20 |
| MRAH.D101C-IgG2 | 371 | 313 | 19 | 20 |
| MRAH.Y102C-IgG2 | 372 | 313 | 19 | 20 |
| MRAH.S112C-IgG2 | 373 | 313 | 19 | 20 |
| MRAH.S113C-IgG2 | 374 | 313 | 19 | 20 |
| G2d.A118C-IgG2 | 17 | 691 | 19 | 20 |
| G2d.S119C-IgG2 | 17 | 692 | 19 | 20 |
| G2d.T120C-IgG2 | 17 | 693 | 19 | 20 |
| G2d.K121C-IgG2 | 17 | 694 | 19 | 20 |
| G2d.G122C-IgG2 | 17 | 695 | 19 | 20 |
| G2d.P123C-IgG2 | 17 | 696 | 19 | 20 |
| G2d.S124C-IgG2 | 17 | 697 | 19 | 20 |
| G2d.V125C-IgG2 | 17 | 698 | 19 | 20 |
| G2d.F126C-IgG2 | 17 | 699 | 19 | 20 |
| G2d.P127C-IgG2 | 17 | 700 | 19 | 20 |
| G2d.S132C-IgG2 | 17 | 701 | 19 | 20 |
| G2d.R133C-IgG2 | 17 | 702 | 19 | 20 |
| G2d.S134C-IgG2 | 17 | 703 | 19 | 20 |
| G2d.T135C-IgG2 | 17 | 704 | 19 | 20 |
| G2d.S136C-IgG2 | 17 | 705 | 19 | 20 |
| G2d.E137C-IgG2 | 17 | 706 | 19 | 20 |
| G2d.S138C-IgG2 | 17 | 707 | 19 | 20 |
| G2d.T139C-IgG2 | 17 | 708 | 19 | 20 |
| G2d.A140C-IgG2 | 17 | 709 | 19 | 20 |
| G2d.A141C-IgG2 | 17 | 710 | 19 | 20 |
| G2d.D148C-IgG2 | 17 | 711 | 19 | 20 |
| G2d.Y149C-IgG2 | 17 | 712 | 19 | 20 |
| G2d.F150C-IgG2 | 17 | 713 | 19 | 20 |
| G2d.P151C-IgG2 | 17 | 714 | 19 | 20 |
| G2d.E152C-IgG2 | 17 | 715 | 19 | 20 |
| G2d.P153C-IgG2 | 17 | 716 | 19 | 20 |
| G2d.V154C-IgG2 | 17 | 717 | 19 | 20 |
| G2d.T155C-IgG2 | 17 | 718 | 19 | 20 |
| G2d.V156C-IgG2 | 17 | 719 | 19 | 20 |
| G2d.S157C-IgG2 | 17 | 720 | 19 | 20 |
| G2d.W158C-IgG2 | 17 | 721 | 19 | 20 |
| G2d.N159C-IgG2 | 17 | 722 | 19 | 20 |
| G2d.S160C-IgG2 | 17 | 723 | 19 | 20 |
| G2d.G161C-IgG2 | 17 | 724 | 19 | 20 |
| G2d.A162C-IgG2 | 17 | 725 | 19 | 20 |
| G2d.L163C-IgG2 | 17 | 726 | 19 | 20 |
| G2d.T164C-IgG2 | 17 | 727 | 19 | 20 |
| G2d.S165C-IgG2 | 17 | 728 | 19 | 20 |
| G2d.G166C-IgG2 | 17 | 729 | 19 | 20 |
| G2d.V167C-IgG2 | 17 | 730 | 19 | 20 |
| G2d.V173C-IgG2 | 17 | 731 | 19 | 20 |
| G2d.L174C-IgG2 | 17 | 732 | 19 | 20 |
| G2d.Q175C-IgG2 | 17 | 733 | 19 | 20 |
| G2d.S176C-IgG2 | 17 | 734 | 19 | 20 |
| G2d.S177C-IgG2 | 17 | 735 | 19 | 20 |
| G2d.G178C-IgG2 | 17 | 736 | 19 | 20 |
| G2d.L179C-IgG2 | 17 | 737 | 19 | 20 |
| G2d.Y180C-IgG2 | 17 | 738 | 19 | 20 |
| G2d.V186C-IgG2 | 17 | 739 | 19 | 20 |
| G2d.T187C-IgG2 | 17 | 740 | 19 | 20 |
| G2d.V188C-IgG2 | 17 | 741 | 19 | 20 |
| G2d.P189C-IgG2 | 17 | 742 | 19 | 20 |
| G2d.S190C-IgG2 | 17 | 743 | 19 | 20 |
| G2d.S191C-IgG2 | 17 | 744 | 19 | 20 |
| G2d.N192C-IgG2 | 17 | 745 | 19 | 20 |
| G2d.F193C-IgG2 | 17 | 746 | 19 | 20 |
| G2d.G194C-IgG2 | 17 | 747 | 19 | 20 |
| G2d.T195C-IgG2 | 17 | 748 | 19 | 20 |
| G2d.Q196C-IgG2 | 17 | 749 | 19 | 20 |
| G2d.T197C-IgG2 | 17 | 750 | 19 | 20 |
| G2d.Y198C-IgG2 | 17 | 751 | 19 | 20 |
| G2d.T199C-IgG2 | 17 | 752 | 19 | 20 |
| G2d.N201C-IgG2 | 17 | 753 | 19 | 20 |
| G2d.V202C-IgG2 | 17 | 754 | 19 | 20 |
| G2d.D203C-IgG2 | 17 | 755 | 19 | 20 |
| G2d.H204C-IgG2 | 17 | 756 | 19 | 20 |
| G2d.K205C-IgG2 | 17 | 757 | 19 | 20 |
| G2d.P206C-IgG2 | 17 | 758 | 19 | 20 |
| G2d.S207C-IgG2 | 17 | 759 | 19 | 20 |
| G2d.N208C-IgG2 | 17 | 760 | 19 | 20 |
| G2d.T209C-IgG2 | 17 | 761 | 19 | 20 |
| G2d.K210C-IgG2 | 17 | 762 | 19 | 20 |
| G2d.V211C-IgG2 | 17 | 763 | 19 | 20 |
| G2d.D212C-IgG2 | 17 | 764 | 19 | 20 |
| G2d.K213C-IgG2 | 17 | 765 | 19 | 20 |
| G2d.T214C-IgG2 | 17 | 766 | 19 | 20 |
| G2d.V215C-IgG2 | 17 | 767 | 19 | 20 |
| G2d.E216C-IgG2 | 17 | 768 | 19 | 20 |
| G2d.R217C-IgG2 | 17 | 769 | 19 | 20 |
| G2d.K218C-IgG2 | 17 | 770 | 19 | 20 |

TABLE 47

| | Heavy | Heavy | Light | Light |
|---|---|---|---|---|
| | chain | chain | chain | chain |
| | variable | constant | variable | constant |
| | region | region | region | region |
| MRA-IgG2 light chain | SEQ | SEQ | SEQ | SEQ |
| variant name | ID NO: | ID NO: | ID NO: | ID NO: |
| MRAL.T5C-IgG2 | 17 | 313 | 457 | 20 |
| MRAL.Q6C-IgG2 | 17 | 313 | 458 | 20 |
| MRAL.S7C-IgG2 | 17 | 313 | 459 | 20 |
| MRAL.P8C-IgG2 | 17 | 313 | 460 | 20 |
| MRAL.S9C-IgG2 | 17 | 313 | 461 | 20 |
| MRAL.S10C-IgG2 | 17 | 313 | 462 | 20 |
| MRAL.L11C-IgG2 | 17 | 313 | 463 | 20 |
| MRAL.S12C-IgG2 | 17 | 313 | 464 | 20 |
| MRAL.A13C-IgG2 | 17 | 313 | 465 | 20 |
| MRAL.S14C-IgG2 | 17 | 313 | 466 | 20 |
| MRAL.V15C-IgG2 | 17 | 313 | 467 | 20 |
| MRAL.G16C-IgG2 | 17 | 313 | 468 | 20 |
| MRAL.D17C-IgG2 | 17 | 313 | 469 | 20 |
| MRAL.R18C-IgG2 | 17 | 313 | 470 | 20 |
| MRAL.V19C-IgG2 | 17 | 313 | 471 | 20 |
| MRAL.T20C-IgG2 | 17 | 313 | 472 | 20 |
| MRAL.I21C-IgG2 | 17 | 313 | 473 | 20 |
| MRAL.T22C-IgG2 | 17 | 313 | 474 | 20 |
| MRAL.A25C-IgG2 | 17 | 313 | 475 | 20 |
| MRAL.S26C-IgG2 | 17 | 313 | 476 | 20 |
| MRAL.Q27C-IgG2 | 17 | 313 | 477 | 20 |
| MRAL.Y32C-IgG2 | 17 | 313 | 478 | 20 |
| MRAL.L33C-IgG2 | 17 | 313 | 479 | 20 |
| MRAL.N34C-IgG2 | 17 | 313 | 480 | 20 |
| MRAL.Y50C-IgG2 | 17 | 313 | 481 | 20 |
| MRAL.T51C-IgG2 | 17 | 313 | 482 | 20 |
| MRAL.H55C-IgG2 | 17 | 313 | 483 | 20 |
| MRAL.S56C-IgG2 | 17 | 313 | 484 | 20 |
| MRAL.G57C-IgG2 | 17 | 313 | 485 | 20 |
| MRAL.V58C-IgG2 | 17 | 313 | 486 | 20 |
| MRAL.P59C-IgG2 | 17 | 313 | 487 | 20 |
| MRAL.S60C-IgG2 | 17 | 313 | 488 | 20 |
| MRAL.R61C-IgG2 | 17 | 313 | 489 | 20 |
| MRAL.F62C-IgG2 | 17 | 313 | 490 | 20 |
| MRAL.S63C-IgG2 | 17 | 313 | 491 | 20 |
| MRAL.S65C-IgG2 | 17 | 313 | 492 | 20 |
| MRAL.S67C-IgG2 | 17 | 313 | 493 | 20 |
| MRAL.G68C-IgG2 | 17 | 313 | 494 | 20 |
| MRAL.T69C-IgG2 | 17 | 313 | 495 | 20 |
| MRAL.D70C-IgG2 | 17 | 313 | 496 | 20 |
| MRAL.T72C-IgG2 | 17 | 313 | 497 | 20 |
| MRAL.F73C-IgG2 | 17 | 313 | 498 | 20 |
| MRAL.T74C-IgG2 | 17 | 313 | 499 | 20 |
| MRAL.I75C-IgG2 | 17 | 313 | 500 | 20 |
| MRAL.S76C-IgG2 | 17 | 313 | 501 | 20 |
| MRAL.S77C-IgG2 | 17 | 313 | 502 | 20 |
| MRAL.L78C-IgG2 | 17 | 313 | 503 | 20 |
| MRAL.Q79C-IgG2 | 17 | 313 | 504 | 20 |
| MRAL.Y96C-IgG2 | 17 | 313 | 505 | 20 |
| MRAL.T97C-IgG2 | 17 | 313 | 506 | 20 |
| MRAL.F98C-IgG2 | 17 | 313 | 507 | 20 |
| MRAL.G99C-IgG2 | 17 | 313 | 508 | 20 |
| MRAL.Q100C-IgG2 | 17 | 313 | 509 | 20 |
| MRAL.G101C-IgG2 | 17 | 313 | 510 | 20 |
| MRAL.T102C-IgG2 | 17 | 313 | 511 | 20 |
| MRAL.K103C-IgG2 | 17 | 313 | 512 | 20 |
| MRAL.V104C-IgG2 | 17 | 313 | 513 | 20 |
| MRAL.E105C-IgG2 | 17 | 313 | 514 | 20 |
| MRAL.I106C-IgG2 | 17 | 313 | 515 | 20 |
| MRAL.K107C-IgG2 | 17 | 313 | 516 | 20 |
| k0.R108C-IgG2 | 17 | 313 | 19 | 517 |
| k0.T109C-IgG2 | 17 | 313 | 19 | 518 |
| k0.V110C-IgG2 | 17 | 313 | 19 | 519 |
| k0.A111C-IgG2 | 17 | 313 | 19 | 520 |
| k0.A112C-IgG2 | 17 | 313 | 19 | 521 |
| k0.P113C-IgG2 | 17 | 313 | 19 | 522 |
| k0.S114C-IgG2 | 17 | 313 | 19 | 523 |
| k0.V115C-IgG2 | 17 | 313 | 19 | 524 |
| k0.F116C-IgG2 | 17 | 313 | 19 | 525 |
| k0.P120C-IgG2 | 17 | 313 | 19 | 526 |
| k0.S121C-IgG2 | 17 | 313 | 19 | 527 |
| k0.D122C-IgG2 | 17 | 313 | 19 | 528 |
| k0.E123C-IgG2 | 17 | 313 | 19 | 529 |
| k0.Q124C-IgG2 | 17 | 313 | 19 | 530 |
| k0.L125C-IgG2 | 17 | 313 | 19 | 531 |
| k0.K126C-IgG2 | 17 | 313 | 19 | 532 |
| k0.S127C-IgG2 | 17 | 313 | 19 | 533 |
| k0.G128C-IgG2 | 17 | 313 | 19 | 534 |
| k0.T129C-IgG2 | 17 | 313 | 19 | 535 |
| k0.A130C-IgG2 | 17 | 313 | 19 | 536 |
| k0.S131C-IgG2 | 17 | 313 | 19 | 537 |
| k0.L136C-IgG2 | 17 | 313 | 19 | 538 |
| k0.N137C-IgG2 | 17 | 313 | 19 | 539 |
| k0.N138C-IgG2 | 17 | 313 | 19 | 540 |
| k0.F139C-IgG2 | 17 | 313 | 19 | 541 |
| k0.Y140C-IgG2 | 17 | 313 | 19 | 542 |
| k0.P141C-IgG2 | 17 | 313 | 19 | 543 |
| k0.R142C-IgG2 | 17 | 313 | 19 | 544 |
| k0.E143C-IgG2 | 17 | 313 | 19 | 545 |
| k0.A144C-IgG2 | 17 | 313 | 19 | 546 |
| k0.K145C-IgG2 | 17 | 313 | 19 | 547 |
| k0.V146C-IgG2 | 17 | 313 | 19 | 548 |
| k0.Q147C-IgG2 | 17 | 313 | 19 | 549 |
| k0.W148C-IgG2 | 17 | 313 | 19 | 550 |
| k0.K149C-IgG2 | 17 | 313 | 19 | 551 |
| k0.V150C-IgG2 | 17 | 313 | 19 | 552 |
| k0.D151C-IgG2 | 17 | 313 | 19 | 553 |
| k0.N152C-IgG2 | 17 | 313 | 19 | 554 |
| k0.A153C-IgG2 | 17 | 313 | 19 | 555 |
| k0.L154C-IgG2 | 17 | 313 | 19 | 556 |
| k0.Q155C-IgG2 | 17 | 313 | 19 | 557 |
| k0.S156C-IgG2 | 17 | 313 | 19 | 558 |
| k0.G157C-IgG2 | 17 | 313 | 19 | 559 |
| k0.N158C-IgG2 | 17 | 313 | 19 | 560 |
| k0.S159C-IgG2 | 17 | 313 | 19 | 561 |
| k0.Q160C-IgG2 | 17 | 313 | 19 | 562 |
| k0.E161C-IgG2 | 17 | 313 | 19 | 563 |
| k0.S162C-IgG2 | 17 | 313 | 19 | 564 |
| k0.V163C-IgG2 | 17 | 313 | 19 | 565 |
| k0.T164C-IgG2 | 17 | 313 | 19 | 566 |
| k0.E165C-IgG2 | 17 | 313 | 19 | 567 |
| k0.Q166C-IgG2 | 17 | 313 | 19 | 568 |
| k0.D167C-IgG2 | 17 | 313 | 19 | 569 |
| k0.S168C-IgG2 | 17 | 313 | 19 | 570 |
| k0.K169C-IgG2 | 17 | 313 | 19 | 571 |
| k0.D170C-IgG2 | 17 | 313 | 19 | 572 |
| k0.S171C-IgG2 | 17 | 313 | 19 | 573 |
| k0.T172C-IgG2 | 17 | 313 | 19 | 574 |
| k0.Y173C-IgG2 | 17 | 313 | 19 | 575 |
| k0.S174C-IgG2 | 17 | 313 | 19 | 576 |
| k0.L175C-IgG2 | 17 | 313 | 19 | 577 |
| k0.T180C-IgG2 | 17 | 313 | 19 | 578 |
| k0.L181C-IgG2 | 17 | 313 | 19 | 579 |
| k0.S182C-IgG2 | 17 | 313 | 19 | 580 |
| k0.K183C-IgG2 | 17 | 313 | 19 | 581 |
| k0.A184C-IgG2 | 17 | 313 | 19 | 582 |
| k0.D185C-IgG2 | 17 | 313 | 19 | 583 |
| k0.Y186C-IgG2 | 17 | 313 | 19 | 584 |
| k0.E187C-IgG2 | 17 | 313 | 19 | 585 |
| k0.K188C-IgG2 | 17 | 313 | 19 | 586 |
| k0.H189C-IgG2 | 17 | 313 | 19 | 587 |
| k0.K190C-IgG2 | 17 | 313 | 19 | 588 |
| k0.V191C-IgG2 | 17 | 313 | 19 | 589 |
| k0.Y192C-IgG2 | 17 | 313 | 19 | 590 |
| k0.A193C-IgG2 | 17 | 313 | 19 | 591 |
| k0.E195C-IgG2 | 17 | 313 | 19 | 592 |
| k0.V196C-IgG2 | 17 | 313 | 19 | 593 |
| k0.T197C-IgG2 | 17 | 313 | 19 | 594 |
| k0.H198C-IgG2 | 17 | 313 | 19 | 595 |
| k0.Q199C-IgG2 | 17 | 313 | 19 | 596 |
| k0.G200C-IgG2 | 17 | 313 | 19 | 597 |
| k0.L201C-IgG2 | 17 | 313 | 19 | 598 |
| k0.S202C-IgG2 | 17 | 313 | 19 | 599 |
| k0.S203C-IgG2 | 17 | 313 | 19 | 600 |
| k0.P204C-IgG2 | 17 | 313 | 19 | 601 |
| k0.V205C-IgG2 | 17 | 313 | 19 | 602 |
| k0.T206C-IgG2 | 17 | 313 | 19 | 603 |
| k0.K207C-IgG2 | 17 | 313 | 19 | 604 |
| k0.S208C-IgG2 | 17 | 313 | 19 | 605 |
| k0.F209C-IgG2 | 17 | 313 | 19 | 606 |
| k0.N210C-IgG2 | 17 | 313 | 19 | 607 |
| k0.R211C-IgG2 | 17 | 313 | 19 | 608 |
| k0.G212C-IgG2 | 17 | 313 | 19 | 609 |
| k0.E213C-IgG2 | 17 | 313 | 19 | 610 |

### Reference Example 10-2 Assessment of Electrophoretic Mobility in Polyacrylamide Gel of Antibodies Having Cysteine Substitution at Various Positions of IgG2

Similarly to Reference Example 8-2, non-reducing SDS-PAGE was carried out with the MRA-IgG2 variants produced in Reference Example 10-1, the gel image was captured, and bands were analyzed.

From the obtained gel image, the variants were classified into 7 groups according to the band pattern of each of the MRA-IgG2 variants: Single (one band at a molecular weight region near 140 kDa), Double (two bands at a molecular weight region near 140 kDa), Triple (three bands at a molecular weight region near 140 kDa), Several (four or more bands at a molecular weight region near 140 kDa), LMW (band(s) at a molecular weight region lower than near 140 kDa), HMW (band(s) at a molecular weight region higher than near 140 kDa), and Faint (band(s) blurry and difficult to determine). Grouping results of the band patterns for MRA-IgG2 heavy chain variants and MRA-IgG2 light chain variants are respectively shown in Tables 48 and 49. From Tables 48 and 49, variants classified into the Double and Triple groups are shown in Table 50. It is noted that while Table 33 indicates “no data” for MRAL.K107C-IgG2, position 107 (Kabat numbering), which is the position of cysteine substitution in this variant, is a position where the residue structurally exposed to the surface is present in the hinge region. Accordingly, this variant may also be classified as “Double”.

TABLE 48

| MRA-IgG2 heavy chain | |
|---|---|
| variant name | Group |
| MRAH.Q5C-IgG2 | Double |
| MRAH.E6C-IgG2 | Double |
| MRAH.S7C-IgG2 | Faint |
| MRAH.G8C-IgG2 | Double |
| MRAH.P9C-IgG2 | Double |
| MRAH.G10C-IgG2 | Double |
| MRAH.L11C-IgG2 | Double |
| MRAH.V12C-IgG2 | Double |
| MRAH.R13C-IgG2 | Double |
| MRAH.P14C-IgG2 | Double |
| MRAH.S15C-IgG2 | Double |
| MRAH.Q16C-IgG2 | Double |
| MRAH.T17C-IgG2 | Double |
| MRAH.L18C-IgG2 | Faint |
| MRAH.S19C-IgG2 | Double |
| MRAH.L20C-IgG2 | Faint |
| MRAH.T21C-IgG2 | Double |
| MRAH.T23C-IgG2 | Double |
| MRAH.S25C-IgG2 | Double |
| MRAH.G26C-IgG2 | Double |
| MRAH.S28C-IgG2 | Double |
| MRAH.T30C-IgG2 | Double |
| MRAH.S31C-IgG2 | Double |
| MRAH.W35C-IgG2 | Double |
| MRAH.S35aC-IgG2 | Faint |
| MRAH.Y50C-IgG2 | Faint |
| MRAH.I51C-IgG2 | Double |
| MRAH.S52C-IgG2 | Double |
| MRAH.S62C-IgG2 | Double |
| MRAH.L63C-IgG2 | Double |
| MRAH.K64C-IgG2 | Double |
| MRAH.S65C-IgG2 | Double |
| MRAH.R66C-IgG2 | Double |
| MRAH.V67C-IgG2 | Faint |
| MRAH.T68C-IgG2 | Double |
| MRAH.L70C-IgG2 | Double |
| MRAH.D72C-IgG2 | Double |
| MRAH.T73C-IgG2 | Double |
| MRAH.S74C-IgG2 | HMW |
| MRAH.K75C-IgG2 | Double |
| MRAH.N76C-IgG2 | no data |
| MRAH.Q77C-IgG2 | Double |
| MRAH.S79C-IgG2 | Double |
| MRAH.L80C-IgG2 | Faint |
| MRAH.R81C-IgG2 | Double |
| MRAH.L82C-IgG2 | Faint |
| MRAH.S82aC-IgG2 | Double |
| MRAH.S82bC-IgG2 | Double |
| MRAH.V82cC-IgG2 | Faint |
| MRAH.D101C-IgG2 | Double |
| MRAH.Y102C-IgG2 | Double |
| MRAH.S112C-IgG2 | Double |
| MRAH.S113C-IgG2 | Double |
| G2d.A118C-IgG2 | LMW |
| G2d.S119C-IgG2 | Double |
| G2d.T120C-IgG2 | Double |
| G2d.K121C-IgG2 | Double |
| G2d.G122C-IgG2 | Double |
| G2d.P123C-IgG2 | LMW |
| G2d.S124C-IgG2 | Double |
| G2d.V125C-IgG2 | LMW |
| G2d.F126C-IgG2 | Double |
| G2d.P127C-IgG2 | Faint |
| G2d.S132C-IgG2 | Double |
| G2d.R133C-IgG2 | Double |
| G2d.S134C-IgG2 | Double |
| G2d.T135C-IgG2 | Double |
| G2d.S136C-IgG2 | Double |
| G2d.E137C-IgG2 | Double |
| G2d.S138C-IgG2 | Double |
| G2d.T139C-IgG2 | Faint |
| G2d.A140C-IgG2 | Double |
| G2d.A141C-IgG2 | Faint |
| G2d.D148C-IgG2 | Double |
| G2d.Y149C-IgG2 | Double |
| G2d.F150C-IgG2 | LMW |
| G2d.P151C-IgG2 | no data |
| G2d.E152C-IgG2 | LMW |
| G2d.P153C-IgG2 | HMW |
| G2d.V154C-IgG2 | Faint |
| G2d.T155C-IgG2 | Double |
| G2d.V156C-IgG2 | Double |
| G2d.S157C-IgG2 | no data |
| G2d.W158C-IgG2 | no data |
| G2d.N159C-IgG2 | Double |
| G2d.S160C-IgG2 | Double |
| G2d.G161C-IgG2 | Double |
| G2d.A162C-IgG2 | Double |
| G2d.L163C-IgG2 | Double |
| G2d.T164C-IgG2 | Double |
| G2d.S165C-IgG2 | LMW |
| G2d.G166C-IgG2 | Faint |
| G2d.V167C-IgG2 | Double |
| G2d.V173C-IgG2 | Double |
| G2d.L174C-IgG2 | LMW |
| G2d.Q175C-IgG2 | Double |
| G2d.S176C-IgG2 | Double |
| G2d.S177C-IgG2 | Double |
| G2d.G178C-IgG2 | Double |
| G2d.L179C-IgG2 | Double |
| G2d.Y180C-IgG2 | LMW |
| G2d.V186C-IgG2 | LMW |
| G2d.T187C-IgG2 | Double |
| G2d.V188C-IgG2 | Double |
| G2d.P189C-IgG2 | Double |
| G2d.S190C-IgG2 | Double |
| G2d.S191C-IgG2 | Double |
| G2d.N192C-IgG2 | Double |
| G2d.F193C-IgG2 | Double |
| G2d.G194C-IgG2 | Double |
| G2d.T195C-IgG2 | Double |
| G2d.Q196C-IgG2 | Double |
| G2d.T197C-IgG2 | Double |
| G2d.Y198C-IgG2 | LMW |
| G2d.T199C-IgG2 | LMW |
| G2d.N201C-IgG2 | LMW |
| G2d.V202C-IgG2 | LMW |
| G2d.D203C-IgG2 | LMW |
| G2d.H204C-IgG2 | LMW |
| G2d.K205C-IgG2 | LMW |
| G2d.P206C-IgG2 | LMW |
| G2d.S207C-IgG2 | LMW |
| G2d.N208C-IgG2 | Double |
| G2d.T209C-IgG2 | Double |
| G2d.K210C-IgG2 | Double |
| G2d.V211C-IgG2 | Double |
| G2d.D212C-IgG2 | Double |
| G2d.K213C-IgG2 | Double |
| G2d.T214C-IgG2 | Single |
| G2d.V215C-IgG2 | Single |
| G2d.E216C-IgG2 | Single |
| G2d.R217C-IgG2 | Double |
| G2d.K218C-IgG2 | Double |

TABLE 49

| MRA-IgG2 light chain | |
|---|---|
| variant name | Group |
| MRAL.T5C-IgG2 | Double |
| MRAL.Q6C-IgG2 | Faint |
| MRAL.S7C-IgG2 | Double |
| MRAL.P8C-IgG2 | no data |
| MRAL.S9C-IgG2 | Double |
| MRAL.S10C-IgG2 | Double |
| MRAL.L11C-IgG2 | Double |
| MRAL.S12C-IgG2 | Double |
| MRAL.A13C-IgG2 | Double |
| MRAL.S14C-IgG2 | Double |
| MRAL.V15C-IgG2 | Double |
| MRAL.G16C-IgG2 | Double |
| MRAL.D17C-IgG2 | Double |
| MRAL.R18C-IgG2 | Double |
| MRAL.V19C-IgG2 | Double |
| MRAL.T20C-IgG2 | Double |
| MRAL.I21C-IgG2 | Double |
| MRAL.T22C-IgG2 | Double |
| MRAL.A25C-IgG2 | Faint |
| MRAL.S26C-IgG2 | Double |
| MRAL.Q27C-IgG2 | Double |
| MRAL.Y32C-IgG2 | Double |
| MRAL.L33C-IgG2 | Faint |
| MRAL.N34C-IgG2 | Faint |
| MRAL.Y50C-IgG2 | Double |
| MRAL.T51C-IgG2 | Double |
| MRAL.H55C-IgG2 | Double |
| MRAL.S56C-IgG2 | Double |
| MRAL.G57C-IgG2 | Double |
| MRAL.V58C-IgG2 | Double |
| MRAL.P59C-IgG2 | Double |
| MRAL.S60C-IgG2 | Double |
| MRAL.R61C-IgG2 | Double |
| MRAL.F62C-IgG2 | Faint |
| MRAL.S63C-IgG2 | Double |
| MRAL.S65C-IgG2 | Double |
| MRAL.S67C-IgG2 | Double |
| MRAL.G68C-IgG2 | Double |
| MRAL.T69C-IgG2 | Double |
| MRAL.D70C-IgG2 | Double |
| MRAL.T72C-IgG2 | Double |
| MRAL.F73C-IgG2 | Faint |
| MRAL.T74C-IgG2 | Double |
| MRAL.I75C-IgG2 | no data |
| MRAL.S76C-IgG2 | Double |
| MRAL.S77C-IgG2 | Double |
| MRAL.L78C-IgG2 | Faint |
| MRAL.Q79C-IgG2 | Double |
| MRAL.Y96C-IgG2 | Faint |
| MRAL.T97C-IgG2 | Double |
| MRAL.F98C-IgG2 | Faint |
| MRAL.G99C-IgG2 | Double |
| MRAL.Q100C-IgG2 | Double |
| MRAL.G101C-IgG2 | Double |
| MRAL.T102C-IgG2 | Faint |
| MRAL.K103C-IgG2 | Double |
| MRAL.V104C-IgG2 | Faint |
| MRAL.E105C-IgG2 | Double |
| MRAL.I106C-IgG2 | Faint |
| MRAL.K107C-IgG2 | no data |
| k0.R108C-IgG2 | Double |
| k0.T109C-IgG2 | Double |
| k0.V110C-IgG2 | Double |
| k0.A111C-IgG2 | Double |
| k0.A112C-IgG2 | Double |
| k0.P113C-IgG2 | Double |
| k0.S114C-IgG2 | Double |
| k0.V115C-IgG2 | Faint |
| k0.F116C-IgG2 | Double |
| k0.P120C-IgG2 | Faint |
| k0.S121C-IgG2 | Faint |
| k0.D122C-IgG2 | LMW |
| k0.E123C-IgG2 | Double |
| k0.Q124C-IgG2 | Faint |
| k0.L125C-IgG2 | Double |
| k0.K126C-IgG2 | Triple |
| k0.S127C-IgG2 | Double |
| k0.G128C-IgG2 | Double |
| k0.T129C-IgG2 | Double |
| k0.A130C-IgG2 | Faint |
| k0.S131C-IgG2 | Faint |
| k0.L136C-IgG2 | Faint |
| k0.N137C-IgG2 | no data |
| k0.N138C-IgG2 | Double |
| k0.F139C-IgG2 | Faint |
| k0.Y140C-IgG2 | Faint |
| k0.P141C-IgG2 | Double |
| k0.R142C-IgG2 | Double |
| k0.E143C-IgG2 | Double |
| k0.A144C-IgG2 | Double |
| k0.K145C-IgG2 | Double |
| k0.V146C-IgG2 | Faint |
| k0.Q147C-IgG2 | Double |
| k0.W148C-IgG2 | no data |
| k0.K149C-IgG2 | Double |
| k0.V150C-IgG2 | Faint |
| k0.D151C-IgG2 | Double |
| k0.N152C-IgG2 | Double |
| k0.A153C-IgG2 | Double |
| k0.L154C-IgG2 | Double |
| k0.Q155C-IgG2 | Double |
| k0.S156C-IgG2 | Double |
| k0.G157C-IgG2 | Double |
| k0.N158C-IgG2 | Double |
| k0.S159C-IgG2 | Double |
| k0.Q160C-IgG2 | Double |
| k0.E161C-IgG2 | Double |
| k0.S162C-IgG2 | Double |
| k0.V163C-IgG2 | Double |
| k0.T164C-IgG2 | Double |
| k0.E165C-IgG2 | Double |
| k0.Q166C-IgG2 | Double |
| k0.D167C-IgG2 | Double |
| k0.S168C-IgG2 | Double |
| k0.K169C-IgG2 | Double |
| k0.D170C-IgG2 | Double |
| k0.S171C-IgG2 | Double |
| k0.T172C-IgG2 | Double |
| k0.Y173C-IgG2 | Faint |
| k0.S174C-IgG2 | Faint |
| k0.L175C-IgG2 | Faint |
| k0.T180C-IgG2 | Double |
| k0.L181C-IgG2 | Double |
| k0.S182C-IgG2 | Double |
| k0.K183C-IgG2 | Double |
| k0.A184C-IgG2 | Double |
| k0.D185C-IgG2 | Double |
| k0.Y186C-IgG2 | Double |
| k0.E187C-IgG2 | LMW |
| k0.K188C-IgG2 | Double |
| k0.H189C-IgG2 | Faint |
| k0.K190C-IgG2 | LMW |
| k0.V191C-IgG2 | Double |
| k0.Y192C-IgG2 | Double |
| k0.A193C-IgG2 | Double |
| k0.E195C-IgG2 | Double |
| k0.V196C-IgG2 | Double |
| k0.T197C-IgG2 | Double |
| k0.H198C-IgG2 | Faint |
| k0.Q199C-IgG2 | Double |
| k0.G200C-IgG2 | Triple |
| k0.L201C-IgG2 | Triple |
| k0.S202C-IgG2 | Double |
| k0.S203C-IgG2 | Triple |
| k0.P204C-IgG2 | Double |
| k0.V205C-IgG2 | Triple |
| k0.T206C-IgG2 | Double |
| k0.K207C-IgG2 | Triple |
| k0.S208C-IgG2 | Double |
| k0.F209C-IgG2 | LMW |
| k0.N210C-IgG2 | LMW |
| k0.R211C-IgG2 | LMW |
| k0.G212C-IgG2 | Double |
| k0.E213C-IgG2 | Double |

TABLE 50

| MRA-IgG2 variant | |
|---|---|
| name | Group |
| MRAH.Q5C-IgG2 | Double |
| MRAH.E6C-IgG2 | Double |
| MRAH.G8C-IgG2 | Double |
| MRAH.P9C-IgG2 | Double |
| MRAH.G10C-IgG2 | Double |
| MRAH.L11C-IgG2 | Double |
| MRAH.V12C-IgG2 | Double |
| MRAH.R13C-IgG2 | Double |
| MRAH.P14C-IgG2 | Double |
| MRAH.S15C-IgG2 | Double |
| MRAH.Q16C-IgG2 | Double |
| MRAH.T17C-IgG2 | Double |
| MRAH.S19C-IgG2 | Double |
| MRAH.T21C-IgG2 | Double |
| MRAH.T23C-IgG2 | Double |
| MRAH.S25C-IgG2 | Double |
| MRAH.G26C-IgG2 | Double |
| MRAH.S28C-IgG2 | Double |
| MRAH.T30C-IgG2 | Double |
| MRAH.S31C-IgG2 | Double |
| MRAH.W35C-IgG2 | Double |
| MRAH.I51C-IgG2 | Double |
| MRAH.S52C-IgG2 | Double |
| MRAH.S62C-IgG2 | Double |
| MRAH.L63C-IgG2 | Double |
| MRAH.K64C-IgG2 | Double |
| MRAH.S65C-IgG2 | Double |
| MRAH.R66C-IgG2 | Double |
| MRAH.T68C-IgG2 | Double |
| MRAH.L70C-IgG2 | Double |
| MRAH.D72C-IgG2 | Double |
| MRAH.T73C-IgG2 | Double |
| MRAH.K75C-IgG2 | Double |
| MRAH.Q77C-IgG2 | Double |
| MRAH.S79C-IgG2 | Double |
| MRAH.R81C-IgG2 | Double |
| MRAH.S82aC-IgG2 | Double |
| MRAH.S82bC-IgG2 | Double |
| MRAH.D101C-IgG2 | Double |
| MRAH.Y102C-IgG2 | Double |
| MRAH.S112C-IgG2 | Double |
| MRAH.S113C-IgG2 | Double |
| G2d.S119C-IgG2 | Double |
| G2d.T120C-IgG2 | Double |
| G2d.K121C-IgG2 | Double |
| G2d.G122C-IgG2 | Double |
| G2d.S124C-IgG2 | Double |
| G2d.F126C-IgG2 | Double |
| G2d.S132C-IgG2 | Double |
| G2d.R133C-IgG2 | Double |
| G2d.S134C-IgG2 | Double |
| G2d.T135C-IgG2 | Double |
| G2d.S136C-IgG2 | Double |
| G2d.E137C-IgG2 | Double |
| G2d.S138C-IgG2 | Double |
| G2d.A140C-IgG2 | Double |
| G2d.D148C-IgG2 | Double |
| G2d.Y149C-IgG2 | Double |
| G2d.T155C-IgG2 | Double |
| G2d.V156C-IgG2 | Double |
| G2d.N159C-IgG2 | Double |
| G2d.S160C-IgG2 | Double |
| G2d.G161C-IgG2 | Double |
| G2d.A162C-IgG2 | Double |
| G2d.L163C-IgG2 | Double |
| G2d.T164C-IgG2 | Double |
| G2d.V167C-IgG2 | Double |
| G2d.V173C-IgG2 | Double |
| G2d.Q175C-IgG2 | Double |
| G2d.S176C-IgG2 | Double |
| G2d.S177C-IgG2 | Double |
| G2d.G178C-IgG2 | Double |
| G2d.L179C-IgG2 | Double |
| G2d.T187C-IgG2 | Double |
| G2d.V188C-IgG2 | Double |
| G2d.P189C-IgG2 | Double |
| G2d.S190C-IgG2 | Double |
| G2d.S191C-IgG2 | Double |
| G2d.N192C-IgG2 | Double |
| G2d.F193C-IgG2 | Double |
| G2d.G194C-IgG2 | Double |
| G2d.T195C-IgG2 | Double |
| G2d.Q196C-IgG2 | Double |
| G2d.T197C-IgG2 | Double |
| G2d.N208C-IgG2 | Double |
| G2d.T209C-IgG2 | Double |
| G2d.K210C-IgG2 | Double |
| G2d.V211C-IgG2 | Double |
| G2d.D212C-IgG2 | Double |
| G2d.K213C-IgG2 | Double |
| G2d.R217C-IgG2 | Double |
| G2d.K218C-IgG2 | Double |
| MRAL.T5C-IgG2 | Double |
| MRAL.S7C-IgG2 | Double |
| MRAL.S9C-IgG2 | Double |
| MRAL.S10C-IgG2 | Double |
| MRAL.L11C-IgG2 | Double |
| MRAL.S12C-IgG2 | Double |
| MRAL.A13C-IgG2 | Double |
| MRAL.S14C-IgG2 | Double |
| MRAL.V15C-IgG2 | Double |
| MRAL.G16C-IgG2 | Double |
| MRAL.D17C-IgG2 | Double |
| MRAL.R18C-IgG2 | Double |
| MRAL.V19C-IgG2 | Double |
| MRAL.T20C-IgG2 | Double |
| MRAL.I21C-IgG2 | Double |
| MRAL.T22C-IgG2 | Double |
| MRAL.S26C-IgG2 | Double |
| MRAL.Q27C-IgG2 | Double |
| MRAL.Y32C-IgG2 | Double |
| MRAL.Y50C-IgG2 | Double |
| MRAL.T51C-IgG2 | Double |
| MRAL.H55C-IgG2 | Double |
| MRAL.S56C-IgG2 | Double |
| MRAL.G57C-IgG2 | Double |
| MRAL.V58C-IgG2 | Double |
| MRAL.P59C-IgG2 | Double |
| MRAL.S60C-IgG2 | Double |
| MRAL.R61C-IgG2 | Double |
| MRAL.S63C-IgG2 | Double |
| MRAL.S65C-IgG2 | Double |
| MRAL.S67C-IgG2 | Double |
| MRAL.G68C-IgG2 | Double |
| MRAL.T69C-IgG2 | Double |
| MRAL.D70C-IgG2 | Double |
| MRAL.T72C-IgG2 | Double |
| MRAL.T74C-IgG2 | Double |
| MRAL.S76C-IgG2 | Double |
| MRAL.S77C-IgG2 | Double |
| MRAL.Q79C-IgG2 | Double |
| MRAL.T97C-IgG2 | Double |
| MRAL.G99C-IgG2 | Double |
| MRAL.Q100C-IgG2 | Double |
| MRAL.G101C-IgG2 | Double |
| MRAL.K103C-IgG2 | Double |
| MRAL.E105C-IgG2 | Double |
| k0.R108C-IgG2 | Double |
| k0.T109C-IgG2 | Double |
| k0.V110C-IgG2 | Double |
| k0.A111C-IgG2 | Double |
| k0.A112C-IgG2 | Double |
| k0.P113C-IgG2 | Double |
| k0.S114C-IgG2 | Double |
| k0.F116C-IgG2 | Double |
| k0.E123C-IgG2 | Double |
| k0.L125C-IgG2 | Double |
| k0.K126C-IgG2 | Triple |
| k0.S127C-IgG2 | Double |
| k0.G128C-IgG2 | Double |
| k0.T129C-IgG2 | Double |
| k0.N138C-IgG2 | Double |
| k0.P141C-IgG2 | Double |
| k0.R142C-IgG2 | Double |
| k0.E143C-IgG2 | Double |
| k0.A144C-IgG2 | Double |
| k0.K145C-IgG2 | Double |
| k0.Q147C-IgG2 | Double |
| k0.K149C-IgG2 | Double |
| k0.D151C-IgG2 | Double |
| k0.N152C-IgG2 | Double |
| k0.A153C-IgG2 | Double |
| k0.L154C-IgG2 | Double |
| k0.Q155C-IgG2 | Double |
| k0.S156C-IgG2 | Double |
| k0.G157C-IgG2 | Double |
| k0.N158C-IgG2 | Double |
| k0.S159C-IgG2 | Double |
| k0.Q160C-IgG2 | Double |
| k0.E161C-IgG2 | Double |
| k0.S162C-IgG2 | Double |
| k0.V163C-IgG2 | Double |
| k0.T164C-IgG2 | Double |
| k0.E165C-IgG2 | Double |
| k0.Q166C-IgG2 | Double |
| k0.D167C-IgG2 | Double |
| k0.S168C-IgG2 | Double |
| k0.K169C-IgG2 | Double |
| k0.D170C-IgG2 | Double |
| k0.S171C-IgG2 | Double |
| k0.T172C-IgG2 | Double |
| k0.T180C-IgG2 | Double |
| k0.L181C-IgG2 | Double |
| k0.S182C-IgG2 | Double |
| k0.K183C-IgG2 | Double |
| k0.A184C-IgG2 | Double |
| k0.D185C-IgG2 | Double |
| k0.Y186C-IgG2 | Double |
| k0.K188C-IgG2 | Double |
| k0.V191C-IgG2 | Double |
| k0.Y192C-IgG2 | Double |
| k0.A193C-IgG2 | Double |
| k0.E195C-IgG2 | Double |
| k0.V196C-IgG2 | Double |
| k0.T197C-IgG2 | Double |
| k0.Q199C-IgG2 | Double |
| k0.G200C-IgG2 | Triple |
| k0.L201C-IgG2 | Triple |
| k0.S202C-IgG2 | Double |
| k0.S203C-IgG2 | Triple |
| k0.P204C-IgG2 | Double |
| k0.V205C-IgG2 | Triple |
| k0.T206C-IgG2 | Double |
| k0.K207C-IgG2 | Triple |
| k0.S208C-IgG2 | Double |
| k0.G212C-IgG2 | Double |
| k0.E213C-IgG2 | Double |

### Reference Example 11 Assessment of Antibodies Having Cysteine Substitution at Various Positions of the Lambda Chain

### Reference Example 11-1 Production of Antibodies Having Cysteine Substitution at Various Positions of the Lambda Chain

The light chain (Lambda chain) of an anti-human CXCL10 neutralizing antibody, G7-IgG1 (heavy chain: G7H-G1T4 (SEQ ID NO: 314), light chain: G7L-LT0 (SEQ ID NO: 316)), was subjected to a study in which an arbitrary amino acid residue structurally exposed to the surface was substituted with cysteine.

Amino acid residues within the G7-IgG1 light chain variable region (G7L, SEQ ID NO: 317) were substituted with cysteine to produce variants of the G7-IgG1 light chain variable region shown in Table 51. These variants of the G7-IgG1 light chain variable region were each linked with the G7-IgG1 light chain constant region (LT0, SEQ ID NO: 318) to produce G7-IgG1 light chain variants, and expression vectors encoding the corresponding genes were produced by a method known to the person skilled in the art. In addition, amino acid residues within the G7-IgG1 light chain constant region (LT0, SEQ ID NO: 318) were substituted with cysteine to produce variants of the G7-IgG1 light chain constant region shown in Table 52. These variants of the G7-IgG1 heavy chain constant region were each linked with the G7-IgG1 light chain variable region (G7L, SEQ ID NO: 317) to produce G7-IgG1 light chain variants, and expression vectors encoding the corresponding genes were produced by a method known to the person skilled in the art.

TABLE 51

| | Position of cysteine | |
|---|---|---|
| Variant of G7-IgG1 light | substitution | SEQ ID |
| chain variable region | (Kabat numbering) | NO: |
| G7L.T5C | 5 | 771 |
| G7L.Q6C | 6 | 772 |
| G7L.P7C | 7 | 773 |
| G7L.P8C | 8 | 774 |
| G7L.S9C | 9 | 775 |
| G7L.A11C | 11 | 776 |
| G7L.S12C | 12 | 777 |
| G7L.G13C | 13 | 778 |
| G7L.T14C | 14 | 779 |
| G7L.P15C | 15 | 780 |
| G7L.G16C | 16 | 781 |
| G7L.Q17C | 17 | 782 |
| G7L.R18C | 18 | 783 |
| G7L.V19C | 19 | 784 |
| G7L.T20C | 20 | 785 |
| G7L.I21C | 21 | 786 |
| G7L.S22C | 22 | 787 |
| G7L.G25C | 25 | 788 |
| G7L.S26C | 26 | 789 |
| G7L.S27C | 27 | 790 |
| G7L.S27aC | 27a | 791 |
| G7L.T32C | 32 | 792 |
| G7L.V33C | 33 | 793 |
| G7L.N34C | 34 | 794 |
| G7L.N50C | 50 | 795 |
| G7L.N51C | 51 | 796 |
| G7L.P55C | 55 | 797 |
| G7L.S56C | 56 | 798 |
| G7L.G57C | 57 | 799 |
| G7L.I58C | 58 | 800 |
| G7L.P59C | 59 | 801 |
| G7L.D60C | 60 | 802 |
| G7L.R61C | 61 | 803 |
| G7L.F62C | 62 | 804 |
| G7L.S63C | 63 | 805 |
| G7L.S65C | 65 | 806 |
| G7L.S67C | 67 | 807 |
| G7L.G68C | 68 | 808 |
| G7L.T69C | 69 | 809 |
| G7L.S70C | 70 | 810 |
| G7L.S72C | 72 | 811 |
| G7L.L73C | 73 | 812 |
| G7L.V74C | 74 | 813 |
| G7L.I75C | 75 | 814 |
| G7L.S76C | 76 | 815 |
| G7L.G77C | 77 | 816 |
| G7L.L78C | 78 | 817 |
| G7L.Q79C | 79 | 818 |
| G7L.R96C | 96 | 819 |
| G7L.V97C | 97 | 820 |
| G7L.F98C | 98 | 821 |
| G7L.G99C | 99 | 822 |
| G7L.G100C | 100 | 823 |
| G7L.G101C | 101 | 824 |
| G7L.T102C | 102 | 825 |
| G7L.K103C | 103 | 826 |
| G7L.L104C | 104 | 827 |
| G7L.T105C | 105 | 828 |
| G7L.V106C | 106 | 829 |
| G7L.L106aC | 106a | 830 |

TABLE 52

| | Position of cysteine | |
|---|---|---|
| Variant of G7-IgG1 light | substitution | SEQ ID |
| chain constant region | (Rabat numbering) | NO: |
| LT0.Q108C | 108 | 831 |
| LT0.P109C | 109 | 832 |
| LT0.K110C | 110 | 833 |
| LT0.A111C | 111 | 834 |
| LT0.A112C | 112 | 835 |
| LT0.P113C | 113 | 836 |
| LT0.S114C | 114 | 837 |
| LT0.V115C | 115 | 838 |
| LT0.T116C | 116 | 839 |
| LT0.P120C | 120 | 840 |
| LT0.S121C | 121 | 841 |
| LT0.S122C | 122 | 842 |
| LT0.E123C | 123 | 843 |
| LT0.E124C | 124 | 844 |
| LT0.L125C | 125 | 845 |
| LT0.Q126C | 126 | 846 |
| LT0.A127C | 127 | 847 |
| LT0.N128C | 128 | 848 |
| LT0.K129C | 129 | 849 |
| LT0.A130C | 130 | 850 |
| LT0.T131C | 131 | 851 |
| LT0.I136C | 136 | 852 |
| LT0.S137C | 137 | 853 |
| LT0.D138C | 138 | 854 |
| LT0.F139C | 139 | 855 |
| LT0.Y140C | 140 | 856 |
| LT0.P141C | 141 | 857 |
| LT0.G142C | 142 | 858 |
| LT0.A143C | 143 | 859 |
| LT0.V144C | 144 | 860 |
| LT0.T145C | 145 | 861 |
| LT0.V146C | 146 | 862 |
| LT0.A147C | 147 | 863 |
| LT0.W148C | 148 | 864 |
| LT0.K149C | 149 | 865 |
| LT0.A150C | 150 | 866 |
| LT0.D151C | 151 | 867 |
| LT0.S152C | 152 | 868 |
| LT0.S153C | 153 | 869 |
| LT0.P154C | 154 | 870 |
| LT0.V155C | 155 | 871 |
| LT0.K156C | 156 | 872 |
| LT0.A157C | 157 | 873 |
| LT0.G158C | 158 | 874 |
| LT0.V159C | 159 | 875 |
| LT0.E160C | 160 | 876 |
| LT0.T161C | 161 | 877 |
| LT0.T162C | 162 | 878 |
| LT0.T163C | 163 | 879 |
| LT0.P164C | 164 | 880 |
| LT0.S165C | 165 | 881 |
| LT0.K166C | 166 | 882 |
| LT0.Q167C | 167 | 883 |
| LT0.S168C | 168 | 884 |
| LT0.N170C | 170 | 885 |
| LT0.N171C | 171 | 886 |
| LT0.K172C | 172 | 887 |
| LT0.Y173C | 173 | 888 |
| LT0.A174C | 174 | 889 |
| LT0.A175C | 175 | 890 |
| LT0.S180C | 180 | 891 |
| LT0.L181C | 181 | 892 |
| LT0.T182C | 182 | 893 |
| LT0.P183C | 183 | 894 |
| LT0.E184C | 184 | 895 |
| LT0.Q185C | 185 | 896 |
| LT0.W186C | 186 | 897 |
| LT0.K187C | 187 | 898 |
| LT0.S188C | 188 | 899 |
| LT0.H189C | 189 | 900 |
| LT0.R190C | 190 | 901 |
| LT0.S191C | 191 | 902 |
| LT0.Y192C | 192 | 903 |
| LT0.S193C | 193 | 904 |
| LT0.Q195C | 195 | 905 |
| LT0.V196C | 196 | 906 |
| LT0.T197C | 197 | 907 |
| LT0.H198C | 198 | 908 |
| LT0.E199C | 199 | 909 |
| LT0.G200C | 200 | 910 |
| LT0.S203C | 203 | 911 |
| LT0.T204C | 204 | 912 |
| LT0.V205C | 205 | 913 |
| LT0.E206C | 206 | 914 |
| LT0.K207C | 207 | 915 |
| LT0.T208C | 208 | 916 |
| LT0.V209C | 209 | 917 |
| LT0.A210C | 210 | 918 |
| LT0.P211C | 211 | 919 |
| LT0.T212C | 212 | 920 |
| LT0.E213C | 213 | 921 |

The G7-IgG1 light chain variants produced above were combined with the G7-IgG1 heavy chain and the resultant G7-IgG1 light chain variants shown in Table 53 were expressed by transient expression using FreeStyle293 cells (Invitrogen) or Expi293 cells (Life technologies) by a method known to the person skilled in the art, and purified with Protein A by a method known to the person skilled in the art.

TABLE 53

| | Heavy | Heavy | Light | Light |
|---|---|---|---|---|
| | chain | chain | chain | chain |
| | variable | constant | variable | constant |
| | region | region | region | region |
| G7-IgG1 light chain | SEQ | SEQ | SEQ | SEQ |
| variant name | ID NO: | ID NO: | ID NO: | ID NO: |
| G7L.T5C-IgG1 | 315 | 18 | 771 | 318 |
| G7L.Q6C-IgG1 | 315 | 18 | 772 | 318 |
| G7L.P7C-IgG1 | 315 | 18 | 773 | 318 |
| G7L.P8C-IgG1 | 315 | 18 | 774 | 318 |
| G7L.S9C-IgG1 | 315 | 18 | 775 | 318 |
| G7L.A11C-IgG1 | 315 | 18 | 776 | 318 |
| G7L.S12C-IgG1 | 315 | 18 | 777 | 318 |
| G7L.G13C-IgG1 | 315 | 18 | 778 | 318 |
| G7L.T14C-IgG1 | 315 | 18 | 779 | 318 |
| G7L.P15C-IgG1 | 315 | 18 | 780 | 318 |
| G7L.G16C-IgG1 | 315 | 18 | 781 | 318 |
| G7L.Q17C-IgG1 | 315 | 18 | 782 | 318 |
| G7L.R18C-IgG1 | 315 | 18 | 783 | 318 |
| G7L.V19C-IgG1 | 315 | 18 | 784 | 318 |
| G7L.T20C-IgG1 | 315 | 18 | 785 | 318 |
| G7L.I21C-IgG1 | 315 | 18 | 786 | 318 |
| G7L.S22C-IgG1 | 315 | 18 | 787 | 318 |
| G7L.G25C-IgG1 | 315 | 18 | 788 | 318 |
| G7L.S26C-IgG1 | 315 | 18 | 789 | 318 |
| G7L.S27C-IgG1 | 315 | 18 | 790 | 318 |
| G7L.S27aC-IgG1 | 315 | 18 | 791 | 318 |
| G7L.T32C-IgG1 | 315 | 18 | 792 | 318 |
| G7L.V33C-IgG1 | 315 | 18 | 793 | 318 |
| G7L.N34C-IgG1 | 315 | 18 | 794 | 318 |
| G7L.N50C-IgG1 | 315 | 18 | 795 | 318 |
| G7L.N51C-IgG1 | 315 | 18 | 796 | 318 |
| G7L.P55C-IgG1 | 315 | 18 | 797 | 318 |
| G7L.S56C-IgG1 | 315 | 18 | 798 | 318 |
| G7L.G57C-IgG1 | 315 | 18 | 799 | 318 |
| G7L.I58C-IgG1 | 315 | 18 | 800 | 318 |
| G7L.P59C-IgG1 | 315 | 18 | 801 | 318 |
| G7L.D60C-IgG1 | 315 | 18 | 802 | 318 |
| G7L.R61C-IgG1 | 315 | 18 | 803 | 318 |
| G7L.F62C-IgG1 | 315 | 18 | 804 | 318 |
| G7L.S63C-IgG1 | 315 | 18 | 805 | 318 |
| G7L.S65C-IgG1 | 315 | 18 | 806 | 318 |
| G7L.S67C-IgG1 | 315 | 18 | 807 | 318 |
| G7L.G68C-IgG1 | 315 | 18 | 808 | 318 |
| G7L.T69C-IgG1 | 315 | 18 | 809 | 318 |
| G7L.S70C-IgG1 | 315 | 18 | 810 | 318 |
| G7L.S72C-IgG1 | 315 | 18 | 811 | 318 |
| G7L.L73C-IgG1 | 315 | 18 | 812 | 318 |
| G7L.V74C-IgG1 | 315 | 18 | 813 | 318 |
| G7L.I75C-IgG1 | 315 | 18 | 814 | 318 |
| G7L.S76C-IgG1 | 315 | 18 | 815 | 318 |
| G7L.G77C-IgG1 | 315 | 18 | 816 | 318 |
| G7L.L78C-IgG1 | 315 | 18 | 817 | 318 |
| G7L.Q79C-IgG1 | 315 | 18 | 818 | 318 |
| G7L.R96C-IgG1 | 315 | 18 | 819 | 318 |
| G7L.V97C-IgG1 | 315 | 18 | 820 | 318 |
| G7L.F98C-IgG1 | 315 | 18 | 821 | 318 |
| G7L.G99C-IgG1 | 315 | 18 | 822 | 318 |
| G7L.G100C-IgG1 | 315 | 18 | 823 | 318 |
| G7L.G101C-IgG1 | 315 | 18 | 824 | 318 |
| G7L.T102C-IgG1 | 315 | 18 | 825 | 318 |
| G7L.K103C-IgG1 | 315 | 18 | 826 | 318 |
| G7L.L104C-IgG1 | 315 | 18 | 827 | 318 |
| G7L.T105C-IgG1 | 315 | 18 | 828 | 318 |
| G7L.V106C-IgG1 | 315 | 18 | 829 | 318 |
| G7L.L106aC-IgG1 | 315 | 18 | 830 | 318 |
| LT0.Q108C-IgG1 | 315 | 18 | 317 | 831 |
| LT0.P109C-IgG1 | 315 | 18 | 317 | 832 |
| LT0.K110C-IgG1 | 315 | 18 | 317 | 833 |
| LT0.A111C-IgG1 | 315 | 18 | 317 | 834 |
| LT0.A112C-IgG1 | 315 | 18 | 317 | 835 |
| LT0.P113C-IgG1 | 315 | 18 | 317 | 836 |
| LT0.S114C-IgG1 | 315 | 18 | 317 | 837 |
| LT0.V115C-IgG1 | 315 | 18 | 317 | 838 |
| LT0.T116C-IgG1 | 315 | 18 | 317 | 839 |
| LT0.P120C-IgG1 | 315 | 18 | 317 | 840 |
| LT0.S121C-IgG1 | 315 | 18 | 317 | 841 |
| LT0.S122C-IgG1 | 315 | 18 | 317 | 842 |
| LT0.E123C-IgG1 | 315 | 18 | 317 | 843 |
| LT0.E124C-IgG1 | 315 | 18 | 317 | 844 |
| LT0.L125C-IgG1 | 315 | 18 | 317 | 845 |
| LT0.Q126C-IgG1 | 315 | 18 | 317 | 846 |
| LT0.A127C-IgG1 | 315 | 18 | 317 | 847 |
| LT0.N128C-IgG1 | 315 | 18 | 317 | 848 |
| LT0.K129C-IgG1 | 315 | 18 | 317 | 849 |
| LT0.A130C-IgG1 | 315 | 18 | 317 | 850 |
| LT0.T131C-IgG1 | 315 | 18 | 317 | 851 |
| LT0.I136C-IgG1 | 315 | 18 | 317 | 852 |
| LT0.S137C-IgG1 | 315 | 18 | 317 | 853 |
| LT0.D138C-IgG1 | 315 | 18 | 317 | 854 |
| LT0.F139C-IgG1 | 315 | 18 | 317 | 855 |
| LT0.Y140C-IgG1 | 315 | 18 | 317 | 856 |
| LT0.P141C-IgG1 | 315 | 18 | 317 | 857 |
| LT0.G142C-IgG1 | 315 | 18 | 317 | 858 |
| LT0.A143C-IgG1 | 315 | 18 | 317 | 859 |
| LT0.V144C-IgG1 | 315 | 18 | 317 | 860 |
| LT0.T145C-IgG1 | 315 | 18 | 317 | 861 |
| LT0.V146C-IgG1 | 315 | 18 | 317 | 862 |
| LT0.A147C-IgG1 | 315 | 18 | 317 | 863 |
| LT0.W148C-IgG1 | 315 | 18 | 317 | 864 |
| LT0.K149C-IgG1 | 315 | 18 | 317 | 865 |
| LT0.A150C-IgG1 | 315 | 18 | 317 | 866 |
| LT0.D151C-IgG1 | 315 | 18 | 317 | 867 |
| LT0.S152C-IgG1 | 315 | 18 | 317 | 868 |
| LT0.S153C-IgG1 | 315 | 18 | 317 | 869 |
| LT0.P154C-IgG1 | 315 | 18 | 317 | 870 |
| LT0.V155C-IgG1 | 315 | 18 | 317 | 871 |
| LT0.K156C-IgG1 | 315 | 18 | 317 | 872 |
| LT0.A157C-IgG1 | 315 | 18 | 317 | 873 |
| LT0.G158C-IgG1 | 315 | 18 | 317 | 874 |
| LT0.V159C-IgG1 | 315 | 18 | 317 | 875 |
| LT0.E160C-IgG1 | 315 | 18 | 317 | 876 |
| LT0.T161C-IgG1 | 315 | 18 | 317 | 877 |
| LT0.T162C-IgG1 | 315 | 18 | 317 | 878 |
| LT0.T163C-IgG1 | 315 | 18 | 317 | 879 |
| LT0.P164C-IgG1 | 315 | 18 | 317 | 880 |
| LT0.S165C-IgG1 | 315 | 18 | 317 | 881 |
| LT0.K166C-IgG1 | 315 | 18 | 317 | 882 |
| LT0.Q167C-IgG1 | 315 | 18 | 317 | 883 |
| LT0.S168C-IgG1 | 315 | 18 | 317 | 884 |
| LT0.N170C-IgG1 | 315 | 18 | 317 | 885 |
| LT0.N171C-IgG1 | 315 | 18 | 317 | 886 |
| LT0.K172C-IgG1 | 315 | 18 | 317 | 887 |
| LT0.Y173C-IgG1 | 315 | 18 | 317 | 888 |
| LT0.A174C-IgG1 | 315 | 18 | 317 | 889 |
| LT0.A175C-IgG1 | 315 | 18 | 317 | 890 |
| LT0.S180C-IgG1 | 315 | 18 | 317 | 891 |
| LT0.L181C-IgG1 | 315 | 18 | 317 | 892 |
| LT0.T182C-IgG1 | 315 | 18 | 317 | 893 |
| LT0.P183C-IgG1 | 315 | 18 | 317 | 894 |
| LT0.E184C-IgG1 | 315 | 18 | 317 | 895 |
| LT0.Q185C-IgG1 | 315 | 18 | 317 | 896 |
| LT0.W186C-IgG1 | 315 | 18 | 317 | 897 |
| LT0.K187C-IgG1 | 315 | 18 | 317 | 898 |
| LT0.S188C-IgG1 | 315 | 18 | 317 | 899 |
| LT0.H189C-IgG1 | 315 | 18 | 317 | 900 |
| LT0.R190C-IgG1 | 315 | 18 | 317 | 901 |
| LT0.S191C-IgG1 | 315 | 18 | 317 | 902 |
| LT0.Y192C-IgG1 | 315 | 18 | 317 | 903 |
| LT0.S193C-IgG1 | 315 | 18 | 317 | 904 |
| LT0.Q195C-IgG1 | 315 | 18 | 317 | 905 |
| LT0.V196C-IgG1 | 315 | 18 | 317 | 906 |
| LT0.T197C-IgG1 | 315 | 18 | 317 | 907 |
| LT0.H198C-IgG1 | 315 | 18 | 317 | 908 |
| LT0.E199C-IgG1 | 315 | 18 | 317 | 909 |
| LT0.G200C-IgG1 | 315 | 18 | 317 | 910 |
| LT0.S203C-IgG1 | 315 | 18 | 317 | 911 |
| LT0.T204C-IgG1 | 315 | 18 | 317 | 912 |
| LT0.V205C-IgG1 | 315 | 18 | 317 | 913 |
| LT0.E206C-IgG1 | 315 | 18 | 317 | 914 |
| LT0.K207C-IgG1 | 315 | 18 | 317 | 915 |
| LT0.T208C-IgG1 | 315 | 18 | 317 | 916 |
| LT0.V209C-IgG1 | 315 | 18 | 317 | 917 |
| LT0.A210C-IgG1 | 315 | 18 | 317 | 918 |
| LT0.P211C-IgG1 | 315 | 18 | 317 | 919 |
| LT0.T212C-IgG1 | 315 | 18 | 317 | 920 |
| LT0.E213C-IgG1 | 315 | 18 | 317 | 921 |

### Reference Example 11-2 Assessment of Electrophoretic Mobility in Polyacrylamide Gel of Antibodies Having Cysteine Substitution at Various Positions of the Lambda Chain

Similarly to Reference Example 8-2, non-reducing SDS-PAGE was carried out with the G7-IgG1 variants produced in Reference Example 11-1, the gel image was captured, and bands were quantified.

From the obtained gel image, the variants were classified into 7 groups according to the band pattern of each of the G7-IgG1 variants: Single (one band at a molecular weight region similar to that of G7-IgG1), Double (two bands at a molecular weight region similar to that of G7-IgG1), Triple (three bands at a molecular weight region similar to that of G7-IgG1), Several (four or more bands at a molecular weight region similar to that of G7-IgG1), LMW (band(s) at a molecular weight region lower than that of G7-IgG1), HMW (band(s) at a molecular weight region higher than that of G7-IgG1), and Faint (band(s) blurry and difficult to determine). Regarding the G7-IgG1 variants classified as “Double”, one of the two bands showed the same electrophoretic mobility as G7-IgG1 while the other band showed slightly faster or slower mobility. Thus, for the G7-IgG1 variants classified as “Double”, the percentage of the bands showing different mobility to G7-IgG1 (percentage of new band (%)) was also calculated. Grouping of the band patterns for G7-IgG1 light chain variants and the calculation results of the band percentage are shown in Table 54. From Table 54, variants classified into the Double and Triple groups are shown in Table 55. In these variants, it is highly likely that cysteine substitution caused structural changes such as crosslinkage of Fabs, which resulted in the change in electrophoretic mobility. In this Reference Example, the variant in which the amino acid residue at position 107a (Kabat numbering) was substituted with cysteine was not assessed. However, position 107a (Kabat numbering) is a position where the residue structurally exposed to the surface is present in the hinge region. Thus, in this variant also, it is highly likely that cysteine substitution causes structural changes such as crosslinkage of Fabs, and results in the change in electrophoretic mobility.

TABLE 54

| G7-IgG1 light chain | | Percentage of |
|---|---|---|
| variant name | Group | new band (%) |
| G7L.T5C-IgG1 | Single | — |
| G7L.Q6C-IgG1 | Triple | — |
| G7L.P7C-IgG1 | Single | — |
| G7L.P8C-IgG1 | Single | — |
| G7L.S9C-IgG1 | Single | — |
| G7L.A11C-IgG1 | Single | — |
| G7L.S12C-IgG1 | Single | — |
| G7L.G13C-IgG1 | Single | — |
| G7L.T14C-IgG1 | Single | — |
| G7L.P15C-IgG1 | Single | — |
| G7L.G16C-IgG1 | Faint | — |
| G7L.Q17C-IgG1 | Single | — |
| G7L.R18C-IgG1 | Single | — |
| G7L.V19C-IgG1 | Double | 32.3 |
| G7L.T20C-IgG1 | Single | — |
| G7L.I21C-IgG1 | Faint | — |
| G7L.S22C-IgG1 | Single | — |
| G7L.G25C-IgG1 | Single | — |
| G7L.S26C-IgG1 | Single | — |
| G7L.S27C-IgG1 | Single | — |
| G7L.S27aC-IgG1 | Single | — |
| G7L.T32C-IgG1 | Single | — |
| G7L.V33C-IgG1 | Triple | — |
| G7L.N34C-IgG1 | Double | 43.8 |
| G7L.N50C-IgG1 | Single | — |
| G7L.N51C-IgG1 | Single | — |
| G7L.P55C-IgG1 | Single | — |
| G7L.S56C-IgG1 | Single | — |
| G7L.G57C-IgG1 | Single | — |
| G7L.I58C-IgG1 | Single | — |
| G7L.P59C-IgG1 | Single | — |
| G7L.D60C-IgG1 | Single | — |
| G7L.R61C-IgG1 | Single | — |
| G7L.F62C-IgG1 | Faint | — |
| G7L.S63C-IgG1 | Single | — |
| G7L.S65C-IgG1 | Single | — |
| G7L.S67C-IgG1 | Single | — |
| G7L.G68C-IgG1 | Single | — |
| G7L.T69C-IgG1 | Single | — |
| G7L.S70C-IgG1 | Single | — |
| G7L.S72C-IgG1 | Single | — |
| G7L.L73C-IgG1 | Faint | — |
| G7L.V74C-IgG1 | Single | — |
| G7L.I75C-IgG1 | Faint | — |
| G7L.S76C-IgG1 | Single | — |
| G7L.G77C-IgG1 | Single | — |
| G7L.L78C-IgG1 | Faint | — |
| G7L.Q79C-IgG1 | Single | — |
| G7L.R96C-IgG1 | Single | — |
| G7L.V97C-IgG1 | Faint | — |
| G7L.F98C-IgG1 | Single | — |
| G7L.G99C-IgG1 | Faint | — |
| G7L.G100C-IgG1 | Single | — |
| G7L.G101C-IgG1 | Single | — |
| G7L.T102C-IgG1 | Faint | — |
| G7L.K103C-IgG1 | Single | — |
| G7L.L104C-IgG1 | Faint | — |
| G7L.T105C-IgG1 | Single | — |
| G7L.V106C-IgG1 | Faint | — |
| G7L.L106aC-IgG1 | Single | — |
| LT0.Q108C-IgG1 | Double | 10.6 |
| LT0.P109C-IgG1 | Double | 42.9 |
| LT0.K110C-IgG1 | Single | — |
| LT0.A111C-IgG1 | Single | — |
| LT0.A112C-IgG1 | Single | — |
| LT0.P113C-IgG1 | LMW | — |
| LT0.S114C-IgG1 | Single | — |
| LT0.V115C-IgG1 | LMW | — |
| LT0.T116C-IgG1 | Single | — |
| LT0.P120C-IgG1 | LMW | — |
| LT0.S121C-IgG1 | LMW | — |
| LT0.S122C-IgG1 | no data | — |
| LT0.E123C-IgG1 | Double | 57.5 |
| LT0.E124C-IgG1 | LMW | — |
| LT0.L125C-IgG1 | LMW | — |
| LT0.Q126C-IgG1 | Triple | — |
| LT0.A127C-IgG1 | Single | — |
| LT0.N128C-IgG1 | Single | — |
| LT0.K129C-IgG1 | Single | — |
| LT0.A130C-IgG1 | LMW | — |
| LT0.T131C-IgG1 | LMW | — |
| LT0.I136C-IgG1 | LMW | — |
| LT0.S137C-IgG1 | Single | — |
| LT0.D138C-IgG1 | Single | — |
| LT0.F139C-IgG1 | LMW | — |
| LT0.Y140C-IgG1 | Single | — |
| LT0.P141C-IgG1 | Single | — |
| LT0.G142C-IgG1 | Single | — |
| LT0.A143C-IgG1 | Single | — |
| LT0.V144C-IgG1 | LMW | — |
| LT0.T145C-IgG1 | Single | — |
| LT0.V146C-IgG1 | LMW | — |
| LT0.A147C-IgG1 | Single | — |
| LT0.W148C-IgG1 | no data | — |
| LT0.K149C-IgG1 | Single | — |
| LT0.A150C-IgG1 | Single | — |
| LT0.D151C-IgG1 | Single | — |
| LT0.S152C-IgG1 | Single | — |
| LT0.S153C-IgG1 | Single | — |
| LT0.P154C-IgG1 | Single | — |
| LT0.V155C-IgG1 | Single | — |
| LT0.K156C-IgG1 | Single | — |
| LT0.A157C-IgG1 | Single | — |
| LT0.G158C-IgG1 | no data | — |
| LT0.V159C-IgG1 | Single | — |
| LT0.E160C-IgG1 | Single | — |
| LT0.T161C-IgG1 | Single | — |
| LT0.T162C-IgG1 | Single | — |
| LT0.T163C-IgG1 | Single | — |
| LT0.P164C-IgG1 | Single | — |
| LT0.S165C-IgG1 | Single | — |
| LT0.K166C-IgG1 | Single | — |
| LT0.Q167C-IgG1 | Single | — |
| LT0.S168C-IgG1 | Single | — |
| LT0.N170C-IgG1 | Single | — |
| LT0.N171C-IgG1 | Single | — |
| LT0.K172C-IgG1 | Single | — |
| LT0.Y173C-IgG1 | Single | — |
| LT0.A174C-IgG1 | LMW | — |
| LT0.A175C-IgG1 | LMW | — |
| LT0.S180C-IgG1 | Single | — |
| LT0.L181C-IgG1 | Single | — |
| LT0.T182C-IgG1 | Single | — |
| LT0.P183C-IgG1 | LMW | — |
| LT0.E184C-IgG1 | Single | — |
| LT0.Q185C-IgG1 | Single | — |
| LT0.W186C-IgG1 | LMW | — |
| LT0.K187C-IgG1 | LMW | — |
| LT0.S188C-IgG1 | LMW | — |
| LT0.H189C-IgG1 | LMW | — |
| LT0.R190C-IgG1 | LMW | — |
| LT0.S191C-IgG1 | Single | — |
| LT0.Y192C-IgG1 | Single | — |
| LT0.S193C-IgG1 | Single | — |
| LT0.Q195C-IgG1 | Double | 30.1 |
| LT0.V196C-IgG1 | Double | 82.9 |
| LT0.T197C-IgG1 | Single | — |
| LT0.H198C-IgG1 | Faint | — |
| LT0.E199C-IgG1 | Single | — |
| LT0.G200C-IgG1 | Double | 15.5 |
| LT0.S203C-IgG1 | Double | 32.4 |
| LT0.T204C-IgG1 | Single | — |
| LT0.V205C-IgG1 | Single | — |
| LT0.E206C-IgG1 | Single | — |
| LT0.K207C-IgG1 | Single | — |
| LT0.T208C-IgG1 | Single | — |
| LT0.V209C-IgG1 | LMW | — |
| LT0.A210C-IgG1 | LMW | — |
| LT0.P211C-IgG1 | Faint | — |
| LT0.T212C-IgG1 | LMW | — |
| LT0.E213C-IgG1 | Single | — |

TABLE 55

| G7-IgG1 light chain | | Percentage of |
|---|---|---|
| variant name | Group | new band (%) |
| G7L.Q6C-IgG1 | Triple | — |
| G7L.V19C-IgG1 | Double | 32.3 |
| G7L.V33C-IgG1 | Triple | — |
| G7L.N34C-IgG1 | Double | 43.8 |
| LT0.Q108C-IgG1 | Double | 10.6 |
| LT0.P109C-IgG1 | Double | 42.9 |
| LT0.E123C-IgG1 | Double | 57.5 |
| LT0.Q126C-IgG1 | Triple | — |
| LT0.Q195C-IgG1 | Double | 30.1 |
| LT0.V196C-IgG1 | Double | 82.9 |
| LT0.G200C-IgG1 | Double | 15.5 |
| LT0.S203C-IgG1 | Double | 32.4 |

### Reference Example 12 Assessment of Antibodies Having Cysteine Substitution at Various Positions of VHH

### Reference Example 12-1 Production of Antibodies Having Cysteine Substitution at Various Positions of VHH

An anti-human IL6R neutralizing VHH, IL6R90 (SEQ ID NO: 319) was fused with a human IgG1 Fc region (G1T3dCH1dC, SEQ ID NO: 320) to produce IL6R90-Fc (IL6R90-G1T3dCH1dC, SEQ ID NO: 321), and this was subjected to a study in which an arbitrary amino acid residue among the IL6R90 region structurally exposed to the surface was substituted with cysteine. Amino acid residues within the IL6R90 region were substituted with cysteine, and expression vectors encoding the genes of IL6R90-Fc VHH region variants shown in Table 56 were produced by a method known to the person skilled in the art. These variants of the IL6R90-Fc VHH region were each linked with the Fc region of human IgG1 (G1T3dCH1dC, SEQ ID NO: 320) to produce IL6R90-Fc variants, and expression vectors encoding the corresponding genes were produced by a method known to the person skilled in the art.

TABLE 56

| | Position of cysteine | |
|---|---|---|
| Variant of IL6R90-Fc | substitution | SEQ |
| VHH region | (Kabat numbering) | ID NO: |
| IL6R90.E1C | 1 | 922 |
| IL6R90.V2C | 2 | 923 |
| IL6R90.Q3C | 3 | 924 |
| IL6R90.LAC | 4 | 925 |
| IL6R90.V5C | 5 | 926 |
| IL6R90.E6C | 6 | 927 |
| IL6R90.S7C | 7 | 928 |
| IL6R90.G8C | 8 | 929 |
| IL6R90.G9C | 9 | 930 |
| IL6R90.G10C | 10 | 931 |
| IL6R90.L11C | 11 | 932 |
| IL6R90.V12C | 12 | 933 |
| IL6R90.Q13C | 13 | 934 |
| IL6R90.P14C | 14 | 935 |
| IL6R90.G15C | 15 | 936 |
| IL6R90.G16C | 16 | 937 |
| IL6R90.S17C | 17 | 938 |
| IL6R90.L18C | 18 | 939 |
| IL6R90.R19C | 19 | 940 |
| IL6R90.L20C | 20 | 941 |
| IL6R90.S21C | 21 | 942 |
| IL6R90.A23C | 23 | 943 |
| IL6R90.A24C | 24 | 944 |
| IL6R90.S25C | 25 | 945 |
| IL6R90.G26C | 26 | 946 |
| IL6R90.F27C | 27 | 947 |
| IL6R90.T28C | 28 | 948 |
| IL6R90.F29C | 29 | 949 |
| IL6R90.D30C | 30 | 950 |
| IL6R90.W36C | 36 | 951 |
| IL6R90.V37C | 37 | 952 |
| IL6R90.R38C | 38 | 953 |
| IL6R90.Q39C | 39 | 954 |
| IL6R90.A40C | 40 | 955 |
| IL6R90.P41C | 41 | 956 |
| IL6R90.G42C | 42 | 957 |
| IL6R90.K43C | 43 | 958 |
| IL6R90.A44C | 44 | 959 |
| IL6R90.L45C | 45 | 960 |
| IL6R90.E46C | 46 | 961 |
| IL6R90.W47C | 47 | 962 |
| IL6R90.V48C | 48 | 963 |
| IL6R90.S49C | 49 | 964 |
| IL6R90.R66C | 66 | 965 |
| IL6R90.F67C | 67 | 966 |
| IL6R90.T68C | 68 | 967 |
| IL6R90.I69C | 69 | 968 |
| IL6R90.S70C | 70 | 969 |
| IL6R90.R71C | 71 | 970 |
| IL6R90.D72C | 72 | 971 |
| IL6R90.N73C | 73 | 972 |
| IL6R90.A74C | 74 | 973 |
| IL6R90.K75C | 75 | 974 |
| IL6R90.N76C | 76 | 975 |
| IL6R90.T77C | 77 | 976 |
| IL6R90.L78C | 78 | 977 |
| IL6R90.Y79C | 79 | 978 |
| IL6R90.L80C | 80 | 979 |
| IL6R90.Q81C | 81 | 980 |
| IL6R90.M82C | 82 | 981 |
| IL6R90.N82aC | 82a | 982 |
| IL6R90.S82bC | 82b | 983 |
| IL6R90.L82cC | 82c | 984 |
| IL6R90.R83C | 83 | 985 |
| IL6R90.P84C | 84 | 986 |
| IL6R90.E85C | 85 | 987 |
| IL6R90.D86C | 86 | 988 |
| IL6R90.T87C | 87 | 989 |
| IL6R90.A88C | 88 | 990 |
| IL6R90.V89C | 89 | 991 |
| IL6R90.Y90C | 90 | 992 |
| IL6R90.Y91C | 91 | 993 |
| IL6R90.V93C | 93 | 994 |
| IL6R90.K94C | 94 | 995 |
| IL6R90.W103C | 103 | 996 |
| IL6R90.G104C | 104 | 997 |
| IL6R90.Q105C | 105 | 998 |
| IL6R90.G106C | 106 | 999 |
| IL6R90.T107C | 107 | 1000 |
| IL6R90.L108C | 108 | 1001 |
| IL6R90.V109C | 109 | 1002 |
| IL6R90.T110C | 110 | 1003 |
| IL6R90.V111C | 111 | 1004 |
| IL6R90.S112C | 112 | 1005 |
| IL6R90.S113C | 113 | 1006 |

IL6R90-Fc variants produced above and shown in Table 57 were expressed by transient expression using FreeStyle293 cells (Invitrogen) or Expi293 cells (Life technologies) by a method known to the person skilled in the art, and purified with Protein A by a method known to the person skilled in the art.

TABLE 57

| | VHH | Fc |
|---|---|---|
| | region | region |
| IL6R90-Fc | SEQ | SEQ |
| variant name | ID NO: | ID NO: |
| IL6R90.E1C-Fc | 922 | 320 |
| IL6R90.V2C-Fc | 923 | 320 |
| IL6R90.Q3C-Fc | 924 | 320 |
| IL6R90.L4C-Fc | 925 | 320 |
| IL6R90.V5C-Fc | 926 | 320 |
| IL6R90.E6C-Fc | 927 | 320 |
| IL6R90.S7C-Fc | 928 | 320 |
| IL6R90.G8C-Fc | 929 | 320 |
| IL6R90.G9C-Fc | 930 | 320 |
| IL6R90.G10C-Fc | 931 | 320 |
| IL6R90.L11C-Fc | 932 | 320 |
| IL6R90.V12C-Fc | 933 | 320 |
| IL6R90.Q13C-Fc | 934 | 320 |
| IL6R90.P14C-Fc | 935 | 320 |
| IL6R90.G15C-Fc | 936 | 320 |
| IL6R90.G16C-Fc | 937 | 320 |
| IL6R90.S17C-Fc | 938 | 320 |
| IL6R90.L18C-Fc | 939 | 320 |
| IL6R90.R19C-Fc | 940 | 320 |
| IL6R90.L20C-Fc | 941 | 320 |
| IL6R90.S21C-Fc | 942 | 320 |
| IL6R90.A23C-Fc | 943 | 320 |
| IL6R90.A24C-Fc | 944 | 320 |
| IL6R90.S25C-Fc | 945 | 320 |
| IL6R90.G26C-Fc | 946 | 320 |
| IL6R90.F27C-Fc | 947 | 320 |
| IL6R90.T28C-Fc | 948 | 320 |
| IL6R90.F29C-Fc | 949 | 320 |
| IL6R90.D30C-Fc | 950 | 320 |
| IL6R90.W36C-Fc | 951 | 320 |
| IL6R90.V37C-Fc | 952 | 320 |
| IL6R90.R38C-Fc | 953 | 320 |
| IL6R90.Q39C-Fc | 954 | 320 |
| IL6R90.A40C-Fc | 955 | 320 |
| IL6R90.P41C-Fc | 956 | 320 |
| IL6R90.G42C-Fc | 957 | 320 |
| IL6R90.K43C-Fc | 958 | 320 |
| IL6R90.A44C-Fc | 959 | 320 |
| IL6R90.L45C-Fc | 960 | 320 |
| IL6R90.E46C-Fc | 961 | 320 |
| IL6R90.W47C-Fc | 962 | 320 |
| IL6R90.V48C-Fc | 963 | 320 |
| IL6R90.S49C-Fc | 964 | 320 |
| IL6R90.R66C-Fc | 965 | 320 |
| IL6R90.F67C-Fc | 966 | 320 |
| IL6R90.T68C-Fc | 967 | 320 |
| IL6R90.I69C-Fc | 968 | 320 |
| IL6R90.S70C-Fc | 969 | 320 |
| IL6R90.R71C-Fc | 970 | 320 |
| IL6R90.D72C-Fc | 971 | 320 |
| IL6R90.N73C-Fc | 972 | 320 |
| IL6R90.A74C-Fc | 973 | 320 |
| IL6R90.K75C-Fc | 974 | 320 |
| IL6R90.N76C-Fc | 975 | 320 |
| IL6R90.T77C-Fc | 976 | 320 |
| IL6R90.L78C-Fc | 977 | 320 |
| IL6R90.Y79C-Fc | 978 | 320 |
| IL6R90.L80C-Fc | 979 | 320 |
| IL6R90.Q81C-Fc | 980 | 320 |
| IL6R90.M82C-Fc | 981 | 320 |
| IL6R90.N82aC-Fc | 982 | 320 |
| IL6R90.S82bC-Fc | 983 | 320 |
| IL6R90.L82cC-Fc | 984 | 320 |
| IL6R90.R83C-Fc | 985 | 320 |
| IL6R90.P84C-Fc | 986 | 320 |
| IL6R90.E85C-Fc | 987 | 320 |
| IL6R90.D86C-Fc | 988 | 320 |
| IL6R90.T87C-Fc | 989 | 320 |
| IL6R90.A88C-Fc | 990 | 320 |
| IL6R90.V89C-Fc | 991 | 320 |
| IL6R90.Y90C-Fc | 992 | 320 |
| IL6R90.Y91C-Fc | 993 | 320 |
| IL6R90.V93C-Fc | 994 | 320 |
| IL6R90.K94C-Fc | 995 | 320 |
| IL6R90.W103C-Fc | 996 | 320 |
| IL6R90.G104C-Fc | 997 | 320 |
| IL6R90.Q105C-Fc | 998 | 320 |
| IL6R90.G106C-Fc | 999 | 320 |
| IL6R90.T107C-Fc | 1000 | 320 |
| IL6R90.L108C-Fc | 1001 | 320 |
| IL6R90.V109C-Fc | 1002 | 320 |
| IL6R90.T110C-Fc | 1003 | 320 |
| IL6R90.V111C-Fc | 1004 | 320 |
| IL6R90.S112C-Fc | 1005 | 320 |
| IL6R90.S113C-Fc | 1006 | 320 |

### Reference Example 12-2 Assessment of Electrophoretic Mobility in Polyacrylamide Gel of Antibodies Having Cysteine Substitution at Various Positions of VHH

It was examined with non-reducing SDS-PAGE whether the IL6R90-Fc variants produced in Reference Example 12-1 show a different electrophoretic mobility to IL6R90-Fc. Sample Buffer Solution (2ME-) (×4) (Wako; 198-13282) was used for preparing electrophoresis samples, the samples were treated for 10 minutes under the condition of specimen concentration 50 microgram/mL and 70 degrees C., and then subjected to non-reducing SDS-PAGE. Mini-PROTEAN TGX Precast gel 4-20% 15 well (BIORAD; 456-1096) was used for non-reducing SDS-PAGE and electrophoresis was carried out at 200 V for 2.5 hours. Then, the gel was stained with CBB stain, the gel image was captured with ChemiDocTouchMP (BIORAD), and the bands were quantified with Image Lab (BIORAD).

From the obtained gel image, the variants were classified into 7 groups according to the band pattern of each of the IL6R90-Fc variants: Single (one band at a molecular weight region similar to that of IL6R90-Fc), Double (two bands at a molecular weight region similar to that of IL6R90-Fc), Triple (three bands at a molecular weight region similar to that of IL6R90-Fc), Several (four or more bands at a molecular weight region similar to that of IL6R90-Fc), LMW (band(s) at a molecular weight region lower than that of IL6R90-Fc), HMW (band(s) at a molecular weight region higher than that of IL6R90-Fc), and Faint (band(s) blurry and difficult to determine). Regarding the IL6R90-Fc variants classified as “Double”, one of the two bands showed the same electrophoretic mobility as IL6R90-Fc while the other band showed slightly faster or slower mobility. Thus, for the IL6R90-Fc variants classified as “Double”, the percentage of the bands showing different electrophoretic mobility to IL6R90-Fc (percentage of new band (%)) was also calculated. Grouping of the band patterns for IL6R90-Fc variants and the calculation results of the band percentage are shown in Table 58. From Table 58, variants classified into the Double and Triple groups are shown in Table 59. In these variants, it is highly likely that cysteine substitution caused structural changes such as crosslinkage of VHHs, which resulted in the change in electrophoretic mobility.

TABLE 58

| IL6R90-Fc | | Percentage of |
|---|---|---|
| variant name | Group | new band (%) |
| IL6R90.E1C-Fc | Single | — |
| IL6R90.V2C-Fc | Single | — |
| IL6R90.Q3C-Fc | Single | — |
| IL6R90.L4C-Fc | Triple | — |
| IL6R90.V5C-Fc | Single | — |
| IL6R90.E6C-Fc | Double | 65.2 |
| IL6R90.S7C-Fc | Double | 16.4 |
| IL6R90.G8C-Fc | Double | 38.4 |
| IL6R90.G9C-Fc | Double | 71.8 |
| IL6R90.G10C-Fc | Double | 9.7 |
| IL6R90.L11C-Fc | Double | 59.8 |
| IL6R90.V12C-Fc | Double | 24.8 |
| IL6R90.Q13C-Fc | no data | — |
| IL6R90.P14C-Fc | Double | 16.8 |
| IL6R90.G15C-Fc | Double | 18.6 |
| IL6R90.G16C-Fc | Single | — |
| IL6R90.S17C-Fc | Double | 16.6 |
| IL6R90.L18C-Fc | Single | — |
| IL6R90.R19C-Fc | Single | — |
| IL6R90.L20C-Fc | Double | 57.4 |
| IL6R90.S21C-Fc | Single | — |
| IL6R90.A23C-Fc | Single | — |
| IL6R90.A24C-Fc | Double | 59.3 |
| IL6R90.S25C-Fc | Single | — |
| IL6R90.G26C-Fc | Single | — |
| IL6R90.F27C-Fc | Double | 61.5 |
| IL6R90.T28C-Fc | Single | — |
| IL6R90.F29C-Fc | Double | 56.7 |
| IL6R90.D30C-Fc | Single | — |
| IL6R90.W36C-Fc | no data | — |
| IL6R90.V37C-Fc | Single | — |
| IL6R90.R38C-Fc | Double | 64.5 |
| IL6R90.Q39C-Fc | Double | 12.9 |
| IL6R90.A40C-Fc | Double | 3.2 |
| IL6R90.P41C-Fc | Double | 15.9 |
| IL6R90.G42C-Fc | HMW | — |
| IL6R90.K43C-Fc | Double | 9.2 |
| IL6R90.A44C-Fc | Double | 17.9 |
| IL6R90.L45C-Fc | Double | 15.4 |
| IL6R90.E46C-Fc | Double | 16.4 |
| IL6R90.W47C-Fc | Double | 12.6 |
| IL6R90.V48C-Fc | Double | 14.7 |
| IL6R90.S49C-Fc | Double | 54.1 |
| IL6R90.R66C-Fc | Single | — |
| IL6R90.F67C-Fc | Double | 34.8 |
| IL6R90.T68C-Fc | Single | — |
| IL6R90.I69C-Fc | Double | 57.5 |
| IL6R90.S70C-Fc | Single | — |
| IL6R90.R71C-Fc | Double | 34.3 |
| IL6R90.D72C-Fc | Single | — |
| IL6R90.N73C-Fc | Single | — |
| IL6R90.A74C-Fc | Single | — |
| IL6R90.K75C-Fc | Single | — |
| IL6R90.N76C-Fc | Single | — |
| IL6R90.T77C-Fc | Single | — |
| IL6R90.L78C-Fc | Double | 40.6 |
| IL6R90.Y79C-Fc | Single | — |
| IL6R90.L80C-Fc | Double | 54.7 |
| IL6R90.Q81C-Fc | Single | — |
| IL6R90.M82C-Fc | Double | 47.7 |
| IL6R90.N82aC-Fc | Single | — |
| IL6R90.S82bC-Fc | HMW | — |
| IL6R90.L82cC-Fc | Double | 73.2 |
| IL6R90.R83C-Fc | Single | — |
| IL6R90.P84C-Fc | Single | — |
| IL6R90.E85C-Fc | Double | 9.4 |
| IL6R90.D86C-Fc | no data | — |
| IL6R90.T87C-Fc | Single | — |
| IL6R90.A88C-Fc | Double | 66.5 |
| IL6R90.V89C-Fc | LMW | — |
| IL6R90.Y90C-Fc | no data | — |
| IL6R90.Y91C-Fc | Triple | — |
| IL6R90.V93C-Fc | Triple | — |
| IL6R90.K94C-Fc | Double | 37.7 |
| IL6R90.W103C-Fc | Single | — |
| IL6R90.G104C-Fc | no data | — |
| IL6R90.Q105C-Fc | Single | — |
| IL6R90.G106C-Fc | no data | — |
| IL6R90.T107C-Fc | Double | 53.6 |
| IL6R90.L108C-Fc | Single | — |
| IL6R90.V109C-Fc | Faint | — |
| IL6R90.T110C-Fc | Single | — |
| IL6R90.V111C-Fc | Single | — |
| IL6R90.S112C-Fc | Single | — |
| IL6R90.S113C-Fc | Single | — |

TABLE 59

| IL6R90-Fc | | Percentage of |
|---|---|---|
| variant name | Group | new band (%) |
| IL6R90.L4C-Fc | Triple | — |
| IL6R90.E6C-Fc | Double | 65.2 |
| IL6R90.S7C-Fc | Double | 16.4 |
| IL6R90.G8C-Fc | Double | 38.4 |
| IL6R90.G9C-Fc | Double | 71.8 |
| IL6R90.G10C-Fc | Double | 9.7 |
| IL6R90.L11C-Fc | Double | 59.8 |
| IL6R90.V12C-Fc | Double | 24.8 |
| IL6R90.P14C-Fc | Double | 16.8 |
| IL6R90.G15C-Fc | Double | 18.6 |
| IL6R90.S17C-Fc | Double | 16.6 |
| IL6R90.L20C-Fc | Double | 57.4 |
| IL6R90.A24C-Fc | Double | 59.3 |
| IL6R90.F27C-Fc | Double | 61.5 |
| IL6R90.F29C-Fc | Double | 56.7 |
| IL6R90.R38C-Fc | Double | 64.5 |
| IL6R90.Q39C-Fc | Double | 12.9 |
| IL6R90.A40C-Fc | Double | 3.2 |
| IL6R90.P41C-Fc | Double | 15.9 |
| IL6R90.K43C-Fc | Double | 9.2 |
| IL6R90.A44C-Fc | Double | 17.9 |
| IL6R90.L45C-Fc | Double | 15.4 |
| IL6R90.E46C-Fc | Double | 16.4 |
| IL6R90.W47C-Fc | Double | 12.6 |
| IL6R90.V48C-Fc | Double | 14.7 |
| IL6R90.S49C-Fc | Double | 54.1 |
| IL6R90.F67C-Fc | Double | 34.8 |
| IL6R90.I69C-Fc | Double | 57.5 |
| IL6R90.R71C-Fc | Double | 34.3 |
| IL6R90.L78C-Fc | Double | 40.6 |
| IL6R90.L80C-Fc | Double | 54.7 |
| IL6R90.M82C-Fc | Double | 47.7 |
| IL6R90.L82cC-Fc | Double | 73.2 |
| IL6R90.E85C-Fc | Double | 9.4 |
| IL6R90.A88C-Fc | Double | 66.5 |
| IL6R90.Y91C-Fc | Triple | — |
| IL6R90.V93C-Fc | Triple | — |
| IL6R90.K94C-Fc | Double | 37.7 |
| IL6R90.T107C-Fc | Double | 53.6 |

### Reference Example 13 Assessment of CD3 Agonist Activity of Antibodies Having Cysteine Substitution within the Fab

### Reference Example 13-1 Production of Antibodies Having Cysteine Substitution at the Constant Region

An anti-human CD3 agonist antibody, OKT3 (heavy chain: OKT3VH0000-G1T4 (SEQ ID NO: 1007), light chain: OKT3VL0000-KT0 (SEQ ID NO: 1008)), was subjected to a study in which an arbitrary amino acid residue structurally exposed to the surface was substituted with cysteine.

Amino acid residues within the OKT3 heavy chain constant region (G1T4, SEQ ID NO: 1009) were substituted with cysteine to produce variants of the OKT3 heavy chain constant region shown in Table 60. These variants of the OKT3 heavy chain constant region were each linked with the OKT3 heavy chain variable region (OKT3VH0000, SEQ ID NO: 1010) to produce OKT3 heavy chain variants, and expression vectors encoding the corresponding genes were produced by a method known to the person skilled in the art.

TABLE 60

| Variant of OKT3 | Position of cysteine | |
|---|---|---|
| heavy chain | substitution | SEQ |
| constant region | (EU numbering) | ID NO: |
| G1T4.T135C | 135 | 1017 |
| G1T4.S136C | 136 | 1018 |
| G1T4.S191C | 191 | 1019 |

Similarly, an amino acid residue within the OKT3 light chain constant region (KT0, SEQ ID NO: 1011) was substituted with cysteine to produce a variant of the OKT3 light chain constant region shown in Table 61. This variant of the OKT3 light chain constant region was linked with the OKT3 light chain variable region (OKT3VL0000, SEQ ID NO: 1012) to produce an OKT3 light chain variant, and an expression vector encoding the corresponding gene was produced by a method known to the person skilled in the art.

TABLE 61

| Variant of OKT3 | Position of cysteine | |
|---|---|---|
| light chain | substitution | SEQ |
| constant region | (Kabat numbering) | ID NO: |
| KT0.K126C | 126 | 1020 |

The above-produced OKT3 heavy chain variants and OKT3 light chain variant were each combined with the OKT3 light chain and OKT3 heavy chain, and the OKT3 variants shown in Table 62 were expressed by transient expression using FreeStyle293 cells (Invitrogen) or Expi293 cells (Life technologies) by a method known to the person skilled in the art, and purified with Protein A by a method known to the person skilled in the art. Further, an anti-KLH antibody, IC17 ((heavy chain: IC17HdK-G1T4 (SEQ ID NO: 1013), light chain: IC17L-k0 (SEQ ID NO: 1014)) was similarly prepared as a negative control.

TABLE 62

| | Heavy | Heavy | Light | Light |
|---|---|---|---|---|
| | chain | chain | chain | chain |
| | variable | constant | variable | constant |
| | region | region | region | region |
| Antibody | SEQ | SEQ | SEQ | SEQ |
| name | ID NO: | ID NO: | ID NO: | ID NO: |
| H_T135C | 1010 | 1017 | 1012 | 1011 |
| H_S136C | 1010 | 1018 | 1012 | 1011 |
| H_S191C | 1010 | 1019 | 1012 | 1011 |
| L_K126C | 1010 | 1009 | 1012 | 1020 |

### Reference Example 13-2 Preparation of Jurkat Cell Suspension

Jurkat cells (TCR/CD3 Effector Cells (NFAT), Promega) were collected from flasks. The cells were washed with Assay Buffer (RPMI 1640 medium (Gibco), 10% FBS (HyClone), 1% MEM Non Essential Amino Acids (Invitrogen), and 1 mM Sodium Pyruvate (Invitrogen)), and then suspended at 3×10⁶ cells/mL in Assay Buffer. This suspension of Jurkat cells was subjected to subsequent experiments.

### Reference Example 13-3 Preparation of Luminescence Reagent Solution

100 mL of Bio-Glo Luciferase Assay Buffer (Promega) was added to the bottle of Bio-Glo Luciferase Assay Substrate (Promega), and mixed by inversion. The bottle was protected from light and frozen at −20 degrees C. This luminescence reagent solution was subjected to subsequent experiments.

### Reference Example 13-4 Assessment of T Cell Activation of Antibodies Having Cysteine Substitution at the Constant Region

T cell activation by agonist signaling was assessed based on the fold change of luciferase luminescence. The aforementioned Jurkat cells are cells transformed with a luciferase reporter gene having an NFAT responsive sequence. When the cells are stimulated by an anti-TCR/CD3 antibody, the NFAT pathway is activated via intracellular signaling, thereby inducing luciferase expression. The Jurkat cell suspension prepared as described above was added to a 384-well flat-bottomed white plate at 10 microliter per well (3×10⁴ cells/well). Next, the antibody solution prepared at each concentration (10,000, 1,000, 100, 10, 1, and 0.1 ng/mL) was added at 20 microliter per well. This plate was allowed to stand in a 5% CO₂ incubator at 37 degrees C. for 24 hours. After the incubation, the luminescence reagent solution was thawed, and 30 microliter of the solution was added to each well. The plate was then allowed to stand at room temperature for 10 minutes. Luciferase luminescence in each well of the plate was measured using a luminometer. The amount of luminescence (fold) was determined by dividing the amount of luminescence in the wells added with the antibody with the amount of luminescence in the wells lacking the antibody.

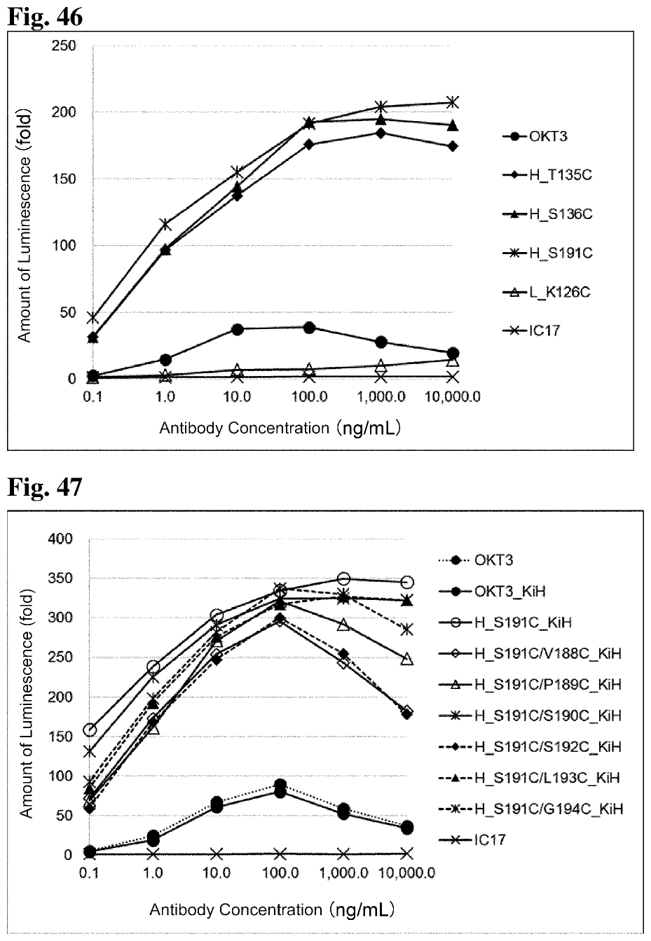

As a result, among the OKT3 variants having cysteine substitution at the constant region, multiple variants greatly increased the T cell activated state as compared to OKT3 as shown in FIG. 46. This result shows that there are multiple cysteine modifications that can crosslink Fabs and enhance CD3 agonist activities.

### Reference Example 14 Assessment of CD3 Agonist Activity of Antibodies Having Different Cysteine Substitutions in the Two Fabs

### Reference Example 14-1 Production of Antibodies Having Heterologous Cysteine Substitution at the Constant Region

An anti-human CD3 agonist antibody, OKT3 (heavy chain: OKT3VH0000-G1T4 (SEQ ID NO: 1007), light chain: OKT3VL0000-KT0 (SEQ ID NO: 1008)), was subjected to a study in which an arbitrary amino acid residue structurally exposed to the surface was substituted with cysteine.

An amino acid residue within the OKT3 heavy chain constant region 1 (G1T4k, SEQ ID NO: 1015) was substituted with cysteine to produce a variant of the OKT3 heavy chain constant region shown in Table 63. This variant of the OKT3 heavy chain constant region was linked with the OKT3 heavy chain variable region (OKT3VH0000, SEQ ID NO: 1010) to produce OKT3 heavy chain variant 1, and an expression vector encoding the corresponding gene was produced by a method known to the person skilled in the art. Similarly, amino acid residues within the OKT3 heavy chain constant region 2 (G1T4h, SEQ ID NO: 1016) were substituted with cysteine to produce variants of the OKT3 heavy chain constant region shown in Table 64. These variants of the OKT3 heavy chain constant region were each linked with the OKT3 heavy chain variable region (OKT3VH0000, SEQ ID NO: 1010) to produce OKT3 heavy chain variant 2, and expression vectors encoding the corresponding genes were produced by a method known to the person skilled in the art. It is noted that heavy-chain constant regions 1 and 2 in this Reference Example are introduced with the Knobs-into-Holes (KiH) modification at the CH3 region for promoting heterodimerization.

TABLE 63

| Variant of OKT3 | Position of cysteine | |
|---|---|---|
| heavy chain | substitution | SEQ |
| constant region 1 | (EU numbering) | ID NO: |
| G1T4k.S191C | 191 | 1022 |

TABLE 64

| Variant of | Position of cysteine | |
|---|---|---|
| OKT3 heavy chain | substitution | SEQ |
| constant region 2 | (EU numbering) | ID NO: |
| G1T4h.V188C | 188 | 1023 |
| G1T4h.P189C | 189 | 1024 |
| G1T4h.S190C | 190 | 1025 |
| G1T4h.S191C | 191 | 1026 |
| G1T4h.S192C | 192 | 1027 |
| G1T4h.L193C | 193 | 1028 |
| G1T4h.G194C | 194 | 1029 |

The above-produced OKT3 heavy chain variant 1 and OKT3 heavy chain variant 2 were combined with the OKT3 light chain, and the OKT3 variants shown in Table 65 were expressed by transient expression using FreeStyle293 cells (Invitrogen) or Expi293 cells (Life technologies) by a method known to the person skilled in the art, and purified with Protein A by a method known to the person skilled in the art. Further, an anti-KLH antibody, IC17 (heavy chain: IC17HdK-G1T4 (SEQ ID NO: 1013), light chain: IC17L-k0 (SEQ ID NO: 1014)) was similarly prepared as a negative control.

TABLE 65

| | Heavy chain variant 1 | | Heavy chain variant 2 | | | |
|---|---|---|---|---|---|---|
| | Heavy | Heavy | Heavy | Heavy | Light | Light |
| | chain | chain | chain | chain | chain | chain |
| | variable | constant | variable | constant | variable | constant |
| | region | region | region | region | region | region |
| | SEQ ID | SEQ ID | SEQ ID | SEQ ID | SEQ ID | SEQ ID |
| Antibody name | NO: | NO: | NO: | NO: | NO: | NO: |
| OKT3_KiH | 1010 | 1015 | 1010 | 1016 | 1012 | 1011 |
| H_S191C_KiH | 1010 | 1022 | 1010 | 1026 | 1012 | 1011 |
| H_S191C/V188C_KiH | 1010 | 1022 | 1010 | 1023 | 1012 | 1011 |
| H_S191C/P189C_KiH | 1010 | 1022 | 1010 | 1024 | 1012 | 1011 |
| H_S191C/S190C_KiH | 1010 | 1022 | 1010 | 1025 | 1012 | 1011 |
| H_S191C/S192C_KiH | 1010 | 1022 | 1010 | 1027 | 1012 | 1011 |
| H_S191C/L193C_KiH | 1010 | 1022 | 1010 | 1028 | 1012 | 1011 |
| H_S191C/G194C_KiH | 1010 | 1022 | 1010 | 1029 | 1012 | 1011 |

### Reference Example 14-2 Preparation of Jurkat Cell Suspension

Jurkat cell suspension was prepared as in Reference Example 13-2.

### Reference Example 14-3 Preparation of Luminescence Reagent Solution

Luminescence reagent solution was prepared as in Reference Example 13-3.

### Reference Example 14-4 Assessment of T Cell Activation of Antibodies Having Heterologous Cysteine Substitution at the Constant Region

T cell activation was assessed as in Reference Example 13-4.

As a result, OKT3 variants having different cysteine substitutions at the two constant regions of the antibody greatly increased the T cell activated state as compared to OKT3, as shown in FIG. 47. This result shows that even different cysteine substitutions between the Fabs can crosslink Fabs and enhance CD3 agonist activities.

### Reference Example 15 Assessment of CD3 Agonist Activity of Antibodies Having Charge Modification within the Fab

### Reference Example 15-1 Production of Antibodies Having Charged Amino Acid Substitution at the Constant Region

The heavy chain of an anti-human CD3 agonist antibody, OKT3 (heavy chain: OKT3VH0000-G1T4 (SEQ ID NO: 1007), light chain: OKT3VL0000-KT0 (SEQ ID NO: 1008)), was subjected to a study in which an arbitrary amino acid residue structurally exposed to the surface was substituted with charged amino acid.

Amino acid residues within the OKT3 heavy chain constant region 1 (G1T4k, SEQ ID NO: 1015) were substituted with arginine (R) or lysine (K) to produce a variant of the OKT3 heavy chain constant region shown in Table 66. This variant of the OKT3 heavy chain constant region was linked with the OKT3 heavy chain variable region (OKT3VH0000, SEQ ID NO: 1010) to produce OKT3 heavy chain variant 1, and an expression vector encoding the corresponding gene was produced by a method known to the person skilled in the art. Similarly, amino acid residues within the OKT3 heavy chain constant region 2 (G1T4h, SEQ ID NO: 1016) were substituted with aspartic acid (D) or glutamic acid (E) to produce variants of the OKT3 heavy chain constant region shown in Table 67. These variants of the OKT3 heavy chain constant region were each linked with the OKT3 heavy chain variable region (OKT3VH0000, SEQ ID NO: 1010) to produce OKT3 heavy chain variant 2, and expression vectors encoding the corresponding genes were produced by a method known to the person skilled in the art. It is noted that the CH3 regions of heavy chain constant regions 1 and 2 in this Reference Example are introduced with the Knobs-into-Holes (KiH) modification for promoting heterodimerization.

TABLE 66

| Variant of | Amino acid | |
|---|---|---|
| OKT3 heavy chain | modification | SEQ |
| constant region 1 | (EU numbering) | ID NO: |
| G1T4k0004 | S134R/T135R/S136R/G137R/S191R/ | 1030 |
| | S192R/L193R/G194R/T195R/Q196R | |

TABLE 67

| Variant of | Amino acid | |
|---|---|---|
| OKT3 heavy chain | modification | SEQ |
| constant region 2 | (EU numbering) | ID NO: |
| G1T4h0004 | S134D/T135D/S136D/G137D/S191D/ | 1031 |
| | S192D/L193D/G194D/T195D/Q196D | |
| G1T4h0006 | S134E/T135E/S136E/G137E/S191E/ | 1032 |
| | S192E/L193E/G194E/T195E/Q196E | |

The above-produced OKT3 heavy chain variant 1 and OKT3 heavy chain variant 2 were combined with the OKT3 light chain, and the OKT3 variants shown in Table 68 were expressed by transient expression using FreeStyle293 cells (Invitrogen) or Expi293 cells (Life technologies) by a method known to the person skilled in the art, and purified with Protein A by a method known to the person skilled in the art. Further, an anti-KLH antibody, IC17 (heavy chain: IC17HdK-G1T4 (SEQ ID NO: 1013), light chain: IC17L-k0 (SEQ ID NO: 1014)) was similarly prepared as a negative control.

TABLE 68

| | Heavy chain variant 1 | | Heavy chain variant 2 | | | |
|---|---|---|---|---|---|---|
| | Heavy | Heavy | Heavy | Heavy | Light | Light |
| | chain | chain | chain | chain | chain | chain |
| | variable | constant | variable | constant | variable | constant |
| | region | region | region | region | region | region |
| Antibody | SEQ ID | SEQ ID | SEQ ID | SEQ ID | SEQ ID | SEQ ID |
| name | NO: | NO: | NO: | NO: | NO: | NO: |
| OKT3_KiH | 1010 | 1015 | 1010 | 1016 | 1012 | 1011 |
| 0004//0004 | 1010 | 1030 | 1010 | 1031 | 1012 | 1011 |
| 0004//0006 | 1010 | 1030 | 1010 | 1032 | 1012 | 1011 |
| 0004//OKT3 | 1010 | 1030 | 1010 | 1016 | 1012 | 1011 |
| OKT3//0004 | 1010 | 1015 | 1010 | 1031 | 1012 | 1011 |
| OKT3//0006 | 1010 | 1015 | 1010 | 1032 | 1012 | 1011 |

### Reference Example 15-2 Preparation of Jurkat Cell Suspension

Jurkat cell suspension was prepared as in Reference Example 13-2.

### Reference Example 15-3 Preparation of Luminescence Reagent Solution

Luminescence reagent solution was prepared as in Reference Example 13-3.

### Reference Example 15-4 Assessment of T Cell Activation of Antibodies Having Substitution with Amino Acids Other than Cysteine at the Constant Region

T cell activation was assessed as in Reference Example 13-4.

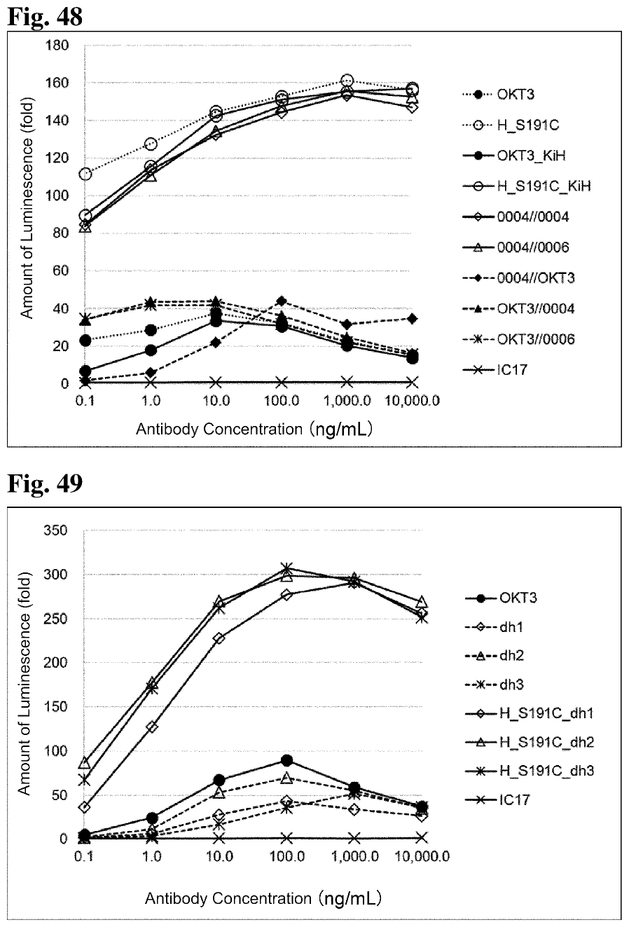

As a result, OKT3 variants introduced with positively charged amino acid substitution at one constant region and with negatively charged amino acid substitution at the other constant region greatly increased the T cell activated state as compared to OKT3 as shown in FIG. 48. Meanwhile, OKT3 variants introduced with positively or negatively charged amino acid substitution at one constant region and with no modification at the other constant region hardly changed the T cell activated state as compared to OKT3. This result shows that not only cysteine substitution but also charged amino acid substitution can crosslink Fabs by noncovalent bond and enhance CD3 agonist activities.

### Reference Example 16 Assessment of CD3 Agonist Activity of Antibodies Having Cysteine Substitution within the Fab and Lacking Disulfide Bonds in the Hinge Region

### Reference Example 16-1 Production of Antibodies Having Cysteine Substitution within the Fab and Lacking Disulfide Bonds in the Hinge Region

The heavy chain of an anti-human CD3 agonist antibody, OKT3 (heavy chain: OKT3VH0000-G1T4 (SEQ ID NO: 1007), light chain: OKT3VL0000-KT0 (SEQ ID NO: 1008)), was subjected to a study in which the disulfide bonds in the hinge region were removed and an amino acid residue structurally exposed to the surface was substituted with cysteine.

Cysteine in the hinge region of OKT3 heavy chain constant region (G1T4, SEQ ID NO: 1009) was substituted with serine to produce variants of the OKT3 heavy chain constant region shown in Table 69. The amino acid residue at position 191 (EU numbering) of these variants of OKT3 heavy chain constant region was substituted with cysteine to produce variants of the OKT3 heavy chain constant region shown in Table 70. These variants of the OKT3 heavy chain constant region were each linked with the OKT3 heavy chain variable region (OKT3VH0000, SEQ ID NO: 1010) to produce OKT3 heavy chain variants, and expression vectors encoding the corresponding genes were produced by a method known to the person skilled in the art.

TABLE 69

| Variant of | Amino acid | |
|---|---|---|
| OKT3 heavy chain | modification | SEQ |
| constant region | (EU numbering) | ID NO: |
| G1T4.dh1 | C226S | 1033 |
| G1T4.dh2 | C229S | 1034 |
| G1T4.dh3 | C226S/C229S | 1035 |

TABLE 70

| Variant of | Amino acid | |
|---|---|---|
| OKT3 heavy chain | modification | SEQ |
| constant region | (EU numbering) | ID NO: |
| G1T4.S191C.dh1 | S191C/C226S | 1036 |
| G1T4.S191C.dh2 | S191C/C229S | 1037 |
| G1T4.S191C.dh3 | S191C/C226S/C229S | 1038 |

The above-produced OKT3 heavy chain variants were combined with the OKT3 light chain, and the OKT3 variants shown in Table 71 were expressed by transient expression using FreeStyle293 cells (Invitrogen) or Expi293 cells (Life technologies) by a method known to the person skilled in the art, and purified with Protein A by a method known to the person skilled in the art. Further, an anti-KLH antibody, IC17 (heavy chain: IC17HdK-G1T4 (SEQ ID NO: 1013), light chain: IC17L-k0 (SEQ ID NO: 1014)) was similarly prepared as a negative control.

TABLE 71

| | Heavy | Heavy | Light | Light |
|---|---|---|---|---|
| | chain | chain | chain | chain |
| | variable | constant | variable | constant |
| | region | region | region | region |
| Antibody | SEQ | SEQ | SEQ | SEQ |
| name | ID NO: | ID NO: | ID NO: | ID NO: |
| dh1 | 1010 | 1033 | 1012 | 1011 |
| dh2 | 1010 | 1034 | 1012 | 1011 |
| dh3 | 1010 | 1035 | 1012 | 1011 |
| H_S191C_dh1 | 1010 | 1036 | 1012 | 1011 |
| H_S191C_dh2 | 1010 | 1037 | 1012 | 1011 |
| H_S191C_dh3 | 1010 | 1038 | 1012 | 1011 |

### Reference Example 16-2 Preparation of Jurkat Cell Suspension

Jurkat cell suspension was prepared as in Reference Example 13-2.

### Reference Example 16-3 Preparation of Luminescence Reagent Solution

Luminescence reagent solution was prepared as in Reference Example 13-3.

### Reference Example 16-4 Assessment of T Cell Activation of Antibodies Having Cysteine Substitution within the Fab and Lacking Disulfide Bonds in the Hinge Region

T cell activation was assessed as in Reference Example 13-4.

As a result, OKT3 variants with only the disulfide bonds in the hinge region removed reduced or hardly changed the T cell activated state as compared to OKT3 as shown in FIG. 49. On the other hand, OKT3 variants with the disulfide bonds in the hinge region removed and introduced with cysteine substitution at the constant region greatly increased the T cell activated state as compared to OKT3. This result shows that even when there is no disulfide bond in the hinge region, cysteine substitution within the Fab can crosslink Fabs and enhance CD3 agonist activities.

### Reference Example 17 Production of Expression Vectors for Modified Antibodies, and Expression and Purification of Modified Antibodies

An antibody gene inserted in an expression vector for animal cells was subjected to amino acid residue sequence substitution by a method known to the person skilled in the art using PCR, the In-Fusion Advantage PCR cloning kit (TAKARA), or such, to construct an expression vector for a modified antibody. The nucleotide sequence of the resulting expression vector was determined by a method known to the person skilled in the art. The produced expression vector was transiently introduced into FreeStyle293 (registered trademark) or Expi293 (registered trademark) cells (Invitrogen) and the cells were allowed to express the modified antibody into culture supernatant. The modified antibody was purified from the obtained culture supernatant by a method known to the person skilled in the art using Protein A and such. Absorbance at 280 nm was measured using a spectrophotometer. An absorption coefficient was calculated from the measured value using the PACE method and used to calculate the antibody concentration (Protein Science 1995; 4:2411-2423).

### Reference Example 18 Preparation of Bispecific Antibodies

The purified antibody was dialyzed into TBS or PBS buffer and its concentration was adjusted to 1 mg/mL. As a 10× reaction buffer, 250 mM 2-MEA (SIGMA) was prepared. Two different homodimeric antibodies prepared in Reference Example 17 were mixed in equal amount. To this mixture, a 1/10 volume of the 10× reaction buffer was added and mixed. The mixture was allowed to stand at 37 degrees C. for 90 minutes. After the reaction, the mixture was dialyzed into TBS or PBS to obtain a solution of a bispecific antibody in which the above two different antibodies were heterodimerized. The antibody concentration was measured by the above-mentioned method, and the antibody was subjected to subsequent experiments.

### Reference Example 19 Assessment of Agonist Activity

### Reference Example 19-1 Preparation of Jurkat Cell Suspension

Jurkat cells (TCR/CD3 Effector Cells (NFAT), Promega) were collected from flasks. The cells were washed with Assay Buffer (RPMI 1640 medium (Gibco), 10% FBS (HyClone), 1% MEM Non Essential Amino Acids (Invitrogen), and 1 mM Sodium Pyruvate (Invitrogen)), and then suspended at 3×10⁶ cells/mL in Assay Buffer. This suspension of Jurkat cells was subjected to subsequent experiments.

### Reference Example 19-2 Preparation of Luminescence Reagent Solution

100 mL of Bio-Glo Luciferase Assay Buffer (Promega) was added to the bottle of Bio-Glo Luciferase Assay Substrate (Promega), and mixed by inversion. The bottle was protected from light and frozen at −20 degrees C. This luminescence reagent solution was subjected to subsequent experiments.

### Reference Example 19-3 T Cell Activation Assay

T cell activation by agonist signaling was assessed based on the fold change of luciferase luminescence. The aforementioned Jurkat cells are cells transformed with a luciferase reporter gene having an NFAT responsive sequence. When the cells are stimulated by an anti-TCR/CD3 antibody, the NFAT pathway is activated via intracellular signaling, thereby inducing luciferase expression. The Jurkat cell suspension prepared as described above was added to a 384-well flat-bottomed white plate at 10 microliter per well (3×10⁴ cells/well). Next, the antibody solution prepared at each concentration (150, 15, 1.5, 0.15, 0.015, 0.0015, 0.00015, and 0.000015 nM) was added at 20 microliter per well. This plate was allowed to stand in a 5% CO₂ incubator at 37 degrees C. for 24 hours. After the incubation, the luminescence reagent solution was thawed, and 30 microliter of the solution was added to each well. The plate was then allowed to stand at room temperature for 10 minutes. Luciferase luminescence in each well of the plate was measured using a luminometer.

### Reference Example 20 Assessment of Agonist Activity of CD3 Biparatopic Antibodies Using Jurkat Cells

Antibodies were prepared and their activities were assessed according to Reference Examples 17, 18, and 19. The antibodies used in this Example are shown in Table 72.

TABLE 72

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| CD3-G1sLL | 1039 | 1040 | — | — | Monospecific antibody |
| CD3//OKT3-G1s | 1041 | 1042 | 1043 | 1044 | Bispecific antibody |
| CD3//OKT3-G1sHH | 1045 | 1046 | 1047 | 1048 | Bispecific antibody |
| CD3//OKT3-G1sLH | 1049 | 1050 | 1051 | 1052 | Bispecific antibody |
| OKT3-G1s | 1053 | 1054 | — | — | Monospecific antibody |
| OKT3-G1sHH | 1055 | 1056 | — | — | Monospecific antibody |
| CD3-G1sLL + | 1057 | 1058 | 1059 | 1060 | Monospecific antibody |
| OKT3-G1s | | | | | |

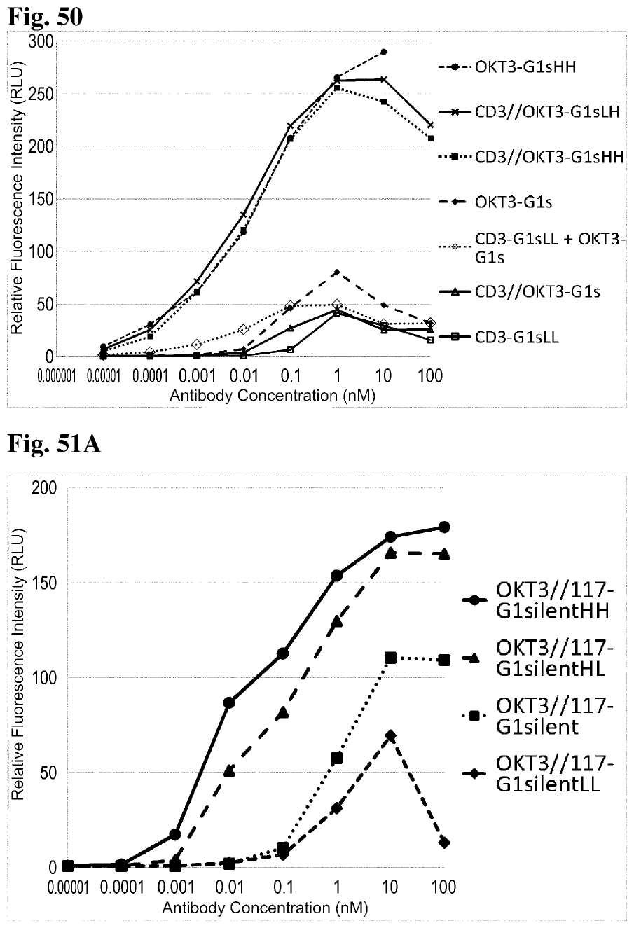

As a result, modified molecules with an additional disulfide bond linking the Fab-Fab of two types of anti-CD3 bispecific antibodies showed varied CD3-mediated signaling compared to bispecific antibodies lacking the additional disulfide bond as shown in FIG. 50.

This result suggests that introducing modifications of the present invention can enhance or diminish agonist activity possessed by bispecific antigen-binding molecules having different epitopes for the same target.

### Reference Example 21 Assessment of CD137 Agonist Activity Using Jurkat Cells

Antibodies were prepared and their activities were assessed according to Reference Examples 17, 18, and 19. The antibodies used in this Reference Example were as follows: an ordinary anti-CD137 antibody, an antibody introduced with a mutation that promotes association of antibodies (hexamerization) in its heavy-chain constant region, and modified antibodies produced by linking the Fab-Fab of each of the above antibodies with an additional disulfide bond.

T cell activation by agonist signaling was assessed based on the fold change of luciferase luminescence. The cells of GloResponse™ NF-kappa B-Luc2/4-1BB Jurkat cell line (Promega) are cells transformed with a luciferase reporter gene having an NFAT responsive sequence. When the cells are stimulated by an anti-CD137 antibody, the NFAT pathway is activated via intracellular signaling, thereby inducing luciferase expression. The Jurkat cell suspension prepared at 2×10⁶ cells/mL with Assay medium (99% RPMI, 1% FBS) was added to a 96-well flat-bottomed white plate at 25 microliter per well (5×10⁴ cells/well). Next, the antibody solution containing ATP or the antibody solution without ATP prepared at each antibody concentration (final concentration: 45, 15, 5, 1.667, 0.556, 0.185, 0.062, and 0.021 microgram/mL) was added at 25 microliter per well. The final concentration of ATP was 250 nM. This plate was allowed to stand in a 5% CO₂ incubator at 37 degrees C. for 6 hours. After the incubation, the luminescence reagent solution was thawed, and 75 microliter of the solution was added to each well. The plate was then allowed to stand at room temperature for 10 minutes. Luciferase luminescence in each well of the plate was measured using a luminometer. The value of the luminescence of each well divided by the value of the luminescence of the well without antibody addition was defined as Luminescence fold, and it served as an indicator for assessing the activity of each antibody.

As a result, antibodies introduced with the hexamerization modification showed increased agonist activity as compared to an ordinary anti-CD137 antibody. Further, in modified antibodies where each of the antibodies was introduced with additional disulfide bonds, synergistic increase in agonist activity was observed. This result suggests that introducing modifications of the present invention can enhance the activity of an anti-CD137 agonist antibody.

### Reference Example 22 Assessment of Agonist Activity of CD3//PD1 Bispecific Antibodies Using Jurkat Cells

### Reference Example 22-1

Antibodies were prepared and their activities were assessed according to Reference Examples 17, 18, and 19. The antibodies used in this Example are shown in Table 74.

TABLE 74

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| OKT3//117-G1silent | 1113 | 1114 | 1115 | 1116 | Bispecific antibody |
| OKT3//117-G1silentLL | 1117 | 1118 | 1119 | 1120 | Bispecific antibody |
| OKT3//117-G1silentHH | 1121 | 1122 | 1123 | 1124 | Bispecific antibody |
| OKT3//117-G1silentHL | 1125 | 1126 | 1127 | 1128 | Bispecific antibody |
| OKT3//10-G1silent | 1129 | 1130 | 1131 | 1132 | Bispecific antibody |
| OKT3//10-G1silentHH | 1133 | 1134 | 1135 | 1136 | Bispecific antibody |
| OKT3//10-G1silentHL | 1137 | 1138 | 1139 | 1140 | Bispecific antibody |
| CD3//949-G1silent | 1141 | 1142 | 1143 | 1144 | Bispecific antibody |
| CD3//949-G1silentLL | 1145 | 1146 | 1147 | 1148 | Bispecific antibody |
| CD3//949-G1silentHH | 1149 | 1150 | 1151 | 1152 | Bispecific antibody |
| CD3//949-G1silentLH | 1153 | 1154 | 1155 | 1156 | Bispecific antibody |
| CD3//949-G1silentHL | 1157 | 1158 | 1159 | 1160 | Bispecific antibody |
| OKT3//949-G1silent | 1161 | 1162 | 1163 | 1164 | Bispecific antibody |
| OKT3//949-G1silentLL | 1165 | 1166 | 1167 | 1168 | Bispecific antibody |
| OKT3//949-G1silentHH | 1169 | 1170 | 1171 | 1172 | Bispecific antibody |
| OKT3//949-G1silentHL | 1173 | 1174 | 1175 | 1176 | Bispecific antibody |

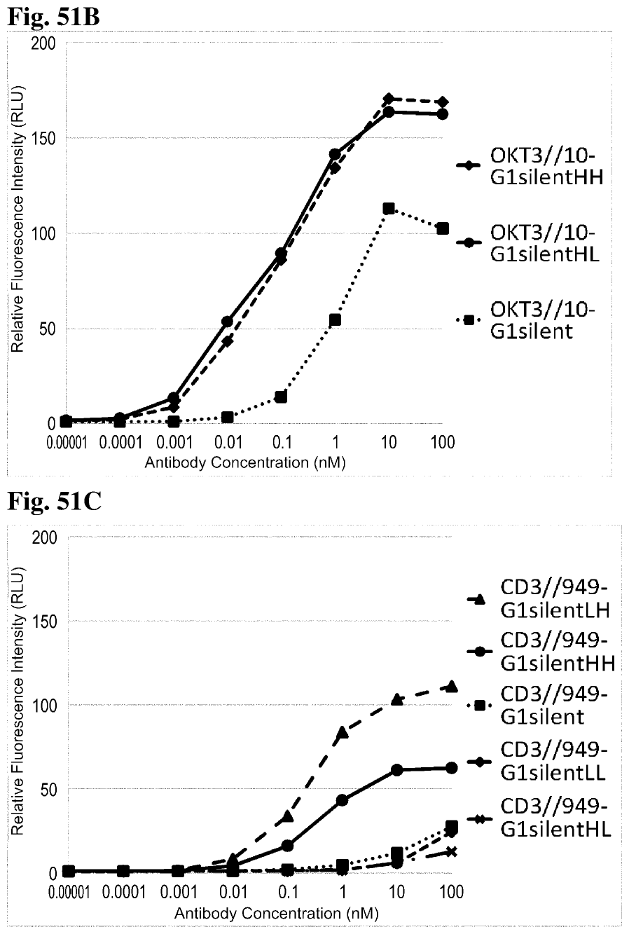

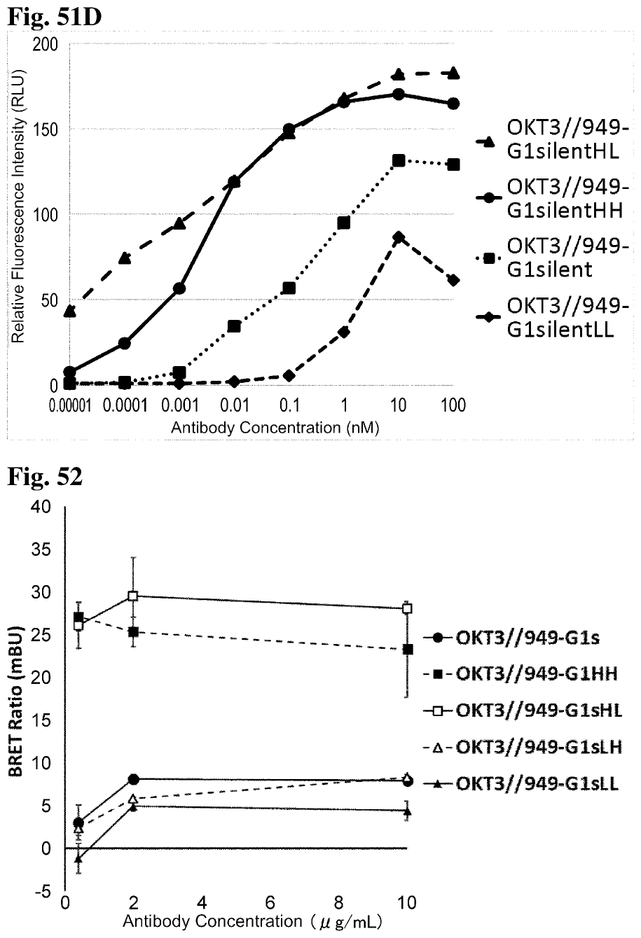

As a result, in multiple bispecific antibodies consisting of a combination of an anti-CD3 antibody and an anti-PD1 antibody, modified molecules with an additional disulfide bond linking the Fab-Fab showed greatly varied CD3- and/or PD1-mediated signaling compared to bispecific antibodies lacking the additional disulfide bond as shown in FIG. 51.

This result suggests that introducing modifications of the present invention can enhance or diminish agonist activity possessed by antigen-binding molecules such as antibodies.

### Reference Example 22-2

Antibodies were prepared and their activities were assessed according to Reference Examples 2, 3, and 4. The antibodies used in this Reference Example are shown in Table 75.

TABLE 75

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| OKT3//949-G1silent | 1161 | 1162 | 1163 | 1164 | Bispecific antibody |
| OKT3//949-G1silentHH | 1169 | 1170 | 1171 | 1172 | Bispecific antibody |
| OKT3//949-G1silentHL | 1173 | 1174 | 1175 | 1176 | Bispecific antibody |
| OKT3//949-G1silentLL | 1165 | 1166 | 1167 | 1168 | Bispecific antibody |
| OKT3//949-G1silentLH | 1177 | 1178 | 1179 | 1180 | Bispecific antibody |

The presence or absence of PD-1 agonist signaling was assessed by the ratio of the fluorescent signal from BRET when PD-1 is in the vicinity of SHP2 (618 nm) and the luminescence originating from SHP2, which is the donor (460 nm). One day before the assay, antigen presenting cells expressing PD-L1 (Promega, #J109A) were seeded into F-12 medium containing 10% FBS (Gibco, 11765-054) in a 96-well plate (Costar, #3917) at 4.0×10⁴ cells/100 microliter/well, and the cells were cultured in a CO₂ incubator for 16-24 hours at 37 degrees C. On the day of the assay, HaloTag nanoBRET 618 Ligand (Promega, #G980A) was diluted 250-fold with Opti-MEM (Gibco, #31985-062). The medium for culturing PD-L1-expressing antigen presenting cells were removed, and the diluted HaloTag nanoBRET 618 Ligand was added at 25 microliter/well. The specimen for assessment diluted with Opti-MEM containing 10 microgram/mL of PD-L1-inhibiting antibodies (40, 8, and 1.6 microgram/mL) was added at 25 microliter/well. PD-1/SHP2 Jurkat cells (Promega, #CS2009A01) were added to the above-noted 96-well plate at 5×10⁴ cells/50 microliter/well, thoroughly suspended, and then incubated in a CO₂ incubator for 2.5 hours at 37 degrees C. nanoBRET Nano-Glo substrate (Promega, #N157A) was diluted 100-fold with Opti-MEM, and this was added at 25 microliter/well to the 96-well plate after incubation. The plate was allowed to stand at room temperature for 30 minutes, and then the Em460 mM and Em618 nm were measured using Envision (PerkinElmer, 2104 EnVision). The obtained values were applied to the following equation to calculate the BRET Ratio (mBU).

618 nm/460 nm=BU

BU×1000=mBU

Mean mBUₑₓₚₑᵣᵢₘₑₙₜₐₗ−Mean mBU_{no PD-L1 block control}=BRET Ratio (mBU)

As a result, in the bispecific antibodies consisting of an anti-CD3 antibody and an anti-PD1 antibody, modified molecules with an additional disulfide bond linking the Fab-Fab showed greatly varied CD3- and/or PD1-mediated signaling compared to bispecific antibodies lacking the additional disulfide bond as shown in FIG. 52.

### Reference Example 23 Assessment of Agonist Activity of CD28/CD3 Clamping Bispecific Antibodies

### Reference Example 23-1 Real-Time Cell Growth Inhibition Assay (xCELLigence Assay)

Antibodies were prepared according to Reference Examples 17 and 18. The antibodies used in this Example are shown in Table 76.

TABLE 76

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| GPC3/attCE115 | 1181 | 1182 | 1183 | 1184 | Bispecific antibody |
| GPC3/attCE115_LL | 1185 | 1186 | 1187 | 1188 | Bispecific antibody |
| KLH/clamp CD3 | 1189 | 1190 | 1191 | 1192 | Bispecific antibody |
| GPC3/clamp CD3 | 1193 | 1194 | 1195 | 1196 | Bispecific antibody |
| CD28/clamp CD3 | 1197 | 1198 | 1199 | 1200 | Bispecific antibody |
| CD28/clamp CD3_HH | 1201 | 1202 | 1203 | 1204 | Bispecific antibody |

T cell-dependent cancer cell growth inhibitory effect of the antibodies was assessed using xCELLigence RTCA MP instrument (ACEA Biosciences). Cells of the human liver cancer cell line SK-Hep-1 forced to express Glypican-3 (GPC3) (SEQ ID NO: 1241) (SK-pca31a) were used as target cells, and human peripheral blood mononuclear cells (PBMC: Cellular Technology Limited (CTL)) were used as effector cells. 1×10⁴ cells of SK-pca31a were seeded onto E-Plate 96 (ACEA Biosciences). On the next day were added 2×10⁵ cells of PBMC and antibodies to make a final concentration of 0.001, 0.01, 0.1, 1, or 10 microgram/mL. Cell growth was monitored every 15 minutes with xCELLigence, and culturing was continued for 72 hours. Cell growth inhibitory effect (CGI: %) was calculated by the following equation.

CGI (%)=100−(CI_{Ab}×100/CI_{NoAb})

In the above equation, “CI_{Ab}” is the Cell index for a well at 72 hours after addition of an antibody (cell growth index measured with xCELLigence). Further, “CI_{NoAb}” is the Cell index for a well after 72 hours without antibody addition.

### Reference Example 23-2 Cytokine Production Assay

Cytokine production from T cells by antibodies was assessed as discussed below.

SK-pca31a was used as the target cell and PBMC (Cellular Technology Limited (CTL)) was used as the effector cell. 1×10⁴ cells of SK-pca31a were seeded onto a 96-well plate. On the next day were added 2×10⁵ cells of PBMC and antibodies to make a final concentration of 0.01, 0.1, 1, or 10 microgram/mL. The culture supernatant was collected after 72 hours, and human IL-6 was measured using AlphaLISA (PerkinElmer).

### Results

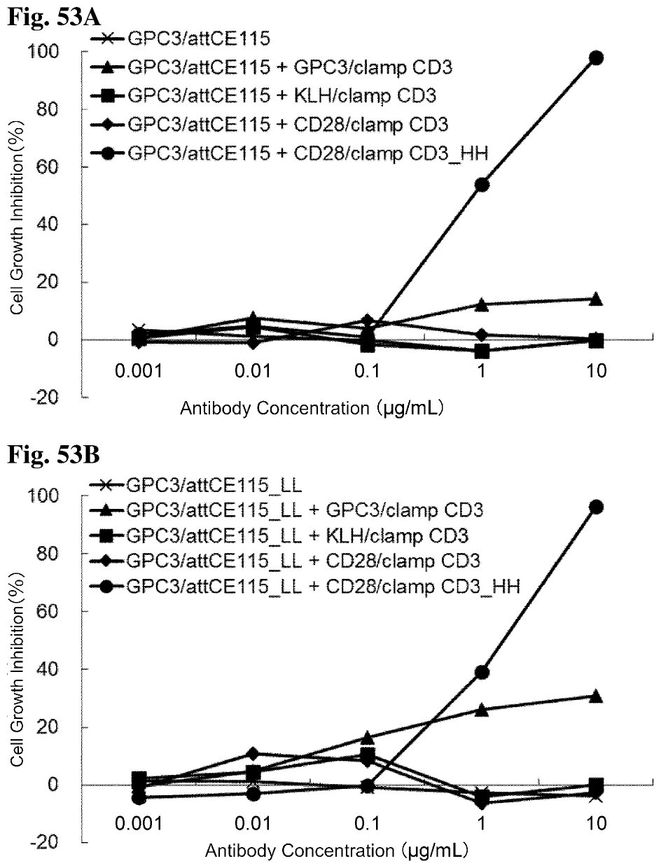

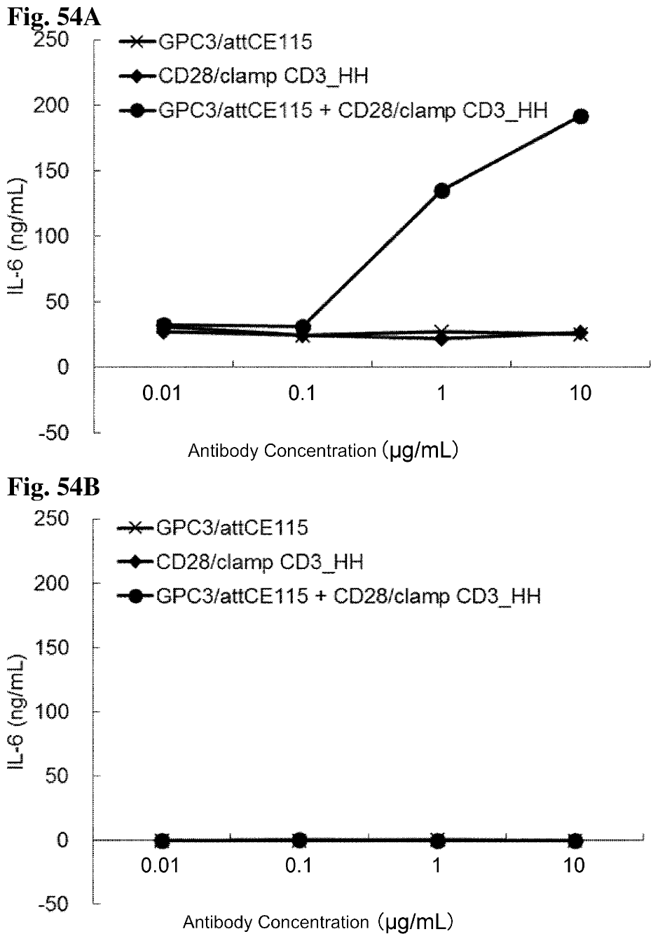

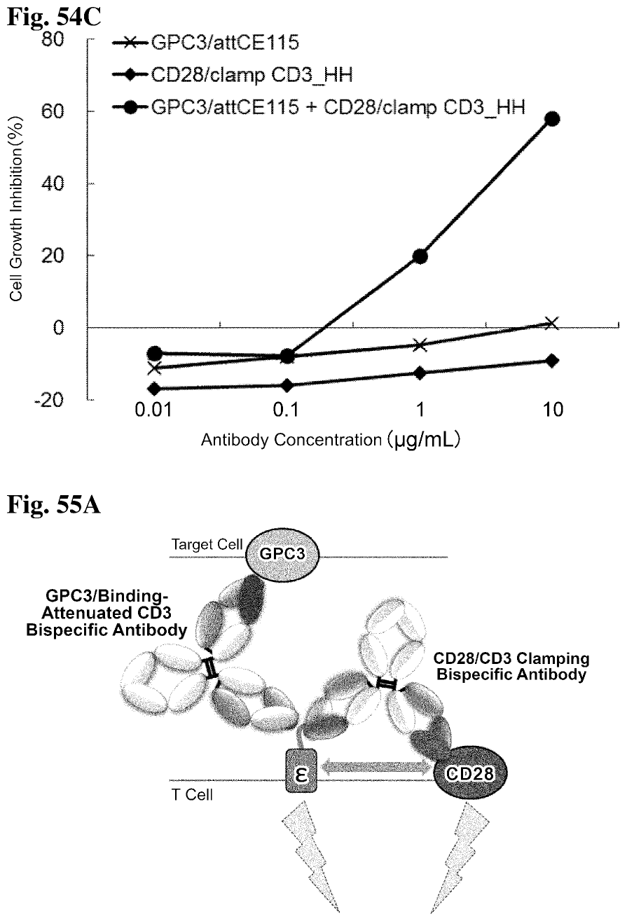

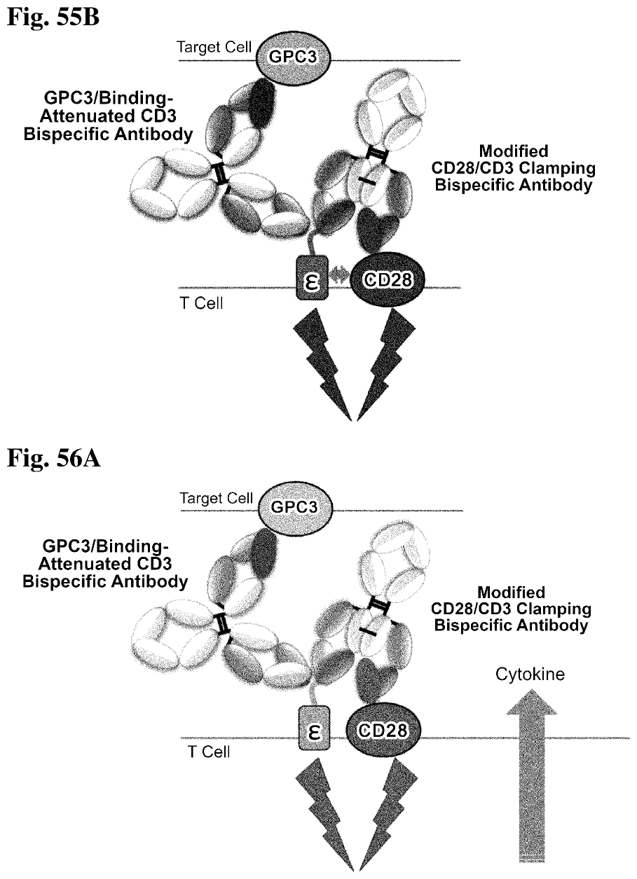

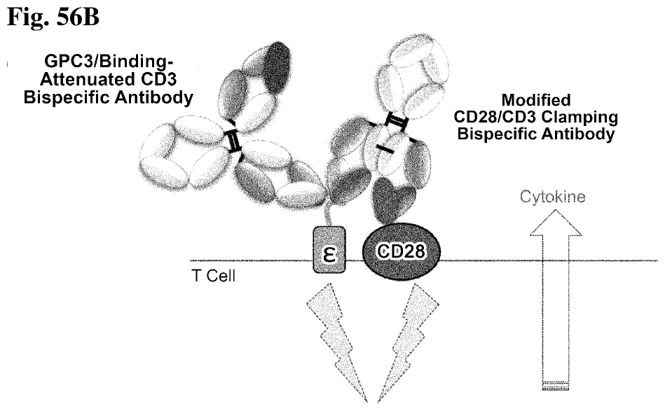

Combined use of CD28/CD3 clamping bispecific antibody and GPC3/binding-attenuated CD3 bispecific antibody did not result in cell growth inhibitory effects. However, inhibitory effects on cancer cell growth were observed by applying modifications for introducing an additional disulfide bond between the Fab-Fab of the CD28/CD3 clamping bispecific antibody (FIGS. 53 and 55). Further, cytokine production was observed when a CD28/CD3 clamping bispecific antibody introduced with the above-noted modification and a GPC3/binding-attenuated CD3 bispecific antibody were cocultured with GPC3 expressing strain and PBMC; however, mere addition of a CD28/CD3 clamping bispecific antibody introduced with the above-noted modification and a GPC3/binding-attenuated CD3 bispecific antibody to PBMC did not result in cytokine production (FIGS. 54 and 56). Accordingly, it was suggested that the effect of the CD28/CD3 clamping bispecific antibody introduced with the above-noted modification and GPC3/binding-attenuated CD3 bispecific antibody on inhibiting cancer cell growth and inducing cytokine production in T cells depends on the expression of cancer antigen.

### Reference Example 24 Assessment of Agonist Activity of CD8/CD28 Bispecific Antibodies

Antibodies were prepared according to Reference Examples 17 and 18. The antibodies used in this Reference Example are shown in Table 77.

TABLE 77

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| KLH-P587 | 1205 | 1206 | — | — | Monospecific antibody |
| CD8/CD28-P587 | 1207 | 1208 | 1209 | 1210 | Bispecific antibody |
| CD8/CD28-P587(HH) | 1211 | 1212 | 1213 | 1214 | Bispecific antibody |
| CD8/CD28-P587(LL) | 1219 | 1220 | 1221 | 1222 | Bispecific antibody |
| CD8/CD28-P587(HL) | 1223 | 1224 | 1225 | 1226 | Bispecific antibody |
| CD8/CD28-P587(LH) | 1227 | 1228 | 1229 | 1230 | Bispecific antibody |

Human peripheral blood mononuclear cells (PBMCs) isolated from healthy volunteer blood samples were used for assessing the prepared specimen. Heparin (0.5 mL) was mixed with 50 mL of blood and was further diluted with 50 mL PBS. Human PBMCs were isolated by the following two steps. In step 1, Leucosep (greiner bio-one) added with Ficoll-Paque PLUS (GE Healthcare) was centrifuged at 1000×g for 1 minute under room temperature, then blood diluted with PBS was added thereto and the mixture was centrifuged at 400×g for 30 minutes under room temperature. In step 2, the buffy coat was collected from the tube after centrifugation and then washed with 60 mL PBS (Wako). The isolated human PBMCs were adjusted to a cell density of 1×10⁷/mL with a medium (5% human serum (SIGMA), 95% AIM-V (Thermo Fischer Scientific)). The resulting cell suspension was seeded onto the wells of a 24-well plate at 1 mL/well and the plate was incubated in a 5% CO₂ incubator at 37 degrees C.

Two days later, the medium was removed from the seeded cells and the cells were washed with 500 microliter PBS, and then collected using accutase (nacalai tesque). Next, the cells were adjusted to make a cell density of 1×10⁶/mL with ViaFluor 405 (Biotium) solution diluted with PBS to make a final concentration of 2 micromolar, and then allowed to stand at 37 degrees C. for 15 minutes. Subsequently, the cells were suspended again with a medium and then seeded onto the wells of a 96-well plate at 2×10⁵ cells per well. Antibody solution was added thereto to make a final concentration of 0.1, 1, and 10 microgram/mL, and the cells were cultured in a 5% CO₂ incubator for 4 days at 37 degrees C.

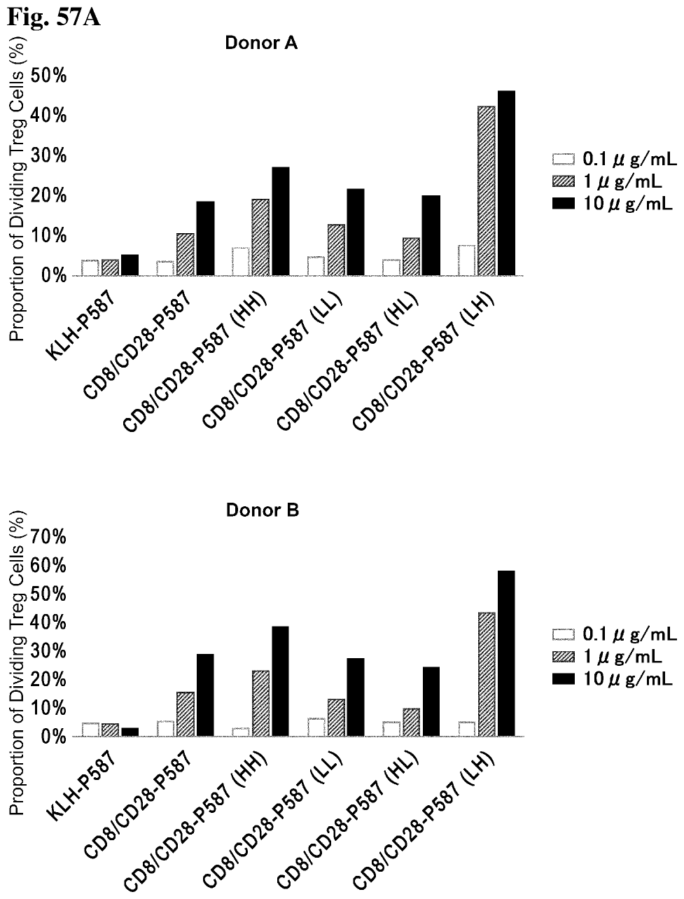

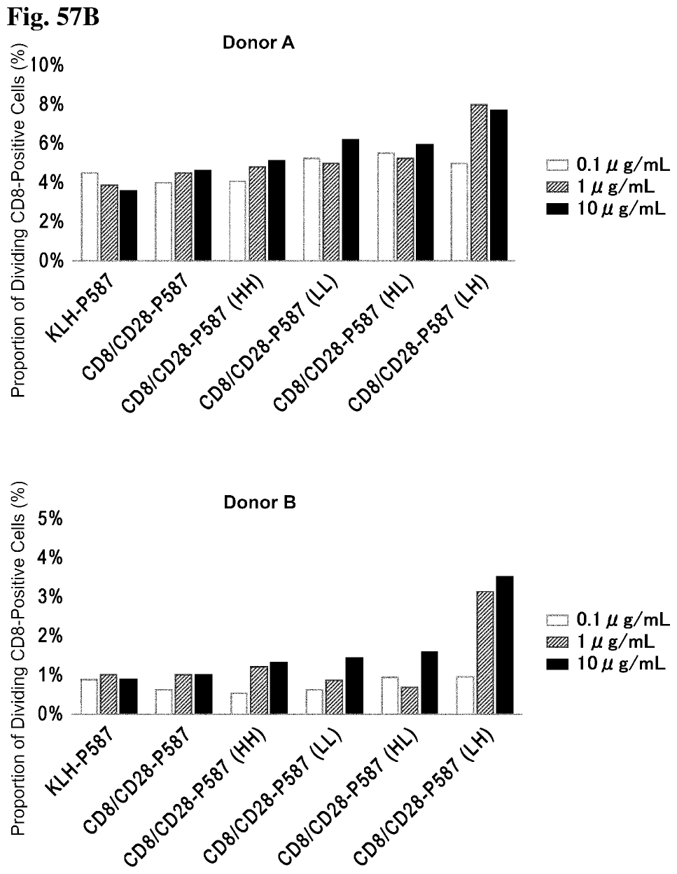

After the end of culturing, the percentage of grown cells was investigated using a flow cytometer (BD LSRFortessa™ X-20 (BD Biosciences)) (FCM). The percentage of grown cells was calculated from the percentage of reduced ViaFluor 405 fluorescence intensity. Fluorescently-labeled anti-CD8 alpha antibody, anti-CD4 antibody, anti-Foxp3 antibody, and such were used for performing an analysis with CD8 alpha positive T cells and regulatory T (Treg) cells. As a result, increase in activity was observed in some specimens as shown in FIG. 57.

### Reference Example 25 Assessment of Disulfide Bond Formation Between the Introduced Cysteines

Modified antibodies were produced by introducing cysteine into the light and heavy chains of a humanized model antibody, and the formation of disulfide bond between the newly introduced cysteines was assessed. Assessment was carried out by incubating sample antibodies in 20 mM phosphate buffer (pH 7.0) with chymotrypsin and detecting the mass of peptides presumed to be produced from the amino acid sequence of each antibody, using LC/MS. Each antibody was prepared according to Reference Examples 17 and 18. The antibodies used in this Example are shown in Table 78.

TABLE 78

| | SEQ ID NO | | SEQ ID NO | | |
|---|---|---|---|---|---|
| | (Antibody 1): | | (Antibody 2): | | |
| Antibody | Heavy | Light | Heavy | Light | Molecular |
| name | chain | chain | chain | chain | form |
| MRA-G1_LL | 1231 | 1232 | — | — | Monospecific antibody |
| MRA-G2_LL | 1233 | 1234 | — | — | Monospecific antibody |
| MRA-G4_LL | 1235 | 1236 | — | — | Monospecific antibody |
| MRA-G1T4.S191C | 1237 | 1238 | — | — | Monospecific antibody |
| MRA-G1T4.A162C | 1239 | 1240 | — | — | Monospecific antibody |

First, modified antibodies of different subclass (IgG1, IgG2, and IgG4) in which lysine at position 126 (Kabat numbering) of the light chain was substituted with cysteine were analyzed. As a result, in all of the antibodies analyzed, components that correspond to the theoretical mass of a peptide having a disulfide bond between the cysteines at position 126 were detected, as shown in Table 79. Further, this component disappeared when tris(2-carboxyethyl)phosphine, which has the reducing effect of disulfide bonds, was added to the IgG1 sample, suggesting that a disulfide bond is formed between the cysteines at position 126 in this peptide. At the same time, it was suggested that the difference in subclass does not affect this disulfide bond formation.

TABLE 79

| | | Theoretical | Measured value (Da) | | | |
|---|---|---|---|---|---|---|
| | | mass | IgG1 | | IgG2 | IgG4 |
| Peptide | Ion | (Da) | unreduced | reduced | unreduced | unreduced |
| ((IFPPSDEQLC¹²⁶SGTASVVCL)- | [M + 4H]⁴⁺ | 1460.2 | 1460.2 | n.d. | 1460.2 | 1460.2 |
| (ACEVTHQGL))2 | [M + 5H]⁵⁺ | 1168.3 | 1168.4 | n.d. | 1168.4 | 1168.3 |
| | [M + 6H]⁶⁺ | 973.8 | 973.8 | n.d. | 973.8 | 973.8 |

n.d.: not detected

Next, analysis was performed on modified antibodies in which alanine at position 162 (EU numbering), or serine at position 191 (EU numbering) of IgG1 heavy chain was substituted with cysteine. As a result, components that correspond to the theoretical mass of a peptide having a disulfide bond between the introduced cysteines were detected, as shown respectively in Tables 80 and 81. Further, this component disappeared when tris(2-carboxyethyl)phosphine was added to the sample of a modified antibody introduced with position 191 cysteine (Table 81). From the above, it was suggested that a disulfide bond is formed between cysteines also introduced into the heavy chain.

TABLE 80

| | | Theoretical | Measured |
|---|---|---|---|
| Peptide | Ion | mass (Da) | value (Da) |
| (NSGC¹⁶²L)₂ | [M + H]⁺ | 983.4 | 983.4 |
| | [M + 2H]²⁺ | 492.2 | 492.2 |

TABLE 81

| | | Theoretical | Measured value (Da) | |
|---|---|---|---|---|
| Peptide | Ion | mass (Da) | unreduced | reduced |
| SLSSVVTVPSC¹⁹¹SLGTQTY)₂ | [M + 2H]²⁺ | 1827.9 | 1827.9 | n.d. |
| | [M = 3H]³⁺ | 1218.9 | 1218.9 | n.d. |

n.d.: not detected

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### INDUSTRIAL APPLICABILITY

In a non-limiting embodiment, the antigen-binding molecule of the present disclosure is useful in that it can hold multiple antigen molecules at spatially close positions, regulate interaction between multiple antigen molecules, and/or regulate activation of multiple antigen molecules which are activated by association with each other. In other embodiments, the antigen-binding molecule of the present disclosure is useful in that it has increased resistance to protease cleavage as compared to conventional antigen-binding molecules.

### Sequence Listing C1-A1928Psq_txt

1. A method for producing an antibody preparation, said method comprising contacting an antibody solution with a reducing reagent, wherein the antibody solution comprises a first antigen-binding domain and a second antigen-binding domain which are capable of being linked with each other via at least one disulfide bond, wherein said first antigen-binding domain and said second antigen-binding domain comprises a CH1 region, a CL region, a VL region, a VH region and/or a VHH region, and wherein said at least one disulfide bond is capable of being formed between amino acid residues which are not in a hinge region.

2. The method of claim 1, wherein the produced antibody preparation comprises two antibody structural isoforms which differ by at least one disulfide bond formed between amino acid residues which are not in a hinge region.

3. The method of claim 1, wherein said method preferentially enriches or increases the population of an antibody structural isoform having at least one disulfide bond formed between amino acid residues which are not in a hinge region.

4. The method of claim 1, wherein said at least one disulfide bond is an interchain disulfide bond.

5. The method of claim 1, wherein said at least one disulfide bond is formed between a CH1 region, a CL region, a VL region, a VH region and/or a VHH region of the first antigen-binding domain and the second antigen-binding domain.

6. The method of claim 1, wherein said at least one disulfide bond is formed between a CH1 region of the first antigen-binding domain and a CH1 region of the second antigen-binding domain.

7. The method of claim 6, wherein said at least one disulfide bond is formed between the amino acid residues at position 191 according to EU numbering in the respective CH1 regions of the first antigen-binding domain and the second antigen-binding domain.

8. The method of claim 1, wherein said antibody is an IgG antibody.

9. The method of claim 1, wherein the pH of said reducing reagent is from about 3 to about 10.

10. The method of claim 1, wherein the reducing agent is selected from the group consisting of tris(2-carboxyethyl)phosphine (TCEP), 2-aminoethanethiol (2-MEA), dithiothreitol (DTT), Cysteine, glutathione (GSH) and Na₂SO₃.

11. The method of claim 1, wherein the contacting step is performed for at least 30 minutes.

12. The method of claim 1, wherein the contacting step is performed at a temperature of about 20 degrees Celsius to 37 degrees Celsius.

13. The method of claim 1, wherein the concentration of the produced antibody is from about 1 mg/ml to about 50 mg/ml.

14. The method of claim 1, wherein said first antigen-binding domain and/or said second antigen-binding domain is partially purified by affinity chromatography prior to said contacting with the reducing agent.

15. The method of claim 1, further comprising a step of removing or chemically inactivating the reducing agent.

16. The method of claim 8, wherein said antibody is an IgG1, IgG2, IgG3 or IgG4 antibody.

17. The method of claim 12, wherein the contacting step is performed at a temperature of about 23 degrees Celsius, about 25 degrees Celsius, or about 37 degrees Celsius.

18. The method of claim 15, wherein the reducing agent is removed by dialysis or a chromatography method.

19. The method of claim 1, further comprising a step of purifying the produced antibodies.

20. The method of claim 2, further comprising a step of separating the structural antibody isoforms.

21. An antibody produced according to the method of claim 1.
